(19) United States
(12) Patent Application Publication
(10) Pub. No.: US 2010/0256040 A1
(43) Pub. Date: Oct. 7, 2010

(54) COMPOSITION AND METHOD FOR TREATING VIRAL INFECTION

(75) Inventors: Scott Morham, Salt Lake City, UT (US); Kenton Zavitz, Salt Lake City, UT (US); Adrian Hobden, Salt Lake City, UT (US)

Correspondence Address: MYRIAD GENETICS INC.;INTELLECTUAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/625,200

(22) Filed: Jan. 19, 2007

Related U.S. Application Data
(63) Continuation of application No. 10/224,999, filed on Aug. 20, 2002, now abandoned.
(60) Provisional application No. 60/313,695, filed on Aug. 20, 2001.

(51) Int. Cl. A61K 38/16; C07K 7/06; C07K 7/08; C07K 14/00; C07K 14/005; C07H 21/04; C12N 5/10; A61K 38/08; A61K 38/10; A61P 31/18

(52) U.S. Cl. 514/1.2; 530/328; 530/327; 530/326; 530/325; 530/324; 536/23.1; 435/325; 536/23.72

(57) ABSTRACT

Methods for inhibiting virus propagation and treating virus infection are provided which include administering to cells infected with viruses a compound capable of inhibiting viral budding from the cells.

### RELATED U.S. APPLICATIONS

This application is a continuation of U.S. patent application Ser. No. 10/224,999 filed on Aug. 20, 2002; which claims the benefit under 35 U.S.C. §119(e) to U.S. Provisional Application Ser. No. 60/313,695 filed on Aug. 20, 2001, which is incorporated herein by reference in its entirety.

### SEQUENCE LISTING

The instant application was filed with a formal Sequence Listing submitted electronically as a text file. This text file, which was named “5004.01 2006-05-19 SEQ LIST (TXT) BGJ.ST25.txt”, was created on May 19, 2006, and is 918,031 bytes in size. Its contents are incorporated by reference herein in their entirety.

### FIELD OF THE INVENTION

The present invention generally relates to pharmaceuticals and methods of treating diseases, particularly to methods and pharmaceutical compositions for treating viral infections.

### BACKGROUND OF THE INVENTION

Viruses are the smallest of parasites, and are completely dependent on the cells they infect for their reproduction. Viruses are composed of an outer coat of protein, which is sometimes surrounded by a lipid envelope, and an inner nucleic acid core consisting of either RNA or DNA. Generally, after docking with the plasma membrane of a susceptible cell, the viral core penetrates the cell membrane to initiate the viral infection. After infecting cells, viruses commandeer the cell's molecular machinery to direct their own replication and packaging. The “replicative phase” of the viral lifecycle may begin immediately upon entry into the cell, or may occur after a period of dormancy or latency. After the infected cell synthesizes sufficient amounts of viral components, the “packaging phase” of the viral life cycle begins and new viral particles are assembled. Some viruses reproduce without killing their host cells, and many of these bud from host cell membranes. Other viruses cause their host cells to lyse or burst, releasing the newly assembled viral particles into the surrounding environment, where they can begin the next round of their infectious cycle.

Several hundred different types of viruses are known to infect humans, however, since many of these have only recently been recognized, their clinical significance is not fully understood. Of these viruses that infect humans, many infect their hosts without producing overt symptoms, while others (e.g., influenza) produce a well-characterized set of symptoms. Importantly, although symptoms can vary with the virulence of the infecting strain, identical viral strains can have drastically different effects depending upon the health and immune response of the host. Despite remarkable achievements in the development of vaccines for certain viral infections (i.e., polio and measles), and the eradication of specific viruses from the human population (e.g., smallpox), viral diseases remain as important medical and public health problems. Indeed, viruses are responsible for several “emerging” (or reemerging) diseases (e.g., West Nile encephalitis & Dengue fever), and also for the largest pandemic in the history of mankind (HIV and AIDS).

Viruses that primarily infect humans are spread mainly via respiratory and enteric excretions. These viruses are found worldwide, but their spread is limited by inborn resistance, prior immunizing infections or vaccines, sanitary and other public health control measures, and prophylactic antiviral drugs. Zoonotic viruses pursue their biologic cycles chiefly in animals, and humans are secondary or accidental hosts. These viruses are limited to areas and environments able to support their nonhuman natural cycles of infection (vertebrates or arthropods or both). However, with increased global travel by humans, and the likely accidental co-transport of arthropod vectors bearing viral payloads, many zoonotic viruses are appearing in new areas and environments as emerging diseases. For example, West Nile virus, which is spread by the bite of an infected mosquito, and can infect people, horses, many types of birds, and other animals, was first isolated from a febrile adult woman in the West Nile District of Uganda in 1937. The virus made its first appearance in the Western Hemisphere, in the New York City area in the autumn of 1999, and during its first year in North America, caused the deaths of 7 people and the hospitalization of 62. At the time of this writing (August, 2002) the virus has been detected in birds in 37 states and the District of Columbia, and confirmed human infections have occurred in Alabama, the District of Columbia, Florida, Illinois, Indiana, Louisiana, Massachusetts, Mississippi, Missouri, New York City, Ohio, and Texas. (See: http://www.cdc.gov/od/oc/media/wncount.htm).

Additionally, some viruses are known to have oncogenic properties. Human T-cell lymphotropic virus type 1 (a retrovirus) is associated with human leukemia and lymphoma. Epstein-Barr virus has been associated with malignancies such as nasopharyngeal carcinoma, Burkitt's lymphoma, Hodgkin's disease, and lymphomas in immunosuppressed organ transplant recipients. Kaposi's sarcoma-associated virus is associated with Kaposi's sarcoma, primary effusion lymphomas, and Castleman's disease (a lymphoproliferative disorder).

Treatment of viral diseases presents unique challenges to modern medicine. Since viruses depend on host cells to provide many functions necessary for their multiplication, it is difficult to inhibit viral replication without at the same time affecting the host cell itself. Consequently, antiviral treatments are often directed at the functions of specific enzymes of particular viruses. However, such antiviral treatments that specifically target viral enzymes (e.g., HIV protease, or HIV reverse transcriptase) often have limited usefulness, because resistant strains of viruses readily arise through genetic drift and mutation.

### SUMMARY OF THE INVENTION

The present invention provides a method for inhibiting viral budding from virus-infected cells and thus inhibiting viral propagation in the cells. The method can be useful in treating infection by viruses that utilize the Tsg101 protein of their host cells for viral budding within and/or out of the cells. In general, the method comprises administering to a patient in need of such treatment a composition comprising a peptide having an amino acid sequence motif PX₁X₂P and is capable of binding the UEV domain of Tsg101, wherein X₁ and X₂ are amino acids, and X₂ is not R. Preferably, X₁ is threonine (T) or serine (S), and X₂ is alanine (A). Preferably the peptide is associated with a transporter that is capable of increasing the uptake of the peptide by a mammalian cell by at least 100%, preferably at least 300%.

Thus, the method can be used in treating infection by viruses such as HIV, Ebola virus, HBV, HSV1, HSV2, HSV5, EBV, Influenza A virus, HPV, HTLV-2, West Nile virus, Measles virus, Rubella virus, Colorado tick fever virus, foot-and-mouth disease virus, human foamy virus, hepatitis E virus, hepatitis G virus, human parechovirus 2, and Semliki forest virus. In a preferred embodiment, the method is used in treating HIV infection and AIDS, and/or preventing AIDS. When the method is used in treating HIV infection, preferably the peptide does not contain a contiguous amino acid sequence of an HIV GAG protein that is sufficient to impart an ability to bind the UEV domain of Tsg101 on said peptide.

In preferred embodiments, the peptide in the composition is covalently linked to the transporter. Advantageously, the transporter is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, fibroblast growth factor and fragments thereof, Galparan and fragments thereof, and HSV-1 structural protein VP22 and fragments thereof, and peptoid analogs thereof. Preferably, the transporter is a peptide having at least six contiguous amino acid residues that are L-arginine, D-arginine, L-lysine, D-lysine, L-histidine, D-histidine, L-ornithine, D-ornithine, or a combination thereof. Alternatively, the transporter can be non-peptidic molecules or structures such as liposomes, dendrimers, and siderophores.

In specific embodiments, the peptide in the composition includes a contiguous amino acid sequence of from 8 to about 100 residues, preferably from 8 to about 50 residues, more preferably from 9 to about 20 residues, of a viral protein selected from the group consisting of HIV GAG, Ebola virus Matrix (EbVp40) protein, HBV PreS1/PreS2/S envelope protein, HSV1 RL2 protein, HSV2 virion glycoprotein K, HSV2 Strain 333 glycoprotein I, EBV nuclear protein EBNA2, Influenza A virus hemagglutinin, HPV L1 proteins, HPV L2 proteins, HPV late proteins, HTLV-2 GAG protein, West Nile virus polyprotein precursor, Measles virus matrix protein, Rubella virus non-structural protein, Colorado tick fever virus VP12, foot-and-mouth disease virus VP1 capsid protein, human foamy virus GAG protein, hepatitis E virus ORF-3 protein, hepatitis G virus polyprotein precursor, HSV5 UL32 protein, human parechovirus 2 polyprotein, and Semliki forest virus polyprotein, and wherein the contiguous amino acid sequence encompasses the P(T/S)AP motif of the viral protein. For example, the peptide used in the composition can include an amino acid sequence selected from the group consisting of SEQ ID NOs:1-37, SEQ ID NOs:38-125, SEQ ID NOs:126-268, SEQ ID NOs:269-554, SEQ ID NOs:555-697, SEQ ID NOs:698-749, SEQ ID NOs:750-892, SEQ ID NOs:893-1035, SEQ ID NOs:1036-1178, SEQ ID NOs:1179-1321, SEQ ID NOs:1322-1464, SEQ ID NOs:1465-1607, SEQ ID NOs:1608-1750, SEQ ID NOs:1751-1893, SEQ ID NOs:1894-2036, SEQ ID NOs:2037-2179, SEQ ID NOs:2180-2322, SEQ ID NOs:2323-2459, SEQ ID NOs:2460-2602, SEQ ID NOs:2603-2745, SEQ ID NOs:2746-2887, SEQ ID NOs:2888-3030, SEQ ID NOs:3031-3173, SEQ ID NOs:3174-3316, and SEQ ID NOs:3317-3459.

In preferred embodiments, the transporter in the composition according to the method of the present invention is capable of increasing the uptake of said peptide by a mammalian cell by at least 100%, preferably at least 300%.

When the transporter used in the method of the present invention is a peptide, a hybrid polypeptide or fusion polypeptide is provided. The hybrid polypeptide includes (a) a first portion having an amino acid sequence motif PX₁X₂P capable of binding the UEV domain of Tsg101, wherein X₁ and X₂ are amino acids, and X₂ is not R, and (b) a second portion which is a peptidic transporter capable of increasing the uptake of the first portion by human cells. Advantageously, the transporter is capable of increasing the uptake of said peptide by a mammalian cell by at least 100%, preferably at least 300%. Preferably, the first portion consists of from 8 to 100, more preferably 8 to 50, even more preferably 9 to 20 amino acid residues. The hybrid polypeptide can be chemically synthesized or produced by recombinant expression. Thus, the present invention also provides isolated nucleic acids encoding the hybrid polypeptides, and host cells recombinantly expressing the hybrid polypeptides.

The peptide of the present invention can be administered to a patient in the presence or absence of a transporter. The peptide with or without a transporter can be administered directly to a patient in a pharmaceutical composition. Alternatively, the peptide or hybrid polypeptide according to the present invention can be introduced into a patient indirectly by administering to the patient a nucleic acid encoding the peptide or hybrid polypeptide.

Various modifications may be made to improve the stability and solubility of the peptides or hybrid polypeptides, and/or optimize its binding affinity to the UEV domain of Tsg101. In particular, various protection groups can be incorporated into the amino acid residues of the peptides or hybrid polypeptides. In addition, the compounds according to the present invention can also be in various pharmaceutically acceptable salt forms.

In another aspect of the present invention, methods of combination therapy for treating or preventing HIV and/or AIDS, and other viral infection are provided. In such methods, both a compound of the present invention (in the presence or absence of a transporter) and one or more other antiviral compounds are administered to a patient in need of treatment. Such other antiviral compounds should be pharmaceutically compatible with the compound of the present invention. Compounds suitable for use in combination therapies with the Tsg101-binding compounds according to the present invention include, but are not limited to, any small molecule drugs, antibodies, immunomodulators, and vaccines.

In accordance with another aspect of the present invention, isolated peptides are provided consisting of a contiguous amino acid sequence of from 8 to about 30 amino acid residues of a viral protein selected from the group consisting of HBV PreS1/PreS2/S envelope protein, HSV1 RL2 protein, HSV2 virion glycoprotein K, HSV2 Strain 333 glycoprotein I, EBV nuclear protein EBNA2, Influenza A virus hemagglutinin, HPV L1 proteins, HPV L2 proteins, HPV late proteins, HTLV-2 GAG protein, West Nile virus polyprotein precursor, Measles virus matrix protein, Rubella virus non-structural protein, Colorado tick fever virus VP12, foot-and-mouth disease virus VP1 capsid protein, human foamy virus GAG protein, hepatitis G virus polyprotein precursor, human parechovirus 2 polyprotein, and Semliki forest virus polyprotein, wherein the contiguous amino acid sequence encompasses the P(T/S)AP motif of the viral protein, and wherein the peptide is capable of binding the UEV domain of Tsg101. Preferably, the peptide does not contain a contiguous amino acid sequence of an HIV GAG protein or Ebola virus Matrix (EbVp40) protein that is sufficient to impart an ability to bind the UEV domain of Tsg101 on the peptide. In addition, the present invention also provides isolated nucleic acids encoding the isolated peptides.

In preferred embodiments, the isolated peptide consists of from 9 to about 20 amino acid residues. For example, such isolated peptides may include an amino acid sequence selected from the group consisting of SEQ ID NOs:38-125, SEQ ID NOs:126-268, SEQ ID NOs:269-554, SEQ ID NOs:555-697, SEQ ID NOs:698-749, SEQ ID NOs:750-892, SEQ ID NOs:893-1035, SEQ ID NOs:1036-1178, SEQ ID NOs:1179-1321, SEQ ID NOs:1322-1464, SEQ ID NOs:1465-1607, SEQ ID NOs:1608-1750, SEQ ID NOs:1751-1893, SEQ ID NOs:1894-2036, SEQ ID NOs:2037-2179, SEQ ID NOs:2180-2322, SEQ ID NOs:2323-2459, SEQ ID NOs:2460-2602, SEQ ID NOs:2603-2745, SEQ ID NOs:2888-3030, SEQ ID NOs:3174-3316, and SEQ ID NOs:3317-3459.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a competitive inhibition curve showing that the p(1-14) peptide having the first 14 amino acid residues is capable of inhibiting protein-protein interaction between GST-p6 and myc-Tsg101(1-207);

FIG. 2 is a Dixon plot showing p6(1-14) inhibition of the interaction between GST-p6 and myc-Tsg101(1-207);

FIG. 3 is another Dixon plot showing p6(1-14) inhibition of the interaction between GST-p6 and myc-Tsg101(1-207);

FIG. 4 is the graphical test results showing the effect of the compound MPI-PEP1 at various concentrations on HIV viral propagation in cell culture and on cell viability in the cell culture;

FIG. 5 is the graphical test results of the compound MPI-PEP2;

FIG. 6 is the graphical test results of the compound MPI-PEP3; and

FIG. 7 is the graphical test results of AZT as a positive control compound.

### DETAILED DESCRIPTION OF THE INVENTION

As used herein, the term “viral infection” generally encompasses infection of an animal host, particularly a human host, by one or more viruses. Thus, treating viral infection will encompass the treatment of a person who is a carrier of one or more specific viruses or a person who is diagnosed of active symptoms caused by and/or associated with infection by the viruses. A carrier of virus may be identified by any methods known in the art. For example, a person can be identified as virus carrier on the basis that the person is antiviral antibody positive, or is virus-positive, or has symptoms of viral infection. That is, “treating viral infection” should be understood as treating a patient who is at any one of the several stages of viral infection progression. In addition, “treating or preventing viral infection” will also encompass treating suspected infection by a particular virus after suspected past exposure to virus by e.g., blood transfusion, exchange of body fluids, bites, accidental needle stick, or exposure to patient blood during surgery, or other contacts with a person with viral infection that may result in transmission of the virus.

Specifically, as used herein, the term “HIV infection” generally encompasses infection of a host animal, particularly a human host, by the human immunodeficiency virus (HIV) family of retroviruses including, but not limited to, HIV I, HIV II, HIV III (a.k.a. HTLV-III, LAV-1, LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD⁴⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., contact with HIV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HIV infection” may also encompass treating a person who has not been diagnosed as having HIV infection but is believed to be at risk of infection by HIV.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, etc. Modified forms also encompass pharmaceutically acceptable salt forms. In addition, modifications also include intra-molecular crosslinking and covalent attachment to various moieties such as lipids, flavin, biotin, polyethylene glycol or derivatives thereof, etc. In addition, modifications may also include cyclization, and branching. Further, amino acids other than the conventional twenty amino acids encoded by genes may also be included in a polypeptide.

As used herein, the term “Tsg101” means human Tsg101 protein, unless otherwise specified.

As disclosed in commonly assigned co-pending applications, mature HIV-1_{NYU/BR5} p6 (gag polyprotein amino acids 449-500) was used as a bait in a yeast two-hybrid system to screen a prey library derived from human spleen cDNA. A gene encoding the tumor suppressor TSG 101 protein (Tsg101; aa 7-390) was isolated as an interactor. The p6 bait used here contains a late domain motif (-PTAP-).

In addition, different p6 point mutants (E6G, P7L, A9R, or P10L) were generated and tested for their ability to bind Tsg101 protein. While the wild-type p6 peptide and the E6G p6 mutant were capable of binding Tsg101 protein, each of the P7L, A9R, and P10L point mutations abolishes the p6 binding affinity to Tsg101. The P7L, A9R, and P10L point mutations alter the PTAP motif in p6 peptide. The same mutations in the PTAP motif of the HIV p6 gag protein prevent HIV particles from budding from the host cells. See Huang et al., J. Virol., 69:6810-6818 (1995).

As is known in the art, the P(T/S)AP motif is conserved among the p6^{gag} domains of all known primate lentiviruses. In nonprimate lentiviruses, which lack a p6^{gag} domain, the P(T/S)AP motif is at the immediate C terminus of the Gag polyprotein. It has been shown that the P(T/S)AP motif is required for efficient pinching off of the lentivirus bud from the host cell surface. It is critical for lentivirus' particularly HIV virus' particle production. See Huang et al., J. Virol., 69:6810-6818 (1995). Specifically, deletion of the motif (PTAP⁻) results in drastic reduction of lentiviral particle production. In addition, the PTAP-deficient HIV proceeded through the typical stages of morphogenesis but failed to complete the process. Rather, they remain tethered to the plasma membrane and thus rendered non-infectious. That is, the lentiviral budding process is stalled. See Huang et al., J. Virol., 69:6810-6818 (1995).

Also as disclosed in commonly assigned co-pending applications, it has been found that Tsg101 binds directly to the P(T/S)AP domain of HIV-1 p6. The Tsg101 prey fragment isolated in yeast two-hybrid assay contains the ubiquitin E2 variant (UEV) domain indicating that the UEV domain is involved in the binding to the P(T/S)AP domain. This is consistent with the fact that ubiquitin is required from retrovirus budding and that proteasome inhibition reduces the level of free ubiquitin in HIV-1-infected cells and interferes with the release and maturation of HIV-1 and HIV-2. See Patnaik et al., Proc. Natl. Acad. Sci. USA, 97(24):13069-74 (2000); Schubert et al., Proc. Natl. Acad. Sci. USA, 97(24):13057-62 (2000); Strack et al., Proc. Natl. Acad. Sci. USA, 97(24):13063-8 (2000).

Tsg101 plays an important role in vacuolar protein sorting (Vps). The Vps pathway sorts membrane-bound proteins for eventual degradation in the lysosome (vacuole in yeast). See Lemmon and Traub, Curr. Opin. Cell. Biol., 12:457-66 (2000). Two alternative entrees into the Vps pathway are via vesicular trafficking from the Golgi (e.g., in degrading misfolded membrane proteins) or via endocytosis from the plasma membrane (e.g., in downregulating surface proteins like epidermal growth factor receptor (EGFR)). Vesicles carrying proteins from either source can enter the Vps pathway by fusing with endosomes. As these endosomes mature, their cargos are sorted for lysosomal degradation via the formation of structures called multivesicular bodies (MVB). MVB are created when surface patches on late endosomes bud into the compartment, forming small (˜50-100 nm) vesicles. A maturing MVB can contain tens or even hundreds of these vesicles. The MVB then fuses with the lysosome, releasing the vesicles for degradation in this hydrolytic organelle. Tsg101 appears to perform important roles in the Vps pathway. For example, deletion of the yeast Tsg101 ortholog (Vps23/Stp22) gives rise to a class E Vps phenotype, blocks vacuolar protein sorting from the golgi, and inhibits surface receptor downregulation. See Babst et al, Traffic, 1:248-258 (2000); Li et al., Mol. Cell Biol., 19:3588-3599 (1999). Mammalian Tsg101 similarly participates in endosomal trafficking. For example, efficient down-regulation of activated EGFR requires Tsg101 function. See Babst et al, Traffic, 1:248-258 (2000); Bishop and Woodman, J. Biol. Chem., 276:11735 (2001).

It is known that short chains of Ub (1-3 molecules) can “mark” surface receptors for endocytosis and degradation in the lysosome. Hicke, Trends Cell Biol., 9:107-112 (1999); Rotin et al., J. Membr. Biol., 176:1-17 (2000). There is also growing evidence that Ub conjugation (and hydrolysis) plays important roles in targeting proteins into the Vps pathway. See Dupre and Haguenauer-Tsapis, Mol. Cell Biol., 12:421-435 (2001); Losko et al., Mol. Cell Biol., 12:1047-1059 (2001). Several classes of proteins that carry the P(T/S)AP motif are surface receptors known to be degraded via the Vps pathway or function in the Vps pathway. Such proteins include connexins 43 and 45, hepatocyte growth factor-regulated tyrosine kinase substrate (Hrs, a homolog of yeast Vps27p), and secretory carrier membrane protein-3 (Scamp-3). See Fan et al., Biochem. J., 345(3):503-509 (2000); Staub and Rotin., Structure, 4:495-499 (1996); Chin et al., J. Biol. Chem., 276:7069-78 (2001); Komada and Kitamura, Biochem. Biophys. Res. Commun., 281:1065-9 (2001). A plausible role for Tsg101 in this process is to recognize ubiquitinated proteins that carry P(T/S)AP motifs and help coordinate their incorporation into vesicles that bud into the MVB.

Interestingly, it has been noted that the topologies of viral budding and multivesicular body (MVB) formation are similar. In particular, both processes involve the membrane invaginating away from (rather than into) the cytoplasm. Indeed, these two processes are the only known examples in which cell buds a vesicle out of the cytoplasm, suggesting that viral budding and MVB formation may employ analogous mechanisms.

In addition, the recruitment of cellular machinery to facilitate virus budding appears to be a general phenomenon, and distinct late domains have been identified in the structural proteins of several other enveloped viruses. See Vogt, Proc. Natl. Acad. Sci. USA, 97:12945-12947 (2000). Two well characterized late domains are the “PY” motif (consensus sequence: PPXY; X=any amino acid) found in membrane-associated proteins from certain enveloped viruses. See Craven et al., J. Virol., 73:3359-3365 (1999); Harty et al., Proc. Natl. Acad. Sci. USA, 97:13871-13876 (2000); Harty et al., J. Virol., 73:2921-2929 (1999); and Jayakar et al., J. Virol., 74:9818-9827 (2000). The cellular target for the PY motif is Nedd4 which also contains a Hect ubiquitin E3 ligase domain. The “YL” motif (YXXL) was found in the Gag protein of equine infectious anemia virus (EIAV). Puffer et al., J. Virol., 71:6541-6546 (1997); Puffer et al., J. Virol., 72:10218-10221 (1998). The cellular receptor for the “YL” motif appears to be the AP-50 subunit of AP-2. Puffer et al., J. Virol., 72:10218-10221 (1998). Interestingly, the late domains such as the P(T/S)AP motif, PY motif and the YL motif can still function when moved to different positions within retroviral Gag proteins, which suggests that they are docking sites for cellular factors rather than structural elements. Parent et al., J. Virol., 69:5455-5460 (1995); Yuan et al., EMBO J., 18:4700-4710 (2000). Moreover, the late domains such as the P(T/S)AP motif, PY motif and the YL motif can function interchangeably. That is one late domain motif can be used in place of another late domain motif without affecting viral budding. Parent et al., J. Virol., 69:5455-5460 (1995); Yuan et al., EMBO J., 18:4700-4710 (2000); Strack et al., Proc. Natl. Acad. Sci. USA, 97:13063-13068 (2000).

Accordingly, while not wishing to be bound by any theory, it is believed that although the three late domain motifs bind to different cellular targets, they utilize common cellular pathways to effect viral budding. In particular, it is believed that the different cellular receptors for viral late domain motifs feed into common downstream steps of the vacuolar protein sorting (VPS) and MVB pathway. As discussed above, Tsg101 functions in the VPS pathway. Another protein, Vps4 functions in Tsg101 cycling and endosomal trafficking Particularly, Vps4 mutants prevent normal Tsg101 trafficking and induce formation of aberrant, highly vacuolated endosomes that are defective in the sorting and recycling of endocytosed substrates. See Babst et al, Traffic, 1:248-258 (2000); Bishop and Woodman, J. Biol. Chem., 276:11735 (2001).

While not wishing to be bound by any theory, it is believed that the binding of the P(T/S)AP motif in viral proteins to Tsg101 enables viruses having the P(T/S)AP motif to usurp cellular machinery normally used for MVB formation to allow viral budding from the plasma membrane. It is also believed that Tsg101 serves as the common docking site for all viruses that utilize the P(T/S)AP motif to bud off host cell cytoplasm membrane. In addition, depletion of Tsg101 or interference with the interaction between TSg101 and the P(T/S)AP motif in virus-infected cells would prevent viral budding from the cells. Moreover, an examination of HIV-1 amino acid sequence variants in GenBank using BLAST (Basic Local Alignment Search Tool) identified a number of HIV strains with the standard P(T/S)AP motif being replaced with variations of the P(T/S)AP motif, indicating that such variations may also enable viral budding and that peptides with such variations may also bind Tsg101. Such identified variations include PIAP (SEQ ID NO:3) (see Zhang et al., J. Virol., 71:6662-6670 (1997); Farrar et al., J. Med. Virol., 34:104-113 (1991)), and PTTP (SEQ ID NO:4) (see Zhang et al., J. Virol., 71:6662-6670 (1997).

In accordance with the present invention, a number of proteins of non-HIV viruses have been found to also contain the P(T/S)AP motif. The proteins are summarized in Table 1 below. The amino acid sequences of such proteins are provided under SEQ ID NOs:3460-3484.

TABLE 1
Viral Proteins Containing the P(T/S)AP Motif

| | P(T/S)AP-Containing | GenBank Accession |
|---|---|---|
| Virus | Protein | No. |
| Ebola Virus | Matrix Protein | AAL25816 |
| HIV | GAG | AF324493 |
| Hepatitis B Virus | PreS1/PreS2/S Envelope | BAA85340 |
| Human Herpesvirus 1 | RL2 | NP_044601 |
| Human Herpesvirus 2 | Virion Glycoprotein K | NP_044524 |
| Human Herpesvirus 2 | Glycoprotein I | P06764 |
| Strain 333 | | |
| Human Herpesvirus | BYRF1, Encodes EBNA-2 | NP_039845 |
| 4/Epstein Barr Virus | | |
| Influenza A Virus | Hemagglutinin | AAG38554 |
| (A/Pintail | | |
| Duck/Alberta/114/79 | | |
| (H8N4)) | | |
| Human Papillomavirus | L1 Protein, My09/My11 | AAA67231 |
| | Region | |
| Human Papillomavirus | Minor Capsid Protein L2 | NP_043365 |
| Type 23 | | |
| Human Papillomavirus | Major Capsid Protein L1 | P27232 |
| Type 35 | | |
| Human Papillomavirus | Minor Capsid Protein L2 | NP_040303 |
| Type 6b | | |
| Human Papillomavirus | Late Protein | NP_041865 |
| Type 9 | | |
| Human T-Cell | Gag Protein | CAA61543 |
| Lymphotropic Virus | | |
| Type 2 | | |
| West Nile Virus | Polyprotein Precursor | NP_041724 |
| Measles Virus | Matrix Protein | CAA34587 |
| Rubella Virus | Non-Structural Protein | BAB32473 |
| Colorado Tick Fever | VP12 | AAB02025 |
| Virus | | |
| Foot-and-Mouth | VP1 Capsid Protein | AAA42637 |
| Disease Virus | | |
| Human Foamy Virus | Gag | NP_044279 |
| Hepatitis E Virus | ORF-3 | AAC35758 |
| Hepatitis G Virus | Polyprotein Precursor | AAB65834 |
| Human Herpesvirus 5 | UL32 | AAG31644 |
| Human Parechovirus 2 | Polyprotein | NP_046804 |
| Semliki Forest Virus | Polyprotein | CAA76683 |

Thus, the inventors of the present invention propose to employ peptides derived from such viral proteins to treat viral infection including HIV infection as well as infection by other viruses listed in the above Table 1.

In accordance with a first aspect of the present invention, a method is provided for inhibiting virus budding from virus-infected cells and thus inhibiting viral propagation in the cells. The method includes administering to the cells a compound comprising an amino acid sequence motif of PX₁X₂P and capable of binding the UEV domain of Tsg101, wherein X₁ is any amino acid or amino acid analog and X₂ is an amino acid or amino acid analog other than arginine (R). The compounds can be administered to cells in vitro or cells in vivo in a human or animal body. In the case of in vivo applications of the method, viral infection can be treated and alleviated by using the compound to inhibit viral propagation.

Preferably, the method is used for inhibiting viral budding of a virus that utilizes the Tsg101 protein of their host cells for viral budding within and/or out of the cells. The method is therefore useful in inhibiting viral propagation. In one embodiment, the method is used for inhibiting viral budding by an animal virus selected from the group consisting of HIV, hepatitis B virus, hepatitis E virus, hepatitis G virus, human papillomavirus, human herpes virus 1 (HSV1), human herpes virus 1 (HSV2), human herpes virus 5 (HSV5), Measles virus, Rubella virus, West Nile virus, human foamy virus, human parechovirus, Colorado tick fever virus, human T-cell lymphotropic virus, influenza A virus, foot-and-mouth disease virus, Ebola virus, and Semliki Forest virus.

In a preferred embodiments, the method is applied to inhibit viral budding by HIV, hepatitis B virus, HSV1 and HSV2. By inhibiting viral propagation in cells in a patient, the viral load in the patient body can be prevented from increasing and can even be decreased. Accordingly, the method of the present invention can also be used in treating viral infection as well as symptoms caused by and/or associated with the viral infection. In addition, when applied at an early stage before a patient develops a full-blown disease caused by viral infection, the method can be used to prevent such a disease by inhibiting viral propagation and decreasing the viral load in the patient. For example, human hepatitis B virus is known to cause hepatitis which may increase the risk of liver cancer. Thus, if the compounds of the present invention is applied to a patient at an early stage of the hepatitis B viral infection before the full development of hepatitis, hepatitis may be prevented and the likelihood of liver cancer in the patient may be reduced. Similarly, human papillomaviruses are believed to cause cervical cancer. Thus, by treating human papillomavirus infection, the risk of cervical cancer can be reduced.

The compound which comprises the amino acid sequence motif PX₁X₂P and is capable of binding the UEV domain of Tsg101 can be of any type of chemical compounds so long as the compound is capable of binding the UEV domain of Tsg101. In the case of viruses such foot-and-mouth disease virus which infects animals such as canine and cattles, the compounds to be administered to the animals should be capable of binding the Tsg101 orthologs in the animals. For example, the compound can be a peptide, a modified peptide, an oligonucleotide-peptide hybrid (e.g., PNA), etc. In a preferred embodiment, the compound administered is capable of binding the UEV domain of human Tsg101.

In one embodiment, in the compound comprising an amino acid sequence motif PX₁X₂P and capable of binding the UEV domain of Tsg101, X₁ is selected from the group consisting of threonine (T), serine (S), and isoleucine (I) and analogs thereof, and X₂ is not R. In another embodiment, the X₂ in the motif is alanine (A) or threonine (T) or an analog thereof. In a more preferred embodiment, the compound administered has the amino acid sequence motif of PX₁X₂P, wherein X₁ is selected from the group consisting of T, S, and I and analogs thereof, and X₂ is A or T or an analog thereof.

Thus, the compound can be a tetrapeptide having an amino acid sequence of PX₁X₂P, wherein X₂ is an amino acid or an amino acid analog other than arginine. In one embodiment, the tetrapeptide has an amino acid sequence of P(T/S/I)(A/T)P (SEQ ID NOs:1-6). In a preferred embodiment, the tetrapeptide has the sequence of PTAP (SEQ ID NO:1). In another preferred embodiment, the tetrapeptide has the sequence of PSAP (SEQ ID NO. 2).

The compound can also include a longer peptide comprising the amino acid sequence motif of PX₁X₂P and capable of binding the UEV domain of Tsg101. Advantageously, the compound is a peptide that contains an amino acid sequence of less than about 400, 375, 350, 325, 300, 275, 250, 225 or 200 residues. Preferably, the peptide contains an amino acid sequence of less than about 175, 150, 125, 115, 100, 95, 90, 85, 80, 75, 70, 65, 60 or 55 residues. More preferably, the peptide contains an amino acid sequence of less than about 50, 48, 45, 42, 40, 38, 35, 33, 32, 31, 30, 29, 28, 27, 26, 25, 24, 23, 22, 21 or 20 residues. In preferred embodiments, the peptide contains an amino acid sequence of from about 4 to about 200, 6 to about 150, 8 to about 100, preferably from about 8 to about 50, more preferably from about 9 to about 50, from about 9 to 45, 9 to 40, 9 to 37, 9 to 35, 9 to 30, 9 to 25 residues. More advantageously, the peptide contains an amino acid sequence of from 9 to about 10, 11, 12, 13, 14, 15, 16, 17, 18, 19, 20, 21, 22, 23 or 24 residues, even more advantageously, from 10 to about 11, 12, 13, 14, 15, 16, 17, 18, 19, 20, 21, 22, 23 or 24 residues. Preferably, the PX₁X₂P motif in the sequence is the P(T/S)AP motif.

In a preferred embodiment, the compound includes a peptide that contains a contiguous amino acid sequence of an HIV GAG protein and is capable of binding the UEV domain of Tsg101. The contiguous amino acid sequence encompasses the late domain motif of the GAG protein, which can be the P(T/S/I)(A/T)P motif or a variant thereof.

In specific embodiments, the compound includes an amino acid sequence selected from the group of EPTAP (SEQ ID NO:7), EPSAP (SEQ ID NO:8), PTAPP (SEQ ID NO:9), PSAPP (SEQ ID NO:10), EPTAPP (SEQ ID NO:11), EPSAPP (SEQ ID NO:12), PEPTAP(SEQ ID NO:13), PEPSAP (SEQ ID NO:14), RPEPTAP (SEQ ID NO:15), RPEPSAP (SEQ ID NO:16), PEPTAPP (SEQ ID NO:17), PEPSAPP (SEQ ID NO:18), EPTAPPEE (SEQ ID NO:19), EPSAPPEE (SEQ ID NO:20), EPTAPPAE (SEQ ID NO:21), PEPTAPPEE (SEQ ID NO:22), PEPTAPPAE (SEQ ID NO:23), PEPSAPPEE (SEQ ID NO:24), PGPTAPPEE (SEQ ID NO:25), PGPTAPPAE (SEQ ID NO:26), PGPSAPPEE (SEQ ID NO:27), RPEPTAPPEE (SEQ ID NO:28), RPEPSAPPEE (SEQ ID NO:29), RPEPTAPPAE (SEQ ID NO:30), RPEPSAPPAE (SEQ ID NO:31), RPGPTAPPEE (SEQ ID NO:32), RPGPSAPPEE (SEQ ID NO:33), RPGPTAPPAE (SEQ ID NO:34), RPGPSAPPAE (SEQ ID NO:35) LQSRPEPTAPPEE (SEQ ID NO:36), LQSRPEPSAPPEE (SEQ ID NO:37).

In another embodiment, the compound includes a contiguous amino acid sequence of a viral protein selected from the group consisting of Ebola virus Matrix (EbVp40) protein, HBV PreS1/PreS2/S envelope protein, HSV1 RL2 protein, HSV2 virion glycoprotein K, HSV2 Strain 333 glycoprotein I, EBV nuclear protein EBNA2, Influenza A virus hemagglutinin, HPV L1 proteins, HPV L2 proteins, HPV late proteins, HTLV-2 GAG protein, West Nile virus polyprotein precursor, Measles virus matrix protein, Rubella virus non-structural protein, Colorado tick fever virus VP12, foot-and-mouth disease virus VP1 capsid protein, human foamy virus GAG protein, hepatitis E virus ORF-3 protein, hepatitis G virus polyprotein precursor, HSV5 UL32 protein, human parechovirus 2 polyprotein, and Semliki forest virus polyprotein, and wherein the contiguous amino acid sequence encompasses the P(T/S)AP motif of the viral protein.

In a specific embodiment, the compound includes a contiguous amino acid sequence of Ebola virus Matrix (EbVp40) protein that encompasses the P(T/S)AP motif of the protein.

Advantageously, the compound is a peptide that contains a contiguous amino acid sequence of less than about 400, 375, 350, 325, 300, 275, 250, 225 or 200 residues of one of the viral proteins in Table 1, which encompasses the P(T/S)AP motif of the viral protein, and is capable of binding the UEV domain of Tsg101. Preferably, the peptide contains a contiguous amino acid sequence of less than about 175, 150, 125, 115, 100, 95, 90, 85, 80, 75, 70, 65, 60 or 55 residues of one of the viral proteins in Table 1, which encompasses the P(T/S)AP motif of the viral protein, and is capable of binding the UEV domain of Tsg101. More preferably, the peptide contains a contiguous amino acid sequence of less than about 50, 48, 45, 42, 40, 38, 35, 33, 32, 31, 30, 29, 28, 27, 26, 25, 24, 23, 22, 21 or 20 residues of one of the viral proteins in Table 1, which encompasses the P(T/S)AP motif of the viral protein, and is capable of binding the UEV domain of Tsg101. In preferred embodiments, the peptide contains a contiguous amino acid sequence of from about 4 to about 50, preferably from about 6 to about 50, from about 8 to about 50, more preferably from about 9 to about 50, from about 9 to 45, 9 to 40, 9 to 37, 9 to 35, 9 to 30, 9 to 25 residues of one of the viral proteins in Table 1, which encompasses the P(T/S)AP motif of the viral protein, and is capable of binding the UEV domain of Tsg101. More advantageously, the peptide contains a contiguous amino acid sequence of from 9 to about 10, 11, 12, 13, 14, 15, 16, 17, 18, 19, 20, 21, 22, 23 or 24 residues of a viral protein in Table 1, even more advantageously, from 10 to about 11, 12, 13, 14, 15, 16, 17, 18, 19, 20, 21, 22, 23 or 24 residues of one of the viral proteins in Table 1, which encompasses the P(T/S)AP motif of the viral protein, and is capable of binding the UEV domain of Tsg101.

In specific embodiment, the peptide has a contiguous amino acid sequence of Ebola virus Matrix protein as provided in SEQ ID NOs:38-125 in Table 2. In another specific embodiment, the peptide has a contiguous amino acid sequence of HBV PreS1/PreS2/S Envelope protein as provided in SEQ ID NOs:126-268 in Table 3. In another specific embodiment, the peptide has a contiguous amino acid sequence of HSV1 RL2 protein as provided in SEQ ID NOs:269-554 in Table 4. In yet another specific embodiment, the peptide has a contiguous amino acid sequence of HSV2 viron glycoprotein K as provided in SEQ ID NOs:555-697 in Table 5. The peptide can also has a contiguous amino acid sequence of HSV2 Strain 333 glycoprotein I as provided in SEQ ID NOs:698-749 in Table 6. The peptide can also has a contiguous amino acid sequence of EBV nuclear protein EBNA2 as provided in SEQ ID NOs:750-892 in Table 7, of Influenza A virus hemagglutinin as provided in SEQ ID NOs:893-1035 in Table 8, of HPV L1 protein (My09/My11 Region) as provided in SEQ ID NOs:1036-1178 in Table 9, of HPV Type 23 L2 proteins as provided in SEQ ID NOs:1179-1321 in Table 10, of HPV Type 35 L1 protein as provided in SEQ ID NOs:1322-1464 in Table 11, of HPV Type 6b L2 protein as provided in SEQ ID NOs:1465-1607 in Table 12, of HPV Type 9 late protein as provided in SEQ ID NOs:1608-1750 in Table 13, of HTLV-2 GAG protein as provided in SEQ ID NOs:1751-1893 in Table 14, of West Nile virus polyprotein precursor as provided in SEQ ID NOs:1894-2036 in Table 15, of Measles virus matrix protein as provided in SEQ ID NOs:2037-2179 in Table 16, of Rubella virus non-structural protein as provided in SEQ ID NOs:2180-2322 in Table 17, of Colorado tick fever virus VP12 as provided in SEQ ID NOs:2323-2459 in Table 18, of foot-and-mouth disease virus VP1 capsid protein as provided in SEQ ID NOs:2460-2602 in Table 19, of human foamy virus GAG protein as provided in SEQ ID NOs:2603-2745 in Table 20, of hepatitis E virus ORF-3 protein as provided in SEQ ID NOs:2746-2887 in Table 21, of hepatitis G virus polyprotein precursor as provided in SEQ ID NOs:2888-3030 in Table 22, of HSV5 UL32 protein as provided in SEQ ID NOs:3031-3173 in Table 23, of human parechovirus 2 polyprotein as provided in SEQ ID NOs:3174-3316 in Table 24, and of Semliki forest virus polyprotein as provided in SEQ ID NOs:3317-3459 in Table 25.

In another embodiment, the PX₁X₂P motif in the compound according to the present invention is within an amino acid sequence that is at least 70 percent, preferably at least 80 percent or 85 percent, more preferably at least 90 percent or 95 percent identical to a contiguous span of at least 6, 7, 8 or 9 amino acids, preferably 10, 11, 12, 13, 14, 15 or more amino acids of one of the proteins in Table 1, which spans the late P(T/S)AP motif of the protein. In other embodiments, the PX₁X₂P motif in the compound according to the present invention is within an amino acid sequence that is at least 70 percent, preferably at least 80 percent or 85 percent, more preferably at least 90 percent or 95 percent identical to a contiguous span of at least 6, 7, 8 or 9 amino acids, preferably 10, 11, 12, 13, 14, 15 or more amino acids of a naturally occurring HIV Gag p6 protein or Ebola virus Matrix protein, which spans the late domain motif P(T/S)AP of the protein. In this respect, the percentage identity is determined by the algorithm of Karlin and Altschul, Proc. Natl. Acad. Sci. USA, 90:5873-77 (1993), which is incorporated into the various BLAST programs. Specifically, the percentage identity is determined by the “BLAST 2 Sequences” tool, which is available on the internet at NCBI's website. See Tatusova and Madden, FEMS Microbiol. Lett., 174(2):247-50 (1999). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). It should be understood that such homologue peptides should retain the ability to bind the UEV domain of Tsg101. Preferably, in such embodiments of the present invention, X₁ in the PX₁X₂P motif is selected from the group consisting of T, S, and I and analog thereof, and X₂ is not R. More preferably, X₁ is selected from the group consisting of T, S, and I and analog thereof, and X₂ is A or T or an analog thereof. Most preferably, X₁ is T or S or an analog thereof, and X₂ is A or an analog thereof.

The homologues can be made by site-directed mutagenesis based on a late domain motif-containing Gag polyprotein sequence of HIV or Ebola matrix protein, or a protein in Table 1. The site-directed mutagenesis can be designed to generate amino acid substitutions, insertions, or deletions. Methods for conducting such mutagenesis should be apparent to skilled artisans in the field of molecular biology. The resultant homologues can be tested for their binding affinity to the UEV domain of Tsg101.

The peptide portion in the compounds according to the present invention can also be in a modified form. Various modifications may be made to improve the stability and solubility of the compound, and/or optimize its binding affinity to the UEV domain of Tsg101. Examples of modified forms include, but are not limited to, glycosylated forms, phosphorylated forms, myristoylated forms, palmitoylated forms, ribosylated forms, acetylated forms, etc. Modifications also include intra-molecular crosslinking and covalent attachment to various moieties such as lipids, flavin, biotin, polyethylene glycol or derivatives thereof, etc. In addition, modifications may also include cyclization, and branching. Amino acids other than the conventional twenty amino acids encoded by genes may also be included in a polypeptide sequence in the compound of the present invention. For example, the compounds may include D-amino acids in place of L-amino acids.

To increase the stability of the compounds according to the present invention, various protection groups can also be incorporated into the amino acid residues of the compounds. In particular, terminal residues are preferably protected. Carboxyl groups may be protected by esters (e.g., methyl, ethyl, benzyl, p-nitrobenzyl, t-butyl or t-amyl esters, etc.), lower alkoxyl groups (e.g., methoxy, ethoxy, propoxy, butoxy, etc.), aralkyloxy groups (e.g., benzyloxy, etc.), amino groups, lower alkylamino or di(lower alkyl)amino groups. The term “lower alkoxy” is intended to mean an alkoxy group having a straight, branched or cyclic hydrocarbon moiety of up to six carbon atoms. Protection groups for amino groups may include lower alkyl, benzyloxycarbonyl, t-butoxycarbonyl, and isobornyloxycarbonyl. “Lower alkyl” is intended to mean an alkyl group having a straight, branched or cyclic hydrocarbon moiety of up to six carbon atoms. In one example, a 5-oxo-L-prolyl residue may be used in place of a prolyl residue. A 5-oxo-L-prolyl residue is especially desirable at the N-terminus of a peptide compound. In another example, when a proline residue is at the C-terminus of a peptide compound, a N-ethyl-L-prolinamide residue may be desirable in place of the proline residue. Various other protection groups known in the art useful in increasing the stability of peptide compounds can also be employed.

In addition, the compounds according to the present invention can also be in various pharmaceutically acceptable salt forms. “Pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, hydrobromide salts, sulfate salts, bisulfate salts, nitrate salts, acetate salts, phosphate salts, nitrate salts, oxalate salts, valerate salts, oleate salts, borate salts, benzoate salts, laurate saltes, stearate salts, palmitate salts, lactate salts, tosylate salts, citrate salts, maleate, salts, succinate salts, tartrate salts, naphthylate salts, fumarate salts, mesylate salts, laurylsulphonate salts, glucoheptonate salts, and the like. See, e.g., Berge, et al. J. Pharm. Sci., 66:1-19 (1977).

Suitable pharmaceutically acceptable salts also include, but are not limited to, alkali metal salts, alkaline earth salts, and ammonium salts. Thus, suitable salts may be salts of aluminum, calcium, lithium, magnesium, potassium, sodium and zinc. In addition, organic salts may also be used including, e.g., salts of lysine, N,N′-dibenzylethylenediamine, chloroprocaine, choline, diethanolamine, ethylenediamine, meglumine (N-methylglucamine), procaine and tris. In addition, metal complex forms (e.g. copper complex compounds, zinc complex compounds, etc.) of the compounds of the present invention may also exhibit improved stability.

Additionally, as will be apparent to skilled artisans apprised of the present disclosure, peptide mimetics can be designed based on the above-described compounds according to the present invention. However, it is noted that the mimetics must be capable of binding the UEV domain of Tsg101. For example, peptoid analogs of the P(T/S)(A/T)P motif can be prepared using known methods. Peptoids are oligomeric N-substituted glycines. Typically, various side chain groups can be included when forming an N-substituted glycine (peptoid monomer) that mimics a particular amino acid. Peptoid monomers can be linked together to form an oligomeric N-substituted glycines-peptoid. Peptoids are easy to synthesize in large amounts. In contrast to peptides, the backbone linkage of peptoids are resistant to hydrolytic enzymes. In addition, since a variety of functional groups can be presented as side chains off of the oligomeric backbone, peptoid analogs corresponding to any peptides can be produced with improved characterics. See Simon et al., Proc. Natl. Acad. Sci. USA, 89:9367-9371 (1992); Figliozzi et al., Methods Enzymol., 267:437-447 (1996); Horwell, Trends Biotechnol., 13:132-134 (1995); and Horwell, Drug Des. Discov., 12:63-75 (1994), all of which are incorporated herein by reference.

Thus, peptoid analogs of the above-described compounds of the present invention can be made using methods known in the art. The thus prepared peptoid analogs can be tested for their binding affinity to Tsg101. They can also be tested in anti-viral assays for their ability to inhibit virus budding from infected host cells and ability to inhibit virus propagation.

Mimetics of the compounds of the present invention can also be selected by rational drug design and/or virtual screening. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990). Structural information on the UEV domain of Tsg101 and/or the binding complex formed by the Tsg101 UEV domain and the HIV Gag p6 P(T/S)AP motif or a protein in Table 1 are obtained. The interacting complex can be studied using various biophysics techniques including, e.g., X-ray crystallography, NMR, computer modeling, mass spectrometry, and the like. Likewise, structural information can also be obtained from protein complexes formed by the Tsg101 UEV domain and a variation of the PTAP motif.

Computer programs are employed to select compounds based on structural models of the binding complex formed by the Tsg101 UEV domain and the HIV Gag p6 P(T/S)AP motif or the P(T/S)AP motif in one of the proteins in Table 1. In addition, once an effective compound is identified, structural analogs or mimetics thereof can be produced based on rational drug design with the aim of improving drug efficacy and stability, and reducing side effects.

In addition, understanding of the interaction between the Tsg101 UEV domain and compounds of the present invention can also be derived from mutagenesis analysis using yeast two-hybrid system or other methods for detection protein-protein interaction. In this respect, various mutations can be introduced into the interacting proteins and the effect of the mutations on protein-protein interaction is examined by a suitable method such as in vitro binding assay or the yeast two-hybrid system.

Various mutations including amino acid substitutions, deletions and insertions can be introduced into the protein sequence of the Tsg101 UEV domain and/or a compound of the present invention using conventional recombinant DNA technologies. Generally, it is particularly desirable to decipher the protein binding sites. Thus, it is important that the mutations introduced only affect protein-protein interaction and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., an in vitro binding assay. In this manner, the domains or residues of the proteins important to compound-target interaction can be identified.

Based on the structural information obtained, structural relationships between the Tsg101 UEV domain and a compound of the present invention are elucidated. The moieties and the three-dimensional structures critical to the interaction are revealed. Medicinal chemists can then design analog compounds having similar moieties and structures.

The residues or domains critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis, similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs the energy minimization and molecular dynamics functions, and QUANTA program which performs the construction, graphic modeling and analysis of molecular structure. Such programs allow interactive construction, visualization and modification of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. These rationally designed compounds are further tested. In this manner, pharmacologically acceptable and stable compounds with improved efficacy and reduced side effect can be developed. The compounds identified in accordance with the present invention can be incorporated into a pharmaceutical formulation suitable for administration to an individual.

The mimetics including peptoid analogs can exhibit optimal binding affinity to the UEV domain of human Tsg101 or animal orthologs thereof. Various known methods can be utilized to test the Tsg101-binding characteristics of a mimetics. For example, the entire Tsg101 protein or a fragment thereof containing the UEV domain may be recombinantly expressed, purified, and contacted with the mimetics to be tested. Binding can be determined using a surface plasmon resonance biosensor. See e.g., Panayotou et al., Mol. Cell. Biol., 13:3567-3576 (1993). Other methods known in the art for estimating and determining binding constants in protein-protein interactions can also be employed. See Phizicky and Fields, et al., Microbiol. Rev., 59:94-123 (1995). For example, protein affinity chromatography may be used. First, columns are prepared with different concentrations of an interacting member, which is covalently bound to the columns. Then a preparation of its interacting partner is run through the column and washed with buffer. The interacting partner bound to the interacting member linked to the column is then eluted. Binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, the two interacting members sediment as a complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), and solution equilibrium measured with immobilized binding protein (See e.g., Nelson and Long, Biochemistry, 30:2384-2390 (1991)).

The compounds capable of binding Tsg101 UEV domain according the present invention can be delivered into cells by direct cell internalization, receptor mediated endocytosis, or via a “transporter.” It is noted that the compound administered to cells in vitro or in vivo in the method of the present invention preferably is delivered into the cells in order to achieve optimal results. Thus, preferably, the compound to be delivered is associated with a transporter capable of increasing the uptake of the compound by an animal cell, preferably a mammalian cell, susceptible to infection by a virus, particularly a virus selected from those in Table 1. As used herein, the term “associated with” means a compound to be delivered is physically associated with a transporter. The compound and the transporter can be covalently linked together, or associated with each other as a result of physical affinities such as forces caused by electrical charge differences, hydrophobicity, hydrogen bonds, van der Waals force, ionic force, or a combination thereof. For example, the compound can be encapsulated within a transporter such as a liposome.

As used herein, the term “transporter” refers to an entity (e.g., a compound or a composition or a physical structure formed from multiple copies of a compound or multiple different compounds) that is capable of facilitating the uptake of a compound of the present invention by a mammalian cell, particularly a human cell. Typically, the cell uptake of a compound of the present invention in the presence of a “transporter” is at least 50% or 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporter.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol., 8:84-87 (1998), which is incorporated herein by reference. Penetratins transport molecules attached thereto across cytoplasm membranes or nucleus membranes efficiently in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminal is tryptophan, while the amino acids at positions 3 and 5 counting from either the N- or C-terminal are not both valine. Preferably, the helix 3 of the homeodomain of Drosophila Antennapedia is used as a transporter. More preferably, a peptide having a sequence of the amino acids 43-58 of the homeodomain Antp is employed as a transporter. In addition, other naturally occurring homologs of the helix 3 of the homeodomain of Drosophila Antennapedia can also be used. For example, homeodomains of Fushi-tarazu and Engrailed have been shown to be capable of transporting peptides into cells. See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l Acad. Sci. USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histidine oligomers, D-histidine oligomers, L-ornithine oligomers, D-ornithine oligomers, and various homologues, derivatives (e.g., modified forms with conjugates linked to the small peptides) and peptoid analogs thereof. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or at least 9 such amino acid residues. In one embodiment, the transporter is a peptide that includes at least six contiguous amino acid residues that are L-arginine, D-arginine, L-lysine, D-lysine, L-histidine, D-histidine, L-ornithine, D-ornithine, or a combination thereof.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J. 12:67-77 (1998)), and HSV-1 structural protein VP22 (See Elliott and O'Hare, Cell, 88:223-233 (1997)).

As the above-described various transporters are generally peptides, fusion proteins can be conveniently made by recombinant expression to contain a transporter peptide covalently linked by a peptide bond to a peptide having the PX₁X₂P motif. Alternatively, conventional methods can be used to chemically synthesize a transporter peptide or a peptide of the present invention or both.

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In one embodiment, the peptide consists of a contiguous amino acid sequence of from 8 to about 30 amino acid residues of a viral protein selected from the group consisting of HBV PreS1/PreS2/S envelope protein, HSV1 RL2 protein, HSV2 virion glycoprotein K, HSV2 Strain 333 glycoprotein I, EBV nuclear protein EBNA2, Influenza A virus hemagglutinin, HPV L1 proteins, HPV L2 proteins, HPV late proteins, HTLV-2 GAG protein, West Nile virus polyprotein precursor, Measles virus matrix protein, Rubella virus non-structural protein, Colorado tick fever virus VP12, foot-and-mouth disease virus VP1 capsid protein, human foamy virus GAG protein, hepatitis G virus polyprotein precursor, human parechovirus 2 polyprotein, and Semliki forest virus polyprotein, wherein the contiguous amino acid sequence encompasses the P(T/S)AP motif of the viral protein, and wherein the peptide is capable of binding the UEV domain of Tsg101. In specific embodiments, the peptide does not contain a contiguous amino acid sequence of an HIV GAG protein, or of an Ebola virus Matrix (EbVp40) protein, or of a polyprotein precursor, or of hepatitis E virus ORF-3 protein that is sufficient to impart an ability to bind the UEV domain of Tsg101 on said peptide.

Advantageously, the isolated peptide consists of from 9 to about 20 amino acid residues. Examples of such isolated peptides include peptides having an amino acid sequence selected from the group consisting of SEQ ID NOs:38-125, SEQ ID NOs:126-268, SEQ ID NOs:269-554, SEQ ID NOs:555-697, SEQ ID NOs:698-749, SEQ ID NOs:750-892, SEQ ID NOs:893-1035, SEQ ID NOs:1036-1178, SEQ ID NOs:1179-1321, SEQ ID NOs:1322-1464, SEQ ID NOs:1465-1607, SEQ ID NOs:1608-1750, SEQ ID NOs:1751-1893, SEQ ID NOs:1894-2036, SEQ ID NOs:2037-2179, SEQ ID NOs:2180-2322, SEQ ID NOs:2323-2459, SEQ ID NOs:2460-2602, SEQ ID NOs:2603-2745, SEQ ID NOs:2888-3030, SEQ ID NOs:3174-3316, and SEQ ID NOs:3317-3459.

Any suitable gene therapy methods may be used for purposes of the present invention. Generally, an exogenous nucleic acid encoding a peptide compound of the present invention is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In this respect, a CD⁴⁺ T cell-specific promoter will be most desirable. In addition, selection markers may also be included in the vector for purposes of selecting, in vitro, those cells that contain the exogenous nucleic acid encoding the peptide compound of the present invention. Various selection markers known in the art may be used including, but not limited to, e.g., genes conferring resistance to neomycin, hygromycin, zeocin, and the like.

In one embodiment, the exogenous nucleic acid is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability, e.g., the ability to replicate in the host cells. The exogenous nucleic acid to be introduced into a patient may be incorporated into the engineered viral genome, e.g., by inserting it into a viral gene that is non-essential to the viral infectivity. Viral vectors are convenient to use as they can be easily introduced into tissue cells by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), viruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avian retroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous nucleic acid fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine, having 3-100 lysine residues), which is itself coupled to an integrin receptor binding moiety (e.g., a cyclic peptide having the amino acid sequence RGD).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily absorb the complex.

The exogenous nucleic acid can be introduced into a patient for purposes of gene therapy by various methods known in the art. For example, the exogenous nucleic acid alone or in a conjugated or complex form described above, or incorporated into viral or DNA vectors, may be administered directly by injection into an appropriate tissue or organ of a patient. Alternatively, catheters or like devices may be used for delivery into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous nucleic acid encoding a peptide compound of the present invention or vectors containing the nucleic acid can be introduced into isolated cells using any known techniques such as calcium phosphate precipitation, microinjection, lipofection, electroporation, gene gun, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., obtained from the patient being treated.

When the transporter used in the method of the present invention is a peptidic transporter, a hybrid polypeptide or fusion polypeptide is provided. In preferred embodiments, the hybrid polypeptide includes (a) a first portion comprising an amino acid sequence motif PX₁X₂P, and capable of binding the UEV domain of Tsg101, wherein X₁ and X₂ are amino acids, and X₂ is not R, and (b) a second portion which is a peptidic transporter capable of increasing the uptake of the first portion by human cells. Preferably, the first portion consists of from about 8 to about 100 amino acid residues, more preferably 9 to 20 amino acid residues. Preferably, the peptidic transporter is capable of increasing the uptake of the first portion by a mammalian cell by at least 100%, more preferably by at least 300%. In one embodiment, the first portion does not contain a contiguous amino acid sequence of an HIV GAG protein that is sufficient to impart an ability to bind the UEV domain of Tsg101 on said peptide.

The hybrid polypeptide can be produced in a patient's body by administering to the patient a nucleic acid encoding the hybrid polypeptide by a gene therapy method as described above. Alternatively, the hybrid polypeptide can be chemically synthesized or produced by recombinantly expression.

Thus, the present invention also provides isolated nucleic acids encoding the hybrid polypeptides and host cells recombinantly expressing the hybrid polypeptides. Such a host cell can be prepared by introducing into a suitable cell an exogenous nucleic acid encoding one of the hybrid polypeptides by standard molecular cloning techniques as described above. The nucleic acids can be prepared by linked a nucleic acid encoding the first portion and a nucleic acid encoding the second portion. Methods for preparing such nucleic acids and for using them in recombinant expression should be apparent to skilled artisans.

The compounds according to the present invention capable of binding Tsg101 are a novel class of antiviral compounds distinct from other commercially available compounds. While not wishing to be bound by any theory or hypothesis, it is believed that the compounds according to the present invention inhibit virus through a mechanism distinct from those of the antiviral compounds known in the art. Therefore, it may be desirable to employ combination therapies to administer to a patient both a compound according to the present invention, with or without a transporter, and another anti-viral compound of a different class. However, it is to be understood that such other antiviral compounds should be pharmaceutically compatible with the compound of the present invention. By “pharmaceutically compatible” it is intended that the other anti-viral agent(s) will not interact or react with the above composition, directly or indirectly, in such a way as to adversely affect the effect of the treatment, or to cause any significant adverse side reaction in the patient. In this combination therapy approach, the two different pharmaceutically active compounds can be administered separately or in the same pharmaceutical composition. Compounds suitable for use in combination therapies with the Tsg101-binding compounds according to the present invention include, but are not limited to, small molecule drugs, antibodies, immunomodulators, and vaccines.

In the case of treating HIV infection and AIDS, and/or preventing AIDS using the compounds of the present invention, another anti-HIV compound may be used with a compound of the present invention in a combination therapy. Compounds suitable for use in combination therapies with the Tsg101-binding compounds according to the present invention include, but are not limited to, HIV protease inhibitors, nucleoside HIV reverse transcriptase inhibitors, non-nucleoside HIV reverse transcriptase inhibitors, HIV integrase inhibitors, immunomodulators, and vaccines.

Examples of nucleoside HIV reverse transcriptase inhibitors include 3′-Azido-3′-deoxythymidine (Zidovudine, also known as AZT and RETROVIR®), 2′,3′-Didehydro-3′-deoxythymidine (Stavudine, also known as 2′,3′-dihydro-3′-deoxythymidine, d4T, and ZERIT®), (2R-cis)-4-Amino-1-[2-(hydroxymethyl)-1,3-oxathiolan-5-yl]-2(1H)-pyrimidinone (Lamivudine, also known as 3TC, and EPIVIR®), and 2′,3′-dideoxyinosine (ddI).

Examples of non-nucleoside HIV reverse transcriptase inhibitors include (−)-6-Chloro-4-cyclopropylethynyl-4-trifluoromethyl-1,4-dihydro-2H-3,1-benzoxazin-2-one (efavirenz, also known as DMP-266 or SUSTIVA®) (see U.S. Pat. No. 5,519,021), 1-[3-[(1-methylethyl)amino-1]-2-pyridinyl]-4-[[5-[(methylsulfonyl)amino]-1H-indol-2-yl]carbonyl]piperazine (Delavirdine, see PCT International Patent Application No. WO 91/09849), and (1S,4R)-cis-4-[2-amino-6-(cyclopropylamino)-9H-purin-9-yl]-2-cyclopentene-1-methanol (Abacavir).

Examples of protease inhibitors include [5S-(5R*,8R*,10R*,11R*)]-10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid 5-thiazolylmethyl ester (Ritonavir, marketed by Abbott as NORVIR®), [3S-[2(2S*,3S*),3a,4ab,8ab]]-N-(1,1-dimethylethyl)decahydro-2-[2-hydroxy-3-[(3-hydroxy-2-methylbenzoyl)amino]-4-(phenylthio)butyl]-3-isoquinolinecarboxamide monomethanesulfonate (Nelfinavir, marketed by Agouron as VIRACEPT®), N-(2(R)-hydroxy-1(S)-indanyl)-2(R)-phenylmethyl-4-(S)-hydroxy-5-(1-(4-(2-benzo[b]furanylmethyl)-2(S)—N′(t-butylcarboxamido)-piperazinyl))-pentaneamide (See U.S. Pat. No. 5,646,148), N-(2(R)-hydroxy-1(S)-indanyl)2(R)-phenylmethyl-4-(S)-hydroxy-5-(1-(4-(3-pyridylmethyl)-2(S)—N′-(t-butylcarboxamido)-piperazinyl))-pentaneamide (Indinavir, marketed by Merck as CRIXIVAN®), 4-amino-N-((2 syn,3S)-2-hydroxy-4-phenyl-3-((S)-tetrahydrofuran-3-yloxycarbonylamino)-butyl)-N-isobutyl-benzenesulfonamide (amprenavir, see U.S. Pat. No. 5,585,397), and N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide (Saquinavir, marketed by Roche Laboratories as INVIRASE®).

Examples of suitable HIV integrase inhibitors are disclosed in U.S. Pat. Nos. 6,110,716; 6,124,327; and 6,245,806, which are incorporated herein by reference.

In addition, antifusogenic peptides disclosed in, e.g., U.S. Pat. No. 6,017,536 can also be included in the combination therapies according to the present invention. Such peptides typically consist of a 16 to 39 amino acid region of a simian immunodeficiency virus (SIV) protein and are identified through computer algorithms capable of recognizing the ALLMOTI5, 107×178×4, or PLZIP amino acid motifs. See U.S. Pat. No. 6,017,536, which is incorporated herein by reference.

Typically, a compound of the present invention is administered to a patient in a pharmaceutical composition, which typically includes one or more pharmaceutically acceptable carriers that are inherently nontoxic and non-therapeutic. That is, the compounds are used in the manufacture of medicaments for use in the methods of treating viral infection provided in the present invention.

The pharmaceutical composition according to the present invention may be administered to a subject needing treatment or prevention through any appropriate routes such as parenteral, oral, or topical administration. The active compounds of this invention are administered at a therapeutically effective amount to achieve the desired therapeutic effect without causing any serious adverse effects in the patient treated. Generally, the toxicity profile and therapeutic efficacy of therapeutic agents can be determined by standard pharmaceutical procedures in suitable cell models or animal models or human clinical trials. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

Typically, the compounds of the present invention can be effective at an amount of from about 0.01 microgram to about 5000 mg per day, preferably from about 1 microgram to about 2500 mg per day. However, the amount can vary with the body weight of the patient treated and the state of disease conditions. The active ingredient may be administered at once, or may be divided into a number of smaller doses to be administered at predetermined intervals of time. The suitable dosage unit for each administration of the compounds of the present invention can be, e.g., from about 0.01 microgram to about 2000 mg, preferably from about 1 microgram to about 1000 mg.

In the case of combination therapy, a therapeutically effective amount of another anti-viral compound can be administered in a separate pharmaceutical composition, or alternatively included in the pharmaceutical composition that contains a compound according to the present invention. The pharmacology and toxicology of many of such other anti-viral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds according to this invention can be administered to patients to be treated through any suitable routes of administration. Advantageously, the active compounds are delivered to the patient parenterally, i.e., by intravenous, intramuscular, intraperitoneal, intracisternal, subcutaneous, or intraarticular injection or infusion.

For parenteral administration, the active compounds can be formulated into solutions or suspensions, or in lyophilized forms for conversion into solutions or suspensions before use. Lyophilized compositions may include pharmaceutically acceptable carriers such as gelatin, DL-lactic and glycolic acids copolymer, D-mannitol, etc. To convert the lyophilized forms into solutions or suspensions, diluent containing, e.g., carboxymethylcellulose sodium, D-mannitol, polysorbate 80, and water may be employed. Lyophilized forms may be stored in, e.g., a dual chamber syringe with one chamber containing the lyophilized composition and the other chamber containing the diluent. In addition, the active ingredient(s) can also be incorporated into sterile lyophilized microspheres for sustained release. Methods for making such microspheres are generally known in the art. See U.S. Pat. Nos. 4,652,441; 4,728,721; 4,849,228; 4,917,893; 4,954,298; 5,330,767; 5,476,663; 5,480,656; 5,575,987; 5,631,020; 5,631,021; 5,643,607; and 5,716,640.

In a solution or suspension form suitable for parenteral administration, the pharmaceutical composition can include, in addition to a therapeutically or prophylactically effective amount of a compound of the present invention, a buffering agent, an isotonicity adjusting agent, a preservative, and/or an anti-absorbent. Examples of suitable buffering agent include, but are not limited to, citrate, phosphate, tartrate, succinate, adipate, maleate, lactate and acetate buffers, sodium bicarbonate, and sodium carbonate, or a mixture thereof. Preferably, the buffering agent adjusts the pH of the solution to within the range of 5-8. Examples of suitable isotonicity adjusting agents include sodium chloride, glycerol, mannitol, and sorbitol, or a mixture thereof. A preservative (e.g., anti-microbial agent) may be desirable as it can inhibit microbial contamination or growth in the liquid forms of the pharmaceutical composition. Useful preservatives may include benzyl alcohol, a paraben and phenol or a mixture thereof. Materials such as human serum albumin, gelatin or a mixture thereof may be used as anti-absorbents. In addition, conventional solvents, surfactants, stabilizers, pH balancing buffers, and antioxidants can all be used in the parenteral formulations, including but not limited to dextrose, fixed oils, glycerine, polyethylene glycol, propylene glycol, ascorbic acid, sodium bisulfite, and the like. The parenteral formulation can be stored in any conventional containers such as vials, ampoules, and syringes.

The active compounds can also be delivered orally in enclosed gelatin capsules or compressed tablets. Capsules and tablets can be prepared in any conventional techniques. For example, the active compounds can be incorporated into a formulation which includes pharmaceutically acceptable carriers such as excipients (e.g., starch, lactose), binders (e.g., gelatin, cellulose, gum tragacanth), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules.

Other forms of oral formulations such as chewing gum, suspension, syrup, wafer, elixir, and the like can also be prepared containing the active compounds used in this invention. Various modifying agents for flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered topically through rectal, vaginal, nasal, bucal, or mucosal applications. Topical formulations are generally known in the art including creams, gels, ointments, lotions, powders, pastes, suspensions, sprays, drops and aerosols. Typically, topical formulations include one or more thickening agents, humectants, and/or emollients including but not limited to xanthan gum, petrolatum, beeswax, or polyethylene glycol, sorbitol, mineral oil, lanolin, squalene, and the like.

A special form of topical administration is delivery by a transdermal patch. Methods for preparing transdermal patches are disclosed, e.g., in Brown, et al., Annual Review of Medicine, 39:221-229 (1988), which is incorporated herein by reference.

The active compounds can also be delivered by subcutaneous implantation for sustained release. This may be accomplished by using aseptic techniques to surgically implant the active compounds in any suitable formulation into the subcutaneous space of the anterior abdominal wall. See, e.g., Wilson et al., J. Clin. Psych. 45:242-247 (1984). Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. See, e.g., Phillips et al., J. Pharmaceut. Sci., 73:1718-1720 (1984).

The active compounds can also be conjugated, i.e., covalently linked, to a water soluble non-immunogenic high molecular weight polymer to form a polymer conjugate. Preferably, such polymers do not undesirably interfere with the cellular uptake of the active compounds. Advantageously, such polymers, e.g., polyethylene glycol, can impart solubility, stability, and reduced immunogenicity to the active compounds. As a result, the active compound in the conjugate when administered to a patient, can have a longer half-life in the body, and exhibit better efficacy. In one embodiment, the polymer is a peptide such as albumin or antibody fragment Fc. PEGylated proteins are currently being used in protein replacement therapies and for other therapeutic uses. For example, PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). A general review of PEG-protein conjugates with clinical efficacy can be found in, e.g., Burnham, Am. J. Hosp. Pharm., 15:210-218 (1994). Preferably, the covalent linkage between the polymer and the active compound is hydrolytically degradable and is susceptible to hydrolysis under physiological conditions. Such conjugates are known as “prodrugs” and the polymer in the conjugate can be readily cleaved off inside the body, releasing the free active compounds.

Alternatively, other forms controlled release or protection including microcapsules and nanocapsules generally known in the art, and hydrogels described above can all be utilized in oral, parenteral, topical, and subcutaneous administration of the active compounds.

Another preferable delivery form is using liposomes as carrier. Liposomes are micelles formed from various lipids such as cholesterol, phospholipids, fatty acids, and derivatives thereof. Active compounds can be enclosed within such micelles. Methods for preparing liposomal suspensions containing active ingredients therein are generally known in the art and are disclosed in, e.g., U.S. Pat. No. 4,522,811, and Prescott, Ed., Methods in Cell Biology, Volume XIV, Academic Press, New York, N.Y. (1976), p. 33 et seq., both of which are incorporated herein by reference. Several anticancer drugs delivered in the form of liposomes are known in the art and are commercially available from Liposome Inc. of Princeton, N.J., U.S.A. It has been shown that liposomes can reduce the toxicity of the active compounds, and increase their stability.

### Example 1

Yeast two-hybrid assays were utilized to determine the effect of amino acid substitution mutations in the PTAP motif of HIV p6gag on the interaction between Tsg101 and p6gag. To prepare a yeast two-hybrid activation domain-Tsg101 construct, a DNA fragment encompassing the full-length coding sequence for Tsg101 according to GenBank Accession No. U82130 was obtained by PCR from a human fetal brain cDNA library and cloned into the EcoRI/Pst1 sites of the activation domain parent plasmid GADpN2 (LEU2, CEN4, ARS1, ADH1p-SV40NLS-GAL4 (768-881)-MCS (multiple cloning site)-PGK1t, AmpR, ColE1_ori).

To prepare the yeast two-hybrid DNA binding domain-HIV1 p6gag construct, a DNA fragment corresponding to the HIV1 p6 peptide derived from the HIV1.NL43 strain GAG protein was obtained by PCR from the NL43 containing plasmid R9Δapa and was cloned into the EcoRI/Sal1 sites of the binding domain parent plasmid pGBT.Q. The sequence of the amplified insert is shown in SEQ ID NO:3485. In addition, the amino acid sequence of the HIV-1_{NYU/BR5} GAG is provided in GenBank under Accession No. AF324493 and is listed in SEQ ID NO:3484.

The following amino acid substitution mutations were introduced by PCR into the HIV1 p6gag sequence in the yeast two-hybrid binding domain-HIV1 p6gag construct described above. The mutations were verified by DNA sequence analysis. Such mutations are summarized in Table 26 below.

TABLE 26
Tested Mutations in p6gag Protein

| Mutant | | | | | | | | | | | | | | | |
|---|---|---|---|---|---|---|---|---|---|---|---|---|---|---|---|
| Construct | p6gag Peptide Sequence Surrounding the PTAP Motif | | | | | | | | | | | | | | |
| p6(wt) | S | R | P | E | P | T | A | P | P | E | E | S | F | R | F |
| p6(E6G) | | | | G | | | | | | | | | | | |
| p6(P7L) | | | | | L | | | | | | | | | | |
| p6(A9R) | | | | | | | R | | | | | | | | |
| p6(P10L) | | | | | | | | L | | | | | | | |

To test the effect of the mutations, yeast cells of the strain Y189 purchased from Clontech (ura3-52 his3*200 ade2-101 trp1-901 leu2-3,112 met gal4 gal80 URA3::GAL1p-lacZ) were co-transformed with the activation domain-Tsg101 construct and one of the binding domain-mutant p6gag constructs or the binding domain-wild type p6gag construct. Filter lift assays for β-Gal activity were conducted by lifting the transformed yeast colonies with filters, lysing the yeast cells by freezing and thawing, and contacting the lysed cells with X-Gal. Positive β-Gal activity indicates that the p6gag wild type or mutant protein interacts with Tsg101. All binding domain constructs were also tested for self-activation of β-Gal activity. The results are shown in Table 27.

TABLE 27
Interactions Between Tsg101 and p6gag

| | p6(wt) | p6(E6G) | p6(P7L) | p6(A9R) | p6(P10L) |
|---|---|---|---|---|---|
| Tsg101 | + | + | − | − | − |
| p6(wt) | − | | | | |
| p6(E6G) | | − | | | |
| p6(P7L) | | | − | | |
| p6(A9R) | | | | − | |
| p6(P10L) | | | | | − |

Thus, as is clear from Table 27, the mutations in the PTAP motif of HIV p6gag abolished the interaction between Tsg101 and HIV p6gag, while the p6/E6G mutation outside the PTAP motif did not result in the elimination of the Tsg101-p6gag interaction.

The interactions between TSG101 and wild-type p6gag (WT) or the p6gag PTAP mutants were further quantitated by performing liquid culture β-galactosidase assays. Cultures were grown overnight in synthetic media (-Leu, -Trp, +glucose) in 96 well plates, normalized for optical density, and lysed by addition of 6× lysis/substrate solution in 6×Z-buffer (60 mM KCl, 6 mM MgSO₄, 360 mM Na₂HPO₄, 240 mM NaH₂PO₄, 6 mg/ml CPRG, 0.12 U/ml lyticase, 0.075% NP-40). Cultures were incubated for 2 hr at 37° C., clarified by centrifugation, and the optical absorbance of each supernatant was measured (575 nm). Full length Tsg101 bound wild-type p6 in the two-hybrid liquid culture assay, resulting in high levels of β-galactosidase activity (>300-fold over background). Three different p6 point mutants were used to test whether the Tsg101 binding interaction required the PTAP late domain motif within HIV-1 p6, and all three (P6L, A9R and P10L) reduced β-galactosidase activity to background levels. Each of these point mutations also arrests HIV-1 budding at a late stage (Huang et al. 1995). These results are consistent with the hypothesis that the interaction between HIV p6gag and the human cellular protein TSG101 is essential for viral budding to occur.

### Example 2

A fusion protein with a GST tag fused to the HIV-1 GAGp6 domain was recombinantly expressed and purified by chromatography. In addition, a GAGp6 peptide containing the first 14 amino acid residues (“p6(1-14)”) was synthesized chemically by standard peptide synthesis methods. The peptide was purified by conventional protein purification techniques, e.g., by chromatography.

Nunc/Nalgene Maxisorp plates were incubated overnight at 4° C. or for 1-2 hrs at room temperature in 100 μl of a protein coupling solution containing purified GST-p6 and 50 mM Carbonate, pH=9.6. This allowed the attachment of the GST-p6 fusion protein to the plates. Liquids in the plates were then emptied and wells filled with 400 μl/well of a blocking buffer (SuperBlock; Pierce-Endogen, Rockford, Ill.). After incubating for 1 hour at room temperature, 100 μl of a mixture containing Drosophila S2 cell lysate myc-tagged Tsg101 (residues 1-207) and a specific amount of the p6(1-14) peptide were applied to the wells of the plate. This mixture was allowed to react for 2 hours at room temperature to form p6:Tsg101 protein-protein complexes.

Plates were then washed 4×100 μl with 1×PBST solution (Invitrogen; Carlsbad, Calif.). After washing, 100 μl of 1 μg/ml solution of anti-myc monoclonal antibody (Clone 9E10; Roche Molecular Biochemicals; Indianapolis, Ind.) in 1×PBST was added to the wells of the plate to detect the myc-epitope tag on the Tsg101 protein. Plates were then washed again with 4×100 μl with 1×PBST solution and 100 μl of 1 μg/ml solution of horseradish peroxidase (HRP) conjugated Goat anti-mouse IgG (Jackson Immunoresearch Labs; West Grove, Pa.) in 1×PBST was added to the wells of the plate to detect bound mouse anti-myc antibodies. Plates were then washed again with 4×100 μl with 1×PBST solution and 100 μl of fluorescent substrate (QuantaBlu; Pierce-Endogen, Rockford, Ill.) was added to all wells. After 30 minutes, 100 μl of stop solution was added to each well to inhibit the function of HRP. Plates were then read on a Packard Fusion instrument at an excitation wavelength of 325 nm and an emission wavelength of 420 nm. The presence of fluorescent signals indicates binding of Tsg101 to the fixed GST-p6. In contrast, the absence of fluorescent signals indicates that the p6(1-14) peptide is capable of disrupting the interaction between Tsg101 and HIV p6.

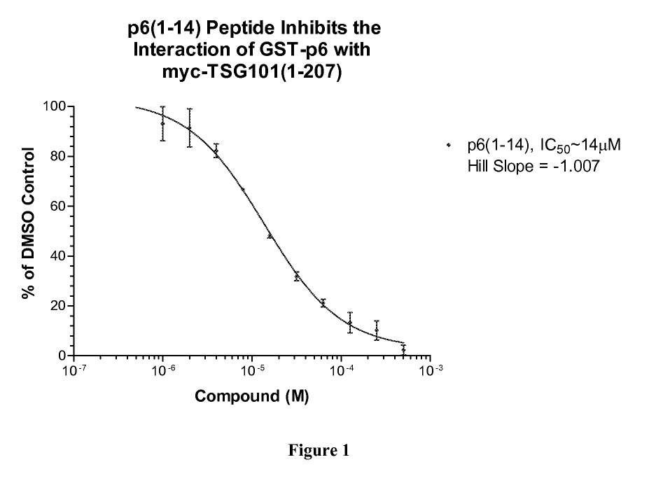

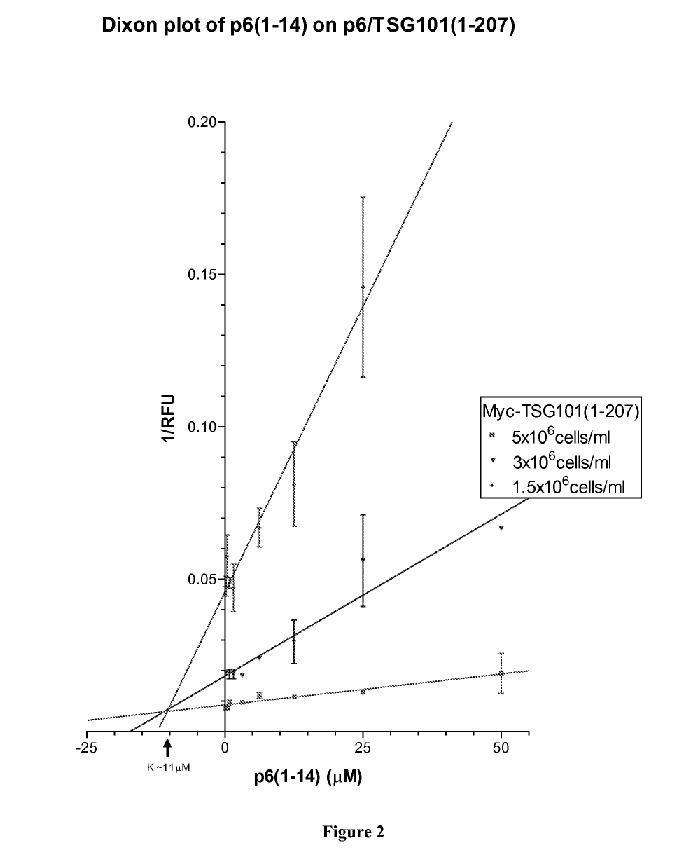

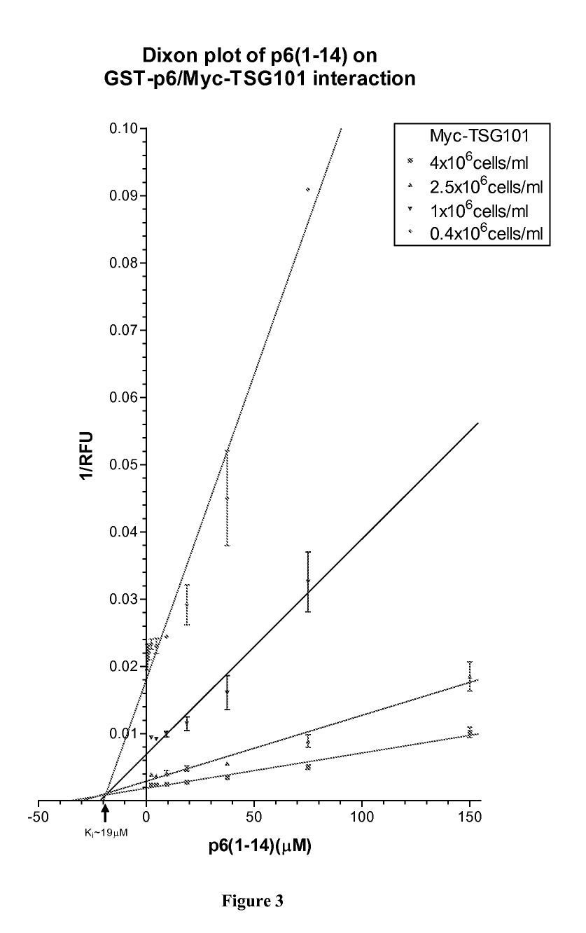

Different concentrations of the p6(1-14) peptide were tested, and the relative intensities of the fluorescence signals obtained at different concentrations were plotted against the peptide concentrations. The competitive inhibition curve is shown in FIG. 1. Two Dixon plots are shown in FIG. 2 and FIG. 3, respectively.

### Example 3

### 1. Materials

For antiviral tests, the following peptidic compounds (in Table 3) were chemically synthesized and purified by conventional protein purification techniques:

TABLE 28

| Compound | Formula | SEQ ID NO: |
|---|---|---|
| MPI-PEP1 | NH₂-(R)₉-PEPTAPEE-COOH | 3485 |
| MPI-PEP2 | NH₂-(R)₉-PEPTALEE-COOH | 3486 |
| MPI-PEP3 | NH₂-RPEPTAP-CO-NH₂ | 3487 |

The compounds were solubilized in sterile RPMI 1640 tissue culture medium to yield 40 mM stock solutions. AZT was used as a positive control antiviral compound.

Fresh human blood was obtained commercially from Interstate Blood Bank, Inc. (Memphis, Tenn.). The lymphotropic clinical isolate HIV-1_{ROJO} was obtained from a pediatric patient attending the AIDS Clinic at the University of Alabama at Birmingham. The laboratory-adapted HIV-1_{IIIB} strain was propagated and tittered in fresh human PBMCs; pre-titered aliquots of HIV-1_{ROJO} and Hiv-1_{IIIB} were removed from the freezer (−80° C.) and thawed rapidly to room temperature in a biological safety cabinet immediately before use. Phytohemagglutinin (PHA-P) was obtained from Sigma (St. Louis, Mo.) and recombinant IL-2 was obtained from Amgen (San Francisco, Calif.).

### 2. Anti-HIV Efficacy Evaluation in Fresh Human PBMCs

Fresh human PBMCs were isolated from screened donors, seronegative for HIV and HBV. Leukophoresed blood was diluted 1:1 with Dulbecco's phosphate buffered saline (PBS), layered over 14 mL of Ficoll-Hypaque density gradient in a 50 mL centrifuge tube and then centrifuged for 30 minutes at 600×g. Banded PBMCs were aspirated from the resulting interface and subsequently washed 2× with PBS by low speed centrifugation. After the final wash, cells were enumerated by trypan blue exclusion and re-suspended at 1×10⁷ cells/mL in RPMI 1640 supplemented with 15% Fetal Bovine Serum (FBS), 2 mM L-glutamine, 4 μg/mL PHA-P. The cells were allowed to incubate for 48-72 hours at 37° C. After incubation, PBMCs were centrifuged and reset in RPMI 1640 with 15% FBS, 2 mM L-glutamine, 100 U/ml penicillin, 100 μg/mL streptomycin, 10 μg/mL gentamycin, and 20 U/mL recombinant human IL-2. PBMCs were maintained in this medium at a concentration of 1-2×10⁶ cells/mL with biweekly medium changes until used in the assay protocol.

For the standard PBMC assay, PHA-P stimulated cells from at least two normal donors were pooled, diluted in fresh medium to a final concentration of 1×10⁶ cells/mL, and plated in the interior wells of 96 well round bottom microplate at 50 μL/well (5×10⁴ cells/well). Test drug dilutions were prepared at a 2× concentration in microtiter tubes and 100 μL of each concentration was placed in appropriate wells in a standard format. 50 μl, of a predetermined dilution of virus stock was placed in each test well (final MOI≈0.1). Wells with cells and virus alone were used for virus control. Separate plates were prepared identically without virus for drug cytotoxicity studies using an XTT assay system. The PBMC cultures were maintained for seven days following infection, at which time cell-free supernate samples were collected and assayed for reverse transcriptase activity as described below.

### 3. Reverse Transcriptase Activity Assay

A microtiter based reverse transcriptase (RT) reaction was utilized. See Buckheit et al., AIDS Research and Human Retroviruses 7:295-302 (1991). Tritiated thymidine triphosphate (NEN) (TTP) was resuspended in distilled H₂O at 5 Ci/ml. Poly rA and oligo dT were prepared as a stock solution which was kept at −20° C. The RT reaction buffer was prepared fresh on a daily basis and consists of 125 μl 1M EGTA, 125 μl dH₂O, 110 μl 10% SDS, 50 μl 1M Tris (pH 7.4), 50 μl 1M DTT, and 40 μl 1M MgCL₂. These three solutions were mixed together in a ratio of 2 parts TTP, 1 part poly rA:oligo dT, and 1 part reaction buffer. Ten microliters of this reactions mixture was placed at a round bottom microtiter plate and 15 μl of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times 5 minutes each in a 5% sodium phosphate buffer, 2 times 1 minute each in distilled water, 2 times for 1 minute each in 70% ethanol, and then dried. Opti-Fluor-O (Packard) was added to each sample and incorporated radioactivity was quantified utilizing a Wallac 1450 MicroBeta Plus liquid scintillation counter.

### 4. Cytotoxicity Measurement by MTS Staining

At assay termination the assay plates were stained with the soluble tetrazolium-based dye MTS (CellTiter Reagent, Promega) to determine cell viability and quantify compound toxicity. MTS is metabolized by the mitochondria enzymes of metabolically active cells to yield a soluble formazan product, allowing the rapid quantitative analysis cell viability and compound cytotoxicity. The MTS is a stable solution that does not require preparation before use. At termination of the assay, 20 μl of MTS reagent was added per well. The wells were incubated overnight for the HIV cytoprotection assay at 37° C. The incubation intervals were chosen based on empirically determined times for optimal dye reduction in each cell type. Adhesive plate sealers were used in place of the lids, the sealed plate was inverted several times to mix the soluble formazan product and the plate was read spectrophotometrically at 490 nm with a Molecular Devices Vmax plate reader.

### 5. Data Analysis

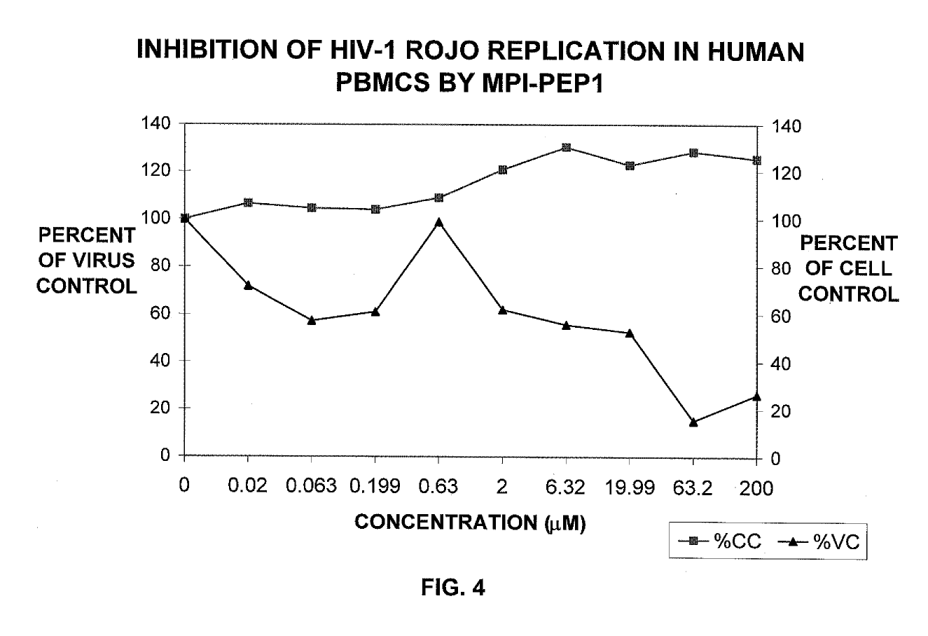

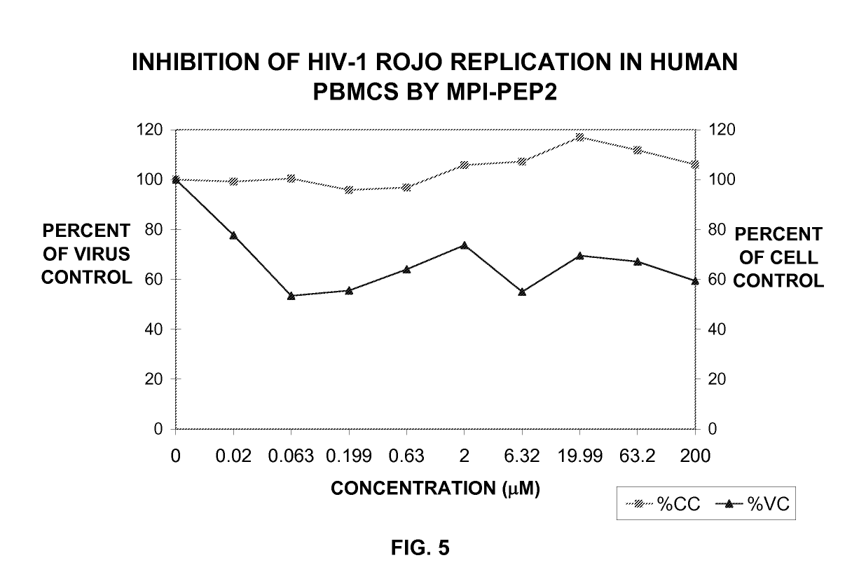

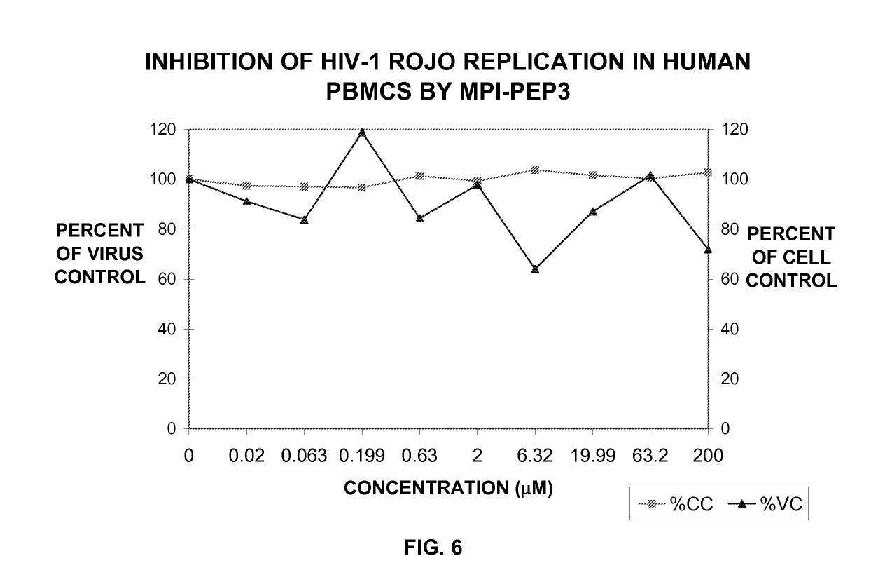

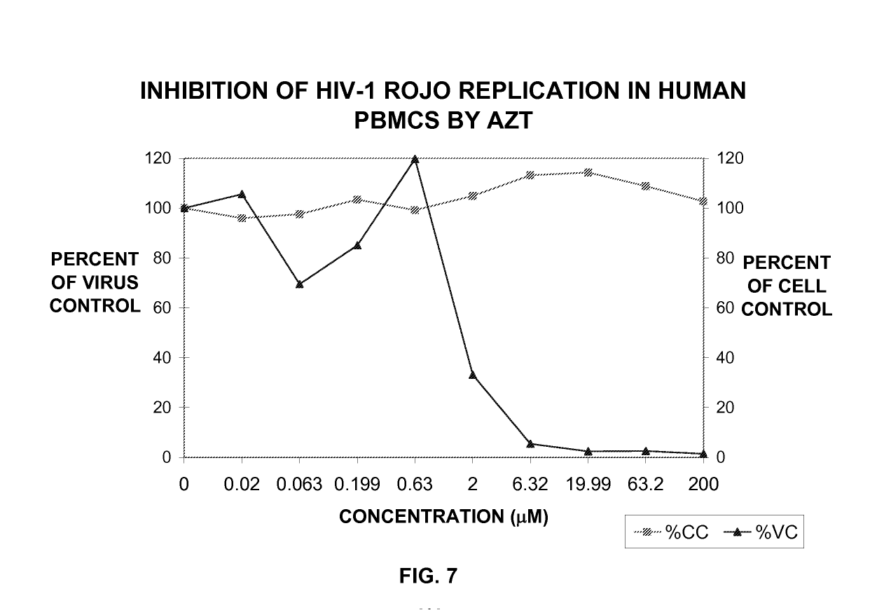

Indices including % CPE Reduction, % Cell Viability, IC₅₀, TC₅₀, and others were calculated and summarized in Table 4 below. The graphical results for the three peptidic compounds tested are displayed in FIGS. 4, 5 and 6, respectively. AZT was evaluated in parallel as a relevant positive control compound in the anti-HIV assay, and the graphical result is shown in FIG. 7.

TABLE 29

| Compound | | | Therapeutic | |
|---|---|---|---|---|
| Name | IC₅₀ (μM) | TC₅₀ (μM) | Index | Comments |
| MPI-PEP1 | 21.7 | >200.0 | 9.2 | Active |
| MPI-PEP2 | >200.0 | >200.0 | N/A | Inactive |
| MPI-PEP3 | >200.0 | >200.0 | N/A | Inactive |
| AZT | 0.008 | >1.0 | >125.00 | Control; Highly |
| | | | | Active |

### Example 4

This demonstrates the efficacy assay for the anti-HBV effect of test compound, e.g., the compounds used in Example 3. The assay is similar to the assay described by Korba and Milman, Antiviral Res., 15:217-228 (1991) and Korba and Gerin, Antiviral Res., 19:55-70 (1992), with the exception that viral DNA detection and quantification is dramatically simplified. Briefly, HepG2-2.2.15 cells are plated in 96-well microtiter plates at an initial density of 2×10⁴ cells/100 μl in DMEM medium supplemented with 10% fetal bovine serum. To promote cell adherence, the 96-well plates have been pre-coated with collagen prior to cell plating. After incubation at 37° C. in a humidified, 5% CO₂ environment for 16-24 hours, the confluent monolayer of HepG2-2.2.15 cells is washed and the medium is replaced with complete medium containing various concentrations of test compound. Every three days, the culture medium is replaced with fresh medium containing the appropriately diluted drug. Nine days following the initial administration of test compounds, the cell culture supernate is collected and clarified by centrifugation (Sorvall RT-6000D centrifuge, 1000 rpm for 5 min). Three microliters of clarified supernate is then subjected to real-time quantitative PCR using conditions described below.

Virion-associated HBV DNA present in the tissue culture supernate is PCR amplified using primers derived from HBV strain ayw. Subsequently, the PCR-amplified HBV DNA is detected in real-time (i.e., at each PCR thermocycle step) by monitoring increases in fluorescence signals that result from exonucleolytic degradation of a quenched fluorescent probe molecule following hybridization of the probe to the amplified HBV DNA. The probe molecule, designed with the aid of Primer Express™ (PE-Applied Biosystems) software, is complementary to DNA sequences present in the HBV DNA region amplified.

Routinely, 3 μl of clarified supernate is analyzed directly (without DNA extraction) in a 50 μl PCR reaction. Reagents and conditions used are per the manufacturers suggestions (PE-Applied Biosystems). For each PCR amplification, a standard curve is simultaneously generated several log dilutions of a purified 1.2 kbp HBVayw subgenomic fragment; routinely, the standard curve ranged from 1×10⁶ to 1×10¹ nominal copy equivalents per PCR reaction.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 2
P(T/S)AP Motif Containing Peptides from
Ebola Virus Matrix Protein
(GenBank Accession No. AAL25816)

| | |
|---|---|
| SEQ ID NO: 38 | RVILPTAP |
| SEQ ID NO: 39 | VILPTAPP |
| SEQ ID NO: 40 | ILPTAPPE |
| SEQ ID NO: 41 | LPTAPPEY |
| SEQ ID NO: 42 | PTAPPEYM |
| SEQ ID NO: 43 | RRVILPTAP |
| SEQ ID NO: 44 | RVILPTAPP |
| SEQ ID NO: 45 | VILPTAPPE |
| SEQ ID NO: 46 | ILPTAPPEY |
| SEQ ID NO: 47 | LPTAPPEYM |
| SEQ ID NO: 48 | PTAPPEYME |
| SEQ ID NO: 49 | MRRVILPTAP |
| SEQ ID NO: 50 | RRVILPTAPP |
| SEQ ID NO: 51 | RVILPTAPPE |
| SEQ ID NO: 52 | VILPTAPPEY |
| SEQ ID NO: 53 | ILPTAPPEYM |
| SEQ ID NO: 54 | LPTAPPEYME |
| SEQ ID NO: 55 | PTAPPEYMEA |
| SEQ ID NO: 56 | MRRVILPTAPP |
| SEQ ID NO: 57 | RRVILPTAPPE |
| SEQ ID NO: 58 | RVILPTAPPEY |
| SEQ ID NO: 59 | VILPTAPPEYM |
| SEQ ID NO: 60 | ILPTAPPEYME |
| SEQ ID NO: 61 | LPTAPPEYMEA |
| SEQ ID NO: 62 | PTAPPEYMEAI |
| SEQ ID NO: 63 | MRRVILPTAPPE |
| SEQ ID NO: 64 | RRVILPTAPPEY |
| SEQ ID NO: 65 | RVILPTAPPEYM |
| SEQ ID NO: 66 | VILPTAPPEYME |
| SEQ ID NO: 67 | ILPTAPPEYMEA |
| SEQ ID NO: 68 | LPTAPPEYMEAI |
| SEQ ID NO: 69 | PTAPPEYMEAIY |
| SEQ ID NO: 70 | MRRVILPTAPPEY |
| SEQ ID NO: 71 | RRVILPTAPPEYM |
| SEQ ID NO: 72 | RVILPTAPPEYME |
| SEQ ID NO: 73 | VILPTAPPEYMEA |
| SEQ ID NO: 74 | ILPTAPPEYMEAI |
| SEQ ID NO: 75 | LPTAPPEYMEAIY |
| SEQ ID NO: 76 | PTAPPEYMEAIYP |
| SEQ ID NO: 77 | MRRVILPTAPPEYM |
| SEQ ID NO: 78 | RRVILPTAPPEYME |
| SEQ ID NO: 79 | RVILPTAPPEYMEA |
| SEQ ID NO: 80 | VILPTAPPEYMEAI |
| SEQ ID NO: 81 | ILPTAPPEYMEAIY |
| SEQ ID NO: 82 | LPTAPPEYMEAIYP |
| SEQ ID NO: 83 | PTAPPEYMEAIYPV |
| SEQ ID NO: 84 | MRRVILPTAPPEYME |
| SEQ ID NO: 85 | RRVILPTAPPEYMEA |
| SEQ ID NO: 86 | RVILPTAPPEYMEAI |
| SEQ ID NO: 87 | VILPTAPPEYMEAIY |
| SEQ ID NO: 88 | ILPTAPPEYMEAIYP |
| SEQ ID NO: 89 | LPTAPPEYMEAIYPV |
| SEQ ID NO: 90 | PTAPPEYMEAIYPVR |
| SEQ ID NO: 91 | MRRVILPTAPPEYMEA |
| SEQ ID NO: 92 | RRVILPTAPPEYMEAI |
| SEQ ID NO: 93 | RVILPTAPPEYMEAIY |
| SEQ ID NO: 94 | VILPTAPPEYMEAIYP |
| SEQ ID NO: 95 | ILPTAPPEYMEAIYPV |
| SEQ ID NO: 96 | LPTAPPEYMEAIYPVR |
| SEQ ID NO: 97 | PTAPPEYMEAIYPVRS |
| SEQ ID NO: 98 | MRRVILPTAPPEYMEAI |
| SEQ ID NO: 99 | RRVILPTAPPEYMEAIY |
| SEQ ID NO: 100 | RVILPTAPPEYMEAIYP |
| SEQ ID NO: 101 | VILPTAPPEYMEAIYPV |
| SEQ ID NO: 102 | ILPTAPPEYMEAIYPVR |
| SEQ ID NO: 103 | LPTAPPEYMEAIYPVRS |
| SEQ ID NO: 104 | PTAPPEYMEAIYPVRSN |
| SEQ ID NO: 105 | MRRVILPTAPPEYMEAIY |
| SEQ ID NO: 106 | RRVILPTAPPEYMEAIYP |
| SEQ ID NO: 107 | RVILPTAPPEYMEAIYPV |
| SEQ ID NO: 108 | VILPTAPPEYMEAIYPVR |
| SEQ ID NO: 109 | ILPTAPPEYMEAIYPVRS |
| SEQ ID NO: 110 | LPTAPPEYMEAIYPVRSN |
| SEQ ID NO: 111 | PTAPPEYMEAIYPVRSNS |
| SEQ ID NO: 112 | MRRVILPTAPPEYMEAIYP |
| SEQ ID NO: 113 | RRVILPTAPPEYMEAIYPV |
| SEQ ID NO: 114 | RVILPTAPPEYMEAIYPVR |
| SEQ ID NO: 115 | VILPTAPPEYMEAIYPVRS |
| SEQ ID NO: 116 | ILPTAPPEYMEAIYPVRSN |
| SEQ ID NO: 117 | LPTAPPEYMEAIYPVRSNS |
| SEQ ID NO: 118 | PTAPPEYMEAIYPVRSNST |
| SEQ ID NO: 119 | MRRVILPTAPPEYMEAIYPVV |
| SEQ ID NO: 120 | RRVILPTAPPEYMEAIYPVR |
| SEQ ID NO: 121 | RVILPTAPPEYMEAIYPVRS |
| SEQ ID NO: 122 | VILPTAPPEYMEAIYPVRSN |
| SEQ ID NO: 123 | ILPTAPPEYMEAIYPVRSNS |
| SEQ ID NO: 124 | LPTAPPEYMEAIYPVRSNST |
| SEQ ID NO: 125 | PTAPPEYMEAIYPVRSNSTI |

TABLE 3
P(T/S)AP Motif Containing Peptides from
Hepatitis B Virus PreS1/PreS2/S Envelope Protein
(GenBank Accession No. BAA85340)

| | |
|---|---|
| SEQ ID NO: 126 | LTTVPTAP |
| SEQ ID NO: 127 | TTVPTAPP |
| SEQ ID NO: 128 | TVPTAPPP |
| SEQ ID NO: 129 | VPTAPPPA |
| SEQ ID NO: 130 | PTAPPPAS |
| SEQ ID NO: 131 | ILTTVPTAP |
| SEQ ID NO: 132 | LTTVPTAPP |
| SEQ ID NO: 133 | TTVPTAPPP |
| SEQ ID NO: 134 | TVPTAPPPA |
| SEQ ID NO: 135 | VPTAPPPAS |
| SEQ ID NO: 136 | PTAPPPAST |
| SEQ ID NO: 137 | GILTTVPTAP |
| SEQ ID NO: 138 | ILTTVPTAPP |
| SEQ ID NO: 139 | LTTVPTAPPP |
| SEQ ID NO: 140 | TTVPTAPPPA |
| SEQ ID NO: 141 | TVPTAPPPAS |
| SEQ ID NO: 142 | VPTAPPPAST |
| SEQ ID NO: 143 | PTAPPPASTN |
| SEQ ID NO: 144 | QGILTTVPTAP |
| SEQ ID NO: 145 | GILTTVPTAPP |
| SEQ ID NO: 146 | ILTTVPTAPPP |
| SEQ ID NO: 147 | LTTVPTAPPPA |
| SEQ ID NO: 148 | TTVPTAPPPAS |
| SEQ ID NO: 149 | TVPTAPPPAST |
| SEQ ID NO: 150 | VPTAPPPASTN |
| SEQ ID NO: 151 | PTAPPPASTNR |
| SEQ ID NO: 152 | AQGILTTVPTAP |
| SEQ ID NO: 153 | QGILTTVPTAPP |
| SEQ ID NO: 154 | GILTTVPTAPPP |
| SEQ ID NO: 155 | ILTTVPTAPPPA |
| SEQ ID NO: 156 | LTTVPTAPPPAS |
| SEQ ID NO: 157 | TTVPTAPPPAST |
| SEQ ID NO: 158 | TVPTAPPPASTN |
| SEQ ID NO: 159 | VPTAPPPASTNR |
| SEQ ID NO: 160 | PTAPPPASTNRQ |
| SEQ ID NO: 161 | QAQGILTTVPTAP |
| SEQ ID NO: 162 | AQGILTTVPTAPP |
| SEQ ID NO: 163 | QGILTTVPTAPPP |
| SEQ ID NO: 164 | GILTTVPTAPPPA |
| SEQ ID NO: 165 | ILTTVPTAPPPAS |
| SEQ ID NO: 166 | LTTVPTAPPPAST |
| SEQ ID NO: 167 | TTVPTAPPPASTN |
| SEQ ID NO: 168 | TVPTAPPPASTNR |
| SEQ ID NO: 169 | VPTAPPPASTNRQ |
| SEQ ID NO: 170 | PTAPPPASTNRQL |
| SEQ ID NO: 171 | PQAQGILTTVPTAP |
| SEQ ID NO: 172 | QAQGILTTVPTAPP |
| SEQ ID NO: 173 | AQGILTTVPTAPPP |
| SEQ ID NO: 174 | QGILTTVPTAPPPA |
| SEQ ID NO: 175 | GILTTVPTAPPPAS |
| SEQ ID NO: 176 | ILTTVPTAPPPAST |
| SEQ ID NO: 177 | LTTVPTAPPPASTN |
| SEQ ID NO: 178 | TTVPTAPPPASTNR |
| SEQ ID NO: 179 | TVPTAPPPASTNRQ |
| SEQ ID NO: 180 | VPTAPPPASTNRQL |
| SEQ ID NO: 181 | PTAPPPASTNRQLG |
| SEQ ID NO: 182 | SPQAQGILTTVPTAP |
| SEQ ID NO: 183 | PQAQGILTTVPTAPP |
| SEQ ID NO: 184 | QAQGILTTVPTAPPP |
| SEQ ID NO: 185 | AQGILTTVPTAPPPA |
| SEQ ID NO: 186 | QGILTTVPTAPPPAS |
| SEQ ID NO: 187 | GILTTVPTAPPPAST |
| SEQ ID NO: 188 | ILTTVPTAPPPASTN |
| SEQ ID NO: 189 | LTTVPTAPPPASTNR |
| SEQ ID NO: 190 | TTVPTAPPPASTNRQ |
| SEQ ID NO: 191 | TVPTAPPPASTNRQL |
| SEQ ID NO: 192 | VPTAPPPASTNRQLG |
| SEQ ID NO: 193 | PTAPPPASTNRQLGR |
| SEQ ID NO: 194 | WSPQAQGILTTVPTAP |
| SEQ ID NO: 195 | SPQAQGILTTVPTAPP |
| SEQ ID NO: 196 | PQAQGILTTVPTAPPP |
| SEQ ID NO: 197 | QAQGILTTVPTAPPPA |
| SEQ ID NO: 198 | AQGILTTVPTAPPPAS |
| SEQ ID NO: 199 | QGILTTVPTAPPPAST |
| SEQ ID NO: 200 | GILTTVPTAPPPASTN |
| SEQ ID NO: 201 | ILTTVPTAPPPASTNR |
| SEQ ID NO: 202 | LTTVPTAPPPASTNRQ |
| SEQ ID NO: 203 | TTVPTAPPPASTNRQL |
| SEQ ID NO: 204 | TVPTAPPPASTNRQLG |
| SEQ ID NO: 205 | VPTAPPPASTNRQLGR |
| SEQ ID NO: 206 | PTAPPPASTNRQLGRK |
| SEQ ID NO: 207 | GWSPQAQGILTTVPTAP |
| SEQ ID NO: 208 | WSPQAQGILTTVPTAPP |
| SEQ ID NO: 209 | SPQAQGILTTVPTAPPP |
| SEQ ID NO: 210 | PQAQGILTTVPTAPPPA |
| SEQ ID NO: 211 | QAQGILTTVPTAPPPAS |
| SEQ ID NO: 212 | AQGILTTVPTAPPPAST |
| SEQ ID NO: 213 | QGILTTVPTAPPPASTN |
| SEQ ID NO: 214 | GILTTVPTAPPPASTNR |
| SEQ ID NO: 215 | ILTTVPTAPPPASTNRQ |
| SEQ ID NO: 216 | LTTVPTAPPPASTNRQL |
| SEQ ID NO: 217 | TTVPTAPPPASTNRQLG |
| SEQ ID NO: 218 | TVPTAPPPASTNRQLGR |
| SEQ ID NO: 219 | VPTAPPPASTNRQLGRK |
| SEQ ID NO: 220 | PTAPPPASTNRQLGRKP |
| SEQ ID NO: 221 | LGWSPQAQGILTTVPTAP |
| SEQ ID NO: 222 | GWSPQAQGILTTVPTAPP |
| SEQ ID NO: 223 | WSPQAQGILTTVPTAPPP |
| SEQ ID NO: 224 | SPQAQGILTTVPTAPPPA |
| SEQ ID NO: 225 | PQAQGILTTVPTAPPPAS |
| SEQ ID NO: 226 | QAQGILTTVPTAPPPAST |
| SEQ ID NO: 227 | AQGILTTVPTAPPPASTN |
| SEQ ID NO: 228 | QGILTTVPTAPPPASTNR |
| SEQ ID NO: 229 | GILTTVPTAPPPASTNRQ |
| SEQ ID NO: 230 | ILTTVPTAPPPASTNRQL |
| SEQ ID NO: 231 | LTTVPTAPPPASTNRQLG |
| SEQ ID NO: 232 | TTVPTAPPPASTNRQLGR |
| SEQ ID NO: 233 | TVPTAPPPASTNRQLGRK |
| SEQ ID NO: 234 | VPTAPPPASTNRQLGRKP |
| SEQ ID NO: 235 | PTAPPPASTNRQLGRKPT |
| SEQ ID NO: 236 | LLGWSPQAQGILTTVPTAP |
| SEQ ID NO: 237 | LGWSPQAQGILTTVPTAPP |
| SEQ ID NO: 238 | GWSPQAQGILTTVPTAPPP |
| SEQ ID NO: 239 | WSPQAQGILTTVPTAPPPA |
| SEQ ID NO: 240 | SPQAQGILTTVPTAPPPAS |
| SEQ ID NO: 241 | PQAQGILTTVPTAPPPAST |
| SEQ ID NO: 242 | QAQGILTTVPTAPPPASTN |
| SEQ ID NO: 243 | AQGILTTVPTAPPPASTNR |
| SEQ ID NO: 244 | QGILTTVPTAPPPASTNRQ |
| SEQ ID NO: 245 | GILTTVPTAPPPASTNRQL |
| SEQ ID NO: 246 | ILTTVPTAPPPASTNRQLG |
| SEQ ID NO: 247 | LTTVPTAPPPASTNRQLGR |
| SEQ ID NO: 248 | TTVPTAPPPASTNRQLGRK |
| SEQ ID NO: 249 | TVPTAPPPASTNRQLGRKP |
| SEQ ID NO: 250 | VPTAPPPASTNRQLGRKPT |
| SEQ ID NO: 251 | PTAPPPASTNRQLGRKPTP |
| SEQ ID NO: 252 | GLLGWSPQAQGILTTVPTAP |
| SEQ ID NO: 253 | LLGWSPQAQGILTTVPTAPP |
| SEQ ID NO: 254 | LGWSPQAQGILTTVPTAPPP |
| SEQ ID NO: 255 | GWSPQAQGILTTVPTAPPPA |
| SEQ ID NO: 256 | WSPQAQGILTTVPTAPPPAS |
| SEQ ID NO: 257 | SPQAQGILTTVPTAPPPAST |
| SEQ ID NO: 258 | PQAQGILTTVPTAPPPASTN |
| SEQ ID NO: 259 | QAQGILTTVPTAPPPASTNR |
| SEQ ID NO: 260 | AQGILTTVPTAPPPASTNRQ |
| SEQ ID NO: 261 | QGILTTVPTAPPPASTNRQL |
| SEQ ID NO: 262 | GILTTVPTAPPPASTNRQLG |
| SEQ ID NO: 263 | ILTTVPTAPPPASTNRQLGR |
| SEQ ID NO: 264 | LTTVPTAPPPASTNRQLGRK |
| SEQ ID NO: 265 | TTVPTAPPPASTNRQLGRKP |
| SEQ ID NO: 266 | TVPTAPPPASTNRQLGRKPT |
| SEQ ID NO: 267 | VPTAPPPASTNRQLGRKPTP |
| SEQ ID NO: 268 | PTAPPPASTNRQLGRKPTPL |

TABLE 4
P(T/S)AP Motif Containing Peptides from
Human Herpesvirus 1 RL2 Protein
(GenBank Accession No. NP_044601)

| | |
|---|---|
| SEQ ID NO: 269 | RTAPPSAP |
| SEQ ID NO: 270 | TAPPSAPI |
| SEQ ID NO: 271 | APPSAPIG |
| SEQ ID NO: 272 | PPSAPIGP |
| SEQ ID NO: 273 | PSAPIGPH |
| SEQ ID NO: 274 | ARTAPPSAP |
| SEQ ID NO: 275 | RTAPPSAPI |
| SEQ ID NO: 276 | TAPPSAPIG |
| SEQ ID NO: 277 | APPSAPIGP |
| SEQ ID NO: 278 | PPSAPIGPH |
| SEQ ID NO: 279 | PSAPIGPHG |
| SEQ ID NO: 280 | AARTAPPSAP |
| SEQ ID NO: 281 | ARTAPPSAPI |
| SEQ ID NO: 282 | RTAPPSAPIG |
| SEQ ID NO: 283 | TAPPSAPIGP |
| SEQ ID NO: 284 | APPSAPIGPH |
| SEQ ID NO: 285 | PPSAPIGPHG |
| SEQ ID NO: 286 | PSAPIGPHGS |
| SEQ ID NO: 287 | AAARTAPPSAP |
| SEQ ID NO: 288 | AARTAPPSAPI |
| SEQ ID NO: 289 | ARTAPPSAPIG |
| SEQ ID NO: 290 | RTAPPSAPIGP |
| SEQ ID NO: 291 | TAPPSAPIGPH |
| SEQ ID NO: 292 | APPSAPIGPHG |
| SEQ ID NO: 293 | PPSAPIGPHGS |
| SEQ ID NO: 294 | PSAPIGPHGSS |
| SEQ ID NO: 295 | PAAARTAPPSAP |
| SEQ ID NO: 296 | AAARTAPPSAPI |
| SEQ ID NO: 297 | AARTAPPSAPIG |
| SEQ ID NO: 298 | ARTAPPSAPIGP |
| SEQ ID NO: 299 | RTAPPSAPIGPH |
| SEQ ID NO: 300 | TAPPSAPIGPHG |
| SEQ ID NO: 301 | APPSAPIGPHGS |
| SEQ ID NO: 302 | PPSAPIGPHGSS |
| SEQ ID NO: 303 | PSAPIGPHGSSN |
| SEQ ID NO: 304 | QPAAARTAPPSAP |
| SEQ ID NO: 305 | PAAARTAPPSAPI |
| SEQ ID NO: 306 | AAARTAPPSAPIG |
| SEQ ID NO: 307 | AARTAPPSAPIGP |
| SEQ ID NO: 308 | ARTAPPSAPIGPH |
| SEQ ID NO: 309 | RTAPPSAPIGPHG |
| SEQ ID NO: 310 | TAPPSAPIGPHGS |
| SEQ ID NO: 311 | APPSAPIGPHGSS |
| SEQ ID NO: 312 | PPSAPIGPHGSSN |
| SEQ ID NO: 313 | PSAPIGPHGSSNT |
| SEQ ID NO: 314 | PQPAAARTAPPSAP |
| SEQ ID NO: 315 | QPAAARTAPPSAPI |
| SEQ ID NO: 316 | PAAARTAPPSAPIG |
| SEQ ID NO: 317 | AAARTAPPSAPIGP |
| SEQ ID NO: 318 | AARTAPPSAPIGPH |
| SEQ ID NO: 319 | ARTAPPSAPIGPHG |
| SEQ ID NO: 320 | RTAPPSAPIGPHGS |
| SEQ ID NO: 321 | TAPPSAPIGPHGSS |
| SEQ ID NO: 322 | APPSAPIGPHGSSN |
| SEQ ID NO: 323 | PPSAPIGPHGSSNT |
| SEQ ID NO: 324 | PSAPIGPHGSSNTN |
| SEQ ID NO: 325 | APQPAAARTAPPSAP |
| SEQ ID NO: 326 | PQPAAARTAPPSAPI |
| SEQ ID NO: 327 | QPAAARTAPPSAPIG |
| SEQ ID NO: 328 | PAAARTAPPSAPIGP |
| SEQ ID NO: 329 | AAARTAPPSAPIGPH |
| SEQ ID NO: 330 | AARTAPPSAPIGPHG |
| SEQ ID NO: 331 | ARTAPPSAPIGPHGS |
| SEQ ID NO: 332 | RTAPPSAPIGPHGSS |
| SEQ ID NO: 333 | TAPPSAPIGPHGSSN |
| SEQ ID NO: 334 | APPSAPIGPHGSSNT |
| SEQ ID NO: 335 | PPSAPIGPHGSSNTN |
| SEQ ID NO: 336 | PSAPIGPHGSSNTNT |
| SEQ ID NO: 337 | AAPQPAAARTAPPSAP |
| SEQ ID NO: 338 | APQPAAARTAPPSAPI |
| SEQ ID NO: 339 | PQPAAARTAPPSAPIG |
| SEQ ID NO: 340 | QPAAARTAPPSAPIGP |
| SEQ ID NO: 341 | PAAARTAPPSAPIGPH |
| SEQ ID NO: 342 | AAARTAPPSAPIGPHG |
| SEQ ID NO: 343 | AARTAPPSAPIGPHGS |
| SEQ ID NO: 344 | ARTAPPSAPIGPHGSS |
| SEQ ID NO: 345 | RTAPPSAPIGPHGSSN |
| SEQ ID NO: 346 | TAPPSAPIGPHGSSNT |
| SEQ ID NO: 347 | APPSAPIGPHGSSNTN |
| SEQ ID NO: 348 | PPSAPIGPHGSSNTNT |
| SEQ ID NO: 349 | PSAPIGPHGSSNTNTT |
| SEQ ID NO: 350 | HAAPQPAAARTAPPSAP |
| SEQ ID NO: 351 | AAPQPAAARTAPPSAPI |
| SEQ ID NO: 352 | APQPAAARTAPPSAPIG |
| SEQ ID NO: 353 | PQPAAARTAPPSAPIGP |
| SEQ ID NO: 354 | QPAAARTAPPSAPIGPH |
| SEQ ID NO: 355 | PAAARTAPPSAPIGPHG |
| SEQ ID NO: 356 | AAARTAPPSAPIGPHGS |
| SEQ ID NO: 357 | AARTAPPSAPIGPHGSS |
| SEQ ID NO: 358 | ARTAPPSAPIGPHGSSN |
| SEQ ID NO: 359 | RTAPPSAPIGPHGSSNT |
| SEQ ID NO: 360 | TAPPSAPIGPHGSSNTN |
| SEQ ID NO: 361 | APPSAPIGPHGSSNTNT |
| SEQ ID NO: 362 | PPSAPIGPHGSSNTNTT |
| SEQ ID NO: 363 | PSAPIGPHGSSNTNTTT |
| SEQ ID NO: 364 | SHAAPQPAAARTAPPSAP |
| SEQ ID NO: 365 | HAAPQPAAARTAPPSAPI |
| SEQ ID NO: 366 | AAPQPAAARTAPPSAPIG |
| SEQ ID NO: 367 | APQPAAARTAPPSAPIGP |
| SEQ ID NO: 368 | PQPAAARTAPPSAPIGPH |
| SEQ ID NO: 369 | QPAAARTAPPSAPIGPHG |
| SEQ ID NO: 370 | PAAARTAPPSAPIGPHGS |
| SEQ ID NO: 371 | AAARTAPPSAPIGPHGSS |
| SEQ ID NO: 372 | AARTAPPSAPIGPHGSSN |
| SEQ ID NO: 373 | ARTAPPSAPIGPHGSSNT |
| SEQ ID NO: 374 | RTAPPSAPIGPHGSSNTN |
| SEQ ID NO: 375 | TAPPSAPIGPHGSSNTNT |
| SEQ ID NO: 376 | APPSAPIGPHGSSNTNTT |
| SEQ ID NO: 377 | PPSAPIGPHGSSNTNTTT |
| SEQ ID NO: 378 | PSAPIGPHGSSNTNTTTN |
| SEQ ID NO: 379 | ASHAAPQPAAARTAPPSAP |
| SEQ ID NO: 380 | SHAAPQPAAARTAPPSAPI |
| SEQ ID NO: 381 | HAAPQPAAARTAPPSAPIG |
| SEQ ID NO: 382 | AAPQPAAARTAPPSAPIGP |
| SEQ ID NO: 383 | APQPAAARTAPPSAPIGPH |
| SEQ ID NO: 384 | PQPAAARTAPPSAPIGPHG |
| SEQ ID NO: 385 | QPAAARTAPPSAPIGPHGS |
| SEQ ID NO: 386 | PAAARTAPPSAPIGPHGSS |
| SEQ ID NO: 387 | AAARTAPPSAPIGPHGSSN |
| SEQ ID NO: 388 | AARTAPPSAPIGPHGSSNT |
| SEQ ID NO: 389 | ARTAPPSAPIGPHGSSNTN |
| SEQ ID NO: 390 | RTAPPSAPIGPHGSSNTNT |
| SEQ ID NO: 391 | TAPPSAPIGPHGSSNTNTT |
| SEQ ID NO: 392 | APPSAPIGPHGSSNTNTTT |
| SEQ ID NO: 393 | PPSAPIGPHGSSNTNTTTN |
| SEQ ID NO: 394 | PSAPIGPHGSSNTNTTTNS |
| SEQ ID NO: 395 | GASHAAPQPAAARTAPPSAP |
| SEQ ID NO: 396 | ASHAAPQPAAARTAPPSAPI |
| SEQ ID NO: 397 | SHAAPQPAAARTAPPSAPIG |
| SEQ ID NO: 398 | HAAPQPAAARTAPPSAPIGP |
| SEQ ID NO: 399 | AAPQPAAARTAPPSAPIGPH |
| SEQ ID NO: 400 | APQPAAARTAPPSAPIGPHG |
| SEQ ID NO: 401 | PQPAAARTAPPSAPIGPHGS |
| SEQ ID NO: 402 | QPAAARTAPPSAPIGPHGSS |
| SEQ ID NO: 403 | PAAARTAPPSAPIGPHGSSN |
| SEQ ID NO: 404 | AAARTAPPSAPIGPHGSSNT |
| SEQ ID NO: 405 | AARTAPPSAPIGPHGSSNTN |
| SEQ ID NO: 406 | ARTAPPSAPIGPHGSSNTNT |
| SEQ ID NO: 407 | RTAPPSAPIGPHGSSNTNTT |
| SEQ ID NO: 408 | TAPPSAPIGPHGSSNTNTTT |
| SEQ ID NO: 409 | APPSAPIGPHGSSNTNTTTN |
| SEQ ID NO: 410 | PPSAPIGPHGSSNTNTTTNS |
| SEQ ID NO: 411 | PSAPIGPHGSSNTNTTTNSS |
| SEQ ID NO: 412 | PPEYPTAP |
| SEQ ID NO: 413 | PEYPTAPA |
| SEQ ID NO: 414 | EYPTAPAS |
| SEQ ID NO: 415 | YPTAPASE |
| SEQ ID NO: 416 | PTAPASEW |
| SEQ ID NO: 417 | MPPEYPTAP |
| SEQ ID NO: 418 | PPEYPTAPA |
| SEQ ID NO: 419 | PEYPTAPAS |
| SEQ ID NO: 420 | EYPTAPASE |
| SEQ ID NO: 421 | YPTAPASEW |
| SEQ ID NO: 422 | PTAPASEWN |
| SEQ ID NO: 423 | VMPPEYPTAP |
| SEQ ID NO: 424 | MPPEYPTAPA |
| SEQ ID NO: 425 | PPEYPTAPAS |
| SEQ ID NO: 426 | PEYPTAPASE |
| SEQ ID NO: 427 | EYPTAPASEW |
| SEQ ID NO: 428 | YPTAPASEWN |
| SEQ ID NO: 429 | PTAPASEWNS |
| SEQ ID NO: 430 | HVMPPEYPTAP |
| SEQ ID NO: 431 | VMPPEYPTAPA |
| SEQ ID NO: 432 | MPPEYPTAPAS |
| SEQ ID NO: 433 | PPEYPTAPASE |
| SEQ ID NO: 434 | PEYPTAPASEW |
| SEQ ID NO: 435 | EYPTAPASEWN |
| SEQ ID NO: 436 | YPTAPASEWNS |
| SEQ ID NO: 437 | PTAPASEWNSL |
| SEQ ID NO: 438 | NHVMPPEYPTAP |
| SEQ ID NO: 439 | HVMPPEYPTAPA |
| SEQ ID NO: 440 | VMPPEYPTAPAS |
| SEQ ID NO: 441 | MPPEYPTAPASE |
| SEQ ID NO: 442 | PPEYPTAPASEW |
| SEQ ID NO: 443 | PEYPTAPASEWN |
| SEQ ID NO: 444 | EYPTAPASEWNS |
| SEQ ID NO: 445 | YPTAPASEWNSL |
| SEQ ID NO: 446 | PTAPASEWNSLW |
| SEQ ID NO: 447 | GNHVMPPEYPTAP |
| SEQ ID NO: 448 | NHVMPPEYPTAPA |
| SEQ ID NO: 449 | HVMPPEYPTAPAS |
| SEQ ID NO: 450 | VMPPEYPTAPASE |
| SEQ ID NO: 451 | MPPEYPTAPASEW |
| SEQ ID NO: 452 | PPEYPTAPASEWN |
| SEQ ID NO: 453 | PEYPTAPASEWNS |
| SEQ ID NO: 454 | EYPTAPASEWNSL |
| SEQ ID NO: 455 | YPTAPASEWNSLW |
| SEQ ID NO: 456 | PTAPASEWNSLWM |
| SEQ ID NO: 457 | AGNHVMPPEYPTAP |
| SEQ ID NO: 458 | GNHVMPPEYPTAPA |
| SEQ ID NO: 459 | NHVMPPEYPTAPAS |
| SEQ ID NO: 460 | HVMPPEYPTAPASE |
| SEQ ID NO: 461 | VMPPEYPTAPASEW |
| SEQ ID NO: 462 | MPPEYPTAPASEWN |
| SEQ ID NO: 463 | PPEYPTAPASEWNS |
| SEQ ID NO: 464 | PEYPTAPASEWNSL |
| SEQ ID NO: 465 | EYPTAPASEWNSLW |
| SEQ ID NO: 466 | YPTAPASEWNSLWM |
| SEQ ID NO: 467 | PTAPASEWNSLWMT |
| SEQ ID NO: 468 | TAGNHVMPPEYPTAP |
| SEQ ID NO: 469 | AGNHVMPPEYPTAPA |
| SEQ ID NO: 470 | GNHVMPPEYPTAPAS |
| SEQ ID NO: 471 | NHVMPPEYPTAPASE |
| SEQ ID NO: 472 | HVMPPEYPTAPASEW |
| SEQ ID NO: 473 | VMPPEYPTAPASEWN |
| SEQ ID NO: 474 | MPPEYPTAPASEWNS |
| SEQ ID NO: 475 | PPEYPTAPASEWNSL |
| SEQ ID NO: 476 | PEYPTAPASEWNSLW |
| SEQ ID NO: 477 | EYPTAPASEWNSLWM |
| SEQ ID NO: 478 | YPTAPASEWNSLWMT |
| SEQ ID NO: 479 | PTAPASEWNSLWMTP |
| SEQ ID NO: 480 | ETAGNHVMPPEYPTAP |
| SEQ ID NO: 481 | TAGNHVMPPEYPTAPA |
| SEQ ID NO: 482 | AGNHVMPPEYPTAPAS |
| SEQ ID NO: 483 | GNHVMPPEYPTAPASE |
| SEQ ID NO: 484 | NHVMPPEYPTAPASEW |
| SEQ ID NO: 485 | HVMPPEYPTAPASEWN |
| SEQ ID NO: 486 | VMPPEYPTAPASEWNS |
| SEQ ID NO: 487 | MPPEYPTAPASEWNSL |
| SEQ ID NO: 488 | PPEYPTAPASEWNSLW |
| SEQ ID NO: 489 | PEYPTAPASEWNSLWM |
| SEQ ID NO: 490 | EYPTAPASEWNSLWMT |
| SEQ ID NO: 491 | YPTAPASEWNSLWMTP |
| SEQ ID NO: 492 | PTAPASEWNSLWMTPV |
| SEQ ID NO: 493 | PETAGNHVMPPEYPTAP |
| SEQ ID NO: 494 | ETAGNHVMPPEYPTAPA |
| SEQ ID NO: 495 | TAGNHVMPPEYPTAPAS |
| SEQ ID NO: 496 | AGNHVMPPEYPTAPASE |
| SEQ ID NO: 497 | GNHVMPPEYPTAPASEW |
| SEQ ID NO: 498 | NHVMPPEYPTAPASEWN |
| SEQ ID NO: 499 | HVMPPEYPTAPASEWNS |
| SEQ ID NO: 500 | VMPPEYPTAPASEWNSL |
| SEQ ID NO: 501 | MPPEYPTAPASEWNSLW |
| SEQ ID NO: 502 | PPEYPTAPASEWNSLWM |
| SEQ ID NO: 503 | PEYPTAPASEWNSLWMT |
| SEQ ID NO: 504 | EYPTAPASEWNSLWMTP |
| SEQ ID NO: 505 | YPTAPASEWNSLWMTPV |
| SEQ ID NO: 506 | PTAPASEWNSLWMTPVG |
| SEQ ID NO: 507 | LPETAGNHVMPPEYPTAP |
| SEQ ID NO: 508 | PETAGNHVMPPEYPTAPA |
| SEQ ID NO: 509 | ETAGNHVMPPEYPTAPAS |
| SEQ ID NO: 510 | TAGNHVMPPEYPTAPASE |
| SEQ ID NO: 511 | AGNHVMPPEYPTAPASEW |
| SEQ ID NO: 512 | GNHVMPPEYPTAPASEWN |
| SEQ ID NO: 513 | NHVMPPEYPTAPASEWNS |
| SEQ ID NO: 514 | HVMPPEYPTAPASEWNSL |
| SEQ ID NO: 515 | VMPPEYPTAPASEWNSLW |
| SEQ ID NO: 516 | MPPEYPTAPASEWNSLWM |
| SEQ ID NO: 517 | PPEYPTAPASEWNSLWMT |
| SEQ ID NO: 518 | PEYPTAPASEWNSLWMTP |
| SEQ ID NO: 519 | EYPTAPASEWNSLWMTPV |
| SEQ ID NO: 520 | YPTAPASEWNSLWMTPVG |
| SEQ ID NO: 521 | PTAPASEWNSLWMTPVGN |
| SEQ ID NO: 522 | LLPETAGNHVMPPEYPTAP |
| SEQ ID NO: 523 | LPETAGNHVMPPEYPTAPA |
| SEQ ID NO: 524 | PETAGNHVMPPEYPTAPAS |
| SEQ ID NO: 525 | ETAGNHVMPPEYPTAPASE |
| SEQ ID NO: 526 | TAGNHVMPPEYPTAPASEW |
| SEQ ID NO: 527 | AGNHVMPPEYPTAPASEWN |
| SEQ ID NO: 528 | GNHVMPPEYPTAPASEWNS |
| SEQ ID NO: 529 | NHVMPPEYPTAPASEWNSL |
| SEQ ID NO: 530 | HVMPPEYPTAPASEWNSLW |
| SEQ ID NO: 531 | VMPPEYPTAPASEWNSLWM |
| SEQ ID NO: 532 | MPPEYPTAPASEWNSLWMT |
| SEQ ID NO: 533 | PPEYPTAPASEWNSLWMTP |
| SEQ ID NO: 534 | PEYPTAPASEWNSLWMTPV |
| SEQ ID NO: 535 | EYPTAPASEWNSLWMTPVG |
| SEQ ID NO: 536 | YPTAPASEWNSLWMTPVGN |
| SEQ ID NO: 537 | PTAPASEWNSLWMTPVGNM |
| SEQ ID NO: 538 | TLLPETAGNHVMPPEYPTAP |
| SEQ ID NO: 539 | LLPETAGNHVMPPEYPTAPA |
| SEQ ID NO: 540 | LPETAGNHVMPPEYPTAPAS |
| SEQ ID NO: 541 | PETAGNHVMPPEYPTAPASE |
| SEQ ID NO: 542 | ETAGNHVMPPEYPTAPASEW |
| SEQ ID NO: 543 | TAGNHVMPPEYPTAPASEWN |
| SEQ ID NO: 544 | AGNHVMPPEYPTAPASEWNS |
| SEQ ID NO: 545 | GNHVMPPEYPTAPASEWNSL |
| SEQ ID NO: 546 | NHVMPPEYPTAPASEWNSLW |
| SEQ ID NO: 547 | HVMPPEYPTAPASEWNSLWM |
| SEQ ID NO: 548 | VMPPEYPTAPASEWNSLWMT |
| SEQ ID NO: 549 | MPPEYPTAPASEWNSLWMTP |
| SEQ ID NO: 550 | PPEYPTAPASEWNSLWMTPV |
| SEQ ID NO: 551 | PEYPTAPASEWNSLWMTPVG |
| SEQ ID NO: 552 | EYPTAPASEWNSLWMTPVGN |
| SEQ ID NO: 553 | YPTAPASEWNSLWMTPVGNM |
| SEQ ID NO: 554 | PTAPASEWNSLWMTPVGNML |
| SEQ ID NO: 555 | FLGPPTAP |
| SEQ ID NO: 556 | LGPPTAPP |
| SEQ ID NO: 557 | GPPTAPPG |
| SEQ ID NO: 558 | PPTAPPGG |
| SEQ ID NO: 559 | PTAPPGGA |
| SEQ ID NO: 560 | LFLGPPTAP |
| SEQ ID NO: 561 | FLGPPTAPP |
| SEQ ID NO: 562 | LGPPTAPPG |
| SEQ ID NO: 563 | GPPTAPPGG |
| SEQ ID NO: 564 | PPTAPPGGA |
| SEQ ID NO: 565 | PTAPPGGAW |
| SEQ ID NO: 566 | LLFLGPPTAP |
| SEQ ID NO: 567 | LFLGPPTAPP |
| SEQ ID NO: 568 | FLGPPTAPPG |
| SEQ ID NO: 569 | LGPPTAPPGG |
| SEQ ID NO: 570 | GPPTAPPGGA |
| SEQ ID NO: 571 | PPTAPPGGAW |
| SEQ ID NO: 572 | PTAPPGGAWT |
| SEQ ID NO: 573 | TLLFLGPPTAP |
| SEQ ID NO: 574 | LLFLGPPTAPP |
| SEQ ID NO: 575 | LFLGPPTAPPG |
| SEQ ID NO: 576 | FLGPPTAPPGG |
| SEQ ID NO: 577 | LGPPTAPPGGA |
| SEQ ID NO: 578 | GPPTAPPGGAW |
| SEQ ID NO: 579 | PPTAPPGGAWT |
| SEQ ID NO: 580 | PTAPPGGAWTP |
| SEQ ID NO: 581 | QTLLFLGPPTAP |
| SEQ ID NO: 582 | TLLFLGPPTAPP |
| SEQ ID NO: 583 | LLFLGPPTAPPG |
| SEQ ID NO: 584 | LFLGPPTAPPGG |
| SEQ ID NO: 585 | FLGPPTAPPGGA |
| SEQ ID NO: 586 | LGPPTAPPGGAW |
| SEQ ID NO: 587 | GPPTAPPGGAWT |
| SEQ ID NO: 588 | PPTAPPGGAWTP |
| SEQ ID NO: 589 | PTAPPGGAWTPH |
| SEQ ID NO: 590 | NQTLLFLGPPTAP |
| SEQ ID NO: 591 | QTLLFLGPPTAPP |
| SEQ ID NO: 592 | TLLFLGPPTAPPG |
| SEQ ID NO: 593 | LLFLGPPTAPPGG |
| SEQ ID NO: 594 | LFLGPPTAPPGGA |
| SEQ ID NO: 595 | FLGPPTAPPGGAW |
| SEQ ID NO: 596 | LGPPTAPPGGAWT |
| SEQ ID NO: 597 | GPPTAPPGGAWTP |
| SEQ ID NO: 598 | PPTAPPGGAWTPH |
| SEQ ID NO: 599 | PTAPPGGAWTPHA |
| SEQ ID NO: 600 | INQTLLFLGPPTAP |
| SEQ ID NO: 601 | NQTLLFLGPPTAPP |
| SEQ ID NO: 602 | QTLLFLGPPTAPPG |
| SEQ ID NO: 603 | TLLFLGPPTAPPGG |
| SEQ ID NO: 604 | LLFLGPPTAPPGGA |
| SEQ ID NO: 605 | LFLGPPTAPPGGAW |
| SEQ ID NO: 606 | FLGPPTAPPGGAWT |
| SEQ ID NO: 607 | LGPPTAPPGGAWTP |
| SEQ ID NO: 608 | GPPTAPPGGAWTPH |
| SEQ ID NO: 609 | PPTAPPGGAWTPHA |
| SEQ ID NO: 610 | PTAPPGGAWTPHAR |
| SEQ ID NO: 611 | KINQTLLFLGPPTAP |
| SEQ ID NO: 612 | INQTLLFLGPPTAPP |
| SEQ ID NO: 613 | NQTLLFLGPPTAPPG |
| SEQ ID NO: 614 | QTLLFLGPPTAPPGG |
| SEQ ID NO: 615 | TLLFLGPPTAPPGGA |
| SEQ ID NO: 616 | LLFLGPPTAPPGGAW |
| SEQ ID NO: 617 | LFLGPPTAPPGGAWT |
| SEQ ID NO: 618 | FLGPPTAPPGGAWTP |
| SEQ ID NO: 619 | LGPPTAPPGGAWTPH |
| SEQ ID NO: 620 | GPPTAPPGGAWTPHA |
| SEQ ID NO: 621 | PPTAPPGGAWTPHAR |
| SEQ ID NO: 622 | PTAPPGGAWTPHARV |
| SEQ ID NO: 623 | MKINQTLLFLGPPTAP |
| SEQ ID NO: 624 | KINQTLLFLGPPTAPP |
| SEQ ID NO: 625 | INQTLLFLGPPTAPPG |
| SEQ ID NO: 626 | NQTLLFLGPPTAPPGG |
| SEQ ID NO: 627 | QTLLFLGPPTAPPGGA |
| SEQ ID NO: 628 | TLLFLGPPTAPPGGAW |
| SEQ ID NO: 629 | LLFLGPPTAPPGGAWT |
| SEQ ID NO: 630 | LFLGPPTAPPGGAWTP |
| SEQ ID NO: 631 | FLGPPTAPPGGAWTPH |
| SEQ ID NO: 632 | LGPPTAPPGGAWTPHA |
| SEQ ID NO: 633 | GPPTAPPGGAWTPHAR |
| SEQ ID NO: 634 | PPTAPPGGAWTPHARV |
| SEQ ID NO: 635 | PTAPPGGAWTPHARVC |
| SEQ ID NO: 636 | WMKINQTLLFLGPPTAP |
| SEQ ID NO: 637 | MKINQTLLFLGPPTAPP |
| SEQ ID NO: 638 | KINQTLLFLGPPTAPPG |
| SEQ ID NO: 639 | INQTLLFLGPPTAPPGG |
| SEQ ID NO: 640 | NQTLLFLGPPTAPPGGA |
| SEQ ID NO: 641 | QTLLFLGPPTAPPGGAW |
| SEQ ID NO: 642 | TLLFLGPPTAPPGGAWT |
| SEQ ID NO: 643 | LLFLGPPTAPPGGAWTP |
| SEQ ID NO: 644 | LFLGPPTAPPGGAWTPH |
| SEQ ID NO: 645 | FLGPPTAPPGGAWTPHA |
| SEQ ID NO: 646 | LGPPTAPPGGAWTPHAR |
| SEQ ID NO: 647 | GPPTAPPGGAWTPHARV |
| SEQ ID NO: 648 | PPTAPPGGAWTPHARVC |
| SEQ ID NO: 649 | PTAPPGGAWTPHARVCY |
| SEQ ID NO: 650 | VWMKINQTLLFLGPPTAP |
| SEQ ID NO: 651 | WMKINQTLLFLGPPTAPP |
| SEQ ID NO: 652 | MKINQTLLFLGPPTAPPG |
| SEQ ID NO: 653 | KINQTLLFLGPPTAPPGG |
| SEQ ID NO: 654 | INQTLLFLGPPTAPPGGA |
| SEQ ID NO: 655 | NQTLLFLGPPTAPPGGAW |
| SEQ ID NO: 656 | QTLLFLGPPTAPPGGAWT |
| SEQ ID NO: 657 | TLLFLGPPTAPPGGAWTP |
| SEQ ID NO: 658 | LLFLGPPTAPPGGAWTPH |
| SEQ ID NO: 659 | LFLGPPTAPPGGAWTPHA |
| SEQ ID NO: 660 | FLGPPTAPPGGAWTPHAR |
| SEQ ID NO: 661 | LGPPTAPPGGAWTPHARV |
| SEQ ID NO: 662 | GPPTAPPGGAWTPHARVC |
| SEQ ID NO: 663 | PPTAPPGGAWTPHARVCY |
| SEQ ID NO: 664 | PTAPPGGAWTPHARVCYA |
| SEQ ID NO: 665 | LVWMKINQTLLFLGPPTAP |
| SEQ ID NO: 666 | VWMKINQTLLFLGPPTAPP |
| SEQ ID NO: 667 | WMKINQTLLFLGPPTAPPG |
| SEQ ID NO: 668 | MKINQTLLFLGPPTAPPGG |
| SEQ ID NO: 669 | KINQTLLFLGPPTAPPGGA |
| SEQ ID NO: 670 | INQTLLFLGPPTAPPGGAW |
| SEQ ID NO: 671 | NQTLLFLGPPTAPPGGAWT |
| SEQ ID NO: 672 | QTLLFLGPPTAPPGGAWTP |
| SEQ ID NO: 673 | TLLFLGPPTAPPGGAWTPH |
| SEQ ID NO: 674 | LLFLGPPTAPPGGAWTPHA |
| SEQ ID NO: 675 | LFLGPPTAPPGGAWTPHAR |
| SEQ ID NO: 676 | FLGPPTAPPGGAWTPHARV |
| SEQ ID NO: 677 | LGPPTAPPGGAWTPHARVC |
| SEQ ID NO: 678 | GPPTAPPGGAWTPHARVCY |
| SEQ ID NO: 679 | PPTAPPGGAWTPHARVCYA |
| SEQ ID NO: 680 | PTAPPGGAWTPHARVCYAN |
| SEQ ID NO: 681 | ALVWMKINQTLLFLGPPTAP |
| SEQ ID NO: 682 | LVWMKINQTLLFLGPPTAPP |
| SEQ ID NO: 683 | VWMKINQTLLFLGPPTAPPG |
| SEQ ID NO: 684 | WMKINQTLLFLGPPTAPPGG |
| SEQ ID NO: 685 | MKINQTLLFLGPPTAPPGGA |
| SEQ ID NO: 686 | KINQTLLFLGPPTAPPGGAW |
| SEQ ID NO: 687 | INQTLLFLGPPTAPPGGAWT |
| SEQ ID NO: 688 | NQTLLFLGPPTAPPGGAWTP |
| SEQ ID NO: 689 | QTLLFLGPPTAPPGGAWTPH |
| SEQ ID NO: 690 | TLLFLGPPTAPPGGAWTPHA |
| SEQ ID NO: 691 | LLFLGPPTAPPGGAWTPHAR |
| SEQ ID NO: 692 | LFLGPPTAPPGGAWTPHARV |
| SEQ ID NO: 693 | FLGPPTAPPGGAWTPHARVC |
| SEQ ID NO: 694 | LGPPTAPPGGAWTPHARVCY |
| SEQ ID NO: 695 | GPPTAPPGGAWTPHARVCYA |
| SEQ ID NO: 696 | PPTAPPGGAWTPHARVCYAN |
| SEQ ID NO: 697 | PTAPPGGAWTPHARVCYANI |

TABLE 5
P(T/S)AP Motif Containing Peptides from Human
Herpesvirus 2 Virion Glycoprotein K
(GenBank Accession No. NP_044524)

| | |
|---|---|
| SEQ ID NO: 555 | FLGPPTAP |
| SEQ ID NO: 556 | LGPPTAPP |
| SEQ ID NO: 557 | GPPTAPPG |
| SEQ ID NO: 558 | PPTAPPGG |
| SEQ ID NO: 559 | PTAPPGGA |
| SEQ ID NO: 560 | LFLGPPTAP |
| SEQ ID NO: 561 | FLGPPTAPP |
| SEQ ID NO: 562 | LGPPTAPPG |
| SEQ ID NO: 563 | GPPTAPPGG |
| SEQ ID NO: 564 | PPTAPPGGA |
| SEQ ID NO: 565 | PTAPPGGAW |
| SEQ ID NO: 566 | LLFLGPPTAP |
| SEQ ID NO: 567 | LFLGPPTAPP |
| SEQ ID NO: 568 | FLGPPTAPPG |
| SEQ ID NO: 569 | LGPPTAPPGG |
| SEQ ID NO: 570 | GPPTAPPGGA |
| SEQ ID NO: 571 | PPTAPPGGAW |
| SEQ ID NO: 572 | PTAPPGGAWT |
| SEQ ID NO: 573 | TLLFLGPPTAP |
| SEQ ID NO: 574 | LLFLGPPTAPP |
| SEQ ID NO: 575 | LFLGPPTAPPG |
| SEQ ID NO: 576 | FLGPPTAPPGG |
| SEQ ID NO: 577 | LGPPTAPPGGA |
| SEQ ID NO: 578 | GPPTAPPGGAW |
| SEQ ID NO: 579 | PPTAPPGGAWT |
| SEQ ID NO: 580 | PTAPPGGAWTP |
| SEQ ID NO: 581 | QTLLFLGPPTAP |
| SEQ ID NO: 582 | TLLFLGPPTAPP |
| SEQ ID NO: 583 | LLFLGPPTAPPG |
| SEQ ID NO: 584 | LFLGPPTAPPGG |
| SEQ ID NO: 585 | FLGPPTAPPGGA |
| SEQ ID NO: 586 | LGPPTAPPGGAW |
| SEQ ID NO: 587 | GPPTAPPGGAWT |
| SEQ ID NO: 588 | PPTAPPGGAWTP |
| SEQ ID NO: 589 | PTAPPGGAWTPH |
| SEQ ID NO: 590 | NQTLLFLGPPTAP |
| SEQ ID NO: 591 | QTLLFLGPPTAPP |
| SEQ ID NO: 592 | TLLFLGPPTAPPG |
| SEQ ID NO: 593 | LLFLGPPTAPPGG |
| SEQ ID NO: 594 | LFLGPPTAPPGGA |
| SEQ ID NO: 595 | FLGPPTAPPGGAW |
| SEQ ID NO: 596 | LGPPTAPPGGAWT |
| SEQ ID NO: 597 | GPPTAPPGGAWTP |
| SEQ ID NO: 598 | PPTAPPGGAWTPH |
| SEQ ID NO: 599 | PTAPPGGAWTPHA |
| SEQ ID NO: 600 | INQTLLFLGPPTAP |
| SEQ ID NO: 601 | NQTLLFLGPPTAPP |
| SEQ ID NO: 602 | QTLLFLGPPTAPPG |
| SEQ ID NO: 603 | TLLFLGPPTAPPGG |
| SEQ ID NO: 604 | LLFLGPPTAPPGGA |
| SEQ ID NO: 605 | LFLGPPTAPPGGAW |
| SEQ ID NO: 606 | FLGPPTAPPGGAWT |
| SEQ ID NO: 607 | LGPPTAPPGGAWTP |
| SEQ ID NO: 608 | GPPTAPPGGAWTPH |
| SEQ ID NO: 609 | PPTAPPGGAWTPHA |
| SEQ ID NO: 610 | PTAPPGGAWTPHAR |
| SEQ ID NO: 611 | KINQTLLFLGPPTAP |
| SEQ ID NO: 612 | INQTLLFLGPPTAPP |
| SEQ ID NO: 613 | NQTLLFLGPPTAPPG |
| SEQ ID NO: 614 | QTLLFLGPPTAPPGG |
| SEQ ID NO: 615 | TLLFLGPPTAPPGGA |
| SEQ ID NO: 616 | LLFLGPPTAPPGGAW |
| SEQ ID NO: 617 | LFLGPPTAPPGGAWT |
| SEQ ID NO: 618 | FLGPPTAPPGGAWTP |
| SEQ ID NO: 619 | LGPPTAPPGGAWTPH |
| SEQ ID NO: 620 | GPPTAPPGGAWTPHA |
| SEQ ID NO: 621 | PPTAPPGGAWTPHAR |
| SEQ ID NO: 622 | PTAPPGGAWTPHARV |
| SEQ ID NO: 623 | MKINQTLLFLGPPTAP |
| SEQ ID NO: 624 | KINQTLLFLGPPTAPP |
| SEQ ID NO: 625 | INQTLLFLGPPTAPPG |
| SEQ ID NO: 626 | NQTLLFLGPPTAPPGG |
| SEQ ID NO: 627 | QTLLFLGPPTAPPGGA |
| SEQ ID NO: 628 | TLLFLGPPTAPPGGAW |
| SEQ ID NO: 629 | LLFLGPPTAPPGGAWT |
| SEQ ID NO: 630 | LFLGPPTAPPGGAWTP |
| SEQ ID NO: 631 | FLGPPTAPPGGAWTPH |
| SEQ ID NO: 632 | LGPPTAPPGGAWTPHA |
| SEQ ID NO: 633 | GPPTAPPGGAWTPHAR |
| SEQ ID NO: 634 | PPTAPPGGAWTPHARV |
| SEQ ID NO: 635 | PTAPPGGAWTPHARVC |
| SEQ ID NO: 636 | WMKINQTLLFLGPPTAP |
| SEQ ID NO: 637 | MKINQTLLFLGPPTAPP |
| SEQ ID NO: 638 | KINQTLLFLGPPTAPPG |
| SEQ ID NO: 639 | INQTLLFLGPPTAPPGG |
| SEQ ID NO: 640 | NQTLLFLGPPTAPPGGA |
| SEQ ID NO: 641 | QTLLFLGPPTAPPGGAW |
| SEQ ID NO: 642 | TLLFLGPPTAPPGGAWT |
| SEQ ID NO: 643 | LLFLGPPTAPPGGAWTP |
| SEQ ID NO: 644 | LFLGPPTAPPGGAWTPH |
| SEQ ID NO: 645 | FLGPPTAPPGGAWTPHA |
| SEQ ID NO: 646 | LGPPTAPPGGAWTPHAR |
| SEQ ID NO: 647 | GPPTAPPGGAWTPHARV |
| SEQ ID NO: 648 | PPTAPPGGAWTPHARVC |
| SEQ ID NO: 649 | PTAPPGGAWTPHARVCY |
| SEQ ID NO: 650 | VWMKINQTLLFLGPPTAP |
| SEQ ID NO: 651 | WMKINQTLLFLGPPTAPP |
| SEQ ID NO: 652 | MKINQTLLFLGPPTAPPG |
| SEQ ID NO: 653 | KINQTLLFLGPPTAPPGG |
| SEQ ID NO: 654 | INQTLLFLGPPTAPPGGA |
| SEQ ID NO: 655 | NQTLLFLGPPTAPPGGAW |
| SEQ ID NO: 656 | QTLLFLGPPTAPPGGAWT |
| SEQ ID NO: 657 | TLLFLGPPTAPPGGAWTP |
| SEQ ID NO: 658 | LLFLGPPTAPPGGAWTPH |
| SEQ ID NO: 659 | LFLGPPTAPPGGAWTPHA |
| SEQ ID NO: 660 | FLGPPTAPPGGAWTPHAR |
| SEQ ID NO: 661 | LGPPTAPPGGAWTPHARV |
| SEQ ID NO: 662 | GPPTAPPGGAWTPHARVC |
| SEQ ID NO: 663 | PPTAPPGGAWTPHARVCY |
| SEQ ID NO: 664 | PTAPPGGAWTPHARVCYA |
| SEQ ID NO: 665 | LVWMKINQTLLFLGPPTAP |
| SEQ ID NO: 666 | VWMKINQTLLFLGPPTAPP |
| SEQ ID NO: 667 | WMKINQTLLFLGPPTAPPG |
| SEQ ID NO: 668 | MKINQTLLFLGPPTAPPGG |
| SEQ ID NO: 669 | KINQTLLFLGPPTAPPGGA |
| SEQ ID NO: 670 | INQTLLFLGPPTAPPGGAW |
| SEQ ID NO: 671 | NQTLLFLGPPTAPPGGAWT |
| SEQ ID NO: 672 | QTLLFLGPPTAPPGGAWTP |
| SEQ ID NO: 673 | TLLFLGPPTAPPGGAWTPH |
| SEQ ID NO: 674 | LLFLGPPTAPPGGAWTPHA |
| SEQ ID NO: 675 | LFLGPPTAPPGGAWTPHAR |
| SEQ ID NO: 676 | FLGPPTAPPGGAWTPHARV |
| SEQ ID NO: 677 | LGPPTAPPGGAWTPHARVC |
| SEQ ID NO: 678 | GPPTAPPGGAWTPHARVCY |
| SEQ ID NO: 679 | PPTAPPGGAWTPHARVCYA |
| SEQ ID NO: 680 | PTAPPGGAWTPHARVCYAN |
| SEQ ID NO: 681 | ALVWMKINQTLLFLGPPTAP |
| SEQ ID NO: 682 | LVWMKINQTLLFLGPPTAPP |
| SEQ ID NO: 683 | VWMKINQTLLFLGPPTAPPG |
| SEQ ID NO: 684 | WMKINQTLLFLGPPTAPPGG |
| SEQ ID NO: 685 | MKINQTLLFLGPPTAPPGGA |
| SEQ ID NO: 686 | KINQTLLFLGPPTAPPGGAW |
| SEQ ID NO: 687 | INQTLLFLGPPTAPPGGAWT |
| SEQ ID NO: 688 | NQTLLFLGPPTAPPGGAWTP |
| SEQ ID NO: 689 | QTLLFLGPPTAPPGGAWTPH |
| SEQ ID NO: 690 | TLLFLGPPTAPPGGAWTPHA |
| SEQ ID NO: 691 | LLFLGPPTAPPGGAWTPHAR |
| SEQ ID NO: 692 | LFLGPPTAPPGGAWTPHARV |
| SEQ ID NO: 693 | FLGPPTAPPGGAWTPHARVC |
| SEQ ID NO: 694 | LGPPTAPPGGAWTPHARVCY |
| SEQ ID NO: 695 | GPPTAPPGGAWTPHARVCYA |
| SEQ ID NO: 696 | PPTAPPGGAWTPHARVCYAN |
| SEQ ID NO: 697 | PTAPPGGAWTPHARVCYANI |

TABLE 6
P(T/S)AP Motif Containing Peptides from Human
Herpesvirus 2 Strain 333 Glycoprotein I
(GenBank Accession No. P06764)

| | |
|---|---|
| SEQ ID NO: 698 | PRSGPTAP |
| SEQ ID NO: 699 | RSGPTAPQ |
| SEQ ID NO: 700 | SGPTAPQE |
| SEQ ID NO: 701 | GPTAPQEV |
| SEQ ID NO: 702 | RPRSGPTAP |
| SEQ ID NO: 703 | PRSGPTAPQ |
| SEQ ID NO: 704 | RSGPTAPQE |
| SEQ ID NO: 705 | SGPTAPQEV |
| SEQ ID NO: 706 | PRPRSGPTAP |
| SEQ ID NO: 707 | RPRSGPTAPQ |
| SEQ ID NO: 708 | PRSGPTAPQE |
| SEQ ID NO: 709 | RSGPTAPQEV |
| SEQ ID NO: 710 | SPRPRSGPTAP |
| SEQ ID NO: 711 | PRPRSGPTAPQ |
| SEQ ID NO: 712 | RPRSGPTAPQE |
| SEQ ID NO: 713 | PRSGPTAPQEV |
| SEQ ID NO: 714 | VSPRPRSGPTAP |
| SEQ ID NO: 715 | SPRPRSGPTAPQ |
| SEQ ID NO: 716 | PRPRSGPTAPQE |
| SEQ ID NO: 717 | RPRSGPTAPQEV |
| SEQ ID NO: 718 | SVSPRPRSGPTAP |
| SEQ ID NO: 719 | VSPRPRSGPTAPQ |
| SEQ ID NO: 720 | SPRPRSGPTAPQE |
| SEQ ID NO: 721 | PRPRSGPTAPQEV |
| SEQ ID NO: 722 | LSVSPRPRSGPTAP |
| SEQ ID NO: 723 | SVSPRPRSGPTAPQ |
| SEQ ID NO: 724 | VSPRPRSGPTAPQE |
| SEQ ID NO: 725 | SPRPRSGPTAPQEV |
| SEQ ID NO: 726 | LLSVSPRPRSGPTAP |
| SEQ ID NO: 727 | LSVSPRPRSGPTAPQ |
| SEQ ID NO: 728 | SVSPRPRSGPTAPQE |
| SEQ ID NO: 729 | VSPRPRSGPTAPQEV |
| SEQ ID NO: 730 | VLLSVSPRPRSGPTAP |
| SEQ ID NO: 731 | LLSVSPRPRSGPTAPQ |
| SEQ ID NO: 732 | LSVSPRPRSGPTAPQE |
| SEQ ID NO: 733 | SVSPRPRSGPTAPQEV |
| SEQ ID NO: 734 | VVLLSVSPRPRSGPTAP |
| SEQ ID NO: 735 | VLLSVSPRPRSGPTAPQ |
| SEQ ID NO: 736 | LLSVSPRPRSGPTAPQE |
| SEQ ID NO: 737 | LSVSPRPRSGPTAPQEV |
| SEQ ID NO: 738 | PVVLLSVSPRPRSGPTAP |
| SEQ ID NO: 739 | VVLLSVSPRPRSGPTAPQ |
| SEQ ID NO: 740 | VLLSVSPRPRSGPTAPQE |
| SEQ ID NO: 741 | LLSVSPRPRSGPTAPQEV |
| SEQ ID NO: 742 | GPVVLLSVSPRPRSGPTAP |
| SEQ ID NO: 743 | PVVLLSVSPRPRSGPTAPQ |
| SEQ ID NO: 744 | VVLLSVSPRPRSGPTAPQE |
| SEQ ID NO: 745 | VLLSVSPRPRSGPTAPQEV |
| SEQ ID NO: 746 | PGPVVLLSVSPRPRSGPTAP |
| SEQ ID NO: 747 | GPVVLLSVSPRPRSGPTAPQ |
| SEQ ID NO: 748 | PVVLLSVSPRPRSGPTAPQE |
| SEQ ID NO: 749 | VVLLSVSPRPRSGPTAPQEV |

TABLE 7
P(T/S)AP Motif Containing Peptides from Human
Herpesvirus 4/Epstein Barr Virus
BYRF1, Encodes EBNA-2 Protein
(GenBank Accession No. NP_039845)

| | |
|---|---|
| SEQ ID NO: 750 | PPLRPTAP |
| SEQ ID NO: 751 | PLRPTAPT |
| SEQ ID NO: 752 | LRPTAPTI |
| SEQ ID NO: 753 | RPTAPTIL |
| SEQ ID NO: 754 | PTAPTILS |
| SEQ ID NO: 755 | VPPLRPTAP |
| SEQ ID NO: 756 | PPLRPTAPT |
| SEQ ID NO: 757 | PLRPTAPTI |
| SEQ ID NO: 758 | LRPTAPTIL |
| SEQ ID NO: 759 | RPTAPTILS |
| SEQ ID NO: 760 | PTAPTILSP |
| SEQ ID NO: 761 | HVPPLRPTAP |
| SEQ ID NO: 762 | VPPLRPTAPT |
| SEQ ID NO: 763 | PPLRPTAPTI |
| SEQ ID NO: 764 | PLRPTAPTIL |
| SEQ ID NO: 765 | LRPTAPTILS |
| SEQ ID NO: 766 | RPTAPTILSP |
| SEQ ID NO: 767 | PTAPTILSPL |
| SEQ ID NO: 768 | PHVPPLRPTAP |
| SEQ ID NO: 769 | HVPPLRPTAPT |
| SEQ ID NO: 770 | VPPLRPTAPTI |
| SEQ ID NO: 771 | PPLRPTAPTIL |
| SEQ ID NO: 772 | PLRPTAPTILS |
| SEQ ID NO: 773 | LRPTAPTILSP |
| SEQ ID NO: 774 | RPTAPTILSPL |
| SEQ ID NO: 775 | PTAPTILSPLS |
| SEQ ID NO: 776 | QPHVPPLRPTAP |
| SEQ ID NO: 777 | PHVPPLRPTAPT |
| SEQ ID NO: 778 | HVPPLRPTAPTI |
| SEQ ID NO: 779 | VPPLRPTAPTIL |
| SEQ ID NO: 780 | PPLRPTAPTILS |
| SEQ ID NO: 781 | PLRPTAPTILSP |
| SEQ ID NO: 782 | LRPTAPTILSPL |
| SEQ ID NO: 783 | RPTAPTILSPLS |
| SEQ ID NO: 784 | PTAPTILSPLSQ |
| SEQ ID NO: 785 | VQPHVPPLRPTAP |
| SEQ ID NO: 786 | QPHVPPLRPTAPT |
| SEQ ID NO: 787 | PHVPPLRPTAPTI |
| SEQ ID NO: 788 | HVPPLRPTAPTIL |
| SEQ ID NO: 789 | VPPLRPTAPTILS |
| SEQ ID NO: 790 | PPLRPTAPTILSP |
| SEQ ID NO: 791 | PLRPTAPTILSPL |
| SEQ ID NO: 792 | LRPTAPTILSPLS |
| SEQ ID NO: 793 | RPTAPTILSPLSQ |
| SEQ ID NO: 794 | PTAPTILSPLSQP |
| SEQ ID NO: 795 | LVQPHVPPLRPTAP |
| SEQ ID NO: 796 | VQPHVPPLRPTAPT |
| SEQ ID NO: 797 | QPHVPPLRPTAPTI |
| SEQ ID NO: 798 | PHVPPLRPTAPTIL |
| SEQ ID NO: 799 | HVPPLRPTAPTILS |
| SEQ ID NO: 800 | VPPLRPTAPTILSP |
| SEQ ID NO: 801 | PPLRPTAPTILSPL |
| SEQ ID NO: 802 | PLRPTAPTILSPLS |
| SEQ ID NO: 803 | LRPTAPTILSPLSQ |
| SEQ ID NO: 804 | RPTAPTILSPLSQP |
| SEQ ID NO: 805 | PTAPTILSPLSQPR |
| SEQ ID NO: 806 | RLVQPHVPPLRPTAP |
| SEQ ID NO: 807 | LVQPHVPPLRPTAPT |
| SEQ ID NO: 808 | VQPHVPPLRPTAPTI |
| SEQ ID NO: 809 | QPHVPPLRPTAPTIL |
| SEQ ID NO: 810 | PHVPPLRPTAPTILS |
| SEQ ID NO: 811 | HVPPLRPTAPTILSP |
| SEQ ID NO: 812 | VPPLRPTAPTILSPL |
| SEQ ID NO: 813 | PPLRPTAPTILSPLS |
| SEQ ID NO: 814 | PLRPTAPTILSPLSQ |
| SEQ ID NO: 815 | LRPTAPTILSPLSQP |
| SEQ ID NO: 816 | RPTAPTILSPLSQPR |
| SEQ ID NO: 817 | PTAPTILSPLSQPRL |
| SEQ ID NO: 818 | ARLVQPHVPPLRPTAP |
| SEQ ID NO: 819 | RLVQPHVPPLRPTAPT |
| SEQ ID NO: 820 | LVQPHVPPLRPTAPTI |
| SEQ ID NO: 821 | VQPHVPPLRPTAPTIL |
| SEQ ID NO: 822 | QPHVPPLRPTAPTILS |
| SEQ ID NO: 823 | PHVPPLRPTAPTILSP |
| SEQ ID NO: 824 | HVPPLRPTAPTILSPL |
| SEQ ID NO: 825 | VPPLRPTAPTILSPLS |
| SEQ ID NO: 826 | PPLRPTAPTILSPLSQ |
| SEQ ID NO: 827 | PLRPTAPTILSPLSQP |
| SEQ ID NO: 828 | LRPTAPTILSPLSQPR |
| SEQ ID NO: 829 | RPTAPTILSPLSQPRL |
| SEQ ID NO: 830 | PTAPTILSPLSQPRLT |
| SEQ ID NO: 831 | QARLVQPHVPPLRPTAP |
| SEQ ID NO: 832 | ARLVQPHVPPLRPTAPT |
| SEQ ID NO: 833 | RLVQPHVPPLRPTAPTI |
| SEQ ID NO: 834 | LVQPHVPPLRPTAPTIL |
| SEQ ID NO: 835 | VQPHVPPLRPTAPTILS |
| SEQ ID NO: 836 | QPHVPPLRPTAPTILSP |
| SEQ ID NO: 837 | PHVPPLRPTAPTILSPL |
| SEQ ID NO: 838 | HVPPLRPTAPTILSPLS |
| SEQ ID NO: 839 | VPPLRPTAPTILSPLSQ |
| SEQ ID NO: 840 | PPLRPTAPTILSPLSQP |
| SEQ ID NO: 841 | PLRPTAPTILSPLSQPR |
| SEQ ID NO: 842 | LRPTAPTILSPLSQPRL |
| SEQ ID NO: 843 | RPTAPTILSPLSQPRLT |
| SEQ ID NO: 844 | PTAPTILSPLSQPRLTP |
| SEQ ID NO: 845 | PQARLVQPHVPPLRPTAP |
| SEQ ID NO: 846 | QARLVQPHVPPLRPTAPT |
| SEQ ID NO: 847 | ARLVQPHVPPLRPTAPTI |
| SEQ ID NO: 848 | RLVQPHVPPLRPTAPTIL |
| SEQ ID NO: 849 | LVQPHVPPLRPTAPTILS |
| SEQ ID NO: 850 | VQPHVPPLRPTAPTILSP |
| SEQ ID NO: 851 | QPHVPPLRPTAPTILSPL |
| SEQ ID NO: 852 | PHVPPLRPTAPTILSPLS |
| SEQ ID NO: 853 | HVPPLRPTAPTILSPLSQ |
| SEQ ID NO: 854 | VPPLRPTAPTILSPLSQP |
| SEQ ID NO: 855 | PPLRPTAPTILSPLSQPR |
| SEQ ID NO: 856 | PLRPTAPTILSPLSQPRL |
| SEQ ID NO: 857 | LRPTAPTILSPLSQPRLT |
| SEQ ID NO: 858 | RPTAPTILSPLSQPRLTP |
| SEQ ID NO: 859 | PTAPTILSPLSQPRLTPP |
| SEQ ID NO: 860 | APQARLVQPHVPPLRPTAP |
| SEQ ID NO: 861 | PQARLVQPHVPPLRPTAPT |
| SEQ ID NO: 862 | QARLVQPHVPPLRPTAPTI |
| SEQ ID NO: 863 | ARLVQPHVPPLRPTAPTIL |
| SEQ ID NO: 864 | RLVQPHVPPLRPTAPTILS |
| SEQ ID NO: 865 | LVQPHVPPLRPTAPTILSP |
| SEQ ID NO: 866 | VQPHVPPLRPTAPTILSPL |
| SEQ ID NO: 867 | QPHVPPLRPTAPTILSPLS |
| SEQ ID NO: 868 | PHVPPLRPTAPTILSPLSQ |
| SEQ ID NO: 869 | HVPPLRPTAPTILSPLSQP |
| SEQ ID NO: 870 | VPPLRPTAPTILSPLSQPR |
| SEQ ID NO: 871 | PPLRPTAPTILSPLSQPRL |
| SEQ ID NO: 872 | PLRPTAPTILSPLSQPRLT |
| SEQ ID NO: 873 | LRPTAPTILSPLSQPRLTP |
| SEQ ID NO: 874 | RPTAPTILSPLSQPRLTPP |
| SEQ ID NO: 875 | PTAPTILSPLSQPRLTPPQ |
| SEQ ID NO: 876 | TAPQARLVQPHVPPLRPTAP |
| SEQ ID NO: 877 | APQARLVQPHVPPLRPTAPT |
| SEQ ID NO: 878 | PQARLVQPHVPPLRPTAPTI |
| SEQ ID NO: 879 | QARLVQPHVPPLRPTAPTIL |
| SEQ ID NO: 880 | ARLVQPHVPPLRPTAPTILS |
| SEQ ID NO: 881 | RLVQPHVPPLRPTAPTILSP |
| SEQ ID NO: 882 | LVQPHVPPLRPTAPTILSPL |
| SEQ ID NO: 883 | VQPHVPPLRPTAPTILSPLS |
| SEQ ID NO: 884 | QPHVPPLRPTAPTILSPLSQ |
| SEQ ID NO: 885 | PHVPPLRPTAPTILSPLSQP |
| SEQ ID NO: 886 | HVPPLRPTAPTILSPLSQPR |
| SEQ ID NO: 887 | VPPLRPTAPTILSPLSQPRL |
| SEQ ID NO: 888 | PPLRPTAPTILSPLSQPRLT |
| SEQ ID NO: 889 | PLRPTAPTILSPLSQPRLTP |
| SEQ ID NO: 890 | LRPTAPTILSPLSQPRLTPP |
| SEQ ID NO: 891 | RPTAPTILSPLSQPRLTPPQ |
| SEQ ID NO: 892 | PTAPTILSPLSQPRLTPPQP |

TABLE 8
P(T/S)AP Motif Containing
Peptides from Influenza A Virus
(A/Pintail Duck/Alberta/114/7(H8N4))
(GenBank Accession No. AAG38554)

| | |
|---|---|
| SEQ ID NO: 893 | IVERPSAP |
| SEQ ID NO: 894 | VERPSAPE |
| SEQ ID NO: 895 | ERPSAPEG |
| SEQ ID NO: 896 | RPSAPEGM |
| SEQ ID NO: 897 | PSAPEGMC |
| SEQ ID NO: 898 | YIVERPSAP |
| SEQ ID NO: 899 | IVERPSAPE |
| SEQ ID NO: 900 | VERPSAPEG |
| SEQ ID NO: 901 | ERPSAPEGM |
| SEQ ID NO: 902 | RPSAPEGMC |
| SEQ ID NO: 903 | PSAPEGMCY |
| SEQ ID NO: 904 | SYIVERPSAP |
| SEQ ID NO: 905 | YIVERPSAPE |
| SEQ ID NO: 906 | IVERPSAPEG |
| SEQ ID NO: 907 | VERPSAPEGM |
| SEQ ID NO: 908 | ERPSAPEGMC |
| SEQ ID NO: 909 | RPSAPEGMCY |
| SEQ ID NO: 910 | PSAPEGMCYP |
| SEQ ID NO: 911 | WSYIVERPSAP |
| SEQ ID NO: 912 | SYIVERPSAPE |
| SEQ ID NO: 913 | YIVERPSAPEG |
| SEQ ID NO: 914 | IVERPSAPEGM |
| SEQ ID NO: 915 | VERPSAPEGMC |
| SEQ ID NO: 916 | ERPSAPEGMCY |
| SEQ ID NO: 917 | RPSAPEGMCYP |
| SEQ ID NO: 918 | PSAPEGMCYPG |
| SEQ ID NO: 919 | GWSYIVERPSAP |
| SEQ ID NO: 920 | WSYIVERPSAPE |
| SEQ ID NO: 921 | SYIVERPSAPEG |
| SEQ ID NO: 922 | YIVERPSAPEGM |
| SEQ ID NO: 923 | IVERPSAPEGMC |
| SEQ ID NO: 924 | VERPSAPEGMCY |
| SEQ ID NO: 925 | ERPSAPEGMCYP |
| SEQ ID NO: 926 | RPSAPEGMCYPG |
| SEQ ID NO: 927 | PSAPEGMCYPGS |
| SEQ ID NO: 928 | QGWSYIVERPSAP |
| SEQ ID NO: 929 | GWSYIVERPSAPE |
| SEQ ID NO: 930 | WSYIVERPSAPEG |
| SEQ ID NO: 931 | SYIVERPSAPEGM |
| SEQ ID NO: 932 | YIVERPSAPEGMC |
| SEQ ID NO: 933 | IVERPSAPEGMCY |
| SEQ ID NO: 934 | VERPSAPEGMCYP |
| SEQ ID NO: 935 | ERPSAPEGMCYPG |
| SEQ ID NO: 936 | RPSAPEGMCYPGS |
| SEQ ID NO: 937 | PSAPEGMCYPGSI |
| SEQ ID NO: 938 | DQGWSYIVERPSAP |
| SEQ ID NO: 939 | QGWSYIVERPSAPE |
| SEQ ID NO: 940 | GWSYIVERPSAPEG |
| SEQ ID NO: 941 | WSYIVERPSAPEGM |
| SEQ ID NO: 942 | SYIVERPSAPEGMC |
| SEQ ID NO: 943 | YIVERPSAPEGMCY |
| SEQ ID NO: 944 | IVERPSAPEGMCYP |
| SEQ ID NO: 945 | VERPSAPEGMCYPG |
| SEQ ID NO: 946 | ERPSAPEGMCYPGS |
| SEQ ID NO: 947 | RPSAPEGMCYPGSI |
| SEQ ID NO: 948 | PSAPEGMCYPGSIE |
| SEQ ID NO: 949 | KDQGWSYIVERPSAP |
| SEQ ID NO: 950 | DQGWSYIVERPSAPE |
| SEQ ID NO: 951 | QGWSYIVERPSAPEG |
| SEQ ID NO: 952 | GWSYIVERPSAPEGM |
| SEQ ID NO: 953 | WSYIVERPSAPEGMC |
| SEQ ID NO: 954 | SYIVERPSAPEGMCY |
| SEQ ID NO: 955 | YIVERPSAPEGMCYP |
| SEQ ID NO: 956 | IVERPSAPEGMCYPG |
| SEQ ID NO: 957 | VERPSAPEGMCYPGS |
| SEQ ID NO: 958 | ERPSAPEGMCYPGSI |
| SEQ ID NO: 959 | RPSAPEGMCYPGSIE |
| SEQ ID NO: 960 | PSAPEGMCYPGSIEN |
| SEQ ID NO: 961 | LKDQGWSYIVERPSAP |
| SEQ ID NO: 962 | KDQGWSYIVERPSAPE |
| SEQ ID NO: 963 | DQGWSYIVERPSAPEG |
| SEQ ID NO: 964 | QGWSYIVERPSAPEGM |
| SEQ ID NO: 965 | GWSYIVERPSAPEGMC |
| SEQ ID NO: 966 | WSYIVERPSAPEGMCY |
| SEQ ID NO: 967 | SYIVERPSAPEGMCYP |
| SEQ ID NO: 968 | YIVERPSAPEGMCYPG |
| SEQ ID NO: 969 | IVERPSAPEGMCYPGS |
| SEQ ID NO: 970 | VERPSAPEGMCYPGSI |
| SEQ ID NO: 971 | ERPSAPEGMCYPGSIE |
| SEQ ID NO: 972 | RPSAPEGMCYPGSIEN |
| SEQ ID NO: 973 | PSAPEGMCYPGSIENL |
| SEQ ID NO: 974 | HLKDQGWSYIVERPSAP |
| SEQ ID NO: 975 | LKDQGWSYIVERPSAPE |
| SEQ ID NO: 976 | KDQGWSYIVERPSAPEG |
| SEQ ID NO: 977 | DQGWSYIVERPSAPEGM |
| SEQ ID NO: 978 | QGWSYIVERPSAPEGMC |
| SEQ ID NO: 979 | GWSYIVERPSAPEGMCY |
| SEQ ID NO: 980 | WSYIVERPSAPEGMCYP |
| SEQ ID NO: 981 | SYIVERPSAPEGMCYPG |
| SEQ ID NO: 982 | YIVERPSAPEGMCYPGS |
| SEQ ID NO: 983 | IVERPSAPEGMCYPGSI |
| SEQ ID NO: 984 | VERPSAPEGMCYPGSIE |
| SEQ ID NO: 985 | ERPSAPEGMCYPGSIEN |
| SEQ ID NO: 986 | RPSAPEGMCYPGSIENL |
| SEQ ID NO: 987 | PSAPEGMCYPGSIENLE |
| SEQ ID NO: 988 | IHLKDQGWSYIVERPSAP |
| SEQ ID NO: 989 | HLKDQGWSYIVERPSAPE |
| SEQ ID NO: 990 | LKDQGWSYIVERPSAPEG |
| SEQ ID NO: 991 | KDQGWSYIVERPSAPEGM |
| SEQ ID NO: 992 | DQGWSYIVERPSAPEGMC |
| SEQ ID NO: 993 | QGWSYIVERPSAPEGMCY |
| SEQ ID NO: 994 | GWSYIVERPSAPEGMCYP |
| SEQ ID NO: 995 | WSYIVERPSAPEGMCYPG |
| SEQ ID NO: 996 | SYIVERPSAPEGMCYPGS |
| SEQ ID NO: 997 | YIVERPSAPEGMCYPGSI |
| SEQ ID NO: 998 | IVERPSAPEGMCYPGSIE |
| SEQ ID NO: 999 | VERPSAPEGMCYPGSIEN |
| SEQ ID NO: 1000 | ERPSAPEGMCYPGSIENL |
| SEQ ID NO: 1001 | RPSAPEGMCYPGSIENLE |
| SEQ ID NO: 1002 | PSAPEGMCYPGSIENLEE |
| SEQ ID NO: 1003 | DIHLKDQGWSYIVERPSAP |
| SEQ ID NO: 1004 | IHLKDQGWSYIVERPSAPE |
| SEQ ID NO: 1005 | HLKDQGWSYIVERPSAPEG |
| SEQ ID NO: 1006 | LKDQGWSYIVERPSAPEGM |
| SEQ ID NO: 1007 | KDQGWSYIVERPSAPEGMC |
| SEQ ID NO: 1008 | DQGWSYIVERPSAPEGMCY |
| SEQ ID NO: 1009 | QGWSYIVERPSAPEGMCYP |
| SEQ ID NO: 1010 | GWSYIVERPSAPEGMCYPG |
| SEQ ID NO: 1011 | WSYIVERPSAPEGMCYPGS |
| SEQ ID NO: 1012 | SYIVERPSAPEGMCYPGSI |
| SEQ ID NO: 1013 | YIVERPSAPEGMCYPGSIE |
| SEQ ID NO: 1014 | IVERPSAPEGMCYPGSIEN |
| SEQ ID NO: 1015 | VERPSAPEGMCYPGSIENL |
| SEQ ID NO: 1016 | ERPSAPEGMCYPGSIENLE |
| SEQ ID NO: 1017 | RPSAPEGMCYPGSIENLEE |
| SEQ ID NO: 1018 | PSAPEGMCYPGSIENLEEL |
| SEQ ID NO: 1019 | CDIHLKDQGWSYIVERPSAP |
| SEQ ID NO: 1020 | DIHLKDQGWSYIVERPSAPE |
| SEQ ID NO: 1021 | IHLKDQGWSYIVERPSAPEG |
| SEQ ID NO: 1022 | HLKDQGWSYIVERPSAPEGM |
| SEQ ID NO: 1023 | LKDQGWSYIVERPSAPEGMC |
| SEQ ID NO: 1024 | KDQGWSYIVERPSAPEGMCY |
| SEQ ID NO: 1025 | DQGWSYIVERPSAPEGMCYP |
| SEQ ID NO: 1026 | QGWSYIVERPSAPEGMCYPG |
| SEQ ID NO: 1027 | GWSYIVERPSAPEGMCYPGS |
| SEQ ID NO: 1028 | WSYIVERPSAPEGMCYPGSI |
| SEQ ID NO: 1029 | SYIVERPSAPEGMCYPGSIE |
| SEQ ID NO: 1030 | YIVERPSAPEGMCYPGSIEN |
| SEQ ID NO: 1031 | IVERPSAPEGMCYPGSIENL |
| SEQ ID NO: 1032 | VERPSAPEGMCYPGSIENLE |
| SEQ ID NO: 1033 | ERPSAPEGMCYPGSIENLEE |
| SEQ ID NO: 1034 | RPSAPEGMCYPGSIENLEEL |
| SEQ ID NO: 1035 | PSAPEGMCYPGSIENLEELR |

TABLE 9
P(T/S)AP Motif Containing Peptides from Human
Papillomavirus L1 Protein My09/My11 Region
(GenBank Accession No. AAA67231)

| | |
|---|---|
| SEQ ID NO: 1036 | CQKGPSAP |
| SEQ ID NO: 1037 | QKGPSAPA |
| SEQ ID NO: 1038 | KGPSAPAP |
| SEQ ID NO: 1039 | GPSAPAPK |
| SEQ ID NO: 1040 | PSAPAPKK |
| SEQ ID NO: 1041 | TCQKGPSAP |
| SEQ ID NO: 1042 | CQKGPSAPA |
| SEQ ID NO: 1043 | QKGPSAPAP |
| SEQ ID NO: 1044 | KGPSAPAPK |
| SEQ ID NO: 1045 | GPSAPAPKK |
| SEQ ID NO: 1046 | PSAPAPKKD |
| SEQ ID NO: 1047 | ITCQKGPSAP |
| SEQ ID NO: 1048 | TCQKGPSAPA |
| SEQ ID NO: 1049 | CQKGPSAPAP |
| SEQ ID NO: 1050 | QKGPSAPAPK |
| SEQ ID NO: 1051 | KGPSAPAPKK |
| SEQ ID NO: 1052 | GPSAPAPKKD |
| SEQ ID NO: 1053 | PSAPAPKKDP |
| SEQ ID NO: 1054 | AITCQKGPSAP |
| SEQ ID NO: 1055 | ITCQKGPSAPA |
| SEQ ID NO: 1056 | TCQKGPSAPAP |
| SEQ ID NO: 1057 | CQKGPSAPAPK |
| SEQ ID NO: 1058 | QKGPSAPAPKK |
| SEQ ID NO: 1059 | KGPSAPAPKKD |
| SEQ ID NO: 1060 | GPSAPAPKKDP |
| SEQ ID NO: 1061 | PSAPAPKKDPY |
| SEQ ID NO: 1062 | RAITCQKGPSAP |
| SEQ ID NO: 1063 | AITCQKGPSAPA |
| SEQ ID NO: 1064 | ITCQKGPSAPAP |
| SEQ ID NO: 1065 | TCQKGPSAPAPK |
| SEQ ID NO: 1066 | CQKGPSAPAPKK |
| SEQ ID NO: 1067 | QKGPSAPAPKKD |
| SEQ ID NO: 1068 | KGPSAPAPKKDP |
| SEQ ID NO: 1069 | GPSAPAPKKDPY |
| SEQ ID NO: 1070 | PSAPAPKKDPYD |
| SEQ ID NO: 1071 | SRAITCQKGPSAP |
| SEQ ID NO: 1072 | RAITCQKGPSAPA |
| SEQ ID NO: 1073 | AITCQKGPSAPAP |
| SEQ ID NO: 1074 | ITCQKGPSAPAPK |
| SEQ ID NO: 1075 | TCQKGPSAPAPKK |
| SEQ ID NO: 1076 | CQKGPSAPAPKKD |
| SEQ ID NO: 1077 | QKGPSAPAPKKDP |
| SEQ ID NO: 1078 | KGPSAPAPKKDPY |
| SEQ ID NO: 1079 | GPSAPAPKKDPYD |
| SEQ ID NO: 1080 | PSAPAPKKDPYDG |
| SEQ ID NO: 1081 | QSRAITCQKGPSAP |
| SEQ ID NO: 1082 | SRAITCQKGPSAPA |
| SEQ ID NO: 1083 | RAITCQKGPSAPAP |
| SEQ ID NO: 1084 | AITCQKGPSAPAPK |
| SEQ ID NO: 1085 | ITCQKGPSAPAPKK |
| SEQ ID NO: 1086 | TCQKGPSAPAPKKD |
| SEQ ID NO: 1087 | CQKGPSAPAPKKDP |
| SEQ ID NO: 1088 | QKGPSAPAPKKDPY |
| SEQ ID NO: 1089 | KGPSAPAPKKDPYD |
| SEQ ID NO: 1090 | GPSAPAPKKDPYDG |
| SEQ ID NO: 1091 | PSAPAPKKDPYDGL |
| SEQ ID NO: 1092 | LQSRAITCQKGPSAP |
| SEQ ID NO: 1093 | QSRAITCQKGPSAPA |
| SEQ ID NO: 1094 | SRAITCQKGPSAPAP |
| SEQ ID NO: 1095 | RAITCQKGPSAPAPK |
| SEQ ID NO: 1096 | AITCQKGPSAPAPKK |
| SEQ ID NO: 1097 | ITCQKGPSAPAPKKD |
| SEQ ID NO: 1098 | TCQKGPSAPAPKKDP |
| SEQ ID NO: 1099 | CQKGPSAPAPKKDPY |
| SEQ ID NO: 1100 | QKGPSAPAPKKDPYD |
| SEQ ID NO: 1101 | KGPSAPAPKKDPYDG |
| SEQ ID NO: 1102 | GPSAPAPKKDPYDGL |
| SEQ ID NO: 1103 | PSAPAPKKDPYDGLV |
| SEQ ID NO: 1104 | YLQSRAITCQKGPSAP |
| SEQ ID NO: 1105 | LQSRAITCQKGPSAPA |
| SEQ ID NO: 1106 | QSRAITCQKGPSAPAP |
| SEQ ID NO: 1107 | SRAITCQKGPSAPAPK |
| SEQ ID NO: 1108 | RAITCQKGPSAPAPKK |
| SEQ ID NO: 1109 | AITCQKGPSAPAPKKD |
| SEQ ID NO: 1110 | ITCQKGPSAPAPKKDP |
| SEQ ID NO: 1111 | TCQKGPSAPAPKKDPY |
| SEQ ID NO: 1112 | CQKGPSAPAPKKDPYD |
| SEQ ID NO: 1113 | QKGPSAPAPKKDPYDG |
| SEQ ID NO: 1114 | KGPSAPAPKKDPYDGL |
| SEQ ID NO: 1115 | GPSAPAPKKDPYDGLV |
| SEQ ID NO: 1116 | PSAPAPKKDPYDGLVF |
| SEQ ID NO: 1117 | RYLQSRAITCQKGPSAP |
| SEQ ID NO: 1118 | YLQSRAITCQKGPSAPA |
| SEQ ID NO: 1119 | LQSRAITCQKGPSAPAP |
| SEQ ID NO: 1120 | QSRAITCQKGPSAPAPK |
| SEQ ID NO: 1121 | SRAITCQKGPSAPAPKK |
| SEQ ID NO: 1122 | RAITCQKGPSAPAPKKD |
| SEQ ID NO: 1123 | AITCQKGPSAPAPKKDP |
| SEQ ID NO: 1124 | ITCQKGPSAPAPKKDPY |
| SEQ ID NO: 1125 | TCQKGPSAPAPKKDPYD |
| SEQ ID NO: 1126 | CQKGPSAPAPKKDPYDG |
| SEQ ID NO: 1127 | QKGPSAPAPKKDPYDGL |
| SEQ ID NO: 1128 | KGPSAPAPKKDPYDGLV |
| SEQ ID NO: 1129 | GPSAPAPKKDPYDGLVF |
| SEQ ID NO: 1130 | PSAPAPKKDPYDGLVFW |
| SEQ ID NO: 1131 | YRYLQSRAITCQKGPSAP |
| SEQ ID NO: 1132 | RYLQSRAITCQKGPSAPA |
| SEQ ID NO: 1133 | YLQSRAITCQKGPSAPAP |
| SEQ ID NO: 1134 | LQSRAITCQKGPSAPAPK |
| SEQ ID NO: 1135 | QSRAITCQKGPSAPAPKK |
| SEQ ID NO: 1136 | SRAITCQKGPSAPAPKKD |
| SEQ ID NO: 1137 | RAITCQKGPSAPAPKKDP |
| SEQ ID NO: 1138 | AITCQKGPSAPAPKKDPY |
| SEQ ID NO: 1139 | ITCQKGPSAPAPKKDPYD |
| SEQ ID NO: 1140 | TCQKGPSAPAPKKDPYDG |
| SEQ ID NO: 1141 | CQKGPSAPAPKKDPYDGL |
| SEQ ID NO: 1142 | QKGPSAPAPKKDPYDGLV |
| SEQ ID NO: 1143 | KGPSAPAPKKDPYDGLVF |
| SEQ ID NO: 1144 | GPSAPAPKKDPYDGLVFW |
| SEQ ID NO: 1145 | PSAPAPKKDPYDGLVFWE |
| SEQ ID NO: 1146 | TYRYLQSRAITCQKGPSAP |
| SEQ ID NO: 1147 | YRYLQSRAITCQKGPSAPA |
| SEQ ID NO: 1148 | RYLQSRAITCQKGPSAPAP |
| SEQ ID NO: 1149 | YLQSRAITCQKGPSAPAPK |
| SEQ ID NO: 1150 | LQSRAITCQKGPSAPAPKK |
| SEQ ID NO: 1151 | QSRAITCQKGPSAPAPKKD |
| SEQ ID NO: 1152 | SRAITCQKGPSAPAPKKDP |
| SEQ ID NO: 1153 | RAITCQKGPSAPAPKKDPY |
| SEQ ID NO: 1154 | AITCQKGPSAPAPKKDPYD |
| SEQ ID NO: 1155 | ITCQKGPSAPAPKKDPYDG |
| SEQ ID NO: 1156 | TCQKGPSAPAPKKDPYDGL |
| SEQ ID NO: 1157 | CQKGPSAPAPKKDPYDGLV |
| SEQ ID NO: 1158 | QKGPSAPAPKKDPYDGLVF |
| SEQ ID NO: 1159 | KGPSAPAPKKDPYDGLVFW |
| SEQ ID NO: 1160 | GPSAPAPKKDPYDGLVFWE |
| SEQ ID NO: 1161 | PSAPAPKKDPYDGLVFWEV |
| SEQ ID NO: 1162 | DTYRYLQSRAITCQKGPSAP |
| SEQ ID NO: 1163 | TYRYLQSRAITCQKGPSAPA |
| SEQ ID NO: 1164 | YRYLQSRAITCQKGPSAPAP |
| SEQ ID NO: 1165 | RYLQSRAITCQKGPSAPAPK |
| SEQ ID NO: 1166 | YLQSRAITCQKGPSAPAPKK |
| SEQ ID NO: 1167 | LQSRAITCQKGPSAPAPKKD |
| SEQ ID NO: 1168 | QSRAITCQKGPSAPAPKKDP |
| SEQ ID NO: 1169 | SRAITCQKGPSAPAPKKDPY |
| SEQ ID NO: 1170 | RAITCQKGPSAPAPKKDPYD |
| SEQ ID NO: 1171 | AITCQKGPSAPAPKKDPYDG |
| SEQ ID NO: 1172 | ITCQKGPSAPAPKKDPYDGL |
| SEQ ID NO: 1173 | TCQKGPSAPAPKKDPYDGLV |
| SEQ ID NO: 1174 | CQKGPSAPAPKKDPYDGLVF |
| SEQ ID NO: 1175 | QKGPSAPAPKKDPYDGLVFW |
| SEQ ID NO: 1176 | KGPSAPAPKKDPYDGLVFWE |
| SEQ ID NO: 1177 | GPSAPAPKKDPYDGLVFWEV |
| SEQ ID NO: 1178 | PSAPAPKKDPYDGLVFWEVD |

TABLE 10
P(T/S)AP Motif Containing Peptides from Human
Papillomavirus Type 23 Minor Capsid Protein L2
(GenBank Accession No. NP_043365)

| | |
|---|---|
| SEQ ID NO: 1179 | IFPLPSAP |
| SEQ ID NO: 1180 | FPLPSAPA |
| SEQ ID NO: 1181 | PLPSAPAV |
| SEQ ID NO: 1182 | LPSAPAVV |
| SEQ ID NO: 1183 | PSAPAVVI |
| SEQ ID NO: 1184 | IIFPLPSAP |
| SEQ ID NO: 1185 | IFPLPSAPA |
| SEQ ID NO: 1186 | FPLPSAPAV |
| SEQ ID NO: 1187 | PLPSAPAVV |
| SEQ ID NO: 1188 | LPSAPAVVI |
| SEQ ID NO: 1189 | PSAPAVVIH |
| SEQ ID NO: 1190 | TIIFPLPSAP |
| SEQ ID NO: 1191 | IIFPLPSAPA |
| SEQ ID NO: 1192 | IFPLPSAPAV |
| SEQ ID NO: 1193 | FPLPSAPAVV |
| SEQ ID NO: 1194 | PLPSAPAVVI |
| SEQ ID NO: 1195 | LPSAPAVVIH |
| SEQ ID NO: 1196 | PSAPAVVIHT |
| SEQ ID NO: 1197 | PTIIFPLPSAP |
| SEQ ID NO: 1198 | TIIFPLPSAPA |
| SEQ ID NO: 1199 | IIFPLPSAPAV |
| SEQ ID NO: 1200 | IFPLPSAPAVV |
| SEQ ID NO: 1201 | FPLPSAPAVVI |
| SEQ ID NO: 1202 | PLPSAPAVVIH |
| SEQ ID NO: 1203 | LPSAPAVVIHT |
| SEQ ID NO: 1204 | PSAPAVVIHTL |
| SEQ ID NO: 1205 | RPTIIFPLPSAP |
| SEQ ID NO: 1206 | PTIIFPLPSAPA |
| SEQ ID NO: 1207 | TIIFPLPSAPAV |
| SEQ ID NO: 1208 | IIFPLPSAPAVV |
| SEQ ID NO: 1209 | IFPLPSAPAVVI |
| SEQ ID NO: 1210 | FPLPSAPAVVIH |
| SEQ ID NO: 1211 | PLPSAPAVVIHT |
| SEQ ID NO: 1212 | LPSAPAVVIHTL |
| SEQ ID NO: 1213 | PSAPAVVIHTLD |
| SEQ ID NO: 1214 | ERPTIIFPLPSAP |
| SEQ ID NO: 1215 | RPTIIFPLPSAPA |
| SEQ ID NO: 1216 | PTIIFPLPSAPAV |
| SEQ ID NO: 1217 | TIIFPLPSAPAVV |
| SEQ ID NO: 1218 | IIFPLPSAPAVVI |
| SEQ ID NO: 1219 | IFPLPSAPAVVIH |
| SEQ ID NO: 1220 | FPLPSAPAVVIHT |
| SEQ ID NO: 1221 | PLPSAPAVVIHTL |
| SEQ ID NO: 1222 | LPSAPAVVIHTLD |
| SEQ ID NO: 1223 | PSAPAVVIHTLDK |
| SEQ ID NO: 1224 | TERPTIIFPLPSAP |
| SEQ ID NO: 1225 | ERPTIIFPLPSAPA |
| SEQ ID NO: 1226 | RPTIIFPLPSAPAV |
| SEQ ID NO: 1227 | PTIIFPLPSAPAVV |
| SEQ ID NO: 1228 | TIIFPLPSAPAVVI |
| SEQ ID NO: 1229 | IIFPLPSAPAVVIH |
| SEQ ID NO: 1230 | IFPLPSAPAVVIHT |
| SEQ ID NO: 1231 | FPLPSAPAVVIHTL |
| SEQ ID NO: 1232 | PLPSAPAVVIHTLD |
| SEQ ID NO: 1233 | LPSAPAVVIHTLDK |
| SEQ ID NO: 1234 | PSAPAVVIHTLDKS |
| SEQ ID NO: 1235 | PTERPTIIFPLPSAP |
| SEQ ID NO: 1236 | TERPTIIFPLPSAPA |
| SEQ ID NO: 1237 | ERPTIIFPLPSAPAV |
| SEQ ID NO: 1238 | RPTIIFPLPSAPAVV |
| SEQ ID NO: 1239 | PTIIFPLPSAPAVVI |
| SEQ ID NO: 1240 | TIIFPLPSAPAVVIH |
| SEQ ID NO: 1241 | IIFPLPSAPAVVIHT |
| SEQ ID NO: 1242 | IFPLPSAPAVVIHTL |
| SEQ ID NO: 1243 | FPLPSAPAVVIHTLD |
| SEQ ID NO: 1244 | PLPSAPAVVIHTLDK |
| SEQ ID NO: 1245 | LPSAPAVVIHTLDKS |
| SEQ ID NO: 1246 | PSAPAVVIHTLDKSF |
| SEQ ID NO: 1247 | GPTERPTIIFPLPSAP |
| SEQ ID NO: 1248 | PTERPTIIFPLPSAPA |
| SEQ ID NO: 1249 | TERPTIIFPLPSAPAV |
| SEQ ID NO: 1250 | ERPTIIFPLPSAPAVV |
| SEQ ID NO: 1251 | RPTIIFPLPSAPAVVI |
| SEQ ID NO: 1252 | PTIIFPLPSAPAVVIH |
| SEQ ID NO: 1253 | TIIFPLPSAPAVVIHT |
| SEQ ID NO: 1254 | IIFPLPSAPAVVIHTL |
| SEQ ID NO: 1255 | IFPLPSAPAVVIHTLD |
| SEQ ID NO: 1256 | FPLPSAPAVVIHTLDK |
| SEQ ID NO: 1257 | PLPSAPAVVIHTLDKS |
| SEQ ID NO: 1258 | LPSAPAVVIHTLDKSF |
| SEQ ID NO: 1259 | PSAPAVVIHTLDKSFD |
| SEQ ID NO: 1260 | PGPTERPTIIFPLPSAP |
| SEQ ID NO: 1261 | GPTERPTIIFPLPSAPA |
| SEQ ID NO: 1262 | PTERPTIIFPLPSAPAV |
| SEQ ID NO: 1263 | TERPTIIFPLPSAPAVV |
| SEQ ID NO: 1264 | ERPTIIFPLPSAPAVVI |
| SEQ ID NO: 1265 | RPTIIFPLPSAPAVVIH |
| SEQ ID NO: 1266 | PTIIFPLPSAPAVVIHT |
| SEQ ID NO: 1267 | TIIFPLPSAPAVVIHTL |
| SEQ ID NO: 1268 | IIFPLPSAPAVVIHTLD |
| SEQ ID NO: 1269 | IFPLPSAPAVVIHTLDK |
| SEQ ID NO: 1270 | FPLPSAPAVVIHTLDKS |
| SEQ ID NO: 1271 | PLPSAPAVVIHTLDKSF |
| SEQ ID NO: 1272 | LPSAPAVVIHTLDKSFD |
| SEQ ID NO: 1273 | PSAPAVVIHTLDKSFDY |
| SEQ ID NO: 1274 | YPGPTERPTIIFPLPSAP |
| SEQ ID NO: 1275 | PGPTERPTIIFPLPSAPA |
| SEQ ID NO: 1276 | GPTERPTIIFPLPSAPAV |
| SEQ ID NO: 1277 | PTERPTIIFPLPSAPAVV |
| SEQ ID NO: 1278 | TERPTIIFPLPSAPAVVI |
| SEQ ID NO: 1279 | ERPTIIFPLPSAPAVVIH |
| SEQ ID NO: 1280 | RPTIIFPLPSAPAVVIHT |
| SEQ ID NO: 1281 | PTIIFPLPSAPAVVIHTL |
| SEQ ID NO: 1282 | TIIFPLPSAPAVVIHTLD |
| SEQ ID NO: 1283 | IIFPLPSAPAVVIHTLDK |
| SEQ ID NO: 1284 | IFPLPSAPAVVIHTLDKS |
| SEQ ID NO: 1285 | FPLPSAPAVVIHTLDKSF |
| SEQ ID NO: 1286 | PLPSAPAVVIHTLDKSFD |
| SEQ ID NO: 1287 | LPSAPAVVIHTLDKSFDY |
| SEQ ID NO: 1288 | PSAPAVVIHTLDKSFDYY |
| SEQ ID NO: 1289 | IYPGPTERPTIIFPLPSAP |
| SEQ ID NO: 1290 | YPGPTERPTIIFPLPSAPA |
| SEQ ID NO: 1291 | PGPTERPTIIFPLPSAPAV |
| SEQ ID NO: 1292 | GPTERPTIIFPLPSAPAVV |
| SEQ ID NO: 1293 | PTERPTIIFPLPSAPAVVI |
| SEQ ID NO: 1294 | TERPTIIFPLPSAPAVVIH |
| SEQ ID NO: 1295 | ERPTIIFPLPSAPAVVIHT |
| SEQ ID NO: 1296 | RPTIIFPLPSAPAVVIHTL |
| SEQ ID NO: 1297 | PTIIFPLPSAPAVVIHTLD |
| SEQ ID NO: 1298 | TIIFPLPSAPAVVIHTLDK |
| SEQ ID NO: 1299 | IIFPLPSAPAVVIHTLDKS |
| SEQ ID NO: 1300 | IFPLPSAPAVVIHTLDKSF |
| SEQ ID NO: 1301 | FPLPSAPAVVIHTLDKSFD |
| SEQ ID NO: 1302 | PLPSAPAVVIHTLDKSFDY |
| SEQ ID NO: 1303 | LPSAPAVVIHTLDKSFDYY |
| SEQ ID NO: 1304 | PSAPAVVIHTLDKSFDYYL |
| SEQ ID NO: 1305 | VIYPGPTERPTIIFPLPSAP |
| SEQ ID NO: 1306 | IYPGPTERPTIIFPLPSAPA |
| SEQ ID NO: 1307 | YPGPTERPTIIFPLPSAPAV |
| SEQ ID NO: 1308 | PGPTERPTIIFPLPSAPAVV |
| SEQ ID NO: 1309 | GPTERPTIIFPLPSAPAVVI |
| SEQ ID NO: 1310 | PTERPTIIFPLPSAPAVVIH |
| SEQ ID NO: 1311 | TERPTIIFPLPSAPAVVIHT |
| SEQ ID NO: 1312 | ERPTIIFPLPSAPAVVIHTL |
| SEQ ID NO: 1313 | RPTIIFPLPSAPAVVIHTLD |
| SEQ ID NO: 1314 | PTIIFPLPSAPAVVIHTLDK |
| SEQ ID NO: 1315 | TIIFPLPSAPAVVIHTLDKS |
| SEQ ID NO: 1316 | IIFPLPSAPAVVIHTLDKSF |
| SEQ ID NO: 1317 | IFPLPSAPAVVIHTLDKSFD |
| SEQ ID NO: 1318 | FPLPSAPAVVIHTLDKSFDY |
| SEQ ID NO: 1319 | PLPSAPAVVIHTLDKSFDYY |
| SEQ ID NO: 1320 | LPSAPAVVIHTLDKSFDYYL |
| SEQ ID NO: 1321 | PSAPAVVIHTLDKSFDYYLH |

TABLE 11
P(T/S)AP Motif Containing Peptides from Human
Papillomavrius Type 35 Major Capsid Protein L1
(GenBank Accession No. P27232)

| | |
|---|---|
| SEQ ID NO: 1322 | TCQKPSAP |
| SEQ ID NO: 1323 | CQKPSAPK |
| SEQ ID NO: 1324 | QKPSAPKP |
| SEQ ID NO: 1325 | KPSAPKPK |
| SEQ ID NO: 1326 | PSAPKPKD |
| SEQ ID NO: 1327 | VTCQKPSAP |
| SEQ ID NO: 1328 | TCQKPSAPK |
| SEQ ID NO: 1329 | CQKPSAPKP |
| SEQ ID NO: 1330 | QKPSAPKPK |
| SEQ ID NO: 1331 | KPSAPKPKD |
| SEQ ID NO: 1332 | PSAPKPKDD |
| SEQ ID NO: 1333 | AVTCQKPSAP |
| SEQ ID NO: 1334 | VTCQKPSAPK |
| SEQ ID NO: 1335 | TCQKPSAPKP |
| SEQ ID NO: 1336 | CQKPSAPKPK |
| SEQ ID NO: 1337 | QKPSAPKPKD |
| SEQ ID NO: 1338 | KPSAPKPKDD |
| SEQ ID NO: 1339 | PSAPKPKDDP |
| SEQ ID NO: 1340 | QAVTCQKPSAP |
| SEQ ID NO: 1341 | AVTCQKPSAPK |
| SEQ ID NO: 1342 | VTCQKPSAPKP |
| SEQ ID NO: 1343 | TCQKPSAPKPK |
| SEQ ID NO: 1344 | CQKPSAPKPKD |
| SEQ ID NO: 1345 | QKPSAPKPKDD |
| SEQ ID NO: 1346 | KPSAPKPKDDP |
| SEQ ID NO: 1347 | PSAPKPKDDPL |
| SEQ ID NO: 1348 | SQAVTCQKPSAP |
| SEQ ID NO: 1349 | QAVTCQKPSAPK |
| SEQ ID NO: 1350 | AVTCQKPSAPKP |
| SEQ ID NO: 1351 | VTCQKPSAPKPK |
| SEQ ID NO: 1352 | TCQKPSAPKPKD |
| SEQ ID NO: 1353 | CQKPSAPKPKDD |
| SEQ ID NO: 1354 | QKPSAPKPKDDP |
| SEQ ID NO: 1355 | KPSAPKPKDDPL |
| SEQ ID NO: 1356 | PSAPKPKDDPLK |
| SEQ ID NO: 1357 | TSQAVTCQKPSAP |
| SEQ ID NO: 1358 | SQAVTCQKPSAPK |
| SEQ ID NO: 1359 | QAVTCQKPSAPKP |
| SEQ ID NO: 1360 | AVTCQKPSAPKPK |
| SEQ ID NO: 1361 | VTCQKPSAPKPKD |
| SEQ ID NO: 1362 | TCQKPSAPKPKDD |
| SEQ ID NO: 1363 | CQKPSAPKPKDDP |
| SEQ ID NO: 1364 | QKPSAPKPKDDPL |
| SEQ ID NO: 1365 | KPSAPKPKDDPLK |
| SEQ ID NO: 1366 | PSAPKPKDDPLKN |
| SEQ ID NO: 1367 | VTSQAVTCQKPSAP |
| SEQ ID NO: 1368 | TSQAVTCQKPSAPK |
| SEQ ID NO: 1369 | SQAVTCQKPSAPKP |
| SEQ ID NO: 1370 | QAVTCQKPSAPKPK |
| SEQ ID NO: 1371 | AVTCQKPSAPKPKD |
| SEQ ID NO: 1372 | VTCQKPSAPKPKDD |
| SEQ ID NO: 1373 | TCQKPSAPKPKDDP |
| SEQ ID NO: 1374 | CQKPSAPKPKDDPL |
| SEQ ID NO: 1375 | QKPSAPKPKDDPLK |
| SEQ ID NO: 1376 | KPSAPKPKDDPLKN |
| SEQ ID NO: 1377 | PSAPKPKDDPLKNY |
| SEQ ID NO: 1378 | YVTSQAVTCQKPSAP |
| SEQ ID NO: 1379 | VTSQAVTCQKPSAPK |
| SEQ ID NO: 1380 | TSQAVTCQKPSAPKP |
| SEQ ID NO: 1381 | SQAVTCQKPSAPKPK |
| SEQ ID NO: 1382 | QAVTCQKPSAPKPKD |
| SEQ ID NO: 1383 | AVTCQKPSAPKPKDD |
| SEQ ID NO: 1384 | VTCQKPSAPKPKDDP |
| SEQ ID NO: 1385 | TCQKPSAPKPKDDPL |
| SEQ ID NO: 1386 | CQKPSAPKPKDDPLK |
| SEQ ID NO: 1387 | QKPSAPKPKDDPLKN |
| SEQ ID NO: 1388 | KPSAPKPKDDPLKNY |
| SEQ ID NO: 1389 | PSAPKPKDDPLKNYT |
| SEQ ID NO: 1390 | RYVTSQAVTCQKPSAP |
| SEQ ID NO: 1391 | YVTSQAVTCQKPSAPK |
| SEQ ID NO: 1392 | VTSQAVTCQKPSAPKP |
| SEQ ID NO: 1393 | TSQAVTCQKPSAPKPK |
| SEQ ID NO: 1394 | SQAVTCQKPSAPKPKD |
| SEQ ID NO: 1395 | QAVTCQKPSAPKPKDD |
| SEQ ID NO: 1396 | AVTCQKPSAPKPKDDP |
| SEQ ID NO: 1397 | VTCQKPSAPKPKDDPL |
| SEQ ID NO: 1398 | TCQKPSAPKPKDDPLK |
| SEQ ID NO: 1399 | CQKPSAPKPKDDPLKN |
| SEQ ID NO: 1400 | QKPSAPKPKDDPLKNY |
| SEQ ID NO: 1401 | KPSAPKPKDDPLKNYT |
| SEQ ID NO: 1402 | PSAPKPKDDPLKNYTF |
| SEQ ID NO: 1403 | YRYVTSQAVTCQKPSAP |
| SEQ ID NO: 1404 | RYVTSQAVTCQKPSAPK |
| SEQ ID NO: 1405 | YVTSQAVTCQKPSAPKP |
| SEQ ID NO: 1406 | VTSQAVTCQKPSAPKPK |
| SEQ ID NO: 1407 | TSQAVTCQKPSAPKPKD |
| SEQ ID NO: 1408 | SQAVTCQKPSAPKPKDD |
| SEQ ID NO: 1409 | QAVTCQKPSAPKPKDDP |
| SEQ ID NO: 1410 | AVTCQKPSAPKPKDDPL |
| SEQ ID NO: 1411 | VTCQKPSAPKPKDDPLK |
| SEQ ID NO: 1412 | TCQKPSAPKPKDDPLKN |
| SEQ ID NO: 1413 | CQKPSAPKPKDDPLKNY |
| SEQ ID NO: 1414 | QKPSAPKPKDDPLKNYT |
| SEQ ID NO: 1415 | KPSAPKPKDDPLKNYTF |
| SEQ ID NO: 1416 | PSAPKPKDDPLKNYTFW |
| SEQ ID NO: 1417 | TYRYVTSQAVTCQKPSAP |
| SEQ ID NO: 1418 | YRYVTSQAVTCQKPSAPK |
| SEQ ID NO: 1419 | RYVTSQAVTCQKPSAPKP |
| SEQ ID NO: 1420 | YVTSQAVTCQKPSAPKPK |
| SEQ ID NO: 1421 | VTSQAVTCQKPSAPKPKD |
| SEQ ID NO: 1422 | TSQAVTCQKPSAPKPKDD |
| SEQ ID NO: 1423 | SQAVTCQKPSAPKPKDDP |
| SEQ ID NO: 1424 | QAVTCQKPSAPKPKDDPL |
| SEQ ID NO: 1425 | AVTCQKPSAPKPKDDPLK |
| SEQ ID NO: 1426 | VTCQKPSAPKPKDDPLKN |
| SEQ ID NO: 1427 | TCQKPSAPKPKDDPLKNY |
| SEQ ID NO: 1428 | CQKPSAPKPKDDPLKNYT |
| SEQ ID NO: 1429 | QKPSAPKPKDDPLKNYTF |
| SEQ ID NO: 1430 | KPSAPKPKDDPLKNYTFW |
| SEQ ID NO: 1431 | PSAPKPKDDPLKNYTFWE |
| SEQ ID NO: 1432 | DTYRYVTSQAVTCQKPSAP |
| SEQ ID NO: 1433 | TYRYVTSQAVTCQKPSAPK |
| SEQ ID NO: 1434 | YRYVTSQAVTCQKPSAPKP |
| SEQ ID NO: 1435 | RYVTSQAVTCQKPSAPKPK |
| SEQ ID NO: 1436 | YVTSQAVTCQKPSAPKPKD |
| SEQ ID NO: 1437 | VTSQAVTCQKPSAPKPKDD |
| SEQ ID NO: 1438 | TSQAVTCQKPSAPKPKDDP |
| SEQ ID NO: 1439 | SQAVTCQKPSAPKPKDDPL |
| SEQ ID NO: 1440 | QAVTCQKPSAPKPKDDPLK |
| SEQ ID NO: 1441 | AVTCQKPSAPKPKDDPLKN |
| SEQ ID NO: 1442 | VTCQKPSAPKPKDDPLKNY |
| SEQ ID NO: 1443 | TCQKPSAPKPKDDPLKNYT |
| SEQ ID NO: 1444 | CQKPSAPKPKDDPLKNYTF |
| SEQ ID NO: 1445 | QKPSAPKPKDDPLKNYTFW |
| SEQ ID NO: 1446 | KPSAPKPKDDPLKNYTFWE |
| SEQ ID NO: 1447 | PSAPKPKDDPLKNYTFWEV |
| SEQ ID NO: 1448 | EDTYRYVTSQAVTCQKPSAP |
| SEQ ID NO: 1449 | DTYRYVTSQAVTCQKPSAPK |
| SEQ ID NO: 1450 | TYRYVTSQAVTCQKPSAPKP |
| SEQ ID NO: 1451 | YRYVTSQAVTCQKPSAPKPK |
| SEQ ID NO: 1452 | RYVTSQAVTCQKPSAPKPKD |
| SEQ ID NO: 1453 | YVTSQAVTCQKPSAPKPKDD |
| SEQ ID NO: 1454 | VTSQAVTCQKPSAPKPKDDP |
| SEQ ID NO: 1455 | TSQAVTCQKPSAPKPKDDPL |
| SEQ ID NO: 1456 | SQAVTCQKPSAPKPKDDPLK |
| SEQ ID NO: 1457 | QAVTCQKPSAPKPKDDPLKN |
| SEQ ID NO: 1458 | AVTCQKPSAPKPKDDPLKNY |
| SEQ ID NO: 1459 | VTCQKPSAPKPKDDPLKNYT |
| SEQ ID NO: 1460 | TCQKPSAPKPKDDPLKNYTF |
| SEQ ID NO: 1461 | CQKPSAPKPKDDPLKNYTFW |
| SEQ ID NO: 1462 | QKPSAPKPKDDPLKNYTFWE |
| SEQ ID NO: 1463 | KPSAPKPKDDPLKNYTFWEV |
| SEQ ID NO: 1464 | PSAPKPKDDPLKNYTFWEVD |

TABLE 12
P(T/S)AP Motif Containing Peptides from Human
Papillomavrius Type 6b Minor Capsid Protein L2
(GenBank Accession No. NP_040303)

| | |
|---|---|
| SEQ ID NO: 1465 | DITFPTAP |
| SEQ ID NO: 1466 | ITFPTAPM |
| SEQ ID NO: 1467 | TFPTAPMG |
| SEQ ID NO: 1468 | FPTAPMGT |
| SEQ ID NO: 1469 | PTAPMGTP |
| SEQ ID NO: 1470 | PDITFPTAP |
| SEQ ID NO: 1471 | DITFPTAPM |
| SEQ ID NO: 1472 | ITFPTAPMG |
| SEQ ID NO: 1473 | TFPTAPMGT |
| SEQ ID NO: 1474 | FPTAPMGTP |
| SEQ ID NO: 1475 | PTAPMGTPF |
| SEQ ID NO: 1476 | GPDITFPTAP |
| SEQ ID NO: 1477 | PDITFPTAPM |
| SEQ ID NO: 1478 | DITFPTAPMG |
| SEQ ID NO: 1479 | ITFPTAPMGT |
| SEQ ID NO: 1480 | TFPTAPMGTP |
| SEQ ID NO: 1481 | FPTAPMGTPF |
| SEQ ID NO: 1482 | PTAPMGTPFS |
| SEQ ID NO: 1483 | SGPDITFPTAP |
| SEQ ID NO: 1484 | GPDITFPTAPM |
| SEQ ID NO: 1485 | PDITFPTAPMG |
| SEQ ID NO: 1486 | DITFPTAPMGT |
| SEQ ID NO: 1487 | ITFPTAPMGTP |
| SEQ ID NO: 1488 | TFPTAPMGTPF |
| SEQ ID NO: 1489 | FPTAPMGTPFS |
| SEQ ID NO: 1490 | PTAPMGTPFSP |
| SEQ ID NO: 1491 | QSGPDITFPTAP |
| SEQ ID NO: 1492 | SGPDITFPTAPM |
| SEQ ID NO: 1493 | GPDITFPTAPMG |
| SEQ ID NO: 1494 | PDITFPTAPMGT |
| SEQ ID NO: 1495 | DITFPTAPMGTP |
| SEQ ID NO: 1496 | ITFPTAPMGTPF |
| SEQ ID NO: 1497 | TFPTAPMGTPFS |
| SEQ ID NO: 1498 | FPTAPMGTPFSP |
| SEQ ID NO: 1499 | PTAPMGTPFSPV |
| SEQ ID NO: 1500 | LQSGPDITFPTAP |
| SEQ ID NO: 1501 | QSGPDITFPTAPM |
| SEQ ID NO: 1502 | SGPDITFPTAPMG |
| SEQ ID NO: 1503 | GPDITFPTAPMGT |
| SEQ ID NO: 1504 | PDITFPTAPMGTP |
| SEQ ID NO: 1505 | DITFPTAPMGTPF |
| SEQ ID NO: 1506 | ITFPTAPMGTPFS |
| SEQ ID NO: 1507 | TFPTAPMGTPFSP |
| SEQ ID NO: 1508 | FPTAPMGTPFSPV |
| SEQ ID NO: 1509 | PTAPMGTPFSPVT |
| SEQ ID NO: 1510 | PLQSGPDITFPTAP |
| SEQ ID NO: 1511 | LQSGPDITFPTAPM |
| SEQ ID NO: 1512 | QSGPDITFPTAPMG |
| SEQ ID NO: 1513 | SGPDITFPTAPMGT |
| SEQ ID NO: 1514 | GPDITFPTAPMGTP |
| SEQ ID NO: 1515 | PDITFPTAPMGTPF |
| SEQ ID NO: 1516 | DITFPTAPMGTPFS |
| SEQ ID NO: 1517 | ITFPTAPMGTPFSP |
| SEQ ID NO: 1518 | TFPTAPMGTPFSPV |
| SEQ ID NO: 1519 | FPTAPMGTPFSPVT |
| SEQ ID NO: 1520 | PTAPMGTPFSPVTP |
| SEQ ID NO: 1521 | LFLQSGPDITFPTAP |
| SEQ ID NO: 1522 | FLQSGPDITFPTAPM |
| SEQ ID NO: 1523 | LQSGPDITFPTAPMG |
| SEQ ID NO: 1524 | QSGPDITFPTAPMGT |
| SEQ ID NO: 1525 | SGPDITFPTAPMGTP |
| SEQ ID NO: 1526 | GPDITFPTAPMGTPF |
| SEQ ID NO: 1527 | PDITFPTAPMGTPFS |
| SEQ ID NO: 1528 | DITFPTAPMGTPFSP |
| SEQ ID NO: 1529 | ITFPTAPMGTPFSPV |
| SEQ ID NO: 1530 | TFPTAPMGTPFSPVT |
| SEQ ID NO: 1531 | FPTAPMGTPFSPVTP |
| SEQ ID NO: 1532 | PTAPMGTPFSPVTPA |
| SEQ ID NO: 1533 | DLFLQSGPDITFPTAP |
| SEQ ID NO: 1534 | LFLQSGPDITFPTAPM |
| SEQ ID NO: 1535 | FLQSGPDITFPTAPMG |
| SEQ ID NO: 1536 | LQSGPDITFPTAPMGT |
| SEQ ID NO: 1537 | QSGPDITFPTAPMGTP |
| SEQ ID NO: 1538 | SGPDITFPTAPMGTPF |
| SEQ ID NO: 1539 | GPDITFPTAPMGTPFS |
| SEQ ID NO: 1540 | PDITFPTAPMGTPFSP |
| SEQ ID NO: 1541 | DITFPTAPMGTPFSPV |
| SEQ ID NO: 1542 | ITFPTAPMGTPFSPVT |
| SEQ ID NO: 1543 | TFPTAPMGTPFSPVTP |
| SEQ ID NO: 1544 | FPTAPMGTPFSPVTPA |
| SEQ ID NO: 1545 | PTAPMGTPFSPVTPAL |
| SEQ ID NO: 1546 | NDLFLQSGPDITFPTAP |
| SEQ ID NO: 1547 | DLFLQSGPDITFPTAPM |
| SEQ ID NO: 1548 | LFLQSGPDITFPTAPMG |
| SEQ ID NO: 1549 | FLQSGPDITFPTAPMGT |
| SEQ ID NO: 1550 | LQSGPDITFPTAPMGTP |
| SEQ ID NO: 1551 | QSGPDITFPTAPMGTPF |
| SEQ ID NO: 1552 | SGPDITFPTAPMGTPFS |
| SEQ ID NO: 1553 | GPDITFPTAPMGTPFSP |
| SEQ ID NO: 1554 | PDITFPTAPMGTPFSPV |
| SEQ ID NO: 1555 | DITFPTAPMGTPFSPVT |
| SEQ ID NO: 1556 | ITFPTAPMGTPFSPVTP |
| SEQ ID NO: 1557 | TFPTAPMGTPFSPVTPA |
| SEQ ID NO: 1558 | FPTAPMGTPFSPVTPAL |
| SEQ ID NO: 1559 | PTAPMGTPFSPVTPALP |
| SEQ ID NO: 1560 | PNDLFLQSGPDITFPTAP |
| SEQ ID NO: 1561 | NDLFLQSGPDITFPTAPM |
| SEQ ID NO: 1562 | DLFLQSGPDITFPTAPMG |
| SEQ ID NO: 1563 | LFLQSGPDITFPTAPMGT |
| SEQ ID NO: 1564 | FLQSGPDITFPTAPMGTP |
| SEQ ID NO: 1565 | LQSGPDITFPTAPMGTPF |
| SEQ ID NO: 1566 | QSGPDITFPTAPMGTPFS |
| SEQ ID NO: 1567 | SGPDITFPTAPMGTPFSP |
| SEQ ID NO: 1568 | GPDITFPTAPMGTPFSPV |
| SEQ ID NO: 1569 | PDITFPTAPMGTPFSPVT |
| SEQ ID NO: 1570 | DITFPTAPMGTPFSPVTP |
| SEQ ID NO: 1571 | ITFPTAPMGTPFSPVTPA |
| SEQ ID NO: 1572 | TFPTAPMGTPFSPVTPAL |
| SEQ ID NO: 1573 | FPTAPMGTPFSPVTPALP |
| SEQ ID NO: 1574 | PTAPMGTPFSPVTPALPT |
| SEQ ID NO: 1575 | LPNDLFLQSGPDITFPTAP |
| SEQ ID NO: 1576 | PNDLFLQSGPDITFPTAPM |
| SEQ ID NO: 1577 | NDLFLQSGPDITFPTAPMG |
| SEQ ID NO: 1578 | DLFLQSGPDITFPTAPMGT |
| SEQ ID NO: 1579 | LFLQSGPDITFPTAPMGTP |
| SEQ ID NO: 1580 | FLQSGPDITFPTAPMGTPF |
| SEQ ID NO: 1581 | LQSGPDITFPTAPMGTPFS |
| SEQ ID NO: 1582 | QSGPDITFPTAPMGTPFSP |
| SEQ ID NO: 1583 | SGPDITFPTAPMGTPFSPV |
| SEQ ID NO: 1584 | GPDITFPTAPMGTPFSPVT |
| SEQ ID NO: 1585 | PDITFPTAPMGTPFSPVTP |
| SEQ ID NO: 1586 | DITFPTAPMGTPFSPVTPA |
| SEQ ID NO: 1587 | ITFPTAPMGTPFSPVTPAL |
| SEQ ID NO: 1588 | TFPTAPMGTPFSPVTPALP |
| SEQ ID NO: 1589 | FPTAPMGTPFSPVTPALPT |
| SEQ ID NO: 1590 | PTAPMGTPFSPVTPALPTG |
| SEQ ID NO: 1591 | SLPNDLFLQSGPDITFPTAP |
| SEQ ID NO: 1592 | LPNDLFLQSGPDITFPTAPM |
| SEQ ID NO: 1593 | PNDLFLQSGPDITFPTAPMG |
| SEQ ID NO: 1594 | NDLFLQSGPDITFPTAPMGT |
| SEQ ID NO: 1595 | DLFLQSGPDITFPTAPMGTP |
| SEQ ID NO: 1596 | LFLQSGPDITFPTAPMGTPF |
| SEQ ID NO: 1597 | FLQSGPDITFPTAPMGTPFS |
| SEQ ID NO: 1598 | LQSGPDITFPTAPMGTPFSP |
| SEQ ID NO: 1599 | QSGPDITFPTAPMGTPFSPV |
| SEQ ID NO: 1600 | SGPDITFPTAPMGTPFSPVT |
| SEQ ID NO: 1601 | GPDITFPTAPMGTPFSPVTP |
| SEQ ID NO: 1602 | PDITFPTAPMGTPFSPVTPA |
| SEQ ID NO: 1603 | DITFPTAPMGTPFSPVTPAL |
| SEQ ID NO: 1604 | ITFPTAPMGTPFSPVTPALP |
| SEQ ID NO: 1605 | TFPTAPMGTPFSPVTPALPT |
| SEQ ID NO: 1606 | FPTAPMGTPFSPVTPALPTG |
| SEQ ID NO: 1607 | PTAPMGTPFSPVTPALPTGP |

TABLE 13
P(T/S)AP Motif Containing Peptides from
Human Papillomavirus Type 9 Late Protein
(GenBank Accession No. NP_041865)

| | |
|---|---|
| SEQ ID NO: 1608 | RPIDPTAP |
| SEQ ID NO: 1609 | PIDPTAPS |
| SEQ ID NO: 1610 | IDPTAPSI |
| SEQ ID NO: 1611 | DPTAPSIV |
| SEQ ID NO: 1612 | PTAPSIVT |
| SEQ ID NO: 1613 | VRPIDPTAP |
| SEQ ID NO: 1614 | RPIDPTAPS |
| SEQ ID NO: 1615 | PIDPTAPSI |
| SEQ ID NO: 1616 | IDPTAPSIV |
| SEQ ID NO: 1617 | DPTAPSIVT |
| SEQ ID NO: 1618 | PTAPSIVTG |
| SEQ ID NO: 1619 | TVRPIDPTAP |
| SEQ ID NO: 1620 | VRPIDPTAPS |
| SEQ ID NO: 1621 | RPIDPTAPSI |
| SEQ ID NO: 1622 | PIDPTAPSIV |
| SEQ ID NO: 1623 | IDPTAPSIVT |
| SEQ ID NO: 1624 | DPTAPSIVTG |
| SEQ ID NO: 1625 | PTAPSIVTGT |
| SEQ ID NO: 1626 | DTVRPIDPTAP |
| SEQ ID NO: 1627 | TVRPIDPTAPS |
| SEQ ID NO: 1628 | VRPIDPTAPSI |
| SEQ ID NO: 1629 | RPIDPTAPSIV |
| SEQ ID NO: 1630 | PIDPTAPSIVT |
| SEQ ID NO: 1631 | IDPTAPSIVTG |
| SEQ ID NO: 1632 | DPTAPSIVTGT |
| SEQ ID NO: 1633 | PTAPSIVTGTD |
| SEQ ID NO: 1634 | LDTVRPIDPTAP |
| SEQ ID NO: 1635 | DTVRPIDPTAPS |
| SEQ ID NO: 1636 | TVRPIDPTAPSI |
| SEQ ID NO: 1637 | VRPIDPTAPSIV |
| SEQ ID NO: 1638 | RPIDPTAPSIVT |
| SEQ ID NO: 1639 | PIDPTAPSIVTG |
| SEQ ID NO: 1640 | IDPTAPSIVTGT |
| SEQ ID NO: 1641 | DPTAPSIVTGTD |
| SEQ ID NO: 1642 | PTAPSIVTGTDS |
| SEQ ID NO: 1643 | PLDTVRPIDPTAP |
| SEQ ID NO: 1644 | LDTVRPIDPTAPS |
| SEQ ID NO: 1645 | DTVRPIDPTAPSI |
| SEQ ID NO: 1646 | TVRPIDPTAPSIV |
| SEQ ID NO: 1647 | VRPIDPTAPSIVT |
| SEQ ID NO: 1648 | RPIDPTAPSIVTG |
| SEQ ID NO: 1649 | PIDPTAPSIVTGT |
| SEQ ID NO: 1650 | IDPTAPSIVTGTD |
| SEQ ID NO: 1651 | DPTAPSIVTGTDS |
| SEQ ID NO: 1652 | PTAPSIVTGTDST |
| SEQ ID NO: 1653 | IPLDTVRPIDPTAP |
| SEQ ID NO: 1654 | PLDTVRPIDPTAPS |
| SEQ ID NO: 1655 | LDTVRPIDPTAPSI |
| SEQ ID NO: 1656 | DTVRPIDPTAPSIV |
| SEQ ID NO: 1657 | TVRPIDPTAPSIVT |
| SEQ ID NO: 1658 | VRPIDPTAPSIVTG |
| SEQ ID NO: 1659 | RPIDPTAPSIVTGT |
| SEQ ID NO: 1660 | PIDPTAPSIVTGTD |
| SEQ ID NO: 1661 | IDPTAPSIVTGTDS |
| SEQ ID NO: 1662 | DPTAPSIVTGTDST |
| SEQ ID NO: 1663 | PTAPSIVTGTDSTV |
| SEQ ID NO: 1664 | LIPLDTVRPIDPTAP |
| SEQ ID NO: 1665 | IPLDTVRPIDPTAPS |
| SEQ ID NO: 1666 | PLDTVRPIDPTAPSI |
| SEQ ID NO: 1667 | LDTVRPIDPTAPSIV |
| SEQ ID NO: 1668 | DTVRPIDPTAPSIVT |
| SEQ ID NO: 1669 | TVRPIDPTAPSIVTG |
| SEQ ID NO: 1670 | VRPIDPTAPSIVTGT |
| SEQ ID NO: 1671 | RPIDPTAPSIVTGTD |
| SEQ ID NO: 1672 | PIDPTAPSIVTGTDS |
| SEQ ID NO: 1673 | IDPTAPSIVTGTDST |
| SEQ ID NO: 1674 | DPTAPSIVTGTDSTV |
| SEQ ID NO: 1675 | PTAPSIVTGTDSTVD |
| SEQ ID NO: 1676 | DLIPLDTVRPIDPTAP |
| SEQ ID NO: 1677 | LIPLDTVRPIDPTAPS |
| SEQ ID NO: 1678 | IPLDTVRPIDPTAPSI |
| SEQ ID NO: 1679 | PLDTVRPIDPTAPSIV |
| SEQ ID NO: 1680 | LDTVRPIDPTAPSIVT |
| SEQ ID NO: 1681 | DTVRPIDPTAPSIVTG |
| SEQ ID NO: 1682 | TVRPIDPTAPSIVTGT |
| SEQ ID NO: 1683 | VRPIDPTAPSIVTGTD |
| SEQ ID NO: 1684 | RPIDPTAPSIVTGTDS |
| SEQ ID NO: 1685 | PIDPTAPSIVTGTDST |
| SEQ ID NO: 1686 | IDPTAPSIVTGTDSTV |
| SEQ ID NO: 1687 | DPTAPSIVTGTDSTVD |
| SEQ ID NO: 1688 | PTAPSIVTGTDSTVDL |
| SEQ ID NO: 1689 | TDLIPLDTVRPIDPTAP |
| SEQ ID NO: 1690 | DLIPLDTVRPIDPTAPS |
| SEQ ID NO: 1691 | LIPLDTVRPIDPTAPSI |
| SEQ ID NO: 1692 | IPLDTVRPIDPTAPSIV |
| SEQ ID NO: 1693 | PLDTVRPIDPTAPSIVT |
| SEQ ID NO: 1694 | LDTVRPIDPTAPSIVTG |
| SEQ ID NO: 1695 | DTVRPIDPTAPSIVTGT |
| SEQ ID NO: 1696 | TVRPIDPTAPSIVTGTD |
| SEQ ID NO: 1697 | VRPIDPTAPSIVTGTDS |
| SEQ ID NO: 1698 | RPIDPTAPSIVTGTDST |
| SEQ ID NO: 1699 | PIDPTAPSIVTGTDSTV |
| SEQ ID NO: 1700 | IDPTAPSIVTGTDSTVD |
| SEQ ID NO: 1701 | DPTAPSIVTGTDSTVDL |
| SEQ ID NO: 1702 | PTAPSIVTGTDSTVDLL |
| SEQ ID NO: 1703 | PTDLIPLDTVRPIDPTAP |
| SEQ ID NO: 1704 | TDLIPLDTVRPIDPTAPS |
| SEQ ID NO: 1705 | DLIPLDTVRPIDPTAPSI |
| SEQ ID NO: 1706 | LIPLDTVRPIDPTAPSIV |
| SEQ ID NO: 1707 | IPLDTVRPIDPTAPSIVT |
| SEQ ID NO: 1708 | PLDTVRPIDPTAPSIVTG |
| SEQ ID NO: 1709 | LDTVRPIDPTAPSIVTGT |
| SEQ ID NO: 1710 | DTVRPIDPTAPSIVTGTD |
| SEQ ID NO: 1711 | TVRPIDPTAPSIVTGTDS |
| SEQ ID NO: 1712 | VRPIDPTAPSIVTGTDST |
| SEQ ID NO: 1713 | RPIDPTAPSIVTGTDSTV |
| SEQ ID NO: 1714 | PIDPTAPSIVTGTDSTVD |
| SEQ ID NO: 1715 | IDPTAPSIVTGTDSTVDL |
| SEQ ID NO: 1716 | DPTAPSIVTGTDSTVDLL |
| SEQ ID NO: 1717 | PTAPSIVTGTDSTVDLLP |
| SEQ ID NO: 1718 | GPTDLIPLDTVRPIDPTAP |
| SEQ ID NO: 1719 | PTDLIPLDTVRPIDPTAPS |
| SEQ ID NO: 1720 | TDLIPLDTVRPIDPTAPSI |
| SEQ ID NO: 1721 | DLIPLDTVRPIDPTAPSIV |
| SEQ ID NO: 1722 | LIPLDTVRPIDPTAPSIVT |
| SEQ ID NO: 1723 | IPLDTVRPIDPTAPSIVTG |
| SEQ ID NO: 1724 | PLDTVRPIDPTAPSIVTGT |
| SEQ ID NO: 1725 | LDTVRPIDPTAPSIVTGTD |
| SEQ ID NO: 1726 | DTVRPIDPTAPSIVTGTDS |
| SEQ ID NO: 1727 | TVRPIDPTAPSIVTGTDST |
| SEQ ID NO: 1728 | VRPIDPTAPSIVTGTDSTV |
| SEQ ID NO: 1729 | RPIDPTAPSIVTGTDSTVD |
| SEQ ID NO: 1730 | PIDPTAPSIVTGTDSTVDL |
| SEQ ID NO: 1731 | IDPTAPSIVTGTDSTVDLL |
| SEQ ID NO: 1732 | DPTAPSIVTGTDSTVDLLP |
| SEQ ID NO: 1733 | PTAPSIVTGTDSTVDLLPG |
| SEQ ID NO: 1734 | IGPTDLIPLDTVRPIDPTAP |
| SEQ ID NO: 1735 | GPTDLIPLDTVRPIDPTAPS |
| SEQ ID NO: 1736 | PTDLIPLDTVRPIDPTAPSI |
| SEQ ID NO: 1737 | TDLIPLDTVRPIDPTAPSIV |
| SEQ ID NO: 1738 | DLIPLDTVRPIDPTAPSIVT |
| SEQ ID NO: 1739 | LIPLDTVRPIDPTAPSIVTG |
| SEQ ID NO: 1740 | IPLDTVRPIDPTAPSIVTGT |
| SEQ ID NO: 1741 | PLDTVRPIDPTAPSIVTGTD |
| SEQ ID NO: 1742 | LDTVRPIDPTAPSIVTGTDS |
| SEQ ID NO: 1743 | DTVRPIDPTAPSIVTGTDST |
| SEQ ID NO: 1744 | TVRPIDPTAPSIVTGTDSTV |
| SEQ ID NO: 1745 | VRPIDPTAPSIVTGTDSTVD |
| SEQ ID NO: 1746 | RPIDPTAPSIVTGTDSTVDL |
| SEQ ID NO: 1747 | PIDPTAPSIVTGTDSTVDLL |
| SEQ ID NO: 1748 | IDPTAPSIVTGTDSTVDLLP |
| SEQ ID NO: 1749 | DPTAPSIVTGTDSTVDLLPG |
| SEQ ID NO: 1750 | PTAPSIVTGTDSTVDLLPGE |

TABLE 14
P(T/S)AP Motif Containing Peptides from
Human T-Cell Lymphotropic Virus Type Gag Protein
(GenBank Accession No. CAA61543)

| | |
|---|---|
| SEQ ID NO: 1751 | NQVSPSAP |
| SEQ ID NO: 1752 | QVSPSAPA |
| SEQ ID NO: 1753 | VSPSAPAA |
| SEQ ID NO: 1754 | SPSAPAAP |
| SEQ ID NO: 1755 | PSAPAAPV |
| SEQ ID NO: 1756 | KNQVSPSAP |
| SEQ ID NO: 1757 | NQVSPSAPA |
| SEQ ID NO: 1758 | QVSPSAPAA |
| SEQ ID NO: 1759 | VSPSAPAAP |
| SEQ ID NO: 1760 | SPSAPAAPV |
| SEQ ID NO: 1761 | PSAPAAPVP |
| SEQ ID NO: 1762 | VKNQVSPSAP |
| SEQ ID NO: 1763 | KNQVSPSAPA |
| SEQ ID NO: 1764 | NQVSPSAPAA |
| SEQ ID NO: 1765 | QVSPSAPAAP |
| SEQ ID NO: 1766 | VSPSAPAAPV |
| SEQ ID NO: 1767 | SPSAPAAPVP |
| SEQ ID NO: 1768 | PSAPAAPVPT |
| SEQ ID NO: 1769 | LVKNQVSPSAP |
| SEQ ID NO: 1770 | VKNQVSPSAPA |
| SEQ ID NO: 1771 | KNQVSPSAPAA |
| SEQ ID NO: 1772 | NQVSPSAPAAP |
| SEQ ID NO: 1773 | QVSPSAPAAPV |
| SEQ ID NO: 1774 | VSPSAPAAPVP |
| SEQ ID NO: 1775 | SPSAPAAPVPT |
| SEQ ID NO: 1776 | PSAPAAPVPTP |
| SEQ ID NO: 1777 | ILVKNQVSPSAP |
| SEQ ID NO: 1778 | LVKNQVSPSAPA |
| SEQ ID NO: 1779 | VKNQVSPSAPAA |
| SEQ ID NO: 1780 | KNQVSPSAPAAP |
| SEQ ID NO: 1781 | NQVSPSAPAAPV |
| SEQ ID NO: 1782 | QVSPSAPAAPVP |
| SEQ ID NO: 1783 | VSPSAPAAPVPT |
| SEQ ID NO: 1784 | SPSAPAAPVPTP |
| SEQ ID NO: 1785 | PSAPAAPVPTPI |
| SEQ ID NO: 1786 | NILVKNQVSPSAP |
| SEQ ID NO: 1787 | ILVKNQVSPSAPA |
| SEQ ID NO: 1788 | LVKNQVSPSAPAA |
| SEQ ID NO: 1789 | VKNQVSPSAPAAP |
| SEQ ID NO: 1790 | KNQVSPSAPAAPV |
| SEQ ID NO: 1791 | NQVSPSAPAAPVP |
| SEQ ID NO: 1792 | QVSPSAPAAPVPT |
| SEQ ID NO: 1793 | VSPSAPAAPVPTP |
| SEQ ID NO: 1794 | SPSAPAAPVPTPI |
| SEQ ID NO: 1795 | PSAPAAPVPTPIC |
| SEQ ID NO: 1796 | INILVKNQVSPSAP |
| SEQ ID NO: 1797 | NILVKNQVSPSAPA |
| SEQ ID NO: 1798 | ILVKNQVSPSAPAA |
| SEQ ID NO: 1799 | LVKNQVSPSAPAAP |
| SEQ ID NO: 1800 | VKNQVSPSAPAAPV |
| SEQ ID NO: 1801 | KNQVSPSAPAAPVP |
| SEQ ID NO: 1802 | NQVSPSAPAAPVPT |
| SEQ ID NO: 1803 | QVSPSAPAAPVPTP |
| SEQ ID NO: 1804 | VSPSAPAAPVPTPI |
| SEQ ID NO: 1805 | SPSAPAAPVPTPIC |
| SEQ ID NO: 1806 | PSAPAAPVPTPICP |
| SEQ ID NO: 1807 | IINILVKNQVSPSAP |
| SEQ ID NO: 1808 | INILVKNQVSPSAPA |
| SEQ ID NO: 1809 | NILVKNQVSPSAPAA |
| SEQ ID NO: 1810 | ILVKNQVSPSAPAAP |
| SEQ ID NO: 1811 | LVKNQVSPSAPAAPV |
| SEQ ID NO: 1812 | VKNQVSPSAPAAPVP |
| SEQ ID NO: 1813 | KNQVSPSAPAAPVPT |
| SEQ ID NO: 1814 | NQVSPSAPAAPVPTP |
| SEQ ID NO: 1815 | QVSPSAPAAPVPTPI |
| SEQ ID NO: 1816 | VSPSAPAAPVPTPIC |
| SEQ ID NO: 1817 | SPSAPAAPVPTPICP |
| SEQ ID NO: 1818 | PSAPAAPVPTPICPT |
| SEQ ID NO: 1819 | EIINILVKNQVSPSAP |
| SEQ ID NO: 1820 | IINILVKNQVSPSAPA |
| SEQ ID NO: 1821 | INILVKNQVSPSAPAA |
| SEQ ID NO: 1822 | NILVKNQVSPSAPAAP |
| SEQ ID NO: 1823 | ILVKNQVSPSAPAAPV |
| SEQ ID NO: 1824 | LVKNQVSPSAPAAPVP |
| SEQ ID NO: 1825 | VKNQVSPSAPAAPVPT |
| SEQ ID NO: 1826 | KNQVSPSAPAAPVPTP |
| SEQ ID NO: 1827 | NQVSPSAPAAPVPTPI |
| SEQ ID NO: 1828 | QVSPSAPAAPVPTPIC |
| SEQ ID NO: 1829 | VSPSAPAAPVPTPICP |
| SEQ ID NO: 1830 | SPSAPAAPVPTPICPT |
| SEQ ID NO: 1831 | PSAPAAPVPTPICPTT |
| SEQ ID NO: 1832 | VEIINILVKNQVSPSAP |
| SEQ ID NO: 1833 | EIINILVKNQVSPSAPA |
| SEQ ID NO: 1834 | IINILVKNQVSPSAPAA |
| SEQ ID NO: 1835 | INILVKNQVSPSAPAAP |
| SEQ ID NO: 1836 | NILVKNQVSPSAPAAPV |
| SEQ ID NO: 1837 | ILVKNQVSPSAPAAPVP |
| SEQ ID NO: 1838 | LVKNQVSPSAPAAPVPT |
| SEQ ID NO: 1839 | VKNQVSPSAPAAPVPTP |
| SEQ ID NO: 1840 | KNQVSPSAPAAPVPTPI |
| SEQ ID NO: 1841 | NQVSPSAPAAPVPTPIC |
| SEQ ID NO: 1842 | QVSPSAPAAPVPTPICP |
| SEQ ID NO: 1843 | VSPSAPAAPVPTPICPT |
| SEQ ID NO: 1844 | SPSAPAAPVPTPICPTT |
| SEQ ID NO: 1845 | PSAPAAPVPTPICPTTT |
| SEQ ID NO: 1846 | VVEIINILVKNQVSPSAP |
| SEQ ID NO: 1847 | VEIINILVKNQVSPSAPA |
| SEQ ID NO: 1848 | EIINILVKNQVSPSAPAA |
| SEQ ID NO: 1849 | IINILVKNQVSPSAPAAP |
| SEQ ID NO: 1850 | INILVKNQVSPSAPAAPV |
| SEQ ID NO: 1851 | NILVKNQVSPSAPAAPVP |
| SEQ ID NO: 1852 | ILVKNQVSPSAPAAPVPT |
| SEQ ID NO: 1853 | LVKNQVSPSAPAAPVPTP |
| SEQ ID NO: 1854 | VKNQVSPSAPAAPVPTPI |
| SEQ ID NO: 1855 | KNQVSPSAPAAPVPTPIC |
| SEQ ID NO: 1856 | NQVSPSAPAAPVPTPICP |
| SEQ ID NO: 1857 | QVSPSAPAAPVPTPICPT |
| SEQ ID NO: 1858 | VSPSAPAAPVPTPICPTT |
| SEQ ID NO: 1859 | SPSAPAAPVPTPICPTTT |
| SEQ ID NO: 1860 | PSAPAAPVPTPICPTTTP |
| SEQ ID NO: 1861 | RVVEIINILVKNQVSPSAP |
| SEQ ID NO: 1862 | VVEIINILVKNQVSPSAPA |
| SEQ ID NO: 1863 | VEIINILVKNQVSPSAPAA |
| SEQ ID NO: 1864 | EIINILVKNQVSPSAPAAP |
| SEQ ID NO: 1865 | IINILVKNQVSPSAPAAPV |
| SEQ ID NO: 1866 | INILVKNQVSPSAPAAPVP |
| SEQ ID NO: 1867 | NILVKNQVSPSAPAAPVPT |
| SEQ ID NO: 1868 | ILVKNQVSPSAPAAPVPTP |
| SEQ ID NO: 1869 | LVKNQVSPSAPAAPVPTPI |
| SEQ ID NO: 1870 | VKNQVSPSAPAAPVPTPIC |
| SEQ ID NO: 1871 | KNQVSPSAPAAPVPTPICP |
| SEQ ID NO: 1872 | NQVSPSAPAAPVPTPICPT |
| SEQ ID NO: 1873 | QVSPSAPAAPVPTPICPTT |
| SEQ ID NO: 1874 | VSPSAPAAPVPTPICPTTT |
| SEQ ID NO: 1875 | SPSAPAAPVPTPICPTTTP |
| SEQ ID NO: 1876 | PSAPAAPVPTPICPTTTPP |
| SEQ ID NO: 1877 | GRVVEIINILVKNQVSPSAP |
| SEQ ID NO: 1878 | RVVEIINILVKNQVSPSAPA |
| SEQ ID NO: 1879 | VVEIINILVKNQVSPSAPAA |
| SEQ ID NO: 1880 | VEIINILVKNQVSPSAPAAP |
| SEQ ID NO: 1881 | EIINILVKNQVSPSAPAAPV |
| SEQ ID NO: 1882 | IINILVKNQVSPSAPAAPVP |
| SEQ ID NO: 1883 | INILVKNQVSPSAPAAPVPT |
| SEQ ID NO: 1884 | NILVKNQVSPSAPAAPVPTP |
| SEQ ID NO: 1885 | ILVKNQVSPSAPAAPVPTPI |
| SEQ ID NO: 1886 | LVKNQVSPSAPAAPVPTPIC |
| SEQ ID NO: 1887 | VKNQVSPSAPAAPVPTPICP |
| SEQ ID NO: 1888 | KNQVSPSAPAAPVPTPICPT |
| SEQ ID NO: 1889 | NQVSPSAPAAPVPTPICPTT |
| SEQ ID NO: 1890 | QVSPSAPAAPVPTPICPTTT |
| SEQ ID NO: 1891 | VSPSAPAAPVPTPICPTTTP |
| SEQ ID NO: 1892 | SPSAPAAPVPTPICPTTTPP |
| SEQ ID NO: 1893 | PSAPAAPVPTPICPTTTPPP |

TABLE 15
P(T/S)AP Motif Containing Peptides from
West Nile Virus Polyprotein
(GenBank Accession No. NP_041724)

| | |
|---|---|
| SEQ ID NO: 1894 | FSITPSAP |
| SEQ ID NO: 1895 | SITPSAPS |
| SEQ ID NO: 1896 | ITPSAPSY |
| SEQ ID NO: 1897 | TPSAPSYT |
| SEQ ID NO: 1898 | PSAPSYTL |
| SEQ ID NO: 1899 | RFSITPSAP |
| SEQ ID NO: 1900 | FSITPSAPS |
| SEQ ID NO: 1901 | SITPSAPSY |
| SEQ ID NO: 1902 | ITPSAPSYT |
| SEQ ID NO: 1903 | TPSAPSYTL |
| SEQ ID NO: 1904 | PSAPSYTLK |
| SEQ ID NO: 1905 | GRFSITPSAP |
| SEQ ID NO: 1906 | RFSITPSAPS |
| SEQ ID NO: 1907 | FSITPSAPSY |
| SEQ ID NO: 1908 | SITPSAPSYT |
| SEQ ID NO: 1909 | ITPSAPSYTL |
| SEQ ID NO: 1910 | TPSAPSYTLK |
| SEQ ID NO: 1911 | PSAPSYTLKL |
| SEQ ID NO: 1912 | AGRFSITPSAP |
| SEQ ID NO: 1913 | GRFSITPSAPS |
| SEQ ID NO: 1914 | RFSITPSAPSY |
| SEQ ID NO: 1915 | FSITPSAPSYT |
| SEQ ID NO: 1916 | SITPSAPSYTL |
| SEQ ID NO: 1917 | ITPSAPSYTLK |
| SEQ ID NO: 1918 | TPSAPSYTLKL |
| SEQ ID NO: 1919 | PSAPSYTLKLG |
| SEQ ID NO: 1920 | QAGRFSITPSAP |
| SEQ ID NO: 1921 | AGRFSITPSAPS |
| SEQ ID NO: 1922 | GRFSITPSAPSY |
| SEQ ID NO: 1923 | RFSITPSAPSYT |
| SEQ ID NO: 1924 | FSITPSAPSYTL |
| SEQ ID NO: 1925 | SITPSAPSYTLK |
| SEQ ID NO: 1926 | ITPSAPSYTLKL |
| SEQ ID NO: 1927 | TPSAPSYTLKLG |
| SEQ ID NO: 1928 | PSAPSYTLKLGE |
| SEQ ID NO: 1929 | TQAGRFSITPSAP |
| SEQ ID NO: 1930 | QAGRFSITPSAPS |
| SEQ ID NO: 1931 | AGRFSITPSAPSY |
| SEQ ID NO: 1932 | GRFSITPSAPSYT |
| SEQ ID NO: 1933 | RFSITPSAPSYTL |
| SEQ ID NO: 1934 | FSITPSAPSYTLK |
| SEQ ID NO: 1935 | SITPSAPSYTLKL |
| SEQ ID NO: 1936 | ITPSAPSYTLKLG |
| SEQ ID NO: 1937 | TPSAPSYTLKLGE |
| SEQ ID NO: 1938 | PSAPSYTLKLGEY |
| SEQ ID NO: 1939 | ATQAGRFSITPSAP |
| SEQ ID NO: 1940 | TQAGRFSITPSAPS |
| SEQ ID NO: 1941 | QAGRFSITPSAPSY |
| SEQ ID NO: 1942 | AGRFSITPSAPSYT |
| SEQ ID NO: 1943 | GRFSITPSAPSYTL |
| SEQ ID NO: 1944 | RFSITPSAPSYTLK |
| SEQ ID NO: 1945 | FSITPSAPSYTLKL |
| SEQ ID NO: 1946 | SITPSAPSYTLKLG |
| SEQ ID NO: 1947 | ITPSAPSYTLKLGE |
| SEQ ID NO: 1948 | TPSAPSYTLKLGEY |
| SEQ ID NO: 1949 | PSAPSYTLKLGEYG |
| SEQ ID NO: 1950 | GATQAGRFSITPSAP |
| SEQ ID NO: 1951 | ATQAGRFSITPSAPS |
| SEQ ID NO: 1952 | TQAGRFSITPSAPSY |
| SEQ ID NO: 1953 | QAGRFSITPSAPSYT |
| SEQ ID NO: 1954 | AGRFSITPSAPSYTL |
| SEQ ID NO: 1955 | GRFSITPSAPSYTLK |
| SEQ ID NO: 1956 | RFSITPSAPSYTLKL |
| SEQ ID NO: 1957 | FSITPSAPSYTLKLG |
| SEQ ID NO: 1958 | SITPSAPSYTLKLGE |
| SEQ ID NO: 1959 | ITPSAPSYTLKLGEY |
| SEQ ID NO: 1960 | TPSAPSYTLKLGEYG |
| SEQ ID NO: 1961 | PSAPSYTLKLGEYGE |
| SEQ ID NO: 1962 | IGATQAGRFSITPSAP |
| SEQ ID NO: 1963 | GATQAGRFSITPSAPS |
| SEQ ID NO: 1964 | ATQAGRFSITPSAPSY |
| SEQ ID NO: 1965 | TQAGRFSITPSAPSYT |
| SEQ ID NO: 1966 | QAGRFSITPSAPSYTL |
| SEQ ID NO: 1967 | AGRFSITPSAPSYTLK |
| SEQ ID NO: 1968 | GRFSITPSAPSYTLKL |
| SEQ ID NO: 1969 | RFSITPSAPSYTLKLG |
| SEQ ID NO: 1970 | FSITPSAPSYTLKLGE |
| SEQ ID NO: 1971 | SITPSAPSYTLKLGEY |
| SEQ ID NO: 1972 | ITPSAPSYTLKLGEYG |
| SEQ ID NO: 1973 | TPSAPSYTLKLGEYGE |
| SEQ ID NO: 1974 | PSAPSYTLKLGEYGEV |
| SEQ ID NO: 1975 | KIGATQAGRFSITPSAP |
| SEQ ID NO: 1976 | IGATQAGRFSITPSAPS |
| SEQ ID NO: 1977 | GATQAGRFSITPSAPSY |
| SEQ ID NO: 1978 | ATQAGRFSITPSAPSYT |
| SEQ ID NO: 1979 | TQAGRFSITPSAPSYTL |
| SEQ ID NO: 1980 | QAGRFSITPSAPSYTLK |
| SEQ ID NO: 1981 | AGRFSITPSAPSYTLKL |
| SEQ ID NO: 1982 | GRFSITPSAPSYTLKLG |
| SEQ ID NO: 1983 | RFSITPSAPSYTLKLGE |
| SEQ ID NO: 1984 | FSITPSAPSYTLKLGEY |
| SEQ ID NO: 1985 | SITPSAPSYTLKLGEYG |
| SEQ ID NO: 1986 | ITPSAPSYTLKLGEYGE |
| SEQ ID NO: 1987 | TPSAPSYTLKLGEYGEV |
| SEQ ID NO: 1988 | PSAPSYTLKLGEYGEVT |
| SEQ ID NO: 1989 | GKIGATQAGRFSITPSAP |
| SEQ ID NO: 1990 | KIGATQAGRFSITPSAPS |
| SEQ ID NO: 1991 | IGATQAGRFSITPSAPSY |
| SEQ ID NO: 1992 | GATQAGRFSITPSAPSYT |
| SEQ ID NO: 1993 | ATQAGRFSITPSAPSYTL |
| SEQ ID NO: 1994 | TQAGRFSITPSAPSYTLK |
| SEQ ID NO: 1995 | QAGRFSITPSAPSYTLKL |
| SEQ ID NO: 1996 | AGRFSITPSAPSYTLKLG |
| SEQ ID NO: 1997 | GRFSITPSAPSYTLKLGE |
| SEQ ID NO: 1998 | RFSITPSAPSYTLKLGEY |
| SEQ ID NO: 1999 | FSITPSAPSYTLKLGEYG |
| SEQ ID NO: 2000 | SITPSAPSYTLKLGEYGE |
| SEQ ID NO: 2001 | ITPSAPSYTLKLGEYGEV |
| SEQ ID NO: 2002 | TPSAPSYTLKLGEYGEVT |
| SEQ ID NO: 2003 | PSAPSYTLKLGEYGEVTV |
| SEQ ID NO: 2004 | HGKIGATQAGRFSITPSAP |
| SEQ ID NO: 2005 | GKIGATQAGRFSITPSAPS |
| SEQ ID NO: 2006 | KIGATQAGRFSITPSAPSY |
| SEQ ID NO: 2007 | IGATQAGRFSITPSAPSYT |
| SEQ ID NO: 2008 | GATQAGRFSITPSAPSYTL |
| SEQ ID NO: 2009 | ATQAGRFSITPSAPSYTLK |
| SEQ ID NO: 2010 | TQAGRFSITPSAPSYTLKL |
| SEQ ID NO: 2011 | QAGRFSITPSAPSYTLKLG |
| SEQ ID NO: 2012 | AGRFSITPSAPSYTLKLGE |
| SEQ ID NO: 2013 | GRFSITPSAPSYTLKLGEY |
| SEQ ID NO: 2014 | RFSITPSAPSYTLKLGEYG |
| SEQ ID NO: 2015 | FSITPSAPSYTLKLGEYGE |
| SEQ ID NO: 2016 | SITPSAPSYTLKLGEYGEV |
| SEQ ID NO: 2017 | ITPSAPSYTLKLGEYGEVT |
| SEQ ID NO: 2018 | TPSAPSYTLKLGEYGEVTV |
| SEQ ID NO: 2019 | PSAPSYTLKLGEYGEVTVD |
| SEQ ID NO: 2020 | SHGKIGATQAGRFSITPSAP |
| SEQ ID NO: 2021 | HGKIGATQAGRFSITPSAPS |
| SEQ ID NO: 2022 | GKIGATQAGRFSITPSAPSY |
| SEQ ID NO: 2023 | KIGATQAGRFSITPSAPSYT |
| SEQ ID NO: 2024 | IGATQAGRFSITPSAPSYTL |
| SEQ ID NO: 2025 | GATQAGRFSITPSAPSYTLK |
| SEQ ID NO: 2026 | ATQAGRFSITPSAPSYTLKL |
| SEQ ID NO: 2027 | TQAGRFSITPSAPSYTLKLG |
| SEQ ID NO: 2028 | QAGRFSITPSAPSYTLKLGE |
| SEQ ID NO: 2029 | AGRFSITPSAPSYTLKLGEY |
| SEQ ID NO: 2030 | GRFSITPSAPSYTLKLGEYG |
| SEQ ID NO: 2031 | RFSITPSAPSYTLKLGEYGE |
| SEQ ID NO: 2032 | FSITPSAPSYTLKLGEYGEV |
| SEQ ID NO: 2033 | SITPSAPSYTLKLGEYGEVT |
| SEQ ID NO: 2034 | ITPSAPSYTLKLGEYGEVTV |
| SEQ ID NO: 2035 | TPSAPSYTLKLGEYGEVTVD |
| SEQ ID NO: 2036 | PSAPSYTLKLGEYGEVTVDC |

TABLE 16
P(T/S)AP Motif Containing Peptides from Measles
Virus Matrix protein
(GenBank Accession No. CAA34587)

| | |
|---|---|
| SEQ ID NO: 2037 | AAPQPSAP |
| SEQ ID NO: 2038 | APQPSAPQ |
| SEQ ID NO: 2039 | PQPSAPQE |
| SEQ ID NO: 2040 | QPSAPQEP |
| SEQ ID NO: 2041 | PSAPQEPR |
| SEQ ID NO: 2042 | QAAPQPSAP |
| SEQ ID NO: 2043 | AAPQPSAPQ |
| SEQ ID NO: 2044 | APQPSAPQE |
| SEQ ID NO: 2045 | PQPSAPQEP |
| SEQ ID NO: 2046 | QPSAPQEPR |
| SEQ ID NO: 2047 | PSAPQEPRT |
| SEQ ID NO: 2048 | IQAAPQPSAP |
| SEQ ID NO: 2049 | QAAPQPSAPQ |
| SEQ ID NO: 2050 | AAPQPSAPQE |
| SEQ ID NO: 2051 | APQPSAPQEP |
| SEQ ID NO: 2052 | PQPSAPQEPR |
| SEQ ID NO: 2053 | QPSAPQEPRT |
| SEQ ID NO: 2054 | PSAPQEPRTH |
| SEQ ID NO: 2055 | RIQAAPQPSAP |
| SEQ ID NO: 2056 | IQAAPQPSAPQ |
| SEQ ID NO: 2057 | QAAPQPSAPQE |
| SEQ ID NO: 2058 | AAPQPSAPQEP |
| SEQ ID NO: 2059 | APQPSAPQEPR |
| SEQ ID NO: 2060 | PQPSAPQEPRT |
| SEQ ID NO: 2061 | QPSAPQEPRTH |
| SEQ ID NO: 2062 | PSAPQEPRTHD |
| SEQ ID NO: 2063 | ARIQAAPQPSAP |
| SEQ ID NO: 2064 | RIQAAPQPSAPQ |
| SEQ ID NO: 2065 | IQAAPQPSAPQE |
| SEQ ID NO: 2066 | QAAPQPSAPQEP |
| SEQ ID NO: 2067 | AAPQPSAPQEPR |
| SEQ ID NO: 2068 | APQPSAPQEPRT |
| SEQ ID NO: 2069 | PQPSAPQEPRTH |
| SEQ ID NO: 2070 | QPSAPQEPRTHD |
| SEQ ID NO: 2071 | PSAPQEPRTHDD |
| SEQ ID NO: 2072 | TARIQAAPQPSAP |
| SEQ ID NO: 2073 | ARIQAAPQPSAPQ |
| SEQ ID NO: 2074 | RIQAAPQPSAPQE |
| SEQ ID NO: 2075 | IQAAPQPSAPQEP |
| SEQ ID NO: 2076 | QAAPQPSAPQEPR |
| SEQ ID NO: 2077 | AAPQPSAPQEPRT |
| SEQ ID NO: 2078 | APQPSAPQEPRTH |
| SEQ ID NO: 2079 | PQPSAPQEPRTHD |
| SEQ ID NO: 2080 | QPSAPQEPRTHDD |
| SEQ ID NO: 2081 | PSAPQEPRTHDDA |
| SEQ ID NO: 2082 | KTARIQAAPQPSAP |
| SEQ ID NO: 2083 | TARIQAAPQPSAPQ |
| SEQ ID NO: 2084 | ARIQAAPQPSAPQE |
| SEQ ID NO: 2085 | RIQAAPQPSAPQEP |
| SEQ ID NO: 2086 | IQAAPQPSAPQEPR |
| SEQ ID NO: 2087 | QAAPQPSAPQEPRT |
| SEQ ID NO: 2088 | AAPQPSAPQEPRTH |
| SEQ ID NO: 2089 | APQPSAPQEPRTHD |
| SEQ ID NO: 2090 | PQPSAPQEPRTHDD |
| SEQ ID NO: 2091 | QPSAPQEPRTHDDA |
| SEQ ID NO: 2092 | PSAPQEPRTHDDAI |
| SEQ ID NO: 2093 | RKTARIQAAPQPSAP |
| SEQ ID NO: 2094 | KTARIQAAPQPSAPQ |
| SEQ ID NO: 2095 | TARIQAAPQPSAPQE |
| SEQ ID NO: 2096 | ARIQAAPQPSAPQEP |
| SEQ ID NO: 2097 | RIQAAPQPSAPQEPR |
| SEQ ID NO: 2098 | IQAAPQPSAPQEPRT |
| SEQ ID NO: 2099 | QAAPQPSAPQEPRTH |
| SEQ ID NO: 2100 | AAPQPSAPQEPRTHD |
| SEQ ID NO: 2101 | APQPSAPQEPRTHDD |
| SEQ ID NO: 2102 | PQPSAPQEPRTHDDA |
| SEQ ID NO: 2103 | QPSAPQEPRTHDDAI |
| SEQ ID NO: 2104 | PSAPQEPRTHDDAIT |
| SEQ ID NO: 2105 | RRKTARIQAAPQPSAP |
| SEQ ID NO: 2106 | RKTARIQAAPQPSAPQ |
| SEQ ID NO: 2107 | KTARIQAAPQPSAPQE |
| SEQ ID NO: 2108 | TARIQAAPQPSAPQEP |
| SEQ ID NO: 2109 | ARIQAAPQPSAPQEPR |
| SEQ ID NO: 2110 | RIQAAPQPSAPQEPRT |
| SEQ ID NO: 2111 | IQAAPQPSAPQEPRTH |
| SEQ ID NO: 2112 | QAAPQPSAPQEPRTHD |
| SEQ ID NO: 2113 | AAPQPSAPQEPRTHDD |
| SEQ ID NO: 2114 | APQPSAPQEPRTHDDA |
| SEQ ID NO: 2115 | PQPSAPQEPRTHDDAI |
| SEQ ID NO: 2116 | QPSAPQEPRTHDDAIT |
| SEQ ID NO: 2117 | PSAPQEPRTHDDAITN |
| SEQ ID NO: 2118 | SRRKTARIQAAPQPSAP |
| SEQ ID NO: 2119 | RRKTARIQAAPQPSAPQ |
| SEQ ID NO: 2120 | RKTARIQAAPQPSAPQE |
| SEQ ID NO: 2121 | KTARIQAAPQPSAPQEP |
| SEQ ID NO: 2122 | TARIQAAPQPSAPQEPR |
| SEQ ID NO: 2123 | ARIQAAPQPSAPQEPRT |
| SEQ ID NO: 2124 | RIQAAPQPSAPQEPRTH |
| SEQ ID NO: 2125 | IQAAPQPSAPQEPRTHD |
| SEQ ID NO: 2126 | QAAPQPSAPQEPRTHDD |
| SEQ ID NO: 2127 | AAPQPSAPQEPRTHDDA |
| SEQ ID NO: 2128 | APQPSAPQEPRTHDDAI |
| SEQ ID NO: 2129 | PQPSAPQEPRTHDDAIT |
| SEQ ID NO: 2130 | QPSAPQEPRTHDDAITN |
| SEQ ID NO: 2131 | PSAPQEPRTHDDAITND |
| SEQ ID NO: 2132 | RSRRKTARIQAAPQPSAP |
| SEQ ID NO: 2133 | SRRKTARIQAAPQPSAPQ |
| SEQ ID NO: 2134 | RRKTARIQAAPQPSAPQE |
| SEQ ID NO: 2135 | RKTARIQAAPQPSAPQEP |
| SEQ ID NO: 2136 | KTARIQAAPQPSAPQEPR |
| SEQ ID NO: 2137 | TARIQAAPQPSAPQEPRT |
| SEQ ID NO: 2138 | ARIQAAPQPSAPQEPRTH |
| SEQ ID NO: 2139 | RIQAAPQPSAPQEPRTHD |
| SEQ ID NO: 2140 | IQAAPQPSAPQEPRTHDD |
| SEQ ID NO: 2141 | QAAPQPSAPQEPRTHDDA |
| SEQ ID NO: 2142 | AAPQPSAPQEPRTHDDAI |
| SEQ ID NO: 2143 | APQPSAPQEPRTHDDAIT |
| SEQ ID NO: 2144 | PQPSAPQEPRTHDDAITN |
| SEQ ID NO: 2145 | QPSAPQEPRTHDDAITND |
| SEQ ID NO: 2146 | PSAPQEPRTHDDAITNDD |
| SEQ ID NO: 2147 | WRSRRKTARIQAAPQPSAP |
| SEQ ID NO: 2148 | RSRRKTARIQAAPQPSAPQ |
| SEQ ID NO: 2149 | SRRKTARIQAAPQPSAPQE |
| SEQ ID NO: 2150 | RRKTARIQAAPQPSAPQEP |
| SEQ ID NO: 2151 | RKTARIQAAPQPSAPQEPR |
| SEQ ID NO: 2152 | KTARIQAAPQPSAPQEPRT |
| SEQ ID NO: 2153 | TARIQAAPQPSAPQEPRTH |
| SEQ ID NO: 2154 | ARIQAAPQPSAPQEPRTHD |
| SEQ ID NO: 2155 | RIQAAPQPSAPQEPRTHDD |
| SEQ ID NO: 2156 | IQAAPQPSAPQEPRTHDDA |
| SEQ ID NO: 2157 | QAAPQPSAPQEPRTHDDAI |
| SEQ ID NO: 2158 | AAPQPSAPQEPRTHDDAIT |
| SEQ ID NO: 2159 | APQPSAPQEPRTHDDAITN |
| SEQ ID NO: 2160 | PQPSAPQEPRTHDDAITND |
| SEQ ID NO: 2161 | QPSAPQEPRTHDDAITNDD |
| SEQ ID NO: 2162 | PSAPQEPRTHDDAITNDDQ |
| SEQ ID NO: 2163 | LWRSRRKTARIQAAPQPSAP |
| SEQ ID NO: 2164 | WRSRRKTARIQAAPQPSAPQ |
| SEQ ID NO: 2165 | RSRRKTARIQAAPQPSAPQE |
| SEQ ID NO: 2166 | SRRKTARIQAAPQPSAPQEP |
| SEQ ID NO: 2167 | RRKTARIQAAPQPSAPQEPR |
| SEQ ID NO: 2168 | RKTARIQAAPQPSAPQEPRT |
| SEQ ID NO: 2169 | KTARIQAAPQPSAPQEPRTH |
| SEQ ID NO: 2170 | TARIQAAPQPSAPQEPRTHD |
| SEQ ID NO: 2171 | ARIQAAPQPSAPQEPRTHDD |
| SEQ ID NO: 2172 | RIQAAPQPSAPQEPRTHDDA |
| SEQ ID NO: 2173 | IQAAPQPSAPQEPRTHDDAI |
| SEQ ID NO: 2174 | QAAPQPSAPQEPRTHDDAIT |
| SEQ ID NO: 2175 | AAPQPSAPQEPRTHDDAITN |
| SEQ ID NO: 2176 | APQPSAPQEPRTHDDAITND |
| SEQ ID NO: 2177 | PQPSAPQEPRTHDDAITNDD |
| SEQ ID NO: 2178 | QPSAPQEPRTHDDAITNDDQ |
| SEQ ID NO: 2179 | PSAPQEPRTHDDAITNDDQG |

TABLE 17
P(T/S)AP Motif Containing Peptides from Rubella
Virus Non-Structural Protein
(GenBank Accession No. BAB32473)

| | |
|---|---|
| SEQ ID NO: 2180 | PRERPSAP |
| SEQ ID NO: 2181 | RERPSAPA |
| SEQ ID NO: 2182 | ERPSAPAG |
| SEQ ID NO: 2183 | RPSAPAGP |
| SEQ ID NO: 2184 | PSAPAGPP |
| SEQ ID NO: 2185 | APRERPSAP |
| SEQ ID NO: 2186 | PRERPSAPA |
| SEQ ID NO: 2187 | RERPSAPAG |
| SEQ ID NO: 2188 | ERPSAPAGP |
| SEQ ID NO: 2189 | RPSAPAGPP |
| SEQ ID NO: 2190 | PSAPAGPPD |
| SEQ ID NO: 2191 | DAPRERPSAP |
| SEQ ID NO: 2192 | APRERPSAPA |
| SEQ ID NO: 2193 | PRERPSAPAG |
| SEQ ID NO: 2194 | RERPSAPAGP |
| SEQ ID NO: 2195 | ERPSAPAGPP |
| SEQ ID NO: 2196 | RPSAPAGPPD |
| SEQ ID NO: 2197 | PSAPAGPPDD |
| SEQ ID NO: 2198 | CDAPRERPSAP |
| SEQ ID NO: 2199 | DAPRERPSAPA |
| SEQ ID NO: 2200 | APRERPSAPAG |
| SEQ ID NO: 2201 | PRERPSAPAGP |
| SEQ ID NO: 2202 | RERPSAPAGPP |
| SEQ ID NO: 2203 | ERPSAPAGPPD |
| SEQ ID NO: 2204 | RPSAPAGPPDD |
| SEQ ID NO: 2205 | PSAPAGPPDDE |
| SEQ ID NO: 2206 | RCDAPRERPSAP |
| SEQ ID NO: 2207 | CDAPRERPSAPA |
| SEQ ID NO: 2208 | DAPRERPSAPAG |
| SEQ ID NO: 2209 | APRERPSAPAGP |
| SEQ ID NO: 2210 | PRERPSAPAGPP |
| SEQ ID NO: 2211 | RERPSAPAGPPD |
| SEQ ID NO: 2212 | ERPSAPAGPPDD |
| SEQ ID NO: 2213 | RPSAPAGPPDDE |
| SEQ ID NO: 2214 | PSAPAGPPDDEA |
| SEQ ID NO: 2215 | PRCDAPRERPSAP |
| SEQ ID NO: 2216 | RCDAPRERPSAPA |
| SEQ ID NO: 2217 | CDAPRERPSAPAG |
| SEQ ID NO: 2218 | DAPRERPSAPAGP |
| SEQ ID NO: 2219 | APRERPSAPAGPP |
| SEQ ID NO: 2220 | PRERPSAPAGPPD |
| SEQ ID NO: 2221 | RERPSAPAGPPDD |
| SEQ ID NO: 2222 | ERPSAPAGPPDDE |
| SEQ ID NO: 2223 | RPSAPAGPPDDEA |
| SEQ ID NO: 2224 | PSAPAGPPDDEAL |
| SEQ ID NO: 2225 | APRCDAPRERPSAP |
| SEQ ID NO: 2226 | PRCDAPRERPSAPA |
| SEQ ID NO: 2227 | RCDAPRERPSAPAG |
| SEQ ID NO: 2228 | CDAPRERPSAPAGP |
| SEQ ID NO: 2229 | DAPRERPSAPAGPP |
| SEQ ID NO: 2230 | APRERPSAPAGPPD |
| SEQ ID NO: 2231 | PRERPSAPAGPPDD |
| SEQ ID NO: 2232 | RERPSAPAGPPDDE |
| SEQ ID NO: 2233 | ERPSAPAGPPDDEA |
| SEQ ID NO: 2234 | RPSAPAGPPDDEAL |
| SEQ ID NO: 2235 | PSAPAGPPDDEALI |
| SEQ ID NO: 2236 | CAPRCDAPRERPSAP |
| SEQ ID NO: 2237 | APRCDAPRERPSAPA |
| SEQ ID NO: 2238 | PRCDAPRERPSAPAG |
| SEQ ID NO: 2239 | RCDAPRERPSAPAGP |
| SEQ ID NO: 2240 | CDAPRERPSAPAGPP |
| SEQ ID NO: 2241 | DAPRERPSAPAGPPD |
| SEQ ID NO: 2242 | APRERPSAPAGPPDD |
| SEQ ID NO: 2243 | PRERPSAPAGPPDDE |
| SEQ ID NO: 2244 | RERPSAPAGPPDDEA |
| SEQ ID NO: 2245 | ERPSAPAGPPDDEAL |
| SEQ ID NO: 2246 | RPSAPAGPPDDEALI |
| SEQ ID NO: 2247 | PSAPAGPPDDEALIP |
| SEQ ID NO: 2248 | ACAPRCDAPRERPSAP |
| SEQ ID NO: 2249 | CAPRCDAPRERPSAPA |
| SEQ ID NO: 2250 | APRCDAPRERPSAPAG |
| SEQ ID NO: 2251 | PRCDAPRERPSAPAGP |
| SEQ ID NO: 2252 | RCDAPRERPSAPAGPP |
| SEQ ID NO: 2253 | CDAPRERPSAPAGPPD |
| SEQ ID NO: 2254 | DAPRERPSAPAGPPDD |
| SEQ ID NO: 2255 | APRERPSAPAGPPDDE |
| SEQ ID NO: 2256 | PRERPSAPAGPPDDEA |
| SEQ ID NO: 2257 | RERPSAPAGPPDDEAL |
| SEQ ID NO: 2258 | ERPSAPAGPPDDEALI |
| SEQ ID NO: 2259 | RPSAPAGPPDDEALIP |
| SEQ ID NO: 2260 | PSAPAGPPDDEALIPP |
| SEQ ID NO: 2261 | CACAPRCDAPRERPSAP |
| SEQ ID NO: 2262 | ACAPRCDAPRERPSAPA |
| SEQ ID NO: 2263 | CAPRCDAPRERPSAPAG |
| SEQ ID NO: 2264 | APRCDAPRERPSAPAGP |
| SEQ ID NO: 2265 | PRCDAPRERPSAPAGPP |
| SEQ ID NO: 2266 | RCDAPRERPSAPAGPPD |
| SEQ ID NO: 2267 | CDAPRERPSAPAGPPDD |
| SEQ ID NO: 2268 | DAPRERPSAPAGPPDDE |
| SEQ ID NO: 2269 | APRERPSAPAGPPDDEA |
| SEQ ID NO: 2270 | PRERPSAPAGPPDDEAL |
| SEQ ID NO: 2271 | RERPSAPAGPPDDEALI |
| SEQ ID NO: 2272 | ERPSAPAGPPDDEALIP |
| SEQ ID NO: 2273 | RPSAPAGPPDDEALIPP |
| SEQ ID NO: 2274 | PSAPAGPPDDEALIPPW |
| SEQ ID NO: 2275 | HCACAPRCDAPRERPSAP |
| SEQ ID NO: 2276 | CACAPRCDAPRERPSAPA |
| SEQ ID NO: 2277 | ACAPRCDAPRERPSAPAG |
| SEQ ID NO: 2278 | CAPRCDAPRERPSAPAGP |
| SEQ ID NO: 2279 | APRCDAPRERPSAPAGPP |
| SEQ ID NO: 2280 | PRCDAPRERPSAPAGPPD |
| SEQ ID NO: 2281 | RCDAPRERPSAPAGPPDD |
| SEQ ID NO: 2282 | CDAPRERPSAPAGPPDDE |
| SEQ ID NO: 2283 | DAPRERPSAPAGPPDDEA |
| SEQ ID NO: 2284 | APRERPSAPAGPPDDEAL |
| SEQ ID NO: 2285 | PRERPSAPAGPPDDEALI |
| SEQ ID NO: 2286 | RERPSAPAGPPDDEALIP |
| SEQ ID NO: 2287 | ERPSAPAGPPDDEALIPP |
| SEQ ID NO: 2288 | RPSAPAGPPDDEALIPPW |
| SEQ ID NO: 2289 | PSAPAGPPDDEALIPPWL |
| SEQ ID NO: 2290 | RHCACAPRCDAPRERPSAP |
| SEQ ID NO: 2291 | HCACAPRCDAPRERPSAPA |
| SEQ ID NO: 2292 | CACAPRCDAPRERPSAPAG |
| SEQ ID NO: 2293 | ACAPRCDAPRERPSAPAGP |
| SEQ ID NO: 2294 | CAPRCDAPRERPSAPAGPP |
| SEQ ID NO: 2295 | APRCDAPRERPSAPAGPPD |
| SEQ ID NO: 2296 | PRCDAPRERPSAPAGPPDD |
| SEQ ID NO: 2297 | RCDAPRERPSAPAGPPDDE |
| SEQ ID NO: 2298 | CDAPRERPSAPAGPPDDEA |
| SEQ ID NO: 2299 | DAPRERPSAPAGPPDDEAL |
| SEQ ID NO: 2300 | APRERPSAPAGPPDDEALI |
| SEQ ID NO: 2301 | PRERPSAPAGPPDDEALIP |
| SEQ ID NO: 2302 | RERPSAPAGPPDDEALIPP |
| SEQ ID NO: 2303 | ERPSAPAGPPDDEALIPPW |
| SEQ ID NO: 2304 | RPSAPAGPPDDEALIPPWL |
| SEQ ID NO: 2305 | PSAPAGPPDDEALIPPWLF |
| SEQ ID NO: 2306 | DRHCACAPRCDAPRERPSAP |
| SEQ ID NO: 2307 | RHCACAPRCDAPRERPSAPA |
| SEQ ID NO: 2308 | HCACAPRCDAPRERPSAPAG |
| SEQ ID NO: 2309 | CACAPRCDAPRERPSAPAGP |
| SEQ ID NO: 2310 | ACAPRCDAPRERPSAPAGPP |
| SEQ ID NO: 2311 | CAPRCDAPRERPSAPAGPPD |
| SEQ ID NO: 2312 | APRCDAPRERPSAPAGPPDD |
| SEQ ID NO: 2313 | PRCDAPRERPSAPAGPPDDE |
| SEQ ID NO: 2314 | RCDAPRERPSAPAGPPDDEA |
| SEQ ID NO: 2315 | CDAPRERPSAPAGPPDDEAL |
| SEQ ID NO: 2316 | DAPRERPSAPAGPPDDEALI |
| SEQ ID NO: 2317 | APRERPSAPAGPPDDEALIP |
| SEQ ID NO: 2318 | PRERPSAPAGPPDDEALIPP |
| SEQ ID NO: 2319 | RERPSAPAGPPDDEALIPPW |
| SEQ ID NO: 2320 | ERPSAPAGPPDDEALIPPWL |
| SEQ ID NO: 2321 | RPSAPAGPPDDEALIPPWLF |
| SEQ ID NO: 2322 | PSAPAGPPDDEALIPPWLFA |

TABLE 18
P(T/S)AP Motif Containing Peptides from
Colorado Tick Fever Virus VP12
(GenBank Accession No. AAB02025)

| | |
|---|---|
| SEQ ID NO: 2323 | TRVAPSAP |
| SEQ ID NO: 2324 | RVAPSAPS |
| SEQ ID NO: 2325 | VAPSAPSA |
| SEQ ID NO: 2326 | APSAPSAS |
| SEQ ID NO: 2327 | PSAPSASL |
| SEQ ID NO: 2328 | STRVAPSAP |
| SEQ ID NO: 2329 | TRVAPSAPS |
| SEQ ID NO: 2330 | RVAPSAPSA |
| SEQ ID NO: 2331 | VAPSAPSAS |
| SEQ ID NO: 2332 | APSAPSASL |
| SEQ ID NO: 2333 | PSAPSASLF |
| SEQ ID NO: 2334 | LSTRVAPSAP |
| SEQ ID NO: 2335 | STRVAPSAPS |
| SEQ ID NO: 2336 | TRVAPSAPSA |
| SEQ ID NO: 2337 | RVAPSAPSAS |
| SEQ ID NO: 2338 | VAPSAPSASL |
| SEQ ID NO: 2339 | APSAPSASLF |
| SEQ ID NO: 2340 | PSAPSASLFT |
| SEQ ID NO: 2341 | PLSTRVAPSAP |
| SEQ ID NO: 2342 | LSTRVAPSAPS |
| SEQ ID NO: 2343 | STRVAPSAPSA |
| SEQ ID NO: 2344 | TRVAPSAPSAS |
| SEQ ID NO: 2345 | RVAPSAPSASL |
| SEQ ID NO: 2346 | VAPSAPSASLF |
| SEQ ID NO: 2347 | APSAPSASLFT |
| SEQ ID NO: 2348 | PSAPSASLFTA |
| SEQ ID NO: 2349 | TPLSTRVAPSAP |
| SEQ ID NO: 2350 | PLSTRVAPSAPS |
| SEQ ID NO: 2351 | LSTRVAPSAPSA |
| SEQ ID NO: 2352 | STRVAPSAPSAS |
| SEQ ID NO: 2353 | TRVAPSAPSASL |
| SEQ ID NO: 2354 | RVAPSAPSASLF |
| SEQ ID NO: 2355 | VAPSAPSASLFT |
| SEQ ID NO: 2356 | APSAPSASLFTA |
| SEQ ID NO: 2357 | PSAPSASLFTAG |
| SEQ ID NO: 2358 | ETPLSTRVAPSAP |
| SEQ ID NO: 2359 | TPLSTRVAPSAPS |
| SEQ ID NO: 2360 | PLSTRVAPSAPSA |
| SEQ ID NO: 2361 | LSTRVAPSAPSAS |
| SEQ ID NO: 2362 | STRVAPSAPSASL |
| SEQ ID NO: 2363 | TRVAPSAPSASLF |
| SEQ ID NO: 2364 | RVAPSAPSASLFT |
| SEQ ID NO: 2365 | VAPSAPSASLFTA |
| SEQ ID NO: 2366 | APSAPSASLFTAG |
| SEQ ID NO: 2367 | PSAPSASLFTAGG |
| SEQ ID NO: 2368 | CETPLSTRVAPSAP |
| SEQ ID NO: 2369 | ETPLSTRVAPSAPS |
| SEQ ID NO: 2370 | TPLSTRVAPSAPSA |
| SEQ ID NO: 2371 | PLSTRVAPSAPSAS |
| SEQ ID NO: 2372 | LSTRVAPSAPSASL |
| SEQ ID NO: 2373 | STRVAPSAPSASLF |
| SEQ ID NO: 2374 | TRVAPSAPSASLFT |
| SEQ ID NO: 2375 | RVAPSAPSASLFTA |
| SEQ ID NO: 2376 | VAPSAPSASLFTAG |
| SEQ ID NO: 2377 | APSAPSASLFTAGG |
| SEQ ID NO: 2378 | PSAPSASLFTAGGI |
| SEQ ID NO: 2379 | ICETPLSTRVAPSAP |
| SEQ ID NO: 2380 | CETPLSTRVAPSAPS |
| SEQ ID NO: 2381 | ETPLSTRVAPSAPSA |
| SEQ ID NO: 2382 | TPLSTRVAPSAPSAS |
| SEQ ID NO: 2383 | PLSTRVAPSAPSASL |
| SEQ ID NO: 2384 | LSTRVAPSAPSASLF |
| SEQ ID NO: 2385 | STRVAPSAPSASLFT |
| SEQ ID NO: 2386 | TRVAPSAPSASLFTA |
| SEQ ID NO: 2387 | RVAPSAPSASLFTAG |
| SEQ ID NO: 2388 | VAPSAPSASLFTAGG |
| SEQ ID NO: 2389 | APSAPSASLFTAGGI |
| SEQ ID NO: 2390 | PSAPSASLFTAGGIG |
| SEQ ID NO: 2391 | HICETPLSTRVAPSAP |
| SEQ ID NO: 2392 | ICETPLSTRVAPSAPS |
| SEQ ID NO: 2393 | CETPLSTRVAPSAPSA |
| SEQ ID NO: 2394 | ETPLSTRVAPSAPSAS |
| SEQ ID NO: 2395 | TPLSTRVAPSAPSASL |
| SEQ ID NO: 2396 | PLSTRVAPSAPSASLF |
| SEQ ID NO: 2397 | LSTRVAPSAPSASLFT |
| SEQ ID NO: 2398 | STRVAPSAPSASLFTA |
| SEQ ID NO: 2399 | TRVAPSAPSASLFTAG |
| SEQ ID NO: 2400 | RVAPSAPSASLFTAGG |
| SEQ ID NO: 2401 | VAPSAPSASLFTAGGI |
| SEQ ID NO: 2402 | APSAPSASLFTAGGIG |
| SEQ ID NO: 2403 | PSAPSASLFTAGGIGL |
| SEQ ID NO: 2404 | PHICETPLSTRVAPSAP |
| SEQ ID NO: 2405 | HICETPLSTRVAPSAPS |
| SEQ ID NO: 2406 | ICETPLSTRVAPSAPSA |
| SEQ ID NO: 2407 | CETPLSTRVAPSAPSAS |
| SEQ ID NO: 2408 | ETPLSTRVAPSAPSASL |
| SEQ ID NO: 2409 | TPLSTRVAPSAPSASLF |
| SEQ ID NO: 2410 | PLSTRVAPSAPSASLFT |
| SEQ ID NO: 2411 | LSTRVAPSAPSASLFTA |
| SEQ ID NO: 2412 | STRVAPSAPSASLFTAG |
| SEQ ID NO: 2413 | TRVAPSAPSASLFTAGG |
| SEQ ID NO: 2414 | RVAPSAPSASLFTAGGI |
| SEQ ID NO: 2415 | VAPSAPSASLFTAGGIG |
| SEQ ID NO: 2416 | APSAPSASLFTAGGIGL |
| SEQ ID NO: 2417 | PSAPSASLFTAGGIGLP |
| SEQ ID NO: 2418 | SPHICETPLSTRVAPSAP |
| SEQ ID NO: 2419 | PHICETPLSTRVAPSAPS |
| SEQ ID NO: 2420 | HICETPLSTRVAPSAPSA |
| SEQ ID NO: 2421 | ICETPLSTRVAPSAPSAS |
| SEQ ID NO: 2422 | CETPLSTRVAPSAPSASL |
| SEQ ID NO: 2423 | ETPLSTRVAPSAPSASLF |
| SEQ ID NO: 2424 | TPLSTRVAPSAPSASLFT |
| SEQ ID NO: 2425 | PLSTRVAPSAPSASLFTA |
| SEQ ID NO: 2426 | LSTRVAPSAPSASLFTAG |
| SEQ ID NO: 2427 | STRVAPSAPSASLFTAGG |
| SEQ ID NO: 2428 | TRVAPSAPSASLFTAGGI |
| SEQ ID NO: 2429 | RVAPSAPSASLFTAGGIG |
| SEQ ID NO: 2430 | VAPSAPSASLFTAGGIGL |
| SEQ ID NO: 2431 | APSAPSASLFTAGGIGLP |
| SEQ ID NO: 2432 | ASPHICETPLSTRVAPSAP |
| SEQ ID NO: 2433 | SPHICETPLSTRVAPSAPS |
| SEQ ID NO: 2434 | PHICETPLSTRVAPSAPSA |
| SEQ ID NO: 2435 | HICETPLSTRVAPSAPSAS |
| SEQ ID NO: 2436 | ICETPLSTRVAPSAPSASL |
| SEQ ID NO: 2437 | CETPLSTRVAPSAPSASLF |
| SEQ ID NO: 2438 | ETPLSTRVAPSAPSASLFT |
| SEQ ID NO: 2439 | TPLSTRVAPSAPSASLFTA |
| SEQ ID NO: 2440 | PLSTRVAPSAPSASLFTAG |
| SEQ ID NO: 2441 | LSTRVAPSAPSASLFTAGG |
| SEQ ID NO: 2442 | STRVAPSAPSASLFTAGGI |
| SEQ ID NO: 2443 | TRVAPSAPSASLFTAGGIG |
| SEQ ID NO: 2444 | RVAPSAPSASLFTAGGIGL |
| SEQ ID NO: 2445 | VAPSAPSASLFTAGGIGLP |
| SEQ ID NO: 2446 | PASPHICETPLSTRVAPSAP |
| SEQ ID NO: 2447 | ASPHICETPLSTRVAPSAPS |
| SEQ ID NO: 2448 | SPHICETPLSTRVAPSAPSA |
| SEQ ID NO: 2449 | PHICETPLSTRVAPSAPSAS |
| SEQ ID NO: 2450 | HICETPLSTRVAPSAPSASL |
| SEQ ID NO: 2451 | ICETPLSTRVAPSAPSASLF |
| SEQ ID NO: 2452 | CETPLSTRVAPSAPSASLFT |
| SEQ ID NO: 2453 | ETPLSTRVAPSAPSASLFTA |
| SEQ ID NO: 2454 | TPLSTRVAPSAPSASLFTAG |
| SEQ ID NO: 2455 | PLSTRVAPSAPSASLFTAGG |
| SEQ ID NO: 2456 | LSTRVAPSAPSASLFTAGGI |
| SEQ ID NO: 2457 | STRVAPSAPSASLFTAGGIG |
| SEQ ID NO: 2458 | TRVAPSAPSASLFTAGGIGL |
| SEQ ID NO: 2459 | RVAPSAPSASLFTAGGIGLP |

TABLE 19
P(T/S)AP Motif Containing Peptides from Foot
and Mouth Disease Virus VP1 Capsid Protein
(GenBank Accession No. AAA42637)

| | |
|---|---|
| SEQ ID NO: 2460 | RLALPTAP |
| SEQ ID NO: 2461 | LALPTAPR |
| SEQ ID NO: 2462 | ALPTAPRV |
| SEQ ID NO: 2463 | LPTAPRVL |
| SEQ ID NO: 2464 | PTAPRVLA |
| SEQ ID NO: 2465 | TRLALPTAP |
| SEQ ID NO: 2466 | RLALPTAPR |
| SEQ ID NO: 2467 | LALPTAPRV |
| SEQ ID NO: 2468 | ALPTAPRVL |
| SEQ ID NO: 2469 | LPTAPRVLA |
| SEQ ID NO: 2470 | PTAPRVLAT |
| SEQ ID NO: 2471 | LTRLALPTAP |
| SEQ ID NO: 2472 | TRLALPTAPR |
| SEQ ID NO: 2473 | RLALPTAPRV |
| SEQ ID NO: 2474 | LALPTAPRVL |
| SEQ ID NO: 2475 | ALPTAPRVLA |
| SEQ ID NO: 2476 | LPTAPRVLAT |
| SEQ ID NO: 2477 | PTAPRVLATV |
| SEQ ID NO: 2478 | ALTRLALPTAP |
| SEQ ID NO: 2479 | LTRLALPTAPR |
| SEQ ID NO: 2480 | TRLALPTAPRV |
| SEQ ID NO: 2481 | RLALPTAPRVL |
| SEQ ID NO: 2482 | LALPTAPRVLA |
| SEQ ID NO: 2483 | ALPTAPRVLAT |
| SEQ ID NO: 2484 | LPTAPRVLATV |
| SEQ ID NO: 2485 | PTAPRVLATVG |
| SEQ ID NO: 2486 | KALTRLALPTAP |
| SEQ ID NO: 2487 | ALTRLALPTAPR |
| SEQ ID NO: 2488 | LTRLALPTAPRV |
| SEQ ID NO: 2489 | TRLALPTAPRVL |
| SEQ ID NO: 2490 | RLALPTAPRVLA |
| SEQ ID NO: 2491 | LALPTAPRVLAT |
| SEQ ID NO: 2492 | ALPTAPRVLATV |
| SEQ ID NO: 2493 | LPTAPRVLATVG |
| SEQ ID NO: 2494 | PTAPRVLATVGE |
| SEQ ID NO: 2495 | AKALTRLALPTAP |
| SEQ ID NO: 2496 | KALTRLALPTAPR |
| SEQ ID NO: 2497 | ALTRLALPTAPRV |
| SEQ ID NO: 2498 | LTRLALPTAPRVL |
| SEQ ID NO: 2499 | TRLALPTAPRVLA |
| SEQ ID NO: 2500 | RLALPTAPRVLAT |
| SEQ ID NO: 2501 | LALPTAPRVLATV |
| SEQ ID NO: 2502 | ALPTAPRVLATVG |
| SEQ ID NO: 2503 | LPTAPRVLATVGE |
| SEQ ID NO: 2504 | PTAPRVLATVGEC |
| SEQ ID NO: 2505 | TAKALTRLALPTAP |
| SEQ ID NO: 2506 | AKALTRLALPTAPR |
| SEQ ID NO: 2507 | KALTRLALPTAPRV |
| SEQ ID NO: 2508 | ALTRLALPTAPRVL |
| SEQ ID NO: 2509 | LTRLALPTAPRVLA |
| SEQ ID NO: 2510 | TRLALPTAPRVLAT |
| SEQ ID NO: 2511 | RLALPTAPRVLATV |
| SEQ ID NO: 2512 | LALPTAPRVLATVG |
| SEQ ID NO: 2513 | ALPTAPRVLATVGE |
| SEQ ID NO: 2514 | LPTAPRVLATVGEC |
| SEQ ID NO: 2515 | PTAPRVLATVGECR |
| SEQ ID NO: 2516 | DTAKALTRLALPTAP |
| SEQ ID NO: 2517 | TAKALTRLALPTAPR |
| SEQ ID NO: 2518 | AKALTRLALPTAPRV |
| SEQ ID NO: 2519 | KALTRLALPTAPRVL |
| SEQ ID NO: 2520 | ALTRLALPTAPRVLA |
| SEQ ID NO: 2521 | LTRLALPTAPRVLAT |
| SEQ ID NO: 2522 | TRLALPTAPRVLATV |
| SEQ ID NO: 2523 | RLALPTAPRVLATVG |
| SEQ ID NO: 2524 | LALPTAPRVLATVGE |
| SEQ ID NO: 2525 | ALPTAPRVLATVGEC |
| SEQ ID NO: 2526 | LPTAPRVLATVGECR |
| SEQ ID NO: 2527 | PTAPRVLATVGECRY |
| SEQ ID NO: 2528 | LDTAKALTRLALPTAP |
| SEQ ID NO: 2529 | DTAKALTRLALPTAPR |
| SEQ ID NO: 2530 | TAKALTRLALPTAPRV |
| SEQ ID NO: 2531 | AKALTRLALPTAPRVL |
| SEQ ID NO: 2532 | KALTRLALPTAPRVLA |
| SEQ ID NO: 2533 | ALTRLALPTAPRVLAT |
| SEQ ID NO: 2534 | LTRLALPTAPRVLATV |
| SEQ ID NO: 2535 | TRLALPTAPRVLATVG |
| SEQ ID NO: 2536 | RLALPTAPRVLATVGE |
| SEQ ID NO: 2537 | LALPTAPRVLATVGEC |
| SEQ ID NO: 2538 | ALPTAPRVLATVGECR |
| SEQ ID NO: 2539 | LPTAPRVLATVGECRY |
| SEQ ID NO: 2540 | PTAPRVLATVGECRYS |
| SEQ ID NO: 2541 | ALDTAKALTRLALPTAP |
| SEQ ID NO: 2542 | LDTAKALTRLALPTAPR |
| SEQ ID NO: 2543 | DTAKALTRLALPTAPRV |
| SEQ ID NO: 2544 | TAKALTRLALPTAPRVL |
| SEQ ID NO: 2545 | AKALTRLALPTAPRVLA |
| SEQ ID NO: 2546 | KALTRLALPTAPRVLAT |
| SEQ ID NO: 2547 | ALTRLALPTAPRVLATV |
| SEQ ID NO: 2548 | LTRLALPTAPRVLATVG |
| SEQ ID NO: 2549 | TRLALPTAPRVLATVGE |
| SEQ ID NO: 2550 | RLALPTAPRVLATVGEC |
| SEQ ID NO: 2551 | LALPTAPRVLATVGECR |
| SEQ ID NO: 2552 | ALPTAPRVLATVGECRY |
| SEQ ID NO: 2553 | LPTAPRVLATVGECRYS |
| SEQ ID NO: 2554 | PTAPRVLATVGECRYSR |
| SEQ ID NO: 2555 | KALDTAKALTRLALPTAP |
| SEQ ID NO: 2556 | ALDTAKALTRLALPTAPR |
| SEQ ID NO: 2557 | LDTAKALTRLALPTAPRV |
| SEQ ID NO: 2558 | DTAKALTRLALPTAPRVL |
| SEQ ID NO: 2559 | TAKALTRLALPTAPRVLA |
| SEQ ID NO: 2560 | AKALTRLALPTAPRVLAT |
| SEQ ID NO: 2561 | KALTRLALPTAPRVLATV |
| SEQ ID NO: 2562 | ALTRLALPTAPRVLATVG |
| SEQ ID NO: 2563 | LTRLALPTAPRVLATVGE |
| SEQ ID NO: 2564 | TRLALPTAPRVLATVGEC |
| SEQ ID NO: 2565 | RLALPTAPRVLATVGECR |
| SEQ ID NO: 2566 | LALPTAPRVLATVGECRY |
| SEQ ID NO: 2567 | ALPTAPRVLATVGECRYS |
| SEQ ID NO: 2568 | LPTAPRVLATVGECRYSR |
| SEQ ID NO: 2569 | PTAPRVLATVGECRYSRN |
| SEQ ID NO: 2570 | EKALDTAKALTRLALPTAP |
| SEQ ID NO: 2571 | KALDTAKALTRLALPTAPR |
| SEQ ID NO: 2572 | ALDTAKALTRLALPTAPRV |
| SEQ ID NO: 2573 | LDTAKALTRLALPTAPRVL |
| SEQ ID NO: 2574 | DTAKALTRLALPTAPRVLA |
| SEQ ID NO: 2575 | TAKALTRLALPTAPRVLAT |
| SEQ ID NO: 2576 | AKALTRLALPTAPRVLATV |
| SEQ ID NO: 2577 | KALTRLALPTAPRVLATVG |
| SEQ ID NO: 2578 | ALTRLALPTAPRVLATVGE |
| SEQ ID NO: 2579 | LTRLALPTAPRVLATVGEC |
| SEQ ID NO: 2580 | TRLALPTAPRVLATVGECR |
| SEQ ID NO: 2581 | RLALPTAPRVLATVGECRY |
| SEQ ID NO: 2582 | LALPTAPRVLATVGECRYS |
| SEQ ID NO: 2583 | ALPTAPRVLATVGECRYSR |
| SEQ ID NO: 2584 | LPTAPRVLATVGECRYSRN |
| SEQ ID NO: 2585 | PTAPRVLATVGECRYSRNA |
| SEQ ID NO: 2586 | PEKALDTAKALTRLALPTAP |
| SEQ ID NO: 2587 | EKALDTAKALTRLALPTAPR |
| SEQ ID NO: 2588 | KALDTAKALTRLALPTAPRV |
| SEQ ID NO: 2589 | ALDTAKALTRLALPTAPRVL |
| SEQ ID NO: 2590 | LDTAKALTRLALPTAPRVLA |
| SEQ ID NO: 2591 | DTAKALTRLALPTAPRVLAT |
| SEQ ID NO: 2592 | TAKALTRLALPTAPRVLATV |
| SEQ ID NO: 2593 | AKALTRLALPTAPRVLATVG |
| SEQ ID NO: 2594 | KALTRLALPTAPRVLATVGE |
| SEQ ID NO: 2595 | ALTRLALPTAPRVLATVGEC |
| SEQ ID NO: 2596 | LTRLALPTAPRVLATVGECR |
| SEQ ID NO: 2597 | TRLALPTAPRVLATVGECRY |
| SEQ ID NO: 2598 | RLALPTAPRVLATVGECRYS |
| SEQ ID NO: 2599 | LALPTAPRVLATVGECRYSR |
| SEQ ID NO: 2600 | ALPTAPRVLATVGECRYSRN |
| SEQ ID NO: 2601 | LPTAPRVLATVGECRYSRNA |
| SEQ ID NO: 2602 | PTAPRVLATVGECRYSRNAP |

TABLE 20
P(T/S)AP Motif Containing Peptides from
Human Foamy Virus Gag Protein
(GenBank Accession No. NP_044279)

| | |
|---|---|
| SEQ ID NO: 2603 | PAPVPSAP |
| SEQ ID NO: 2604 | APVPSAPP |
| SEQ ID NO: 2605 | PVPSAPPM |
| SEQ ID NO: 2606 | VPSAPPMI |
| SEQ ID NO: 2607 | PSAPPMIQ |
| SEQ ID NO: 2608 | LPAPVPSAP |
| SEQ ID NO: 2609 | PAPVPSAPP |
| SEQ ID NO: 2610 | APVPSAPPM |
| SEQ ID NO: 2611 | PVPSAPPMI |
| SEQ ID NO: 2612 | VPSAPPMIQ |
| SEQ ID NO: 2613 | PSAPPMIQY |
| SEQ ID NO: 2614 | ILPAPVPSAP |
| SEQ ID NO: 2615 | LPAPVPSAPP |
| SEQ ID NO: 2616 | PAPVPSAPPM |
| SEQ ID NO: 2617 | APVPSAPPMI |
| SEQ ID NO: 2618 | PVPSAPPMIQ |
| SEQ ID NO: 2619 | VPSAPPMIQY |
| SEQ ID NO: 2620 | PSAPPMIQYI |
| SEQ ID NO: 2621 | EILPAPVPSAP |
| SEQ ID NO: 2622 | ILPAPVPSAPP |
| SEQ ID NO: 2623 | LPAPVPSAPPM |
| SEQ ID NO: 2624 | PAPVPSAPPMI |
| SEQ ID NO: 2625 | APVPSAPPMIQ |
| SEQ ID NO: 2626 | PVPSAPPMIQY |
| SEQ ID NO: 2627 | VPSAPPMIQYI |
| SEQ ID NO: 2628 | PSAPPMIQYIP |
| SEQ ID NO: 2629 | REILPAPVPSAP |
| SEQ ID NO: 2630 | EILPAPVPSAPP |
| SEQ ID NO: 2631 | ILPAPVPSAPPM |
| SEQ ID NO: 2632 | LPAPVPSAPPMI |
| SEQ ID NO: 2633 | PAPVPSAPPMIQ |
| SEQ ID NO: 2634 | APVPSAPPMIQY |
| SEQ ID NO: 2635 | PVPSAPPMIQYI |
| SEQ ID NO: 2636 | VPSAPPMIQYIP |
| SEQ ID NO: 2637 | PSAPPMIQYIPV |
| SEQ ID NO: 2638 | RREILPAPVPSAP |
| SEQ ID NO: 2639 | REILPAPVPSAPP |
| SEQ ID NO: 2640 | EILPAPVPSAPPM |
| SEQ ID NO: 2641 | ILPAPVPSAPPMI |
| SEQ ID NO: 2642 | LPAPVPSAPPMIQ |
| SEQ ID NO: 2643 | PAPVPSAPPMIQY |
| SEQ ID NO: 2644 | APVPSAPPMIQYI |
| SEQ ID NO: 2645 | PVPSAPPMIQYIP |
| SEQ ID NO: 2646 | VPSAPPMIQYIPV |
| SEQ ID NO: 2647 | PSAPPMIQYIPVP |
| SEQ ID NO: 2648 | RRREILPAPVPSAP |
| SEQ ID NO: 2649 | RREILPAPVPSAPP |
| SEQ ID NO: 2650 | REILPAPVPSAPPM |
| SEQ ID NO: 2651 | EILPAPVPSAPPMI |
| SEQ ID NO: 2652 | ILPAPVPSAPPMIQ |
| SEQ ID NO: 2653 | LPAPVPSAPPMIQY |
| SEQ ID NO: 2654 | PAPVPSAPPMIQYI |
| SEQ ID NO: 2655 | APVPSAPPMIQYIP |
| SEQ ID NO: 2656 | PVPSAPPMIQYIPV |
| SEQ ID NO: 2657 | VPSAPPMIQYIPVP |
| SEQ ID NO: 2658 | PSAPPMIQYIPVPP |
| SEQ ID NO: 2659 | ERRREILPAPVPSAP |
| SEQ ID NO: 2660 | RRREILPAPVPSAPP |
| SEQ ID NO: 2661 | RREILPAPVPSAPPM |
| SEQ ID NO: 2662 | REILPAPVPSAPPMI |
| SEQ ID NO: 2663 | EILPAPVPSAPPMIQ |
| SEQ ID NO: 2664 | ILPAPVPSAPPMIQY |
| SEQ ID NO: 2665 | LPAPVPSAPPMIQYI |
| SEQ ID NO: 2666 | PAPVPSAPPMIQYIP |
| SEQ ID NO: 2667 | APVPSAPPMIQYIPV |
| SEQ ID NO: 2668 | PVPSAPPMIQYIPVP |
| SEQ ID NO: 2669 | VPSAPPMIQYIPVPP |
| SEQ ID NO: 2670 | PSAPPMIQYIPVPPP |
| SEQ ID NO: 2671 | RERRREILPAPVPSAP |
| SEQ ID NO: 2672 | ERRREILPAPVPSAPP |
| SEQ ID NO: 2673 | RRREILPAPVPSAPPM |
| SEQ ID NO: 2674 | RREILPAPVPSAPPMI |
| SEQ ID NO: 2675 | REILPAPVPSAPPMIQ |
| SEQ ID NO: 2676 | EILPAPVPSAPPMIQY |
| SEQ ID NO: 2677 | ILPAPVPSAPPMIQYI |
| SEQ ID NO: 2678 | LPAPVPSAPPMIQYIP |
| SEQ ID NO: 2679 | PAPVPSAPPMIQYIPV |
| SEQ ID NO: 2680 | APVPSAPPMIQYIPVP |
| SEQ ID NO: 2681 | PVPSAPPMIQYIPVPP |
| SEQ ID NO: 2682 | VPSAPPMIQYIPVPPP |
| SEQ ID NO: 2683 | PSAPPMIQYIPVPPPP |
| SEQ ID NO: 2684 | SRERRREILPAPVPSAP |
| SEQ ID NO: 2685 | RERRREILPAPVPSAPP |
| SEQ ID NO: 2686 | ERRREILPAPVPSAPPM |
| SEQ ID NO: 2687 | RRREILPAPVPSAPPMI |
| SEQ ID NO: 2688 | RREILPAPVPSAPPMIQ |
| SEQ ID NO: 2689 | REILPAPVPSAPPMIQY |
| SEQ ID NO: 2690 | EILPAPVPSAPPMIQYI |
| SEQ ID NO: 2691 | ILPAPVPSAPPMIQYIP |
| SEQ ID NO: 2692 | LPAPVPSAPPMIQYIPV |
| SEQ ID NO: 2693 | PAPVPSAPPMIQYIPVP |
| SEQ ID NO: 2694 | APVPSAPPMIQYIPVPP |
| SEQ ID NO: 2695 | PVPSAPPMIQYIPVPPP |
| SEQ ID NO: 2696 | VPSAPPMIQYIPVPPPP |
| SEQ ID NO: 2697 | PSAPPMIQYIPVPPPPP |
| SEQ ID NO: 2698 | QSRERRREILPAPVPSAP |
| SEQ ID NO: 2699 | SRERRREILPAPVPSAPP |
| SEQ ID NO: 2700 | RERRREILPAPVPSAPPM |
| SEQ ID NO: 2701 | ERRREILPAPVPSAPPMI |
| SEQ ID NO: 2702 | RRREILPAPVPSAPPMIQ |
| SEQ ID NO: 2703 | RREILPAPVPSAPPMIQY |
| SEQ ID NO: 2704 | REILPAPVPSAPPMIQYI |
| SEQ ID NO: 2705 | EILPAPVPSAPPMIQYIP |
| SEQ ID NO: 2706 | ILPAPVPSAPPMIQYIPV |
| SEQ ID NO: 2707 | LPAPVPSAPPMIQYIPVP |
| SEQ ID NO: 2708 | PAPVPSAPPMIQYIPVPP |
| SEQ ID NO: 2709 | APVPSAPPMIQYIPVPPP |
| SEQ ID NO: 2710 | PVPSAPPMIQYIPVPPPP |
| SEQ ID NO: 2711 | VPSAPPMIQYIPVPPPPP |
| SEQ ID NO: 2712 | PSAPPMIQYIPVPPPPPI |
| SEQ ID NO: 2713 | SQSRERRREILPAPVPSAP |
| SEQ ID NO: 2714 | QSRERRREILPAPVPSAPP |
| SEQ ID NO: 2715 | SRERRREILPAPVPSAPPM |
| SEQ ID NO: 2716 | RERRREILPAPVPSAPPMI |
| SEQ ID NO: 2717 | ERRREILPAPVPSAPPMIQ |
| SEQ ID NO: 2718 | RRREILPAPVPSAPPMIQY |
| SEQ ID NO: 2719 | RREILPAPVPSAPPMIQYI |
| SEQ ID NO: 2720 | REILPAPVPSAPPMIQYIP |
| SEQ ID NO: 2721 | EILPAPVPSAPPMIQYIPV |
| SEQ ID NO: 2722 | ILPAPVPSAPPMIQYIPVP |
| SEQ ID NO: 2723 | LPAPVPSAPPMIQYIPVPP |
| SEQ ID NO: 2724 | PAPVPSAPPMIQYIPVPPP |
| SEQ ID NO: 2725 | APVPSAPPMIQYIPVPPPP |
| SEQ ID NO: 2726 | PVPSAPPMIQYIPVPPPPP |
| SEQ ID NO: 2727 | VPSAPPMIQYIPVPPPPPI |
| SEQ ID NO: 2728 | PSAPPMIQYIPVPPPPPIG |
| SEQ ID NO: 2729 | RSQSRERRREILPAPVPSAP |
| SEQ ID NO: 2730 | SQSRERRREILPAPVPSAPP |
| SEQ ID NO: 2731 | QSRERRREILPAPVPSAPPM |
| SEQ ID NO: 2732 | SRERRREILPAPVPSAPPMI |
| SEQ ID NO: 2733 | RERRREILPAPVPSAPPMIQ |
| SEQ ID NO: 2734 | ERRREILPAPVPSAPPMIQY |
| SEQ ID NO: 2735 | RRREILPAPVPSAPPMIQYI |
| SEQ ID NO: 2736 | RREILPAPVPSAPPMIQYIP |
| SEQ ID NO: 2737 | REILPAPVPSAPPMIQYIPV |
| SEQ ID NO: 2738 | EILPAPVPSAPPMIQYIPVP |
| SEQ ID NO: 2739 | ILPAPVPSAPPMIQYIPVPP |
| SEQ ID NO: 2740 | LPAPVPSAPPMIQYIPVPPP |
| SEQ ID NO: 2741 | PAPVPSAPPMIQYIPVPPPP |
| SEQ ID NO: 2742 | APVPSAPPMIQYIPVPPPPP |
| SEQ ID NO: 2743 | PVPSAPPMIQYIPVPPPPPI |
| SEQ ID NO: 2744 | VPSAPPMIQYIPVPPPPPIG |
| SEQ ID NO: 2745 | PSAPPMIQYIPVPPPPPIGT |

TABLE 21
P(T/S)AP Motif Containing Peptides from
Hepatitis E Virus ORF-3 Protein
(GenBank Accession No. AAC35758)

| | |
|---|---|
| SEQ ID NO: 2746 | GVTRPSAP |
| SEQ ID NO: 2747 | VTRPSAPP |
| SEQ ID NO: 2748 | TRPSAPPL |
| SEQ ID NO: 2749 | RPSAPPLP |
| SEQ ID NO: 2750 | PSAPPLPH |
| SEQ ID NO: 2751 | LGVTRPSAP |
| SEQ ID NO: 2752 | GVTRPSAPP |
| SEQ ID NO: 2753 | VTRPSAPPL |
| SEQ ID NO: 2754 | TRPSAPPLP |
| SEQ ID NO: 2755 | RPSAPPLPH |
| SEQ ID NO: 2756 | PSAPPLPHV |
| SEQ ID NO: 2757 | PLGVTRPSAP |
| SEQ ID NO: 2758 | LGVTRPSAPP |
| SEQ ID NO: 2759 | GVTRPSAPPL |
| SEQ ID NO: 2760 | VTRPSAPPLP |
| SEQ ID NO: 2761 | TRPSAPPLPH |
| SEQ ID NO: 2762 | RPSAPPLPHV |
| SEQ ID NO: 2763 | PSAPPLPHVV |
| SEQ ID NO: 2764 | APLGVTRPSAP |
| SEQ ID NO: 2765 | PLGVTRPSAPP |
| SEQ ID NO: 2766 | LGVTRPSAPPL |
| SEQ ID NO: 2767 | GVTRPSAPPLP |
| SEQ ID NO: 2768 | VTRPSAPPLPH |
| SEQ ID NO: 2769 | TRPSAPPLPHV |
| SEQ ID NO: 2770 | RPSAPPLPHVV |
| SEQ ID NO: 2771 | PSAPPLPHVVD |
| SEQ ID NO: 2772 | SAPLGVTRPSAP |
| SEQ ID NO: 2773 | APLGVTRPSAPP |
| SEQ ID NO: 2774 | PLGVTRPSAPPL |
| SEQ ID NO: 2775 | LGVTRPSAPPLP |
| SEQ ID NO: 2776 | GVTRPSAPPLPH |
| SEQ ID NO: 2777 | VTRPSAPPLPHV |
| SEQ ID NO: 2778 | TRPSAPPLPHVV |
| SEQ ID NO: 2779 | RPSAPPLPHVVD |
| SEQ ID NO: 2780 | PSAPPLPHVVDL |
| SEQ ID NO: 2781 | HSAPLGVTRPSAP |
| SEQ ID NO: 2782 | SAPLGVTRPSAPP |
| SEQ ID NO: 2783 | APLGVTRPSAPPL |
| SEQ ID NO: 2784 | PLGVTRPSAPPLP |
| SEQ ID NO: 2785 | LGVTRPSAPPLPH |
| SEQ ID NO: 2786 | GVTRPSAPPLPHV |
| SEQ ID NO: 2787 | VTRPSAPPLPHVV |
| SEQ ID NO: 2788 | TRPSAPPLPHVVD |
| SEQ ID NO: 2789 | RPSAPPLPHVVDL |
| SEQ ID NO: 2790 | PSAPPLPHVVDLP |
| SEQ ID NO: 2791 | DHSAPLGVTRPSAP |
| SEQ ID NO: 2792 | HSAPLGVTRPSAPP |
| SEQ ID NO: 2793 | SAPLGVTRPSAPPL |
| SEQ ID NO: 2794 | APLGVTRPSAPPLP |
| SEQ ID NO: 2795 | PLGVTRPSAPPLPH |
| SEQ ID NO: 2796 | LGVTRPSAPPLPHV |
| SEQ ID NO: 2797 | GVTRPSAPPLPHVV |
| SEQ ID NO: 2798 | VTRPSAPPLPHVVD |
| SEQ ID NO: 2799 | TRPSAPPLPHVVDL |
| SEQ ID NO: 2800 | RPSAPPLPHVVDLP |
| SEQ ID NO: 2801 | PSAPPLPHVVDLPQ |
| SEQ ID NO: 2802 | PDHSAPLGVTRPSAP |
| SEQ ID NO: 2803 | DHSAPLGVTRPSAPP |
| SEQ ID NO: 2804 | HSAPLGVTRPSAPPL |
| SEQ ID NO: 2805 | SAPLGVTRPSAPPLP |
| SEQ ID NO: 2806 | APLGVTRPSAPPLPH |
| SEQ ID NO: 2807 | PLGVTRPSAPPLPHV |
| SEQ ID NO: 2808 | LGVTRPSAPPLPHVV |
| SEQ ID NO: 2809 | GVTRPSAPPLPHVVD |
| SEQ ID NO: 2810 | VTRPSAPPLPHVVDL |
| SEQ ID NO: 2811 | TRPSAPPLPHVVDLP |
| SEQ ID NO: 2812 | RPSAPPLPHVVDLPQ |
| SEQ ID NO: 2813 | PSAPPLPHVVDLPQL |
| SEQ ID NO: 2814 | LPDHSAPLGVTRPSAP |
| SEQ ID NO: 2815 | PDHSAPLGVTRPSAPP |
| SEQ ID NO: 2816 | DHSAPLGVTRPSAPPL |
| SEQ ID NO: 2817 | HSAPLGVTRPSAPPLP |
| SEQ ID NO: 2818 | SAPLGVTRPSAPPLPH |
| SEQ ID NO: 2819 | APLGVTRPSAPPLPHV |
| SEQ ID NO: 2820 | PLGVTRPSAPPLPHVV |
| SEQ ID NO: 2821 | LGVTRPSAPPLPHVVD |
| SEQ ID NO: 2822 | GVTRPSAPPLPHVVDL |
| SEQ ID NO: 2823 | VTRPSAPPLPHVVDLP |
| SEQ ID NO: 2824 | TRPSAPPLPHVVDLPQ |
| SEQ ID NO: 2825 | RPSAPPLPHVVDLPQL |
| SEQ ID NO: 2826 | PSAPPLPHVVDLPQLG |
| SEQ ID NO: 2827 | NLPDHSAPLGVTRPSAP |
| SEQ ID NO: 2828 | LPDHSAPLGVTRPSAPP |
| SEQ ID NO: 2829 | PDHSAPLGVTRPSAPPL |
| SEQ ID NO: 2830 | DHSAPLGVTRPSAPPLP |
| SEQ ID NO: 2831 | HSAPLGVTRPSAPPLPH |
| SEQ ID NO: 2832 | SAPLGVTRPSAPPLPHV |
| SEQ ID NO: 2833 | APLGVTRPSAPPLPHVV |
| SEQ ID NO: 2834 | PLGVTRPSAPPLPHVVD |
| SEQ ID NO: 2835 | LGVTRPSAPPLPHVVDL |
| SEQ ID NO: 2836 | GVTRPSAPPLPHVVDLP |
| SEQ ID NO: 2837 | VTRPSAPPLPHVVDLPQ |
| SEQ ID NO: 2838 | TRPSAPPLPHVVDLPQL |
| SEQ ID NO: 2839 | RPSAPPLPHVVDLPQLG |
| SEQ ID NO: 2840 | PSAPPLPHVVDLPQLGP |
| SEQ ID NO: 2841 | ANLPDHSAPLGVTRPSAP |
| SEQ ID NO: 2842 | NLPDHSAPLGVTRPSAPP |
| SEQ ID NO: 2843 | LPDHSAPLGVTRPSAPPL |
| SEQ ID NO: 2844 | PDHSAPLGVTRPSAPPLP |
| SEQ ID NO: 2845 | DHSAPLGVTRPSAPPLPH |
| SEQ ID NO: 2846 | HSAPLGVTRPSAPPLPHV |
| SEQ ID NO: 2847 | SAPLGVTRPSAPPLPHVV |
| SEQ ID NO: 2848 | APLGVTRPSAPPLPHVVD |
| SEQ ID NO: 2849 | PLGVTRPSAPPLPHVVDL |
| SEQ ID NO: 2850 | LGVTRPSAPPLPHVVDLP |
| SEQ ID NO: 2851 | GVTRPSAPPLPHVVDLPQ |
| SEQ ID NO: 2852 | VTRPSAPPLPHVVDLPQL |
| SEQ ID NO: 2853 | TRPSAPPLPHVVDLPQLG |
| SEQ ID NO: 2854 | RPSAPPLPHVVDLPQLGP |
| SEQ ID NO: 2855 | PSAPPLPHVVDLPQLGPR |
| SEQ ID NO: 2856 | FANLPDHSAPLGVTRPSAP |
| SEQ ID NO: 2857 | ANLPDHSAPLGVTRPSAPP |
| SEQ ID NO: 2858 | NLPDHSAPLGVTRPSAPPL |
| SEQ ID NO: 2859 | LPDHSAPLGVTRPSAPPLP |
| SEQ ID NO: 2860 | PDHSAPLGVTRPSAPPLPH |
| SEQ ID NO: 2861 | DHSAPLGVTRPSAPPLPHV |
| SEQ ID NO: 2862 | HSAPLGVTRPSAPPLPHVV |
| SEQ ID NO: 2863 | SAPLGVTRPSAPPLPHVVD |
| SEQ ID NO: 2864 | APLGVTRPSAPPLPHVVDL |
| SEQ ID NO: 2865 | PLGVTRPSAPPLPHVVDLP |
| SEQ ID NO: 2866 | LGVTRPSAPPLPHVVDLPQ |
| SEQ ID NO: 2867 | GVTRPSAPPLPHVVDLPQL |
| SEQ ID NO: 2868 | VTRPSAPPLPHVVDLPQLG |
| SEQ ID NO: 2869 | TRPSAPPLPHVVDLPQLGP |
| SEQ ID NO: 2870 | RPSAPPLPHVVDLPQLGPR |
| SEQ ID NO: 2871 | PSAPPLPHVVDLPQLGPRR |
| SEQ ID NO: 2872 | VFANLPDHSAPLGVTRPSAP |
| SEQ ID NO: 2873 | FANLPDHSAPLGVTRPSAPP |
| SEQ ID NO: 2874 | ANLPDHSAPLGVTRPSAPPL |
| SEQ ID NO: 2875 | NLPDHSAPLGVTRPSAPPLP |
| SEQ ID NO: 2876 | LPDHSAPLGVTRPSAPPLPH |
| SEQ ID NO: 2877 | PDHSAPLGVTRPSAPPLPHV |
| SEQ ID NO: 2878 | DHSAPLGVTRPSAPPLPHVV |
| SEQ ID NO: 2879 | HSAPLGVTRPSAPPLPHVVD |
| SEQ ID NO: 2880 | SAPLGVTRPSAPPLPHVVDL |
| SEQ ID NO: 2881 | APLGVTRPSAPPLPHVVDLP |
| SEQ ID NO: 2882 | PLGVTRPSAPPLPHVVDLPQ |
| SEQ ID NO: 2883 | LGVTRPSAPPLPHVVDLPQL |
| SEQ ID NO: 2884 | GVTRPSAPPLPHVVDLPQLG |
| SEQ ID NO: 2885 | VTRPSAPPLPHVVDLPQLGP |
| SEQ ID NO: 2886 | TRPSAPPLPHVVDLPQLGPR |
| SEQ ID NO: 2887 | RPSAPPLPHVVDLPQLGPRR |

TABLE 22
P(T/S)AP Motif Containing Peptides from
Hepatitis G Virus Polyprotein
(GenBank Accession No. AAB65834)

| | |
|---|---|
| SEQ ID NO: 2888 | PGFVPTAP |
| SEQ ID NO: 2889 | GFVPTAPV |
| SEQ ID NO: 2890 | FVPTAPVV |
| SEQ ID NO: 2891 | VPTAPVVI |
| SEQ ID NO: 2892 | PTAPVVIR |
| SEQ ID NO: 2893 | PPGFVPTAP |
| SEQ ID NO: 2894 | PGFVPTAPV |
| SEQ ID NO: 2895 | GFVPTAPVV |
| SEQ ID NO: 2896 | FVPTAPVVI |
| SEQ ID NO: 2897 | VPTAPVVIR |
| SEQ ID NO: 2898 | PTAPVVIRR |
| SEQ ID NO: 2899 | LPPGFVPTAP |
| SEQ ID NO: 2900 | PPGFVPTAPV |
| SEQ ID NO: 2901 | PGFVPTAPVV |
| SEQ ID NO: 2902 | GFVPTAPVVI |
| SEQ ID NO: 2903 | FVPTAPVVIR |
| SEQ ID NO: 2904 | VPTAPVVIRR |
| SEQ ID NO: 2905 | PTAPVVIRRC |
| SEQ ID NO: 2906 | HLPPGFVPTAP |
| SEQ ID NO: 2907 | LPPGFVPTAPV |
| SEQ ID NO: 2908 | PPGFVPTAPVV |
| SEQ ID NO: 2909 | PGFVPTAPVVI |
| SEQ ID NO: 2910 | GFVPTAPVVIR |
| SEQ ID NO: 2911 | FVPTAPVVIRR |
| SEQ ID NO: 2912 | VPTAPVVIRRC |
| SEQ ID NO: 2913 | PTAPVVIRRCG |
| SEQ ID NO: 2914 | NHLPPGFVPTAP |
| SEQ ID NO: 2915 | HLPPGFVPTAPV |
| SEQ ID NO: 2916 | LPPGFVPTAPVV |
| SEQ ID NO: 2917 | PPGFVPTAPVVI |
| SEQ ID NO: 2918 | PGFVPTAPVVIR |
| SEQ ID NO: 2919 | GFVPTAPVVIRR |
| SEQ ID NO: 2920 | FVPTAPVVIRRC |
| SEQ ID NO: 2921 | VPTAPVVIRRCG |
| SEQ ID NO: 2922 | PTAPVVIRRCGK |
| SEQ ID NO: 2923 | VNHLPPGFVPTAP |
| SEQ ID NO: 2924 | NHLPPGFVPTAPV |
| SEQ ID NO: 2925 | HLPPGFVPTAPVV |
| SEQ ID NO: 2926 | LPPGFVPTAPVVI |
| SEQ ID NO: 2927 | PPGFVPTAPVVIR |
| SEQ ID NO: 2928 | PGFVPTAPVVIRR |
| SEQ ID NO: 2929 | GFVPTAPVVIRRC |
| SEQ ID NO: 2930 | FVPTAPVVIRRCG |
| SEQ ID NO: 2931 | VPTAPVVIRRCGK |
| SEQ ID NO: 2932 | PTAPVVIRRCGKG |
| SEQ ID NO: 2933 | DVNHLPPGFVPTAP |
| SEQ ID NO: 2934 | VNHLPPGFVPTAPV |
| SEQ ID NO: 2935 | NHLPPGFVPTAPVV |
| SEQ ID NO: 2936 | HLPPGFVPTAPVVI |
| SEQ ID NO: 2937 | LPPGFVPTAPVVIR |
| SEQ ID NO: 2938 | PPGFVPTAPVVIRR |
| SEQ ID NO: 2939 | PGFVPTAPVVIRRC |
| SEQ ID NO: 2940 | GFVPTAPVVIRRCG |
| SEQ ID NO: 2941 | FVPTAPVVIRRCGK |
| SEQ ID NO: 2942 | VPTAPVVIRRCGKG |
| SEQ ID NO: 2943 | PTAPVVIRRCGKGF |
| SEQ ID NO: 2944 | QDVNHLPPGFVPTAP |
| SEQ ID NO: 2945 | DVNHLPPGFVPTAPV |
| SEQ ID NO: 2946 | VNHLPPGFVPTAPVV |
| SEQ ID NO: 2947 | NHLPPGFVPTAPVVI |
| SEQ ID NO: 2948 | HLPPGFVPTAPVVIR |
| SEQ ID NO: 2949 | LPPGFVPTAPVVIRR |
| SEQ ID NO: 2950 | PPGFVPTAPVVIRRC |
| SEQ ID NO: 2951 | PGFVPTAPVVIRRCG |
| SEQ ID NO: 2952 | GFVPTAPVVIRRCGK |
| SEQ ID NO: 2953 | FVPTAPVVIRRCGKG |
| SEQ ID NO: 2954 | VPTAPVVIRRCGKGF |
| SEQ ID NO: 2955 | PTAPVVIRRCGKGFL |
| SEQ ID NO: 2956 | FQDVNHLPPGFVPTAP |
| SEQ ID NO: 2957 | QDVNHLPPGFVPTAPV |
| SEQ ID NO: 2958 | DVNHLPPGFVPTAPVV |
| SEQ ID NO: 2959 | VNHLPPGFVPTAPVVI |
| SEQ ID NO: 2960 | NHLPPGFVPTAPVVIR |
| SEQ ID NO: 2961 | HLPPGFVPTAPVVIRR |
| SEQ ID NO: 2962 | LPPGFVPTAPVVIRRC |
| SEQ ID NO: 2963 | PPGFVPTAPVVIRRCG |
| SEQ ID NO: 2964 | PGFVPTAPVVIRRCGK |
| SEQ ID NO: 2965 | GFVPTAPVVIRRCGKG |
| SEQ ID NO: 2966 | FVPTAPVVIRRCGKGF |
| SEQ ID NO: 2967 | VPTAPVVIRRCGKGFL |
| SEQ ID NO: 2968 | PTAPVVIRRCGKGFLG |
| SEQ ID NO: 2969 | VFQDVNHLPPGFVPTAP |
| SEQ ID NO: 2970 | FQDVNHLPPGFVPTAPV |
| SEQ ID NO: 2971 | QDVNHLPPGFVPTAPVV |
| SEQ ID NO: 2972 | DVNHLPPGFVPTAPVVI |
| SEQ ID NO: 2973 | VNHLPPGFVPTAPVVIR |
| SEQ ID NO: 2974 | NHLPPGFVPTAPVVIRR |
| SEQ ID NO: 2975 | HLPPGFVPTAPVVIRRC |
| SEQ ID NO: 2976 | LPPGFVPTAPVVIRRCG |
| SEQ ID NO: 2977 | PPGFVPTAPVVIRRCGK |
| SEQ ID NO: 2978 | PGFVPTAPVVIRRCGKG |
| SEQ ID NO: 2979 | GFVPTAPVVIRRCGKGF |
| SEQ ID NO: 2980 | FVPTAPVVIRRCGKGFL |
| SEQ ID NO: 2981 | VPTAPVVIRRCGKGFLG |
| SEQ ID NO: 2982 | PTAPVVIRRCGKGFLGV |
| SEQ ID NO: 2983 | GVFQDVNHLPPGFVPTAP |
| SEQ ID NO: 2984 | VFQDVNHLPPGFVPTAPV |
| SEQ ID NO: 2985 | FQDVNHLPPGFVPTAPVV |
| SEQ ID NO: 2986 | QDVNHLPPGFVPTAPVVI |
| SEQ ID NO: 2987 | DVNHLPPGFVPTAPVVIR |
| SEQ ID NO: 2988 | VNHLPPGFVPTAPVVIRR |
| SEQ ID NO: 2989 | NHLPPGFVPTAPVVIRRC |
| SEQ ID NO: 2990 | HLPPGFVPTAPVVIRRCG |
| SEQ ID NO: 2991 | LPPGFVPTAPVVIRRCGK |
| SEQ ID NO: 2992 | PPGFVPTAPVVIRRCGKG |
| SEQ ID NO: 2993 | PGFVPTAPVVIRRCGKGF |
| SEQ ID NO: 2994 | GFVPTAPVVIRRCGKGFL |
| SEQ ID NO: 2995 | FVPTAPVVIRRCGKGFLG |
| SEQ ID NO: 2996 | VPTAPVVIRRCGKGFLGV |
| SEQ ID NO: 2997 | PTAPVVIRRCGKGFLGVT |
| SEQ ID NO: 2998 | IGVFQDVNHLPPGFVPTAP |
| SEQ ID NO: 2999 | GVFQDVNHLPPGFVPTAPV |
| SEQ ID NO: 3000 | VFQDVNHLPPGFVPTAPVV |
| SEQ ID NO: 3001 | FQDVNHLPPGFVPTAPVVI |
| SEQ ID NO: 3002 | QDVNHLPPGFVPTAPVVIR |
| SEQ ID NO: 3003 | DVNHLPPGFVPTAPVVIRR |
| SEQ ID NO: 3004 | VNHLPPGFVPTAPVVIRRC |
| SEQ ID NO: 3005 | NHLPPGFVPTAPVVIRRCG |
| SEQ ID NO: 3006 | HLPPGFVPTAPVVIRRCGK |
| SEQ ID NO: 3007 | LPPGFVPTAPVVIRRCGKG |
| SEQ ID NO: 3008 | PPGFVPTAPVVIRRCGKGF |
| SEQ ID NO: 3009 | PGFVPTAPVVIRRCGKGFL |
| SEQ ID NO: 3010 | GFVPTAPVVIRRCGKGFLG |
| SEQ ID NO: 3011 | FVPTAPVVIRRCGKGFLGV |
| SEQ ID NO: 3012 | VPTAPVVIRRCGKGFLGVT |
| SEQ ID NO: 3013 | PTAPVVIRRCGKGFLGVTK |
| SEQ ID NO: 3014 | LIGVFQDVNHLPPGFVPTAP |
| SEQ ID NO: 3015 | IGVFQDVNHLPPGFVPTAPV |
| SEQ ID NO: 3016 | GVFQDVNHLPPGFVPTAPVV |
| SEQ ID NO: 3017 | VFQDVNHLPPGFVPTAPVVI |
| SEQ ID NO: 3018 | FQDVNHLPPGFVPTAPVVIR |
| SEQ ID NO: 3019 | QDVNHLPPGFVPTAPVVIRR |
| SEQ ID NO: 3020 | DVNHLPPGFVPTAPVVIRRC |
| SEQ ID NO: 3021 | VNHLPPGFVPTAPVVIRRCG |
| SEQ ID NO: 3022 | NHLPPGFVPTAPVVIRRCGK |
| SEQ ID NO: 3023 | HLPPGFVPTAPVVIRRCGKG |
| SEQ ID NO: 3024 | LPPGFVPTAPVVIRRCGKGF |
| SEQ ID NO: 3025 | PPGFVPTAPVVIRRCGKGFL |
| SEQ ID NO: 3026 | PGFVPTAPVVIRRCGKGFLG |
| SEQ ID NO: 3027 | GFVPTAPVVIRRCGKGFLGV |
| SEQ ID NO: 3028 | FVPTAPVVIRRCGKGFLGVT |
| SEQ ID NO: 3029 | VPTAPVVIRRCGKGFLGVTK |
| SEQ ID NO: 3030 | PTAPVVIRRCGKGFLGVTKA |

TABLE 23
P(T/S)AP Motif Containing Peptides from Human
Herpesvirus 5 UL32
(GenBank Accession No. AAG31644)

| | |
|---|---|
| SEQ ID NO: 3031 | SPWAPTAP |
| SEQ ID NO: 3032 | PWAPTAPL |
| SEQ ID NO: 3033 | WAPTAPLP |
| SEQ ID NO: 3034 | APTAPLPG |
| SEQ ID NO: 3035 | PTAPLPGD |
| SEQ ID NO: 3036 | NSPWAPTAP |
| SEQ ID NO: 3037 | SPWAPTAPL |
| SEQ ID NO: 3038 | PWAPTAPLP |
| SEQ ID NO: 3039 | WAPTAPLPG |
| SEQ ID NO: 3040 | APTAPLPGD |
| SEQ ID NO: 3041 | PTAPLPGDM |
| SEQ ID NO: 3042 | GNSPWAPTAP |
| SEQ ID NO: 3043 | NSPWAPTAPL |
| SEQ ID NO: 3044 | SPWAPTAPLP |
| SEQ ID NO: 3045 | PWAPTAPLPG |
| SEQ ID NO: 3046 | WAPTAPLPGD |
| SEQ ID NO: 3047 | APTAPLPGDM |
| SEQ ID NO: 3048 | PTAPLPGDMN |
| SEQ ID NO: 3049 | NGNSPWAPTAP |
| SEQ ID NO: 3050 | GNSPWAPTAPL |
| SEQ ID NO: 3051 | NSPWAPTAPLP |
| SEQ ID NO: 3052 | SPWAPTAPLPG |
| SEQ ID NO: 3053 | PWAPTAPLPGD |
| SEQ ID NO: 3054 | WAPTAPLPGDM |
| SEQ ID NO: 3055 | APTAPLPGDMN |
| SEQ ID NO: 3056 | PTAPLPGDMNP |
| SEQ ID NO: 3057 | VNGNSPWAPTAP |
| SEQ ID NO: 3058 | NGNSPWAPTAPL |
| SEQ ID NO: 3059 | GNSPWAPTAPLP |
| SEQ ID NO: 3060 | NSPWAPTAPLPG |
| SEQ ID NO: 3061 | SPWAPTAPLPGD |
| SEQ ID NO: 3062 | PWAPTAPLPGDM |
| SEQ ID NO: 3063 | WAPTAPLPGDMN |
| SEQ ID NO: 3064 | APTAPLPGDMNP |
| SEQ ID NO: 3065 | PTAPLPGDMNPA |
| SEQ ID NO: 3066 | PVNGNSPWAPTAP |
| SEQ ID NO: 3067 | VNGNSPWAPTAPL |
| SEQ ID NO: 3068 | NGNSPWAPTAPLP |
| SEQ ID NO: 3069 | GNSPWAPTAPLPG |
| SEQ ID NO: 3070 | NSPWAPTAPLPGD |
| SEQ ID NO: 3071 | SPWAPTAPLPGDM |
| SEQ ID NO: 3072 | PWAPTAPLPGDMN |
| SEQ ID NO: 3073 | WAPTAPLPGDMNP |
| SEQ ID NO: 3074 | APTAPLPGDMNPA |
| SEQ ID NO: 3075 | PTAPLPGDMNPAN |
| SEQ ID NO: 3076 | TPVNGNSPWAPTAP |
| SEQ ID NO: 3077 | PVNGNSPWAPTAPL |
| SEQ ID NO: 3078 | VNGNSPWAPTAPLP |
| SEQ ID NO: 3079 | NGNSPWAPTAPLPG |
| SEQ ID NO: 3080 | GNSPWAPTAPLPGD |
| SEQ ID NO: 3081 | NSPWAPTAPLPGDM |
| SEQ ID NO: 3082 | SPWAPTAPLPGDMN |
| SEQ ID NO: 3083 | PWAPTAPLPGDMNP |
| SEQ ID NO: 3084 | WAPTAPLPGDMNPA |
| SEQ ID NO: 3085 | APTAPLPGDMNPAN |
| SEQ ID NO: 3086 | PTAPLPGDMNPANW |
| SEQ ID NO: 3087 | QTPVNGNSPWAPTAP |
| SEQ ID NO: 3088 | TPVNGNSPWAPTAPL |
| SEQ ID NO: 3089 | PVNGNSPWAPTAPLP |
| SEQ ID NO: 3090 | VNGNSPWAPTAPLPG |
| SEQ ID NO: 3091 | NGNSPWAPTAPLPGD |
| SEQ ID NO: 3092 | GNSPWAPTAPLPGDM |
| SEQ ID NO: 3093 | NSPWAPTAPLPGDMN |
| SEQ ID NO: 3094 | SPWAPTAPLPGDMNP |
| SEQ ID NO: 3095 | PWAPTAPLPGDMNPA |
| SEQ ID NO: 3096 | WAPTAPLPGDMNPAN |
| SEQ ID NO: 3097 | APTAPLPGDMNPANW |
| SEQ ID NO: 3098 | PTAPLPGDMNPANWP |
| SEQ ID NO: 3099 | TQTPVNGNSPWAPTAP |
| SEQ ID NO: 3100 | QTPVNGNSPWAPTAPL |
| SEQ ID NO: 3101 | TPVNGNSPWAPTAPLP |
| SEQ ID NO: 3102 | PVNGNSPWAPTAPLPG |
| SEQ ID NO: 3103 | VNGNSPWAPTAPLPGD |
| SEQ ID NO: 3104 | NGNSPWAPTAPLPGDM |
| SEQ ID NO: 3105 | GNSPWAPTAPLPGDMN |
| SEQ ID NO: 3106 | NSPWAPTAPLPGDMNP |
| SEQ ID NO: 3107 | SPWAPTAPLPGDMNPA |
| SEQ ID NO: 3108 | PWAPTAPLPGDMNPAN |
| SEQ ID NO: 3109 | WAPTAPLPGDMNPANW |
| SEQ ID NO: 3110 | APTAPLPGDMNPANWP |
| SEQ ID NO: 3111 | PTAPLPGDMNPANWPR |
| SEQ ID NO: 3112 | GTQTPVNGNSPWAPTAP |
| SEQ ID NO: 3113 | TQTPVNGNSPWAPTAPL |
| SEQ ID NO: 3114 | QTPVNGNSPWAPTAPLP |
| SEQ ID NO: 3115 | TPVNGNSPWAPTAPLPG |
| SEQ ID NO: 3116 | PVNGNSPWAPTAPLPGD |
| SEQ ID NO: 3117 | VNGNSPWAPTAPLPGDM |
| SEQ ID NO: 3118 | NGNSPWAPTAPLPGDMN |
| SEQ ID NO: 3119 | GNSPWAPTAPLPGDMNP |
| SEQ ID NO: 3120 | NSPWAPTAPLPGDMNPA |
| SEQ ID NO: 3121 | SPWAPTAPLPGDMNPAN |
| SEQ ID NO: 3122 | PWAPTAPLPGDMNPANW |
| SEQ ID NO: 3123 | WAPTAPLPGDMNPANWP |
| SEQ ID NO: 3124 | APTAPLPGDMNPANWPR |
| SEQ ID NO: 3125 | PTAPLPGDMNPANWPRE |
| SEQ ID NO: 3126 | AGTQTPVNGNSPWAPTAP |
| SEQ ID NO: 3127 | GTQTPVNGNSPWAPTAPL |
| SEQ ID NO: 3128 | TQTPVNGNSPWAPTAPLP |
| SEQ ID NO: 3129 | QTPVNGNSPWAPTAPLPG |
| SEQ ID NO: 3130 | TPVNGNSPWAPTAPLPGD |
| SEQ ID NO: 3131 | PVNGNSPWAPTAPLPGDM |
| SEQ ID NO: 3132 | VNGNSPWAPTAPLPGDMN |
| SEQ ID NO: 3133 | NGNSPWAPTAPLPGDMNP |
| SEQ ID NO: 3134 | GNSPWAPTAPLPGDMNPA |
| SEQ ID NO: 3135 | NSPWAPTAPLPGDMNPAN |
| SEQ ID NO: 3136 | SPWAPTAPLPGDMNPANW |
| SEQ ID NO: 3137 | PWAPTAPLPGDMNPANWP |
| SEQ ID NO: 3138 | WAPTAPLPGDMNPANWPR |
| SEQ ID NO: 3139 | APTAPLPGDMNPANWPRE |
| SEQ ID NO: 3140 | PTAPLPGDMNPANWPRER |
| SEQ ID NO: 3141 | FAGTQTPVNGNSPWAPTAP |
| SEQ ID NO: 3142 | AGTQTPVNGNSPWAPTAPL |
| SEQ ID NO: 3143 | GTQTPVNGNSPWAPTAPLP |
| SEQ ID NO: 3144 | TQTPVNGNSPWAPTAPLPG |
| SEQ ID NO: 3145 | QTPVNGNSPWAPTAPLPGD |
| SEQ ID NO: 3146 | TPVNGNSPWAPTAPLPGDM |
| SEQ ID NO: 3147 | PVNGNSPWAPTAPLPGDMN |
| SEQ ID NO: 3148 | VNGNSPWAPTAPLPGDMNP |
| SEQ ID NO: 3149 | NGNSPWAPTAPLPGDMNPA |
| SEQ ID NO: 3150 | GNSPWAPTAPLPGDMNPAN |
| SEQ ID NO: 3151 | NSPWAPTAPLPGDMNPANW |
| SEQ ID NO: 3152 | SPWAPTAPLPGDMNPANWP |
| SEQ ID NO: 3153 | PWAPTAPLPGDMNPANWPR |
| SEQ ID NO: 3154 | WAPTAPLPGDMNPANWPRE |
| SEQ ID NO: 3155 | APTAPLPGDMNPANWPRER |
| SEQ ID NO: 3156 | PTAPLPGDMNPANWPRERA |
| SEQ ID NO: 3157 | TFAGTQTPVNGNSPWAPTAP |
| SEQ ID NO: 3158 | FAGTQTPVNGNSPWAPTAPL |
| SEQ ID NO: 3159 | AGTQTPVNGNSPWAPTAPLP |
| SEQ ID NO: 3160 | GTQTPVNGNSPWAPTAPLPG |
| SEQ ID NO: 3161 | TQTPVNGNSPWAPTAPLPGD |
| SEQ ID NO: 3162 | QTPVNGNSPWAPTAPLPGDM |
| SEQ ID NO: 3163 | TPVNGNSPWAPTAPLPGDMN |
| SEQ ID NO: 3164 | PVNGNSPWAPTAPLPGDMNP |
| SEQ ID NO: 3165 | VNGNSPWAPTAPLPGDMNPA |
| SEQ ID NO: 3166 | NGNSPWAPTAPLPGDMNPAN |
| SEQ ID NO: 3167 | GNSPWAPTAPLPGDMNPANW |
| SEQ ID NO: 3168 | NSPWAPTAPLPGDMNPANWP |
| SEQ ID NO: 3169 | SPWAPTAPLPGDMNPANWPR |
| SEQ ID NO: 3170 | PWAPTAPLPGDMNPANWPRE |
| SEQ ID NO: 3171 | WAPTAPLPGDMNPANWPRER |
| SEQ ID NO: 3172 | APTAPLPGDMNPANWPRERA |
| SEQ ID NO: 3173 | PTAPLPGDMNPANWPRERAW |

TABLE 24
P(T/S)AP Motif Containing Peptides from
Human Parechovirus 2 Polyprotein
(GenBank Accession No. NP_046804)

| | |
|---|---|
| SEQ ID NO: 3174 | LTQHPSAP |
| SEQ ID NO: 3175 | TQHPSAPT |
| SEQ ID NO: 3176 | QHPSAPTL |
| SEQ ID NO: 3177 | HPSAPTLP |
| SEQ ID NO: 3178 | PSAPTLPF |
| SEQ ID NO: 3179 | NLTQHPSAP |
| SEQ ID NO: 3180 | LTQHPSAPT |
| SEQ ID NO: 3181 | TQHPSAPTL |
| SEQ ID NO: 3182 | QHPSAPTLP |
| SEQ ID NO: 3183 | HPSAPTLPF |
| SEQ ID NO: 3184 | PSAPTLPFT |
| SEQ ID NO: 3185 | TNLTQHPSAP |
| SEQ ID NO: 3186 | NLTQHPSAPT |
| SEQ ID NO: 3187 | LTQHPSAPTL |
| SEQ ID NO: 3188 | TQHPSAPTLP |
| SEQ ID NO: 3189 | QHPSAPTLPF |
| SEQ ID NO: 3190 | HPSAPTLPFT |
| SEQ ID NO: 3191 | PSAPTLPFTP |
| SEQ ID NO: 3192 | TTNLTQHPSAP |
| SEQ ID NO: 3193 | TNLTQHPSAPT |
| SEQ ID NO: 3194 | NLTQHPSAPTL |
| SEQ ID NO: 3195 | LTQHPSAPTLP |
| SEQ ID NO: 3196 | TQHPSAPTLPF |
| SEQ ID NO: 3197 | QHPSAPTLPFT |
| SEQ ID NO: 3198 | HPSAPTLPFTP |
| SEQ ID NO: 3199 | PSAPTLPFTPD |
| SEQ ID NO: 3200 | NTTNLTQHPSAP |
| SEQ ID NO: 3201 | TTNLTQHPSAPT |
| SEQ ID NO: 3202 | TNLTQHPSAPTL |
| SEQ ID NO: 3203 | NLTQHPSAPTLP |
| SEQ ID NO: 3204 | LTQHPSAPTLPF |
| SEQ ID NO: 3205 | TQHPSAPTLPFT |
| SEQ ID NO: 3206 | QHPSAPTLPFTP |
| SEQ ID NO: 3207 | HPSAPTLPFTPD |
| SEQ ID NO: 3208 | PSAPTLPFTPDF |
| SEQ ID NO: 3209 | VNTTNLTQHPSAP |
| SEQ ID NO: 3210 | NTTNLTQHPSAPT |
| SEQ ID NO: 3211 | TTNLTQHPSAPTL |
| SEQ ID NO: 3212 | TNLTQHPSAPTLP |
| SEQ ID NO: 3213 | NLTQHPSAPTLPF |
| SEQ ID NO: 3214 | LTQHPSAPTLPFT |
| SEQ ID NO: 3215 | TQHPSAPTLPFTP |
| SEQ ID NO: 3216 | QHPSAPTLPFTPD |
| SEQ ID NO: 3217 | HPSAPTLPFTPDF |
| SEQ ID NO: 3218 | PSAPTLPFTPDFS |
| SEQ ID NO: 3219 | TVNTTNLTQHPSAP |
| SEQ ID NO: 3220 | VNTTNLTQHPSAPT |
| SEQ ID NO: 3221 | NTTNLTQHPSAPTL |
| SEQ ID NO: 3222 | TTNLTQHPSAPTLP |
| SEQ ID NO: 3223 | TNLTQHPSAPTLPF |
| SEQ ID NO: 3224 | NLTQHPSAPTLPFT |
| SEQ ID NO: 3225 | LTQHPSAPTLPFTP |
| SEQ ID NO: 3226 | TQHPSAPTLPFTPD |
| SEQ ID NO: 3227 | QHPSAPTLPFTPDF |
| SEQ ID NO: 3228 | HPSAPTLPFTPDFS |
| SEQ ID NO: 3229 | PSAPTLPFTPDFSN |
| SEQ ID NO: 3230 | TTVNTTNLTQHPSAP |
| SEQ ID NO: 3231 | TVNTTNLTQHPSAPT |
| SEQ ID NO: 3232 | VNTTNLTQHPSAPTL |
| SEQ ID NO: 3233 | NTTNLTQHPSAPTLP |
| SEQ ID NO: 3234 | TTNLTQHPSAPTLPF |
| SEQ ID NO: 3235 | TNLTQHPSAPTLPFT |
| SEQ ID NO: 3236 | NLTQHPSAPTLPFTP |
| SEQ ID NO: 3237 | LTQHPSAPTLPFTPD |
| SEQ ID NO: 3238 | TQHPSAPTLPFTPDF |
| SEQ ID NO: 3239 | QHPSAPTLPFTPDFS |
| SEQ ID NO: 3240 | HPSAPTLPFTPDFSN |
| SEQ ID NO: 3241 | PSAPTLPFTPDFSNV |
| SEQ ID NO: 3242 | TTTVNTTNLTQHPSAP |
| SEQ ID NO: 3243 | TTVNTTNLTQHPSAPT |
| SEQ ID NO: 3244 | TVNTTNLTQHPSAPTL |
| SEQ ID NO: 3245 | VNTTNLTQHPSAPTLP |
| SEQ ID NO: 3246 | NTTNLTQHPSAPTLPF |
| SEQ ID NO: 3247 | TTNLTQHPSAPTLPFT |
| SEQ ID NO: 3248 | TNLTQHPSAPTLPFTP |
| SEQ ID NO: 3249 | NLTQHPSAPTLPFTPD |
| SEQ ID NO: 3250 | LTQHPSAPTLPFTPDF |
| SEQ ID NO: 3251 | TQHPSAPTLPFTPDFS |
| SEQ ID NO: 3252 | QHPSAPTLPFTPDFSN |
| SEQ ID NO: 3253 | HPSAPTLPFTPDFSNV |
| SEQ ID NO: 3254 | PSAPTLPFTPDFSNVD |
| SEQ ID NO: 3255 | ATTTVNTTNLTQHPSAP |
| SEQ ID NO: 3256 | TTTVNTTNLTQHPSAPT |
| SEQ ID NO: 3257 | TTVNTTNLTQHPSAPTL |
| SEQ ID NO: 3258 | TVNTTNLTQHPSAPTLP |
| SEQ ID NO: 3259 | VNTTNLTQHPSAPTLPF |
| SEQ ID NO: 3260 | NTTNLTQHPSAPTLPFT |
| SEQ ID NO: 3261 | TTNLTQHPSAPTLPFTP |
| SEQ ID NO: 3262 | TNLTQHPSAPTLPFTPD |
| SEQ ID NO: 3263 | NLTQHPSAPTLPFTPDF |
| SEQ ID NO: 3264 | LTQHPSAPTLPFTPDFS |
| SEQ ID NO: 3265 | TQHPSAPTLPFTPDFSN |
| SEQ ID NO: 3266 | QHPSAPTLPFTPDFSNV |
| SEQ ID NO: 3267 | HPSAPTLPFTPDFSNVD |
| SEQ ID NO: 3268 | PSAPTLPFTPDFSNVDT |
| SEQ ID NO: 3269 | QATTTVNTTNLTQHPSAP |
| SEQ ID NO: 3270 | ATTTVNTTNLTQHPSAPT |
| SEQ ID NO: 3271 | TTTVNTTNLTQHPSAPTL |
| SEQ ID NO: 3272 | TTVNTTNLTQHPSAPTLP |
| SEQ ID NO: 3273 | TVNTTNLTQHPSAPTLPF |
| SEQ ID NO: 3274 | VNTTNLTQHPSAPTLPFT |
| SEQ ID NO: 3275 | NTTNLTQHPSAPTLPFTP |
| SEQ ID NO: 3276 | TTNLTQHPSAPTLPFTPD |
| SEQ ID NO: 3277 | TNLTQHPSAPTLPFTPDF |
| SEQ ID NO: 3278 | NLTQHPSAPTLPFTPDFS |
| SEQ ID NO: 3279 | LTQHPSAPTLPFTPDFSN |
| SEQ ID NO: 3280 | TQHPSAPTLPFTPDFSNV |
| SEQ ID NO: 3281 | QHPSAPTLPFTPDFSNVD |
| SEQ ID NO: 3282 | HPSAPTLPFTPDFSNVDT |
| SEQ ID NO: 3283 | PSAPTLPFTPDFSNVDTF |
| SEQ ID NO: 3284 | VQATTTVNTTNLTQHPSAP |
| SEQ ID NO: 3285 | QATTTVNTTNLTQHPSAPT |
| SEQ ID NO: 3286 | ATTTVNTTNLTQHPSAPTL |
| SEQ ID NO: 3287 | TTTVNTTNLTQHPSAPTLP |
| SEQ ID NO: 3288 | TTVNTTNLTQHPSAPTLPF |
| SEQ ID NO: 3289 | TVNTTNLTQHPSAPTLPFT |
| SEQ ID NO: 3290 | VNTTNLTQHPSAPTLPFTP |
| SEQ ID NO: 3291 | NTTNLTQHPSAPTLPFTPD |
| SEQ ID NO: 3292 | TTNLTQHPSAPTLPFTPDF |
| SEQ ID NO: 3293 | TNLTQHPSAPTLPFTPDFS |
| SEQ ID NO: 3294 | NLTQHPSAPTLPFTPDFSN |
| SEQ ID NO: 3295 | LTQHPSAPTLPFTPDFSNV |
| SEQ ID NO: 3296 | TQHPSAPTLPFTPDFSNVD |
| SEQ ID NO: 3297 | QHPSAPTLPFTPDFSNVDT |
| SEQ ID NO: 3298 | HPSAPTLPFTPDFSNVDTF |
| SEQ ID NO: 3299 | PSAPTLPFTPDFSNVDTFH |
| SEQ ID NO: 3300 | VVQATTTVNTTNLTQHPSAP |
| SEQ ID NO: 3301 | VQATTTVNTTNLTQHPSAPT |
| SEQ ID NO: 3302 | QATTTVNTTNLTQHPSAPTL |
| SEQ ID NO: 3303 | ATTTVNTTNLTQHPSAPTLP |
| SEQ ID NO: 3304 | TTTVNTTNLTQHPSAPTLPF |
| SEQ ID NO: 3305 | TTVNTTNLTQHPSAPTLPFT |
| SEQ ID NO: 3306 | TVNTTNLTQHPSAPTLPFTP |
| SEQ ID NO: 3307 | VNTTNLTQHPSAPTLPFTPD |
| SEQ ID NO: 3308 | NTTNLTQHPSAPTLPFTPDF |
| SEQ ID NO: 3309 | TTNLTQHPSAPTLPFTPDFS |
| SEQ ID NO: 3310 | TNLTQHPSAPTLPFTPDFSN |
| SEQ ID NO: 3311 | NLTQHPSAPTLPFTPDFSNV |
| SEQ ID NO: 3312 | LTQHPSAPTLPFTPDFSNVD |
| SEQ ID NO: 3313 | TQHPSAPTLPFTPDFSNVDT |
| SEQ ID NO: 3314 | QHPSAPTLPFTPDFSNVDTF |
| SEQ ID NO: 3315 | HPSAPTLPFTPDFSNVDTFH |
| SEQ ID NO: 3316 | PSAPTLPFTPDFSNVDTFHS |

TABLE 25
P(T/S)AP Motif Containing Peptides
from Semliki Forest Virus Polyprotein
(GenBank Accession No. CAA76683)

| | |
|---|---|
| SEQ ID NO: 3317 | LKIRPSAP |
| SEQ ID NO: 3318 | KIRPSAPY |
| SEQ ID NO: 3319 | IRPSAPYK |
| SEQ ID NO: 3320 | RPSAPYKT |
| SEQ ID NO: 3321 | PSAPYKTT |
| SEQ ID NO: 3322 | GLKIRPSAP |
| SEQ ID NO: 3323 | LKIRPSAPY |
| SEQ ID NO: 3324 | KIRPSAPYK |
| SEQ ID NO: 3325 | IRPSAPYKT |
| SEQ ID NO: 3326 | RPSAPYKTT |
| SEQ ID NO: 3327 | PSAPYKTTV |
| SEQ ID NO: 3328 | EGLKIRPSAP |
| SEQ ID NO: 3329 | GLKIRPSAPY |
| SEQ ID NO: 3330 | LKIRPSAPYK |
| SEQ ID NO: 3331 | KIRPSAPYKT |
| SEQ ID NO: 3332 | IRPSAPYKTT |
| SEQ ID NO: 3333 | RPSAPYKTTV |
| SEQ ID NO: 3334 | PSAPYKTTVV |
| SEQ ID NO: 3335 | YEGLKIRPSAP |
| SEQ ID NO: 3336 | EGLKIRPSAPY |
| SEQ ID NO: 3337 | GLKIRPSAPYK |
| SEQ ID NO: 3338 | LKIRPSAPYKT |
| SEQ ID NO: 3339 | KIRPSAPYKTT |
| SEQ ID NO: 3340 | IRPSAPYKTTV |
| SEQ ID NO: 3341 | RPSAPYKTTVV |
| SEQ ID NO: 3342 | PSAPYKTTVVG |
| SEQ ID NO: 3343 | AYEGLKIRPSAP |
| SEQ ID NO: 3344 | YEGLKIRPSAPY |
| SEQ ID NO: 3345 | EGLKIRPSAPYK |
| SEQ ID NO: 3346 | GLKIRPSAPYKT |
| SEQ ID NO: 3347 | LKIRPSAPYKTT |
| SEQ ID NO: 3348 | KIRPSAPYKTTV |
| SEQ ID NO: 3349 | IRPSAPYKTTVV |
| SEQ ID NO: 3350 | RPSAPYKTTVVG |
| SEQ ID NO: 3351 | PSAPYKTTVVGV |
| SEQ ID NO: 3352 | FAYEGLKIRPSAP |
| SEQ ID NO: 3353 | AYEGLKIRPSAPY |
| SEQ ID NO: 3354 | YEGLKIRPSAPYK |
| SEQ ID NO: 3355 | EGLKIRPSAPYKT |
| SEQ ID NO: 3356 | GLKIRPSAPYKTT |
| SEQ ID NO: 3357 | LKIRPSAPYKTTV |
| SEQ ID NO: 3358 | KIRPSAPYKTTVV |
| SEQ ID NO: 3359 | IRPSAPYKTTVVG |
| SEQ ID NO: 3360 | RPSAPYKTTVVGV |
| SEQ ID NO: 3361 | PSAPYKTTVVGVF |
| SEQ ID NO: 3362 | EFAYEGLKIRPSAP |
| SEQ ID NO: 3363 | FAYEGLKIRPSAPY |
| SEQ ID NO: 3364 | AYEGLKIRPSAPYK |
| SEQ ID NO: 3365 | YEGLKIRPSAPYKT |
| SEQ ID NO: 3366 | EGLKIRPSAPYKTT |
| SEQ ID NO: 3367 | GLKIRPSAPYKTTV |
| SEQ ID NO: 3368 | LKIRPSAPYKTTVV |
| SEQ ID NO: 3369 | KIRPSAPYKTTVVG |
| SEQ ID NO: 3370 | IRPSAPYKTTVVGV |
| SEQ ID NO: 3371 | RPSAPYKTTVVGVF |
| SEQ ID NO: 3372 | PSAPYKTTVVGVFG |
| SEQ ID NO: 3373 | HEFAYEGLKIRPSAP |
| SEQ ID NO: 3374 | EFAYEGLKIRPSAPY |
| SEQ ID NO: 3375 | FAYEGLKIRPSAPYK |
| SEQ ID NO: 3376 | AYEGLKIRPSAPYKT |
| SEQ ID NO: 3377 | YEGLKIRPSAPYKTT |
| SEQ ID NO: 3378 | EGLKIRPSAPYKTTV |
| SEQ ID NO: 3379 | GLKIRPSAPYKTTVV |
| SEQ ID NO: 3380 | LKIRPSAPYKTTVVG |
| SEQ ID NO: 3381 | KIRPSAPYKTTVVGV |
| SEQ ID NO: 3382 | IRPSAPYKTTVVGVF |
| SEQ ID NO: 3383 | RPSAPYKTTVVGVFG |
| SEQ ID NO: 3384 | PSAPYKTTVVGVFGV |
| SEQ ID NO: 3385 | FHEFAYEGLKIRPSAP |
| SEQ ID NO: 3386 | HEFAYEGLKIRPSAPY |
| SEQ ID NO: 3387 | EFAYEGLKIRPSAPYK |
| SEQ ID NO: 3388 | FAYEGLKIRPSAPYKT |
| SEQ ID NO: 3389 | AYEGLKIRPSAPYKTT |
| SEQ ID NO: 3390 | YEGLKIRPSAPYKTTV |
| SEQ ID NO: 3391 | EGLKIRPSAPYKTTVV |
| SEQ ID NO: 3392 | GLKIRPSAPYKTTVVG |
| SEQ ID NO: 3393 | LKIRPSAPYKTTVVGV |
| SEQ ID NO: 3394 | KIRPSAPYKTTVVGVF |
| SEQ ID NO: 3395 | IRPSAPYKTTVVGVFG |
| SEQ ID NO: 3396 | RPSAPYKTTVVGVFGV |
| SEQ ID NO: 3397 | PSAPYKTTVVGVFGVP |
| SEQ ID NO: 3398 | PFHEFAYEGLKIRPSAP |
| SEQ ID NO: 3399 | FHEFAYEGLKIRPSAPY |
| SEQ ID NO: 3400 | HEFAYEGLKIRPSAPYK |
| SEQ ID NO: 3401 | EFAYEGLKIRPSAPYKT |
| SEQ ID NO: 3402 | FAYEGLKIRPSAPYKTT |
| SEQ ID NO: 3403 | AYEGLKIRPSAPYKTTV |
| SEQ ID NO: 3404 | YEGLKIRPSAPYKTTVV |
| SEQ ID NO: 3405 | EGLKIRPSAPYKTTVVG |
| SEQ ID NO: 3406 | GLKIRPSAPYKTTVVGV |
| SEQ ID NO: 3407 | LKIRPSAPYKTTVVGVF |
| SEQ ID NO: 3408 | KIRPSAPYKTTVVGVFG |
| SEQ ID NO: 3409 | IRPSAPYKTTVVGVFGV |
| SEQ ID NO: 3410 | RPSAPYKTTVVGVFGVP |
| SEQ ID NO: 3411 | PSAPYKTTVVGVFGVPG |
| SEQ ID NO: 3412 | PPFHEFAYEGLKIRPSAP |
| SEQ ID NO: 3413 | PFHEFAYEGLKIRPSAPY |
| SEQ ID NO: 3414 | FHEFAYEGLKIRPSAPYK |
| SEQ ID NO: 3415 | HEFAYEGLKIRPSAPYKT |
| SEQ ID NO: 3416 | EFAYEGLKIRPSAPYKTT |
| SEQ ID NO: 3417 | FAYEGLKIRPSAPYKTTV |
| SEQ ID NO: 3418 | AYEGLKIRPSAPYKTTVV |
| SEQ ID NO: 3419 | YEGLKIRPSAPYKTTVVG |
| SEQ ID NO: 3420 | EGLKIRPSAPYKTTVVGV |
| SEQ ID NO: 3421 | GLKIRPSAPYKTTVVGVF |
| SEQ ID NO: 3422 | LKIRPSAPYKTTVVGVFG |
| SEQ ID NO: 3423 | KIRPSAPYKTTVVGVFGV |
| SEQ ID NO: 3424 | IRPSAPYKTTVVGVFGVP |
| SEQ ID NO: 3425 | RPSAPYKTTVVGVFGVPG |
| SEQ ID NO: 3426 | PSAPYKTTVVGVFGVPGS |
| SEQ ID NO: 3427 | NPPFHEFAYEGLKIRPSAP |
| SEQ ID NO: 3428 | PPFHEFAYEGLKIRPSAPY |
| SEQ ID NO: 3429 | PFHEFAYEGLKIRPSAPYK |
| SEQ ID NO: 3430 | FHEFAYEGLKIRPSAPYKT |
| SEQ ID NO: 3431 | HEFAYEGLKIRPSAPYKTT |
| SEQ ID NO: 3432 | EFAYEGLKIRPSAPYKTTV |
| SEQ ID NO: 3433 | FAYEGLKIRPSAPYKTTVV |
| SEQ ID NO: 3434 | AYEGLKIRPSAPYKTTVVG |
| SEQ ID NO: 3435 | YEGLKIRPSAPYKTTVVGV |
| SEQ ID NO: 3436 | EGLKIRPSAPYKTTVVGVF |
| SEQ ID NO: 3437 | GLKIRPSAPYKTTVVGVFG |
| SEQ ID NO: 3438 | LKIRPSAPYKTTVVGVFGV |
| SEQ ID NO: 3439 | KIRPSAPYKTTVVGVFGVP |
| SEQ ID NO: 3440 | IRPSAPYKTTVVGVFGVPG |
| SEQ ID NO: 3441 | RPSAPYKTTVVGVFGVPGS |
| SEQ ID NO: 3442 | PSAPYKTTVVGVFGVPGSG |
| SEQ ID NO: 3443 | TNPPFHEFAYEGLKIRPSAP |
| SEQ ID NO: 3444 | NPPFHEFAYEGLKIRPSAPY |
| SEQ ID NO: 3445 | PPFHEFAYEGLKIRPSAPYK |
| SEQ ID NO: 3446 | PFHEFAYEGLKIRPSAPYKT |
| SEQ ID NO: 3447 | FHEFAYEGLKIRPSAPYKTT |
| SEQ ID NO: 3448 | HEFAYEGLKIRPSAPYKTTV |
| SEQ ID NO: 3449 | EFAYEGLKIRPSAPYKTTVV |
| SEQ ID NO: 3450 | FAYEGLKIRPSAPYKTTVVG |
| SEQ ID NO: 3451 | AYEGLKIRPSAPYKTTVVGV |
| SEQ ID NO: 3452 | YEGLKIRPSAPYKTTVVGVF |
| SEQ ID NO: 3453 | EGLKIRPSAPYKTTVVGVFG |
| SEQ ID NO: 3454 | GLKIRPSAPYKTTVVGVFGV |
| SEQ ID NO: 3455 | LKIRPSAPYKTTVVGVFGVP |
| SEQ ID NO: 3456 | KIRPSAPYKTTVVGVFGVPG |
| SEQ ID NO: 3457 | IRPSAPYKTTVVGVFGVPGS |
| SEQ ID NO: 3458 | RPSAPYKTTVVGVFGVPGSG |
| SEQ ID NO: 3459 | PSAPYKTTVVGVFGVPGSGK |

SEQ ID NO: 3460 (Ebola Virus Matrix Protein (AAL25816)):
MRRVILPTAPPEYMEAIYPVRSNSTIARGGNSNTGFLTPESVNGDTPSNPLRPIADDTID
HASHIPGSVSSAFILEAMVNVISGPKVLMKQIPIWLPLGVADQKTYSFDSTTAAIMLASY
TITHFGKATNPLVRVNRLGPGIPDHPLRLLRIGNQAFLQEFVLPPVQLPQYFTFDLTALK
LITQPLPAATWTDDTPTGSNGALRPGISFHPKLRPILLPNKSGKKGNSADLTSPEKIQAI
MTSLQDFKIVPIDPTKNIMGIEVPETLVHKLTGKKVTSKNGQPIIPVLLPKYIGLDPVAP
GDLTMVITQDCDTCHSPASLPAVIEK
SEQ ID NO: 3461 (Hepatitis B Virus PreS1/PreS2/S Envelope
Protein (BAA85340)):
MGGWSSKPRKGMGTNLSVPNPLGFFPDHQLDPAFKANSDNPDWDLNPHKDNWPDSNKVGV
GAFGPGFTPPHGGLLGWSPQAQGILTTVPTAPPPASTNRQLGRKPTPLSPPLRDTHPQAM
QWNSTTFHQTLQDPRVRALYFPAGGSSSGTVNPVQNTASSISSILSTTGDPVPNMENIAS
GLLGPLLVLQAGFFSLTKILTIPQSLDSWWTSLNFLGGTPVCLGQNSQSQISSHSPTCCP
PICPGYRWMCLRRFIIFLCILLLCLIFLLVLLDYQGMLPVCPLIPGSSTTSTGPCKTCTT
PAQGTSMFPSCCCIKPTDGNCTCIPIPSSWAFAKYLWEWASVRFSWLSLLVPFVQWFVGL
SPTVWLSVIWMMWFWGPSLYNILSPFMPLLPIFFCLWAYI
SEQ ID NO: 3462 (Human Herpesvirus 1 RL2 Protein (NP_044601))
MEPRPGASTRRPEGRPQREPAPDVWVFPCDRDLPDSSDSEAETEVGGRGDADHHDDDSAS
EADSTDTELFETGLLGPQGVDGGAVSGGSPPREEDPGSCGGAPPREDGGSDEGDVCAVCT
DEIAPHLRCDTFPCMHRFCIPCMKTWMQLRNTCPLCNAKLVYLIVGVTPSGSFSTIPIVN
DPQTRMEAEEAVRAGTAVDFIWTGNQRFAPRYLTLGGHTVRALSPTHPEPTTDEDDDDLD
DADYVPPAPRRTPRAPPRRGAAAPPVTGGASHAAPQPAAARTAPPSAPIGPHGSSNTNTT
TNSSGGGGSRQSRAAAPRGASGPSGGVGVGVGVVEAEAGRPRGRTGPLVNRPAPLANNRD
PIVISDSPPASPHRPPAAPMPGSAPRPGPPASAAASGPARPRAAVAPCVRAPPPGPGPRA
PAPGAEPAARPADARRVPQSHSSLAQAANQEQSLCRARATVARGSGGPGVEGGHGPSRGA
APSGAAPLPSAASVEQEAAVRPRKRRGSGQENPSPQSTRPPLAPAGAKRAATHPPSDSGP
GGRGQGGPGTPLTSSAASASSSSASSSSAPTPAGAASSAAGAASSSASASSGGAVGALGG
RQEETSLGPRAASGPRGPRKCARKTRHAETSGAVPAGGLTRYLPISGVSSVVALSPYVNK
TITGDCLPILDMETGNIGAYVVLVDQTGNMATRLRAAVPGWSRRTLLPETAGNHVMPPEY
PTAPASEWNSLWMTPVGNMLFDQGTLVGALDFRSLRSRHPWSGEQGASTRDEGKQ
SEQ ID NO: 3463 (Human Herpesvirus 2 Virion Glycoprotein K
(NP_044524))
MLAVRSLQHLTTVIFITAYGLVLAWYIVFGASPLHRCIYAVRPAGAHNDTALVWMKINQT
LLFLGPPTAPPGGAWTPHARVCYANIIEGRAVSLPAIPGAMSRRVMNVHEAVNCLEALWD
TQMRLVVVGWFLYLAFVALHQRRCMFGVVSPAHSMVAPATYLLNYAGRIVSSVFLQYPYT
KITRLLCELSVQRQTLVQLFEADPVTFLYHRPAIGVIVGCELLLRFVALGLIVGTALISR
GACAITHPLFLTITTWCFVSIIALTELYFILRRGSAPKNAEPAAPRGRSKGWSGVCGRCC
SIILSGIAVRLCYIAVVAGVVLVALRYEQEIQRRLFDL
SEQ ID NO: 3464 (Human Herpesvirus 2 Strain 333 Glycoprotein
I (P06764))
MPGRSLQGLAILGLWVCATGLVVRGPTVSLVSDSLVDAGAVGPQGFVEEDLRVFGELHFV
GAQVPHTNYYDGIIELFHYPLGNHCPRVVHVVTLTACPRRPAVAFTLCRSTHHAHSPAYP
TLELGLARQPLLRVRTATRDYAGLYVLRVWVGSATNASLFVLGVALSANGTFVYNGSDYG
SCDPAQLPFSAPRLGPSSVYTPGASRPTPPRTTTSPSSPRDPTPAPGDTGTPAPASGERA
PPNSTRSASESRHRLTVAQVIQIAIPASIIAFVFLGSCICFIHRCQRRYRRPRGQIYNPG
GVSCAVNEAAMARLGAELRSHPNTPPKPRRRSSSSTTMPSLTSIAEESEPGPVVLLSVSP
RPRSGPTAPQEV
SEQ ID NO: 3465 (Human Herpesvirus 4 - Eptein Barr Virus
EBNA2 (NP_039845))
MPTFYLALHGGQTYHLIVDTDSLGNPSLSVIPSNPYQEQLSDTPLIPLTIFVGENTGVPP
PLPPPPPPPPPPPPPPPPPPPPPPPPPPSPPPPPPPPPPPQRRDAWTQEPSPLDRDPLGY
DVGHGPLASAMRMLWMANYIVRQSRGDRGLILPQGPQTAPQARLVQPHVPPLRPTAPTIL
SPLSQPRLTPPQPLMMPPRPTPPTPLPPATLTVPPRPTRPTTLPPTPLLTVLQRPTELQP
TPSPPRMHLPVLHVPDQSMHPLTHQSTPNDPDSPEPRSPTVFYNIPPMPLPPSQLPPPAA
PAQPPPGVINDQQLHHLPSGPPWWPPICDPPQPSKTQGQSRGQSRGRGRGRGRGRGKGKS
RDKQRKPGGPWRPEPNTSSPSMPELSPVLGLHQGQGAGDSPTPGPSNAAPVCRNSHTATP
NVSPIHEPESHNSPEAPILFPDDWYPPSIDPADLDESWDYIFETTESPSSDEDYVEGPSK
RPRPSIQ
SEQ ID NO: 3466 (Influenza A Virus (A/Pintail
Duck/Alberta/114/79 (H8N4) Hemagglutinin (AAG38554))
SKAGVTMEKLIVIAMLLASTNAYDRICIGYQSNNSTDTVNTLIEQNVPVTQTMELVETEK
HPAYCNTDLGAPLELRDCKIEAVIYGNPKCDIHLKDQGWSYIVERPSAPEGMCYPGSIEN
LEELRFVFSSAASYKRIRLFDYSRWNVTRSGTSKACNASTGGQSFYRSINWLTKKKPDTY
DFNEGTYVNNEDGDIIFLWGIHHPPDTKEQTTLYKNANTLTSVTTNTINRNFQPNIGPRP
LVRGQQGRMDYYWGILKRGETLKIRTNGNLIAPEFGYLLKGESHGRIIQNEDIPIGNCNT
KCQTYAGAINSSKPFQNASRHYMGECPKYVKKASLRLAVGLRNTPSVEPRGLFGAIAGFI
EGGWSGMIDGWYGFHHSNSEGTGMAADQKSTQEAIDKITNKVNNIVDKMNRE
SEQ ID NO: 3467 (Human Papilomavirus L1 Protein, My09/My11
Region (AAA67231))
AQGHNNGICWFNELFVTVVDTTRSTNITISAAATQANEYTASNFKEYLRHTEEYDLQVIL
QLCKIHLTPEIMAYLHSMNEHLLDEWNFGVLPPPSTSLDDTYRYLQSRAITCQKGPSAPA
PKKDPYDGLVFWEVDLKDKLSTDLDQFPLGR
SEQ ID NO: 3468 (Human Papilomavirus Type 23 Minor Capsid
Protein L2 (NP_043365)
MVRAQRTKRASVTDIYKGCKASGTCPPDVLNKVEQNTLADKILKYGSVGVFFGGLGIGTG
KGTGGATGYVPLRPGVRVGGTPTVVRPAVIPEIIGPTELIPVDSIAPIDPEAPSIVSLTD
SGAAADLFPSEAETIAEVHPTPVDIGIDTPIVAGGRDAILEVVDTNPPTRFSVTRTQYDN
PSFQIISESTPITGEASLADHVFVFEGSGGQHVGAVTEEIELDTYPSRYSFEIEEATPPR
RTSTPIERISQEFRNLRRALYNRRLTEQVQVKNPLFLTTPSKLVRFQFDNPVFDEEVTQI
FERDVAEVEEPPDRDFLDIDRLGRPLLTESTEGRIRLSRLGQRASIQTRSGTRVGSRVHF
YTDLSTINTEEPIELELLGEHSGDASVIEEPLQSTVIDMNLDDVEAIQDTIDTADDYNSA
DLLLDNAIEEFNNSQLVFGTSDRSSSAYSIPRFESPRETIVYVQDIEGNQVIYPGPTERP
TIIFPLPSAPAVVIHTLDKSFDYYLHPSLRKKRRKRKYL
SEQ ID NO: 3469 (Human Papilomavirus Type 35 Major Capsid
Protein L1 (P27232))
MSLWRSNEATVYLPPVSVSKVVSTDEYVTRTNIYYHAGSSRLLAVGHPYYAIKKQDSNKI
AVPKVSGLQYRVFRVKLPDPNKFGFPDTSFYDPASQRLVWACTGVEVGRGQPLGVGISGH
PLLNKLDDTENSNKYVGNSGTDNRECISMDYKQTQLCLIGCRPPIGEHWGKGTPCNANQV
KAGECPPLELLNTVLQDGDMVDTGFGAMDFTTLQANKSDVPLDICSSICKYPDYLKMVSE
PYGDMLFFYLRREQMFVRHLFNRAGTVGETVPADLYIKGTTGTLPSTSYFPTPSGSMVTS
DAQIFNKPYWLQRAQGHNNGICWSNQLFVTVVDTTRSTNMSVCSAVSSSDSTYKNDNFKE
YLRHGEEYDLQFIFQLCKITLTADVMTYIHSMNPSILEDWNFGLTPPPSGTLEDTYRYVT
SQAVTCQKPSAPKPKDDPLKNYTFWEVDLKEKFSADLDQFPLGRKFLLQAGLKARPNFRL
GKRAAPASTSKKSSTKRRKVKS
SEQ ID NO: 3470 (Human Papilomavirus Type 6b Minor Capsid
Protein L2 (NP_040303))
MAHSRARRRKRASATQLYQTCKLTGTCPPDVIPKVEHNTIADQILKWGSLGVFFGGLGIG
TGSGTGGRTGYVPLQTSAKPSITSGPMARPPVVVEPVAPSDPSIVSLIEESAIINAGAPE
IVPPAHGGFTITSSETTTPAILDVSVTSHTTTSIFRNPVFTEPSVTQPQPPVEANGHILI
SAPTVTSHPIEEIPLDTFVVSSSDSGPTSSTPVPGTAPRPRVGLYSRALHQVQVTDPAFL
STPQRLITYDNPVYEGEDVSVQFSHDSIHNAPDEAFMDIIRLHRPAIASRRGLVRYSRIG
QRGSMHTRSGKHIGARIHYFYDISPIAQAAEEIEMHPLVAAQDDTFDIYAESFEPGINPT
QHPVTNISDTYLTSTPNTVTQPWGNTTVPLSLPNDLFLQSGPDITFPTAPMGTPFSPVTP
ALPTGPVFITGSGFYLHPAWYFARKRRKRIPLFFSDVAA
SEQ ID NO: 3471 (Human Papilomavirus Type 9 Late Protein
(NP_041865))
MVRAKRTKRASVTDIYRGCKAAGTCPPDVINKVEHTTIADKILQYGSAGVFFGGLGISTG
RGTGGATGYVPLGEGPGVRVGGTPTIVRPGVIPEIIGPTDLIPLDTVRPIDPTAPSIVTG
TDSTVDLLPGEIESIAEIHPVPVDNAVVDTPVVTEGRRGSSAILEVADPSPPMRTRVART
QYHNPAFQIISESTPMSGESSLADHIIVFEGSGGQLVGGPRESYTASSENIELQEFPSRY
SFEIDEGTPPRTSTPVQRAVQSLSSLRRALYNRRLTEQVAVTDPLFLSRPSRLVQFQFDN
PAFEDEVTQIFERDLSTVEEPPDRQFLDVQRLSRPLYTETPQGYVRVSRLGRRATIRTRS
GAQVGAQVHFYRDLSTINTEEPIEMQLLGEHSGDSTIVQGPVESSIVDVNIDEPDGLEVG
RQETPSVEDVDFNSEDLLLDEGVEDFSGSQLVVGTRRSTNTLTVPRFETPRDTSFYIQDI
QGYTVSYPESRQTTDIIFPHPDTPTVVIHINDTSGDYYLHPSLQRKKRKRKYL
SEQ ID NO: 3472 (Human T-cell Lymphotropic Virus Type 2 Gag
Protein (CAA61543))
MGQIHGLSPTPIPKAPRGLSTHHWLNFLQAAYRLQPGPSDFDFQQLRRFLKLALKTPIWL
NPIDYSLLASLVPKGYPGRVVEIINILVKNQVSPSAPAAPVPTPICPTTTPPPPPPPSPE
AHVPPPYVEPTSTKCFPILHPPGAPSAHRPWQMKDLQAIKQEVSSSAPGSPQFMQTLRLA
VQQFDPTAKDLQDLLQYLCSSLVVSLHHQQLNTLITEAETRGVTGYNPMAGPLRMQANNP
AQQGLRREYQNLWLAAFSTLPGNTRDPSWAAILQGLEEPYCAFVERLNVALDNGLPEGTP
KEPILRSLAYSNANKECQKILQARGPTNSPLGEMLRACQAWTPKDKTKVLVVQPRRPPPT
QPCFRCGKIGHWSRDCTQPRPPPGPCPLCQDPSHWKRDCPQPKPPQEEGEPLLLDLSSTS
GTTEEKNSLRGEI
SEQ ID NO: 3473 (West Nile Virus Polyprotein(NP_041724))
MSKKPGGPGKNRAVNMLKRGMPRGLSLIGLKRAMLSLIDGKGPIRFVLALLAFFRFTAIA
PTRAVLDRWRGVNKQTAMKHLLSFKKELGTLTSAINRRSTKQKKRGGTAGFTILLGLIAC
AGAVTLSNFQGKVMMTVNATDVTDVITIPTAAGKNLCIVRAMDVGYLCEDTITYECPVLA
AGNDPEDIDCWCTKSSVYVRYGRCTKTRHSRRSRRSLTVQTHGESTLANKKGAWLDSTKA
TRYLVKTESWILRNPGYALVAAVIGWMLGSNTMQRVVFAILLLLVAPAYSFNCLGMSNRD
FLEGVSGATWVDLVLEGDSCVTIMSKDKPTIDVKMMNMEAANLADVRSYCYLASVSDLST
RAACPTMGEAHNEKRADPAFVCKQGVVDRGWGNGCGLFGKGSIDTCAKFACTTKATGWII
QKENIKYEVAIFVHGPTTVESHGKIGATQAGRFSITPSAPSYTLKLGEYGEVTVDCEPRS
GIDTSAYYVMSVGEKSFLVHREWFMDLNLPWSSAGSTTWRNRETLMEFEEPHATKQSVVA
LGSQEGALHQALAGAIPVEFSSNTVKLTSGHLKCRVKMEKLQLKGTTYGVCSKAFKFART
PADTGHGTVVLELQYTGTDGPCKVPISSVASLNDLTPVGRLVTVNPFVSVATANSKVLIE
LEPPFGDSYIVVGRGEQQINHHWHKSGSSIGKAFTTTLRGAQRLAALGDTAWDFGSVGGV
FTSVGKAIHQVFGGAFRSLFGGMSWITQGLLGALLLWMGINARDRSIAMTFLAVGGVLLF
LSVNVHADTGCAIDIGRQELRCGSGVFIHNDVEAWMDRYKFYPETPQGLAKIIQKAHAEG
VCGLRSVSRLEHQMWEAIKDELNTLLKENGVDLSVVVEKQNGMYKAAPKRLAATTEKLEM
GWKAWGKSIIFAPELANNTFVIDGPETEECPTANRAWNSMEVEDFGFGLTSTRMFLRIRE
TNTTECDSKIIGTAVKNNMAVHSDLSYWIESGLNDTWKLERAVLGEVKSCTWPETHTLWG
DGVLESDLIIPITLAGPRSNHNRRPGYKTQNQGPWDEGRVEIDFDYCPGTTVTISDSCEH
RGPAARTTTESGKLITDWCCRSCTLPPLRFQTENGCWYGMEIRPTRHDEKTLVQSRVNAY
NADMIDPFQLGLMVVFLATQEVLRKRWTAKISIPAIMLALLVLVFGGITYTDVLRYVILV
GAAFAEANSGGDVVHLALMATFKIQPVFLVASFLKARWTNQESILLMLAAAFFQMAYYDA
KNVLSWEVPDVLNSLSVAWMILRAISFTNTSNVVVPLLALLTPGLKCLNLDVYRILLLMV
GVGSLIKEKRSSAAKKKGACLICLALASTGVFNPMILAAGLMACDPNRKRGWPATEVMTA
VGLMFAIVGGLAELDIDSMAIPMTIAGLMFAAFVISGKSTDMWIERTADITWESDAEITG
SSERVDVRLDDDGNFQLMNDPGAPWKIWMLRMACLAISAYTPWAILPSVIGFWITLQYTK
RGGVLWDTPSPKEYKKGDTTTGVYRIMTRGLLGSYQAGAGVMVEGVFHTLWHTTKGAALM
SGEGRLDPYWGSVKEDRLCYGGPWKLQHKWNGHDEVQMIVVEPGKNVKNVQTKPGVFKTP
EGEIGAVTLDYPTGTSGSPIVDKNGDVIGLYGNGVIMPNGSYISAIVQGERMEEPAPAGF
EPEMLRKKQITVLDLHPGAGKTRKILPQIIKEAINKRLRTAVLAPTRVVAAEMSEALRGL
PIRYQTSAVHREHSGNEIVDVMCHATLTHRLMSPHRVPNYNLFIMDEAHFTDPASIAARG
YIATKVELGEAAAIFMTATPPGTSDPFPESNAPISDMQTEIPDRAWNTGYEWITEYVGKT
VWFVPSVKMGNEIALCLQRAGKKVIQLNRKSYETEYPKCKNDDWDFVITTDISEMGANFK
ASRVIDSRKSVKPTIIEEGDGRVILGEPSAITAASAAQRRGRIGRNPSQVGDEYCYGGHT
NEDDSNFAHWTEARIMLDNINMPNGLVAQLYQPEREKVYTMDGEYRLRGEERKNFLEFLR
TADLPVWLAYKVAAAGISYHDRKWCFDGPRTNTILEDNNEVEVITKLGERKILRPRWADA
RVYSDHQALKSFKDFASGKRSQIGLVEVLGRMPEHFMVKTWEALDTMYVVATAEKGGRAH
RMALEELPDALQTIVLIALLSVMSLGVFFLLMQRKGIGKIGLGGVILGAATFFCWMAEVP
GTKIAGMLLLSLLLMIVLIPEPEKQRSQTDNQLAVFLICVLTLVGAVAANEMGWLDKTKN
DIGSLLGHRPEARETTLGVESFLLDLRPATAWSLYAVTTAVLTPLLKHLITSDYINTSLT
SINVQASALFTLARGFPFVDVGVSALLLAVGCWGQVTLTVTVTAAALLFCHYAYMVPGWQ
AEAMRSAQRRTAAGIMKNVVVDGIVATDVPELERTTPVMQKKVGQIILILVSMAAVVVNP
SVRTVREAGILTTAAAVTLWENGASSVWNATTAIGLCHIMRGGWLSCLSIMWTLIKNMEK
PGLKRGGAKGRTLGEVWKERLNHMTKEEFTRYRKEAITEVDRSAAKHARREGNITGGHPV
SRGTAKLRWLVERRFLEPVGKVVDLGCGRGGWCYYMATQKRVQEVKGYTKGGPGHEEPQL
VQSYGWNIVTMKSGVDVFYRPSEASDTLLCDIGESSSSAEVEEHRTVRVLEMVEDWLHRG
PKEFCIKVLCPYMPKVIEKMETLQRRYGGGLIRNPLSRNSTHEMYWVSHASGNIVHSVNM
TSQVLLGRMEKKTWKGPQFEEDVNLGSGTRAVGKPLLNSDTSKIKNRIERLKKEYSSTWH
QDANHPYRTWNYHGSYEVKPTGSASSLVNGVVRLLSKPWDTITNVTTMAMTDTTPFGQQR
VFKEKVDTKAPEPPEGVKYVLNETTNWLWAFLARDKKPRMCSREEFIGKVNSNAALGAMF
EEQNQWKNAREAVEDPKFWEMVDEEREAHLRGECNTCIYNMMGKREKKPGEFGKAKGSRA
IWFMWLGARFLEFEALGFLNEDHWLGRKNSGGGVEGLGLQKLGYILKEVGTKPGGKVYAD
DTAGWDTRITKADLENEAKVLELLDGEHRRLARSIIELTYRHKVVKVMRPAADGKTVMDV
ISREDQRGSGQVVTYALNTFTNLAVQLVRMMEGEGVIGPDDVEKLGKGKGPKVRTWLFEN
GEERLSRMAVSGDDCVVKPLDDRFATSLHFLNAMSKVRKDIQEWKPSTGWYDWQQVPFCS
NHFTELIMKDGRTLVVPCRGQDELIGRARISPGAGWNVRDTACLAKSYAQMWLLLYFHRR
DLRLMANAICSAVPANWVPTGRTTWSIHAKGEWMTTEDMLAVWNRVWIEENEWMEDKTPV
ERWSDVPYSGKREDIWCGSLIGTRTRATWAENIHVAINQVRSVIGEEKYVDYMSSLRRYE
DTIVVEDTVL
SEQ ID NO: 3474 (Measles Matrix Protein (CAA34587))
MHTPPPGAAEDSDPPGPPIGRAPGSPPPGAGRPTAKPEELPKEATEPDTVARRTAGPNEK
PVLHNNTPPTLPTPRRKAPTTGSVPNANQVCNAANLTPLDTPQRLRAVYMSITRLSDNGY
YTVPRRMLEFRSVNAVALNLLATLRTDKAIGPGKTTDNAEQPPEATFLVHIGNPRRKKSE
VHSADHCKMKIEKMGPVSAPGGIGGTSLHIRSTGKTSKTLHAQLGLKKTPCYPPMDINED
LNRSLWRSRRKTARIQAAPQPSAPQEPRTHDDAITNDDQGPFKALQTAVTSNARKRPPSQ
RQPEGPDKKAPSEELHGPSERSASS
SEQ ID NO: 3475 (Rubella Non-structural Protein (BAB32473))
MEKLLDEVLAPGGPYNLTVGSWVRDHVRSIVEGAWEVRDVVTAAQKRAIVAVIPRPVFTQ
MQVSDHPALHAISRYTRRHWIEWGPKEALHVLIDPSPGLLREVARVERRWVALCLHRTAR
KLATALAETASEAWHADYVCALRGAPSGPFYVHPEDVPHGGRAVADRCLLYYTPMQMCEL
MRTIDATLLVAVDLWPVALAAHVGDDWDDLGIAWHLDHDGGCPADCRGAGAGPTPGYTRP
CTTRIYQVLPDTAHPGRLYRCGPRLWTRDCAVAELSWEVAQHCGHQARVRAVRCTLPIRH
VRSLQPSARVRLPDLVHLAAVGRWRWFSLPRPVFQRMLSYCKTLSPDAYYSERVFKFKNA
LSHSITLAGNVLQEGWKGTCAEEDALCAYVAFRAWQSNARLAGIMKSAKRCAADSLSVAG
WLDTIWDAIKRFFGSVPLAERMEEWEQDAAVAAFDRGPLEDGGRHLDTVQPPKSPPRPEI
AATWIVHAASADRHCACAPRCDAPRERPSAPAGPPDDEALIPPWLFAERRALRCREWDFE
ALRARADTAAAPAPLAPRPARCPTVLYRHPAHHGPWLTLDEPGEADAALVLCDPLGQPLR
GPERHFAAGAHMCAQARGLQAFVRVVPPPERPWADGGARAWAKFFRGCAWAQRLLGEPAV
MHLPYTDGDVPQLIALALRTLAQQGAALALSVRDLPGGAAFDAHAVTAAVRAGPGQSAAT
SPPPGDPPPPRRARRSQRHLDARGTPPPAPARDPPPPAPSPPAPPRAGDPVLPTSAGPAD
RARHAELEVAYEPSDPPTPTKADPDSDIVESYARAAGPVHLRVRDIMDPPPGCKVVVNAA
NEGLLAGSGVCGAIFANATAALAADCRRLAPCPTGEAVATPGHGCGYTHIIHAVAPRRPR
DPAALEEGEALLERAYRSIVALAAARRWACVACPLLGAGVYGWSAAESLRAALAATRAEP
AERVSLHICHPDRATLTHASVLVGAGLAARRVSPPPTEPLASCPAGDPGRPAQRSASPPA
TPLGDATAPEPRGCQGCELCRYTRVTNDRAYVNLWLERDRGATSWAMRIPEVVVYGPEHL
ATHFPLNHYSVLKPAEVRPPRGMCGSDMWRCRGWQGMPQVRCTPSNAHAALCRTGVPPRV
STRGGELDPNTCWFRAAANVAQAARACGAYTSAGCPKCAYGRALSEARTHEDFAALSQRW
SASHADASPDGTGDPLDPLMETVGCACSRVWVGSEHEAPPDHLLVSLHRAPNGPWGVVLE
VRARPEGGNPTGHFVCAVGGGPRRVSDRPHLWLAVPLSRGGGTCAATDEGLAQAYYDDLE
VRRLGDDAMARAALASVQRPRKGPYNIRVWNMAAGAGKTTRILAAFTREDLYVCPTNALL
HEIQAKLRARDIDIKNAATYERALTKPLAAYRRIYIDEAFTLGGEYCAFVASQTTAEVIC
VGDRDQCGPHYANNCRTPVPDRWPTERSRHTWRFPDCWAARLRAGLDYDIEGERTGTFAC
NLWDGRQVDLHLAFSRETVRRLHEAGIRAYTVREAQGMSVGTACIHVGRDGTDVALALVR
DLAIVSLTRASDALYLHELEDGSLRAAGLSAFLDAGALAELKEVPAGIDRVVAVEQAPPP
LPPADGIPEAQDVPPFCPRTLEELVFGRAGHPHYADLNRVTEGEREVRYMRISRHLLNKN
HTEMPGTERVLSAVCAVRRYRAGEDGSTLRTAVARQHPRPFRQIPPPRVTAGVAQEWRMT
YLRERIDLTDVYTQMGVAARELTDRYARRYPEIFAGMCTAQSLSVPAFLKATLKCVDAAL
GPRDTEDCHAAQGKAGLEIRAWAKEWVQVMSPHFRAIQKIIMRALRPQFLVAAGHTEPEV
DAWWQAHYTTNAIEVDFTEFDMNQTLATRDVELEISAALLGLPCAEDYRALRAGSYCTLR
ELGSTETGCERTSGEPATLLHNTTVAMCMAMRMVPKGVRWAGIFQGDDMVIFLPEGARSA
ALKWTPAEVGLFGFHIPVKHVSTPTPSFCGHVGTAAGLFHDVMHQAIKVLCRRFDPDVLE
EQQVALLDRLRGVYAALPDTVAANAAYYDYSAERVLAIVRELTAYARGRGLDHPATIGAL
EEIQTPYARANLHDAD
SEQ ID NO: 3476 (Colorado Tick Fever Virus VP12 (AAB02025))
GAFVLALLISLQSVYFKLYEFYKNNETARNTSVAGFLKRHEVAVNVIVEFSFDILFFLCG
LLGFELSPTARRLIFRRTASAEKADTVELEHVSSRRRNDSRDDSTVRNVSKTSPLASQRS
RDHFDGDPREPAPPAYSPADFYPPPASPHICETPLSTRVAPSAPSASLFTAGGIGLP
SEQ ID NO: 3478 (Human Foamy Virus Gag Protein (NP_044279))
MASGSNVEEYELDVEALVVILRDRNIPRNPLHGEVIGLRLTEGWWGQIERFQMVRLILQD
DDNEPLQRPRYEVIQRAVNPHTMFMISGPLAELQLAFQDLDLPEGPLRFGPLANGHYVQG
DPYSSSYRPVTMAETAQMTRDELEDVLNTQSEIEIQMINLLELYEVETRALRRQLAERSS
TGQGGISPGAPRSRPPVSSFSGLPSLPSIPGIHPRAPSPPRATSTPGNIPWSLGDDSPPS
SSFPGPSQPRVSFHPGNPFVEEEGHRPRSQSRERRREILPAPVPSAPPMIQYIPVPPPPP
IGTVIPIQHIRSVTGEPPRNPREIPIWLGRNAPAIDGVFPVTTPDLRCRIINAILGGNIG
LSLTPGDCLTWDSAVATLFIRTHGTFPMHQLGNVIKGIVDQEGVATAYTLGMMLSGQNYQ
LVSGIIRGYLPGQAVVTALQQRLDQEIDDQTRAETFIQHLNAVYEILGLNARGQSIRASV
TPQPRPSRGRGRGQNTSRPSQGPANSGRGRQRPASGQSNRGSSTQNQNQDNLNQGGYNLR
PRTYQPQRYGGGRGRRWNDNTNNQESRPSDQGSQTPRPNQAGSGVRGNQSQTPRPAAGRG
GRGNHNRNQRSSGAGDSRAVNTVTQSATSSTDESSSAVTAASGGDQRD
SEQ ID NO: 3479 (Hepatitis E Virus ORF3 (AAC35758))
MNNMSFAAPMGSRPCALGLFCCCSSCFCLCCPRHRPVSRLAAVVGGAAAVPAVVSGVTGL
ILSPSQSPIFIQPTPSPPMSPLRPGLDLVFANLPDHSAPLGVTRPSAPPLPHVVDLPQLG
PRR
SEQ ID NO: 3480 (Hepatitis G Virus Polyprotein Precursor
(AAB65834))
MAVLLLLLVVEAGAILAPATHVCRASGQYFLTNCCALENIGFCLEGGCLVPLGCTVCTDR
CWPLYQAGLAVRPGKSAAQLVGELGSLYGPLSVSAYVAGILGLGEVYSGVLTVGVALTRR
AYPVPNLTCSVECELKWESEFWRWTEQLASNYWILEYLWKVPFDFWRGVMSLTPLLVCVA
ALLLLEQRIVMVFLLVTMAGMSQGAPASVLGSRPFEAGLTWQSCSCEANGSRVPTGERVW
DRGNVTLLCDCPNGPWVWLPAVCQAIGWGDPITHWSHGQNQWPLSCPQFVYGAVSVTCVW
GSVSWFASTGGRDSKIDVWSLVPVGSASCTIAALGSSDRDTVVELSEWGIPCATCILDRR
PASCGTCVRDCWPETGSVRFPFHRCGAGPRLTKDLEAVPFVNRTTPFTIRGPLGNQGKGN
PVRSPLGFGSYTMTKIRDSLHLVKCPTPAIEPPTGTFGFFPGTPPLNNCMLLGTEVSEVL
GGAGLTGGFYEPLVRRCSELAGRRNPVCPGFAWLSSGRPDGFIHVQGHLQEVGAGNFIPP
PRWLLLDFVFVLLYLVKLAEARLVPLILLLLWWWVNQLAVLGLPTAHAAVAGEVFAGPAL
SWCLGLPFVSMILGLANLVLYFRWMGPQRLMFLVLWKLARGAFPLALLMGIPATRGRTSV
LGAEFCFDVTFEVDTSVLGWVVASVVAWAIALLSSMSAGGWRHKAVIYRTWCKGYQALRQ
RVVRSPLGEGRPTKPLTFAWCLASYIWPDAVMLVVVGLVLLFGLFDALDWALEELLVSRP
SLRRLARVVECCVMAGEKATTIRLVSKMCARGAYLFDHMGSLSRAVKERLPEWDAALEPL
SFTRTDCRIIRDAARTLSCGQCVMGLPVVARRGDEVLIGVFQDVNHLPPGFVPTAPVVIR
RCGKGFLGVTKAALTGRDPDLHPGNVMVLGTATSRSMGTCLNGLLFTTFHGASSRTIATP
VGALNPRWWSASDDVTVYPLPDGANSLTPCTCQAESCWVIRSDGALCHGLSKGDKVELDV
AMEVSDFRGSSGSPVLCDEGHAVGMLVSVLHSGGRVTAARFIRPWTQVPTDAKTTTEPPP
VPAKGVFKEAPLFMPTGAGKSTRVPLEYGNMGHKVLILNPSVATVRAMGPYMERLAGRHP
SIYCGHDTTAFTRITDSPLTYSTYGRFLANPRQMLRGVSVVICDECHSHDSTVLLGIGRV
RELARGCGVQLVLYATATPPGSPMVQHPSIIETKLDVGEIPFYGHGISLERMRTGRHLVF
CHSKAECERLAGQFSSRGVNAIAYYRGKDSSIIKDGDLVVCATDALSTGYTGNFDSVTDC
GLVVEEVVEVTLDPTITISLRTVPASAELSMQRRSRTGRGRSGRYYYAGVGKAPAGVVRS
GPVWSAVEAGMTWYGMEPDLTANLLRLYDDCPYTAAIAADIGEAAVFFAGLAPLRMHPDV
SWAKVRGVNWPLLVGVQRTMCRETLSPGPSDDPQWAGLKGPNPVPLLLRWGNDLPSKVAG
HHIVDDLVRRLGVAEGYVRCDAGPILMVGLAIAGGMIYASYTGSLVVVTDWDVKGGGNPL
YRNGDQATPQPVVQVPPVDHRPGGESAPSDAKTVTDAVAAIQVNCDWSVMTLSIGEVLAL
AQAKTAEAYTATAKWLAGCYTGTRAVPTVSIVDKLFAGGWAAVVGHCHSVIAAAVAAYGA
SRSPPLAAAASYLMGLGDGGNAQARLASALLLGAAGTALGTPVVGLTMAGAFMGGASVSP
SLVTVLLGAVGGWEGVVNAASLVFDFMAGKLSTDDLWYAIPVLTSPGAGLAGIALGLVLY
SANNSGTTTWLNRLLTTLPRSSCIPDSYFQQADYCDKVSAMLRRLSLTRTVVALVNREPR
VDEVQVGYVWDLWEWIMRQVRMVIARVRALCPVVSLPLWHCGEGWSGEWLLDGHVESRCL
CGCVITGDVFNGQLKDPVYSTKLCRHYWMGTVPVNMLGYGETSLLLASDTPKVVPFGTSG
WAEVVVTPTHVVIRRTSCYKLLRQQILSAAVAEPYYVDGIPVSWEADARAPAMVYGPGQS
ATIDGERYTLPHQLRMRNVAPSEVPSEVSIEIGTETEDSELTEADLPPAAAALQAIENAA
RILEPHIDVIMEDCSTPSLCGSSREMPVWGEDVPRTPSPALISVTESSSDEKTPSASSSQ
EDTPSSDSFEVIQESDTAESEDSVFNVALSVPKALFPQSDATRKLTVRMSCCVEKSVTRF
FSLGLTVADVASLCEMEIQNHTAYCDKVRTPLELQVGCLVGNELTFECDKCEARQETLAS
FSYIWSGVPLTRATPAKPPVVRPVGSLLVADTTKVYVTNPHNVGRRVDNVTFWRAPRVHD
KFLVDSIERARRAAQACLSMGYTYEEAIRTVRPHAAMGWGSKVSVKDLATPAGKMSVHDR
FQEIAEGTPVPFTLTVKKEVFFKDRKEEKAPRFIVFPPLNFRIAAKLILGDPARVPKAVL
GGAYAFQYTPNQRVKEMLKLWESKKTPCAICVDATCFDSSITEEDVALETELYALASDHP
EWVRALGKYYASGTMVTPEGVPVGERYCRSSGVLTTSASNCLTCYIKVKAACDRVGLKNV
SFLIAGDDCLIICERPMCDPSEALGRALASYGYACEPSYHASLDAAPFCSTWLAECNADG
KRHFFLTTDFRRPLARMSSEYSDPMASAIGYILLYPWHPITRWVIIPHVLTCAFRGGGTP
SDPVWCQVHGNYYKFPLDKLPNIIVALHGPAALRVTADTTKTKMEAGKVLSDLKLPGLAV
HRKKAGALRTRMLRSHDWAELARGLLWHPGLRLPPPEIAGIPGGFPLSPPYMGVVHQLDF
TAQRSRWWWLGFLTLLIVALFG
SEQ ID NO: 3481 (Human Herpesvirus 5 UL32 (AAG31644))
MSLQFIGLQRRDVVALVNFLRHLTQKPDVDLEAHPKILKKCGEKRLHRRTVLFNELMLWL
GYYRELRFHNPDLSSVLEEFEVRCAAVARRGYTYPFGDRGKARDHLAVLDRTEFDTDVRH
DAEIVERALVSAVILAKMSVRETLVTAIGQTEPIAFVHLKDTEVQRIEENLEGVRRNMFC
VKPLDLNLDRHANTALVNAVNKLVYTGRLIMNVRRSWEELERKCLARIQERCKLLVKELR
MCLSFDSNYCRNILKHAVENGDSADTLLELLIEDFDIYVDSFPQSAHTFLGARPPSLEFD
DDANLLSLGGGSAFSSVPKKHVPTQPLDGWSWIASPWKGHKPFRFEAHGSLAPAADAHAA
RSAAVGYYDEEEKRRERQKRVDDEVVQREKQQLKAWEERQQNLQQRQQQPPPPTRKPGAS
RRLFGSSADEDDDDDDDEKNIFTPIKKPGTSGKGAASGNGVSSIFSGMLSSGSQKPTSGP
LNIPQQQQRHAAFSLVSPQVTKASPGRVRRDSAWDVRPLTETRGDLFSGDEDSDSSDGYP
PNRQDPRFTDTPVDITDTETSAKPPVTTAYKFEQPTLTFGAGVNVPAGAGAAILTPTPVN
PSTAPAPAPTPTFAGTQTPVNGNSPWAPTAPLPGDMNPANWPRERAWALKNPHLAYNPFR
MPTTSTTSQNNVSTTPRRPSTPRAAVTQTASQNAADEVWALRDQTAESPVEDSEEEDDDS
SDTGSVVSLGHTTPSSDYNDVISPPSQTPEQSTPSRIRKAKLSSPMTTTSTSQKPVLGKR
VATPHASARAQTVTSTPVQGRVEKQVSGTPSTVPATLLQPQPASSKTTSSRNVTSGARTS
SASARQPSASASVLSPTEDDVVSPVTSPLSMLSSASPSPAKSAPPSPVKGRGSRVGVPSL
KPTLGGKAVVGRPPSVPVSGSAPGRLSGTSRAASTTPTYPAVTTVYPPSSTAKSSVSNAP
PVASPSILKPGASAALQSRRSTGTAAVGSPVKSTTGMKTVAFDLSSPQKSGTGPQPGSAG
MGGAKTPSDAVQNILQKIEKIKNTEE
SEQ ID NO: 3482 (Human Parechovirus 2 Polyprotein
(NP_041865))
METIKSIADMATGVTKTIDATINSVNEIITNTDNASGGDILTKVADDASNILGPNCYATT
SEPENKDVVQATTTVNTTNLTQHPSAPTLPFTPDFSNVDTFHSMAYDTTTGSKNPNKLVR
LTTHAWASTLQRGHQIDHVNLPVDFWDEQRKPAYGHAKYFAAVRCGFHFQVQVNVNQGTA
GSALVVYEPKPVVDYDKDLEFGAFTNLPHVLMNLAETTQADLCIPYVADTNYVKTDSSDL
GQLKVYVWTPLSIPSGSSNQVDVTILGSLLQLDFQNPRVYGQNVDIYDTAPSKPIPLRKT
KYLTMSTKYKWTRNKVDIAEGPGSMNMANVLSTTAAQSVALVGERAFYDPRTAGSKSRFD
DLVKISQLFSVMADSTTPSANHGIDQKGYFKWSANSDPQAIVHRNLVHLNLFPNLKVFEN
SYSYFRGSLIIRLSVYASTFNRGRLNGFFPNSSTDETSEIDNAIYTICDIGSDNSFEITI
PYSFSTWMRKTHGKPIGLFQIEVLNRLTYNYSSPNEVYCIVQGKMGQDAKFFCPTGSLVT
FQNSWGSQMDLTDPLCIEDSVEDCKQTITPTELGLTSAQDDGPLGNDKPNYFLNFKSMNV
DIFTVSHTKVDNIFGRAWFAHVHDFTNDGLWRQGLEFPKEGHGALSLLFAYFTGELNIHV
LFLSDRGFLRVGHTYDTETNRTNFLSSSGIITVPAGEQMTLSVPSYSNKPLRTVRSSNAL
GYLLCKPLLTGTSSGRIEIFLSLRCPNFFFPLPAPKPATRKYRGDLATWSDQSPYGRQGK
KQLMKLAYLDRGFYKHYGIVVGDDVYQLDSDDIFKTALTGKAKFTKTRLTPDWVVEEECE
LDYFRIKYLESSVNSEHIFSVDNNCETIAKDIFGSHSLSQHQQIGLIGTILLTAGLMSTI
KTPVNPTTIKEFFNHAIEGDEQGLSLLVQKCTTFFSSAATELLDNDLVKFIIKILVRILC
YMVLYCHKPNILTTACLSTLLVMDVTSSSVLSPSCKALMQCLMDGDVKKLAEVVAESMSN
TDDDEIKEQICDTVKYTKQILSNQGPFKGFNEISTAFRHIDWWIQTLLKIKDMVLSVFKP
SVEKRAVEWLERNKEHVCSILDYASDIIVKSKDQTKMKTQEFYQRYNDCLSKFKPIMAMC
FRSCHNSISNTVYRLFQELARIPNRMATQNDLIRVEPIGIWIQGEPGQGKSFLTHTLSKQ
LQKTCGLQGIYTNPTASEFMDGYDNQDIHLIDDLGQTRKERDIEMLCNCISSDPDIVPMA
HLEEKGKFYTSKLVIATTNKPDFSSTVLLDSGALRRRFPYIMHIRAAKHYSKSGKLNVSQ
AMPHMSTGECWEVSKNGRDWETLKLKELIDKITVDYKERIANYNTWKKQLEDQTLDDLDD
AVSYIKHNYPDAIPYIDEYLNIEMSTLIEQMEAFIEPKPSVFKCFASRVGDKIKEASREV
VKWFSDKLKSMLNFVERNKAWLTVVSAVTSAIGILLLVTKIFKKEESKDERAYNPTLPVA
KPKGTFPVSQREFKNEAPYDGQLEHIISQMAYITGSTTGHITHCAGYQHDEIILHGHSIK
YLEQEEELTLHYKNKVFPIEQPSVTQVTLGGKPMDLAIVKCKLPFRFKKNSKYYTNKIGT
ESMLIWMTEQGIITKEVQRVHHSGGIKTREGTESTKTISYTVKSCKGMCGGLLISKVEGN
FKILGMHIAGNGEMGVAIPFNFLKNDMSDQGIVTEVTPIQPMYINTKSQIHKSPVYGAVE
VKMGPAVLSKSDTRLEEPVDCLVKKSASKYRVNKFQVNNELWQGVKACVKSKFREIFGVN
GIVDMKTAILGTSHVNSMDLSTSAGYSFVKSGYKKKDLICLEPFSVSPMLEKLVQEKFHN
LLKGNQITTIFNTCLKDELRKLDKIATGKTRCIEACEIDYCIVYRMIMMEIYDKIYQTPC
YYSGLAVGINPYRDWHFMINALNDYNYEMDYSQYDGSLSSMLLWEAVQVLAYCHDSPDLV
MQLHKPVIDSDHVVFNERWLIHGGMPSGSPCTTVLNSLCNLMMCIYTTNLISPGIDCLPI
VYGDDVILSLDKEIEPERLQSIMAESFGAEVTGSRKDEPPSLKPRMEVEFLKRKPGYFPE
STFIVGKLDTENMIQHLMWMKNFSTFKQQLQSYLMELCLHGKDTYQHYVKILNPYLKEWN
IPVDDYEVVIGKLVPMVFD
SEQ ID NO: 3483 (Semliki Forest Virus Polyprotein(CAA76683))
MAAKVHVDIEADSPFIKSLQKAFPSFEVESLQVTPNDHANARAFSHLATKLIEQETDKDT
LILDIGSAPSRRMMSTHKYHCVCPMRSAEDPERLVCYAKKLAAASGKVLDREIAGKITDL
QTVMATPDAESPTFCLHTDVTCRTAAEVAVYQDVYAVHAPTSLYHQAMKGVRTAYWIGFD
TTPFMFDALAGAYPTYATNWADEQVLQARNIGLCAASLTEGRLGKLSILRKKQLKPSDTV
MFSVGSTLYTESRKLLRSWHLPSVFHLKGKQSFTCRCDTIVSCEGYVVKKITMCPGLYGK
TVGYAVTHHAEGFLVCKTTDTVKGERVSFPVCTYVPSTICDQMTGILATDVTPEDAQKLL
VGLNQRIVVNGRTQRNTNTMKNYLLPVVAVAFSKWAREYKADLDDEKPLGVRERSLTCCC
LWAFKTKKMHTMYKKPDTQTIVKVPSEFNSFVIPSLWSTGLAIPVRSRIKMLLAKKTKRE
LIPALDASSARDAEQEEKERLEAELTREALPPLVPIAPAETGVVDVDVEELEYRAGAGVV
ETPRSALKVTAQPNDVLLGNYVVLSPQTVLKSSKLAPVHPLAEQVKIITHNGRAGRYQVD
GYDGRVLLPCGSAIPVPEFQALSESATMVYNEREFVNRKLYHIAVHGPSLNTDEENYEKV
RAERTDAEYVFDVDKKCCIKREEASGLVLVGELTNPPFHEFAYEGLKIRPSAPYKTTVVG
VFGVPGSGKSAIIKSLVTKHDLVTSGKKENCQEIVNDVKKHRGLDIQAKTVDSILLNGCR
RAVDILYVDEAFACHSGTLLALIALVKPRSKVVLCGDPKQCGFFNMMQLKVNFNHNICTE
VCHKSISRRCTRPVTAIVSTLHYGGKMRTTNPCNKPIIIDTTGQTKPKPGDIVLTCFRGW
VKQLQLDYRGHEVMTAAASQGLTRKGVYAVRQKVNENPLYAPASEHVNVLLTRTEDRLVW
KTLAGDPWIKVLSNIPQGNFTATLEEWQEEHDKIMKVIEGPAAPVDAFQNKANVCWAKSL
VPVLDTAGIRLTAEEWSTIITAFKEDRAYSPEVALNEICTKYYGVDLDSGLFSAPKVSLY
YENNHWDNRPGGRMYGFNAATAARLEARHTFLKGQWHTGKQAVIAERKIQPLSVLDNVIP
INRRLPHALVAEYKTVKGSRVEWLVNKVRGYHVLLVSEYNLALPRRRVTWLSPLNVTGAD
RCYDLSLGLPADAGRYDLVFVNIHTEFRIHHYQQCVDHAMKLQMLGGDALRLLKPGGNLL
MRAYGYADKISEAVVSSLSRKFSSARVLRPDCVTSNTEVFLLFSNFDNGKRPSTLHQMNT
KLSAVYAGEAMHTAGC
SEQ ID NO: 3484 (HIV GAG protein (AF324493))
MGARASVLSGGELDKWEKIRLRPGGKKQYKLKHIVWASRELERFAVNPGLLETSEGCRQILGQLQ
PSLQTGSEELRSLYNTIAVLYCVHQRIDVKDTKEALDKIEEEQNKSKKKAQQAAA
DTGNNSQVSQNYPIVQNLQGQMVHQAISPRTLNAWVKVVEEKAFSPEVIPMFSALSEGAT
PQDLNTMLNTVGGHQAAMQMLKETINEEAAEWDRLHPVHAGPIAPGQMREPRGSDIAGTT
STLQEQIGWMTHNPPIPVGEIYKRWIILGLNKIVRMYSPTSILDIRQGPKEPFRDYVDRF
YKTLRAEQASQEVKNWMTETLLVQNANPDCKTILKALGPGATLEEMMTACQGVGGPGHKA
RVLAEAMSQVTNPATIMIQKGNFRNQRKTVKCFNCGKEGHIAKNCRAPRKKGCWKCGKEG
HQMKDCTERQANFLGKIWPSHKGRPGNFLQSRPEPTAPPEESFRFGEETTTPSQKQEPID
KELYPLASLRSLFGSDPSSQ
SEQ ID NO: 3485
CTTCAGAGCAGACCAGAGCCAACAGCCCCACCAGAAGAGAGCTTCAGGTTTG
GGGAAGAGACAACAACTCCCTCTCAGAAGCAGGAGCCGATAGACAAGGAAC
TGTATCCTTTAGCTTCCCTCAGATCACTCTTTGGCAGCGACCCCTCGTCACAAT

What is claimed is:

1. A composition comprising a peptide associated with a transporter that is capable of increasing the uptake of said peptide by a mammalian cell,
wherein said peptide includes an amino acid sequence motif PX₁X₂P and is capable of binding the UEV domain of Tsg101,
wherein X₁ and X₂ are amino acids, and X₂ is not arginine, and
wherein said peptide does not contain a contiguous amino acid sequence of an HIV GAG protein that is sufficient to impart an ability to bind the UEV domain of Tsg101 on said peptide.

2. The composition according to claim 1, wherein said peptide does not contain a contiguous amino acid sequence of 10 or more residues of an HIV GAG protein that encompasses the late domain motif of said GAG protein.

3. The composition of claim 1, wherein said peptide is covalently linked to said transporter.

4. The composition of claim 1, wherein the transporter is capable of increasing the uptake of said peptide by said mammalian cell by at least 100%.

5. The composition according to claim 1, wherein said transporter is capable of increasing the uptake of said peptide by said mammalian cell by at least 300%.

6. The composition according to claim 1, wherein X₁ is threonine (T) or serine (S), and X₂ is alanine (A).

7. The composition according to claim 1, wherein said peptide consists of from 8 to about 50 amino acids.

8. An isolated nucleic acid encoding a hybrid polypeptide hybrid polypeptide, said hybrid polypeptide consists of a peptide covalently linked to a peptidic transporter that is capable of increasing the uptake of said peptide by a mammalian cell by at least 100%,
wherein said peptide consists of from about 8 to about 100 amino acid residues, comprises an amino acid sequence motif PX₁X₂P, and is capable of binding the UEV domain of Tsg101, wherein X₁ and X₂ are amino acids, and X₂ is not R, and
wherein said peptide does not contain a contiguous amino acid sequence of an HIV GAG protein that is sufficient to impart an ability to bind the UEV domain of Tsg101 on said peptide.

9. A host cell comprising the isolated nucleic acid according to claim 8.

10. An isolated peptide consisting of a contiguous amino acid sequence of from 8 to about 30 amino acid residues of a viral protein selected from the group consisting of HBV PreS1/PreS2/S envelope protein, HSV1 RL2 protein, HSV2 virion glycoprotein K, HSV2 Strain 333 glycoprotein I, EBV nuclear protein EBNA2, Influenza A virus hemagglutinin, HPV L1 proteins, HPV L2 proteins, HPV late proteins, HTLV-2 GAG protein, West Nile virus polyprotein precursor, Measles virus matrix protein, Rubella virus non-structural protein, Colorado tick fever virus VP12, foot-and-mouth disease virus VP1 capsid protein, human foamy virus GAG protein, hepatitis G virus polyprotein precursor, human parechovirus 2 polyprotein, and Semliki forest virus polyprotein,
wherein said contiguous amino acid sequence encompasses the P(T/S)AP motif of said viral protein,
wherein said peptide is capable of binding the UEV domain of Tsg101,
and wherein said peptide does not contain a contiguous amino acid sequence of an HIV GAG protein or Ebola virus Matrix (EbVp40) protein that is sufficient to impart an ability to bind the UEV domain of Tsg101 on said peptide.

11. An isolated nucleic acid encoding the isolated peptide according to claim 10.

12. A method for inhibiting HIV budding from cells, comprising administering to cells the composition of claim 1.

13. A method for inhibiting HIV budding from cells, comprising administering to cells the peptide of claim 10.

14. A method for inhibiting HIV budding from cells, comprising introducing into cells infected with HIV the peptide consisting of from 8 to about 30 amino acid residues and having an amino acid sequence motif PX₁X₂P, wherein X₁ and X₂ are amino acids, and X₂ is not R,
wherein said peptide is capable of binding the UEV domain of Tsg101, and
wherein said peptide does not contain a contiguous amino acid sequence of an HIV GAG protein that is sufficient to impart an ability to bind the UEV domain of Tsg101 on said peptide.
